(19) United States
(12) Patent Application Publication
(10) Pub. No.: US 2026/0218231 A1
(43) Pub. Date: Jul. 30, 2026

(54) PLAKOPHILIN-2 (PKP2) GENE THERAPY USING AAV VECTOR

(71) Applicant: SPACECRAFT SEVEN, LLC, Cranbury, NJ (US)

(72) Inventors: Christopher Dean HERZOG, Cranbury, NJ (US); Chester Bittencort SACRAMENTO, Cranbury, NJ (US); Raj PRABHAKAR, Cranbury, NJ (US); David RICKS, Cranbury, NJ (US)

(21) Appl. No.: 19/145,801

(22) PCT Filed: Jan. 5, 2024
(86) PCT No.: PCT/US2024/010587

§ 371 Date: Jul. 3, 2025

Related U.S. Application Data
(60) Provisional application No. 63/437,616, filed on Jan. 6, 2023.
(60) Provisional application No. 63/460,557, filed on Apr. 19, 2023.
(60) Provisional application No. 63/525,426, filed on Jul. 7, 2023.

(51) Int. Cl. C12N 15/86; A61K 38/17; A61K 48/00; A61P 9/06; C07K 14/47

(52) U.S. Cl. CPC C12N 15/86; A61K 38/1709; A61K 48/0058; A61K 48/0075; A61P 9/06; C07K 14/47; C12N 2750/14143; C12N 2830/008; C12N 2830/48; C12N 2830/50

(57) ABSTRACT

Provided herein is a gene therapy for PKP2 (Plakophilin-2), e.g., using an adeno-associated virus (AAV) vector. The promoter of the vector may be a MHCK7 promoter or a cardiac troponin T (HTNNT2) promoter. The capsid may be an AAV9 or AAVrh. 74 or comprise an AAV9 or AAVrh74 capsid protein or a functional variant thereof. Other promoters or capsids may be used. Further provided are methods of treatment, such as by intravenous, intracoronary, intracarotid or intracardiac administration of the rAAV vector, and other compositions and methods.

### CROSS-REFERENCE TO RELATED APPLICATIONS

This application is claims benefit of priority to U.S. Provisional Patent Application No. 63/437,616, filed on Jan. 6, 2023, U.S. Provisional Patent Application No. 63/460,557, filed on Apr. 19, 2023, and U.S. Provisional Patent Application No. 63/525,426, filed on Jul. 7, 2023, which are incorporated herein by reference in their entireties.

### REFERENCE TO AN ELECTRONIC SEQUENCE LISTING

The contents of the electronic sequence listing (ROPA_029_01WO_SeqList ST26.xml; Size: 232,839 bytes; and Date of Creation: Jan. 3, 2024) are herein incorporated by reference in its entirety.

### BACKGROUND

Arrhythmogenic cardiomyopathy (ACM) is a form of adult-onset heart disease, which impacts an estimated 1 in 1,000 to 1 in 1,250 people. Because this cardiomyopathy often manifests in the right ventricular free wall, ACM has also been referred to as arrhythmogenic right ventricular cardiomyopathy (ARVC) or arrhythmogenic right ventricular dysplasia (ARVD). However, since left dominant and biventricular forms have also been observed, this has led more recently to the use of the term “ACM” to encompass all forms of the disease. It manifests as breakdown of the muscular wall of the heart (the myocardium) over time, which leads to increased risk of abnormal heartbeat (arrhythmia) and an increased risk of sudden death when an affected individual exercises strenuously. Individuals may also experience a sensation of fluttering or pounding in the chest (palpitations), light-headedness, fainting (syncope), shortness of breath, and abnormal swelling in the legs or abdomen. Over time, ACM can lead to heart failure.

At least 13 genes are implicated in ACM, many of which are involved in the biogenesis of desmosomes, which are intracellular junctions that provide strong adhesion between cells. When desmosomes fail to form properly, myocardial cells may detach from one another and die. The right ventricle in particular may develop weakness, while fatty deposits and scar tissue may replace the damaged myocardium, leading to distension of the right ventricle. These alterations ultimately prevent effective heart pumping and disrupt the electrical signals that control the heartbeat, leading to arrhythmia. Autosomal dominant plakophilin-2 (PKP2) cardiomyopathy (also known as ARVD or ARVC) is an inherited form of ACM in which mutations affecting PKP2 are detected.

There remains, therefore, an unmet need in the art for treatments for PKP2-related diseases and disorders, including ACM. The compositions and methods disclosed herein address this need.

### SUMMARY

The present disclosure relates generally to gene therapy for a disease or disorder, e.g., a cardiac disease or disorder, using a vector expressing PKP2 or a functional variant thereof.

In one aspect, the disclosure provides a polynucleotide, comprising an expression cassette and optionally flanking adeno-associated virus (AAV) inverted terminal repeats (ITRs), wherein the polynucleotide comprises a polynucleotide sequence encoding a Plakophilin-2 (PKP2) or a functional variant thereof, operatively linked to a promoter.

In some embodiments, the promoter is a cardiac-specific promoter.

In some embodiments, the promoter is a muscle-specific promoter.

In some embodiments, the promoter is a cardiomyocyte-specific promoter.

In some embodiments, the promoter is a Myosin Heavy-chain Creatine Kinase 7 (MHCK7) promoter.

In some embodiments, the MHCK7 promoter shares at least 75%, at least 80%, at least 85%, at least 90%, at least 95%, at least 96%, at least 97%, at least 98%, at least 99% or 100% identity with SEQ ID NO: 31.

In some embodiments, the promoter is a cardiac troponin T (hTNNT2) promoter.

In some embodiments, the hTNTNT2 promoter shares at least 75%, at least 80%, at least 85%, at least 90%, at least 95%, at least 96%, at least 97%, at least 98%, at least 99% or 100% identity with SEQ TD NO: 32.

In some embodiments, the expression cassette comprises exon 1 of the cardiac troponin T (hTNNT2) gene, wherein optionally the hTNNT2 promoter and exon 1 together share at least 75%, at least 80%, at least 85%, at least 90%, at least 95%, at least 96%, at least 97%, at least 98%, at least 99% or 100% identity with SEQ ID NO: 32.

In some embodiments, the promoter is a ubiquitous promoter, optionally a CMV promoter or a CAG promoter.

In some embodiments, the expression cassette comprises a polyA signal.

In some embodiments, the polyA signal is a human growth hormone (hGH) polyA.

In some embodiments, the expression cassette comprises a Woodchuck Hepatitis Virus Posttranscriptional Regulatory Element (WPRE), optionally a WPRE(x) containing mutations which abrogate potential synthesis of the woodchuck hepatitis virus X protein.

In some embodiments, the Piakophilin-2 (PKP2) or functional variant thereof is a PKP2.

In some embodiments, the PKP2 is a functional PKP2.

In some embodiments, the PKP2 is a human PKP2.

In some embodiments, the PKP2 is PKP2 isoform A.

In some embodiments, the PKP2 isoform A shares at least 75%, at least 80%, at least 85%, at least 90%, at least 95%, at least 96%, at least 97%, at least 98%, at least 99% or 100% identity with SEQ ID NO: 1.

In some embodiments, the PKP2 is PKP2 isoform B.

In some embodiments, the PKP2 shares at least 75%, at least 80%, at least 85%, at least 90%, at least 95%, at least 96%, at least 97%, at least 98%, at least 99% or 100% identity with SEQ ID NO: 2.

In some embodiments, the polynucleotide sequence encoding PKP2 is a human PKP2 polynucleotide.

In some embodiments, the polynucleotide sequence encoding PKP2 shares at least 75%, at least 80%, at least 85%, at least 90%, at least 95%, at least 96%, at least 97%, at least 98%, at least 99% or 100% identity with SEQ ID NO: 3.

In some embodiments, the polynucleotide sequence encoding PKP2 shares at least 75%, at least 80%, at least 85%, at least 90%, at least 95%, at least 96%, at least 97%, at least 98%, at least 99% or 100% identity with SEQ ID NO: 4.

In some embodiments, the polynucleotide comprises at least about 4.0 kb, at least about 4.1 kb, at least about 4.2 kb, at least about 4.3 kb, at least about 4.4 kb, or at least about 4.5 kb.

In some embodiments, the polynucleotide comprises at most about 4.1 kb, at most about 4.2 kb, at most about 4.3 kb, at most about 4.4 kb, at most about 4.5 kb, or at most about 4.6 kb.

In some embodiments, the polynucleotide comprises 4.0 kb to 4.6 kb, 4,0 kb to 4.5 kb, or 4.0 kb to 4.4 kb or wherein the polynucleotide comprises 4.0 kb to 4.3 kb, 4.0 kb to 4.2 kb, or 4.0 kb to 4.1 kb.

In some embodiments, the PKP2 or functional variant thereof comprises at least 800 or at least 830 amino acids.

In some embodiments, the polynucleotide shares at least 75%, at least 80%, at least 85%, at least 90%, at least 95%, at least 96%, at least 97%, at least 98%, at least 99% or 100% identity with any one of SEQ ID NOs: 8-15, 89-96, and 97-102.

In some embodiments, the expression cassette is flanked by 5′ and 3′ inverted terminal repeats (ITRs)

In some embodiments, the ITRs are A AV2 ITRs and/or the ITRs share at least 75%, at least 80%, at least 85%, at least 90%, at least 95%, at least 96%, at least 97%, at least 98%, at least 99% or 100% identity with any one of SEQ ID NO: 20-26.

In another aspect, the disclosure provides a gene therapy vector, comprising the polynucleotide of any one of the preceding embodiments.

In some embodiments, the gene therapy vector is a recombinant adeno-associated virus (rAAV) vector.

In some embodiments, the rAAV vector is an AAV9 or a functional variant thereof.

In some embodiments, the rAAV vector comprises a capsid protein that shares at least or about 90%, at least or about 91%, at least or about 92%, at least or about 93%, at least or about 94%, at least or about 95%, at least or about 96%, at least or about 97%, at least or about 98%, at least or about 99%, or 100% identity to SEQ ID NO: 77.

In some embodiments, the rAAV vector is an AAVrh. 10 or a functional variant thereof.

In some embodiments, the rAAV vector comprises a capsid protein that shares at least or about 90%, at least or about 91%, at least or about 92%, at least or about 93%, at least or about 94%, at least or about 95%, at least or about 96%, at least or about 97%, at least or about 98%, at least or about 99%, or 100% identity to SEQ ID NO: 79.

In some embodiments, the rAAV vector is an A AV6 or a functional variant thereof.

In some embodiments, the rAAV vector comprises a capsid protein that shares at least or about 90%, at least or about 91%, at least or about 92%, at least or about 93%, at least or about 94%, at least or about 95%, at least or about 96%, at least or about 97%, at least or about 98%, at least or about 99%, or 100% identity to SEQ ID NO: 78.

In some embodiments, the rAAV vector is an AAVrh 74 or a functional variant thereof.

In some embodiments, the rAAV vector comprises a capsid protein that shares at least or about 90%, at least or about 91%, at least or about 92%, at least or about 93%, at least or about 94%, at least or about 95%, at least or about 96%, at least or about 97%, at least or about 98%, at least or about 99%, or 100% identity to SEQ ID NOs: 81-83.

In another aspect, the disclosure provides a method of treating and/or preventing a disease or disorder in a subject in need thereof, comprising administering the vector of any one of the preceding embodiments to the subject.

In some embodiments, the disease or disorder is a cardiac disorder.

In some embodiments, the disease or disorder is cardiomyopathy.

In some embodiments, the cardiomyopathy is arrhythmogenic cardiomyopathy (ACM).

In some embodiments, the cardiomyopathy is arrhythmogenic right ventricular cardiomyopathy (ARVC) or arrhythmogenic right ventricular dysplasia (ARVD).

In some embodiments, the cardiomyopathy is hypertrophic cardiomyopathy or dilated cardiomyopathy,

In some embodiments, the disease or disorder is characterized by fibrofatty infiltration of myocardium.

In some embodiments, the disease or disorder is heart failure.

In some embodiments, the subject is a mammal.

In some embodiments, the subject is a primate.

In some embodiments, the subject is a human.

In some embodiments, the subject has a mutation in a PKP2 gene.

In some embodiments, the mutation is a PKP2 gene “stop-gain” variant mutation.

In some embodiments, the subject has an implantable cardioverter-defibrillator (ICD).

In some embodiments, the vector is administered by intravenous injection, intracardiac injection, intracardiac infusion, and/or cardiac catheterization.

In some embodiments, the administration increases PKP2 expression by at least about 5%.

In some embodiments, the administration increases PKP2 expression by at least about 30%.

In some embodiments, the administration increases PKP2 expression by at least about 70%.

In some embodiments, the administration increases PKP2 expression by about 5% to about 10%.

In some embodiments, the administration increases PKP2 expression by about 30% to about 50%.

In some embodiments, the administration increases PKP2 expression by about 50% to about 70%.

In some embodiments, the administration increases PKP2 expression by about 70% to about 100%,

In particular embodiments, the increased PKP2 expression is PKP2 expression in the heart or cardiac tissue in a subject to whom the vector is administered. In particular embodiments, the increased PKP2 expression is PKP2 expression in the left ventricle. In particular embodiments, the increased PKP2 expression is PKP2 expression in the right ventricle.

In some embodiments, the method treats and/or prevents the disease or disorder.

In some embodiments, the method comprises administering an effective amount of the vector.

In some embodiments, the disease or disorder is related to or caused by loss of function in PKP2 in the subject.

In some embodiments, the disease or disorder is related to or caused by gain of function in PKP2 in the subject.

In some embodiments, the subject has a mutation that causes an amino acid substitution selected from Arg490Trp, Asp26Asn, Thr50_Val5ISerfsX60, Arg79X, Tyr86X, GIn133X, Val406SerfsX3, Tvr616X, Trp676X, Cvs796Arg, Cys796E, Tvr807X, Glu62Lvs, S688P, Trp848X, Y86X, V406X, Y616X, W848X, and Y807X, relative to a human PKP2 gene encoding a human PKP2 having the sequence of SEQ ID NO: 2.

In some embodiments, the method comprises administering a pharmaceutical composition comprising an effective amount of the vector.

In some embodiments, the method comprises administering between about 1×10¹¹ vector genomes/kg and about 1×10¹³vector genomes/kg of the vector to the subject, administering between about 1×10¹² vector genomes/kg and about 1×10¹⁴ vector genomes/kg of the vector to the subject, or administering between about 1×10¹³ vector genomes/kg and about 1×10¹⁵ vector genomes/kg of the vector to the subject. In some embodiments, the vector is an AAV9, optionally comprising a sequence having at least 90% or at least 95% identity to any one of SEQ ID NOs: 12, 8, 13, 9, 97, 100, 89, 93, 90, or 94, and the method comprises administering between about 1×10¹³ and about 1×10¹⁴ genomes of the vector/kg to the subject, e.g., about 1×10¹⁵. about 2×10¹³, about 3×10¹³, about 4×10¹³ about 5×10¹³, about 6×10¹³, about 7×10¹³, about 8×10¹³, about 9×10¹³, or about 1×10¹⁴ vg/kg. In some embodiments, the vector is an AAV.rh 74, optionally comprising a sequence having at least 90% or at least 95% identity to any one of SEQ ID NOs: 12, 8, 13, 9, 97 100, 89, 93, 90, or 94, and the method comprises administering between about 5×10¹³ and about 5×10¹⁴ genomes of the vector/kg to the subject, e.g., about 5×10¹³, about 6×10¹³, about 7×10¹³, about 8×10¹³, about 9×10¹³, about 1×10¹⁴ vg/kg, about 2×10¹⁴ vg/kg, about 3×10¹⁴ vg/kg, about 4×10¹⁴ vg/kg, or about 5×10¹⁴ vg/kg. In some embodiments, the vector is administered intravenously or systemically or locally to the heart.

In another aspect, the disclosure provides a pharmaceutical composition comprising the vector of any one of the preceding embodiments,

In another aspect, the disclosure provides a kit comprising the vector of any one of the preceding embodiments or the pharmaceutical composition of the preceding embodiment and optionally instructions for use.

In another aspect, the disclosure provides a use of the vector of any one of the preceding embodiments in treating a disease or disorder, optionally according to the method of any one of the preceding embodiments.

In another aspect, the disclosure provides a vector according to any one of the preceding embodiments for use in treating a disease or disorder, optionally according to the method of any one of the preceding embodiments.

In another aspect, the disclosure provides a polynucleotide, comprising a polynucleotide sequence that shares at least 90%, 91%, 92%, 93%, 94%, 95%, 96%, 97%, 98%, 99%, or 100% identity to any one of SEQ ID NOs: 12-15, 89-92, and 97-99, or to any one of SEQ ID NOs: 8-11, 93-96, and 100-102.

In some embodiments, the promoter is a MHCK7 promoter.

In some embodiments, the MHCK7 promoter shares at least 75%, 80%, 85%, 90%, 95%, 96%, 97%, 98%, 99% or 100% identity with SEQ ID NO: 31.

In some embodiments, the PKP2 is a human PKP2.

In some embodiments, the PKP2 is PKP2 isoform A.

In some embodiments, the PKP2 isoform A shares at least 80%, 90%, 95%, 99% or 100% identity with SEQ ID NO: 1.

In another aspect, the disclosure provides a gene therapy vector, comprising the polynucleotide of any one of the preceding embodiments.

In some embodiments, the gene therapy vector is a recombinant adeno-associated virus (rAAV) vector.

In some embodiments, the rAAV vector is an AAV9 vector.

In some embodiments, the rAAV vector is an AAVrh.74 vector.

In another aspect, the disclosure provides a method of treating and/or preventing a cardiac disorder in a subject identified as having a mutation in the PKP2 gene, comprising administering the vector ofany one of the preceding embodiments to the subject.

In some embodiments, the cardiac disorder is cardiomyopathy, optionally arrhythmogenic cardiomyopathy (ACM), hypertrophic cardiomyopathy, or dilated cardiomyopathy,

In some embodiments, the cardiac disorder is heart failure.

In some embodiments, the subject is a mammal.

In some embodiments, the vector is administered by intravenous injection, intracardiac injection, intracardiac infusion, and/or cardiac catheterization.

In some embodiments, the method prevents or reduces a decrease in left ventricle ejection fraction percentage (LVEF %), optionally by at least or about 50%, at least or about 60%, at least or about 70%, at least or about 80%, at least or about 90%, or at least or about 100% compared to the decrease observed in an untreated subject identified as having a mutation in the PKP2 gene.

In some embodiments, the method prevents or reduces a decrease in left ventricle fractional shortening percentage (FS %), optionally by at least or about 50%, at least or about 60%, at least or about 70%, at least or about 80%, at least or about 90%, or at least or about 100% compared to the decrease observed in an untreated subject identified as having a mutation in the PKP2 gene.

In some embodiments, the method prevents or reduces an increase in right ventricle area in millimeters squared RV Area (mm2), optionally by at least or about 50%, at least or about 60%, at least or about 70%, at least or about 80%, at least or about 90%, or at least or about 100% compared to the increase observed in an untreated subject identified as having a mutation in the PKP2 gene.

In some embodiments, the method prevents or reduces a decrease in right ventricle velocity time integral in millimeters per second RV % VTI (mm/sec), optionally by at least or about 50%, at least or about 60%, at least or about 70%, at least or about 80%, at least or about 90%, or at least or about 100% compared to the decrease observed in an untreated subject identified as having a mutation in the PKP2 gene.

In some embodiments, the method prevents or reduces an increase in left ventricle or right ventricle fibrosis, optionally by at least or about 50%, at least or about 60%, at least or about 70%, at least or about 80%, at least or about 90%, or at least or about 100% compared to the increase observed in an untreated subject identified as having a mutation in the PKP2 gene.

Various other aspects and embodiments are disclosed in the detailed description that follows. The disclosure is limited solely by the appended claims.

### BRIEF DESCRIPTION OF FIGURES

FIGS. 1A-1B show diagrams illustrating a non-limiting example of a vector genome. FIG. 1A shows a version without SV40 intron. The full polynucleotide sequence of one embodiment of the vector genome is SEQ ID NO: 12, and the associated expression cassette is SEQ ID NO: 8. FIG. 1 shows a version with SV40 intron. The full polynucleotide sequence of one embodiment of this vector genome is SEQ ID NO: 89, and the associated expression cassette is SEQ ID NO: 93. The MHCK7 promoter as described herein is labelled “Enhancer/MHCK7” in the diagrams.

FIG. 2 shows a diagram illustrating a non-limiting example of a vector genome. Although not shown, the hTNNT2 element also includes Exon 1. The full polynucleotide sequence of one embodiment of the vector genome is SEQ ID NO: 13, and the associated expression cassette is SEQ ID NO: 9, The full polynucleotide sequence of another embodiment of the vector genome is SEQ ID NO: 97, and the associated expression cassette is SEQ ID NO: 100.

FIG. 3 shows a diagram illustrating a non-limiting example of a vector genome. The full polynucleotide sequence of one embodiment of the vector genome is SEQ ID NO: 14, and the associated expression cassette is SEQ ID NO: 10. The full polynucleotide sequence of another embodiment of the vector genome is SEQ TD NO: 98, and the associated expression cassette is SEQ ID NO: 101. The MHCK7 promoter as described herein is labelled “Enhancer/MHCK7” in the diagram.

FIG. 4 shows a diagram illustrating a non-limiting example of a vector genome. The full polynucleotide sequence of one embodiment of the vector genome is SEQ ID NO: 15, and the associated expression cassette is SEQ ID NO: 11. The full polynucleotide sequence of another embodiment of the vector genome is SEQ ID NO: 99, and the associated expression cassette is SEQ ID NO: 102.

FIGS. 5A-5B show PKP2 protein expression in CHO-Lee2 cells. FIG. 5A shows Western Blots (WB) of PKP2 (top panel) or loading control, GAPDH (bottom panel). FIG. 5B show a bar graph of the Western Blot. The AAV vector serotype (AAV9 or AAVrh 74) and the promoter (MHCK7 or hTnT) are noted (AA9 indicates AAV9 and rh.74 indicates AAVrh.74). Controls included a GFP vector (CON-GFP) and no transduction (No Tdxn).

FIG. 6 shows left ventricle ejection fraction percentage (LVEF %) for normal mice (control) or PKP2 knockout mice (cKO PKP2) that received intravenous formulation control (FB), or mice that received intravenous AAV vectors delivering the indicated human PKP2a. All AAV doses were 3E13 vg/kg. LVEF was determined using echocardiography in Control-FB and PKP2-cKO mice at 28 days post-tamoxifen induction and 56 days post-injection with FB, AV9-MHCK7-PKP2a (3E13 vg/kg), AAV9-hTnT-PKP2a (3E13 vg/kg), AAVrh.74-MHCK7-PKP2a (3E13 vg/kg) or AAVrh 74-hTnT-PKP2a (3E13 vg/kg). Statistical analyses were performed using a One-way ANOVA) followed by Dunnett's post-hoc comparisons (****p<0.0001) relative to the PKP2-cKO-FB control group). Data are plotted as Mean±SEM with number per group (N).

FIG. 7 shows left ventricle fractional shortening percentage (FS %) for normal mice (control) or untreated PKP2 knockout mice (cKO PKP2) that received intravenous formulation control (FB), or mice that received the indicated intravenous AAV vectors delivering human PKP2a. All AAV doses were 3E13 vg/kg. FS in the left ventricle was determined from echocardiography at 28 days post-tamoxifen induction, which was 56 days post-A AV (AAV9-MHCK7-PKP2a, AAV9-hTnT-PKP2a, A AVrh.74-MHCK7-PKP2a or AAVrh.74-hTnT-PKP2a at a dose of 3E13 vg/kg) or FB control injections. Statistical analyses were performed using a One-Way ANOVA) with Dunnett's post-hoc comparisons against the PKP2-cKO-FB control group (*p<0.05, ****p<0.0001). Data are plotted as Mean±. SEM with number per group (N).

FIG. 8 shows right ventricle area in millimeters squared (RV Area (mm²)) for normal mice (control) or untreated PKP2 knockout mice (cKO PKP2) that received intravenous formulation control (FB), or mice that received the indicated intravenous AAV vectors delivering human PKP2a (right bars). All AAV doses were 3E13 vg/kg. RV area was calculated from echocardiography at 28 days post-tamoxifen treatment, which was 56 days post-AAV (AAV9-MHCK7-PKP2a, AAV9-hTnT-PKP2a, AAVrh.74-MHCK7-PKP2a or AAVrh.74-hTnT-PKP2a at a dose of 3E13 vg/kg) or FB control injections. Statistical analyses were performed (One-Way ANOVA) followed by Dunnett's post-hoc comparisons against the PKP2-cKO-FB control group (*p S 0.05, **p K 0.005, * p<0.0005, ****p<0.0001). Data are presented as Mean±SEM with number per group (N).

FIG. 9 shows right ventricle velocity time integral in millimeters per second (RV VTI (mm/sec)) for normal mice (control) or untreated PKP2 knockout mice (cKO PKP2) that received intravenous formulation control (FB), or mice that received the indicated intravenous AAV vectors delivering human PKP2a. All AAV doses were 3E13 vg/kg. Calculated from Echocardiography. RV-VTI was measured at 28 days post-tamoxifen treatment and 56 days post-injection with the FB control or AAV (AA V9-MHCK7-PKP2a, AAV9-hTnT-PKP2a, AAVrh.74-MHCK7-PKP2a or AAVrh.74-hTnT-PKP2a at a dose of 3E13 vg/kg). Statistical analyses were performed using One-way ANOVA, followed by Tukey's post-hoc multiple comparisons test relative to PKP2-cKO-FB controls (*p<0.05, **p<0.01) Data are presented as Mean±SEM with number per group (N).

FIGS. 10A-10B illustrate the degree of fibrosis in left and right ventricles based on quantitation of the Percent Collagen following trichrome histological staining of the heart for normal mice (control) or untreated PKP2 knockout mice (cKO PKP2) that received intravenous formulation control (FE), or mice that received the indicated intravenous AAV vectors delivering human PKP2a. All AAV doses were 3E13 vg/kg. Control animals without the conditional PKP2 gene knock-out (Cre Neg group) were found to have very little collagen, while control PKP2 knock-out animals receiving Formulation Buffer (CKO FB group) were found to have substantially greater proportion of collagen in both left and right ventricles. A AV-mediated overexpression of PKP2 resulted in robust attenuation of collagen, to varying degrees, in all A AV-injected groups [n=4 for all groups; p-values reflect results from One-way ANOVA with Bonferroni post-hoc analyses]. FIG. 10A is a bar graph of percent fibrosis in the left ventricle. FIG. 10B is a bar graph of percent fibrosis in the right ventricle. The percentage of fibrosis was assessed in the heart left and right ventricles by trichrome staining performed on Control and PKP2-cKO mice at 28 days post-tamoxifen treatment, which was 56 days post-FB or AAV (AAV9-MHCK7-PKP2a, AAV9-hTnT-PKP2a, AAVrh.74-MHCK7-PKP2a or AAVrh.74-hTnT-PKP2a at a dose of 3E13 vg/kg) injection.

FIGS. 11A-11B show PKP2 protein expression in mouse heart. All AAV doses were 3E13 vg/kg. FIG. 11A show's Western Blots (WE) of PKP2 (top panel) or loading control, GAPDH (bottom panel). FIG. 11 shows a bar graph of the Western Blot. The AAV vector serotype (AAV9 or rh.74 rh.74) and the promoter (MHCK7 or hTnT) are noted. Gel images (FIG. 11A) and quantitation of PKP2 normalized to GAPDH (FIG. 11E, quantified from FIG. 11A top gels acquired at the same time) detected in the heart at 28 days post-tamoxifen injection, which was 56 days post-AAV (AAV9-MHCK7-PKP2a, AAV9-hTnT-PKP2a, AAVrL.74-MHCK7-PKP2a or AAVrh 74-hTnT-PKP2a at a dose of 3E13 vg/kg) injection. Data are presented as Mean±SEM with number per group (N).

FIGS. 12A-12B show LVEF and FS in mice. The mice were dosed with AAV vectors or formulation control (FB) and 4 weeks after they were treated with Tamoxifen. FIG. 12A shows left ventricle ejection fraction percentage (LVEF %) at baseline (left bars) and 28 days after Tamoxifen treatment, AAV9 dose was aE13 vg/kg, and AAVrh.74 dose was 6E13 vg/kg. LVEF was measured from echocardiograms in PKP2-cKO mice taken at 28 days post-tamoxifen injection and 56 days post-injection with FB, AAV9-hTnT-PKP2a (1E13 vg/kg), or AAVrh.74-hTnT-PKP2a (6E13 vg/kg). Statistical analyses were performed using a repeated measures two-way ANOVA ***p<0,001, ****p<0.000 1). Data are plotted as Mean±. SEM with number per group (N); dpi TAM=days post-injection tamoxifen. FIG. 12B shows left ventricle fractional shortening percentage (FS %) at baseline (left bars) and 28 days after Tamoxifen treatment. AAV9 dose was aE13 vg/kg, and AAVrh.74 dose was 6E13 vg/kg. FS was measured from echocardiograms in PKP2-cKO mice taken at 28 days post-tamoxifen injection and 56 days post-injection with FB, AAV9-hTnT-PKP2a (1E13 vg/kg), or AAVrh.74-hTnT-PKP2a (6E13 vg/kg). Statistical analyses were performed using a repeated measures two-way ANOVA **:*p 0.001, **** <0.0001). Data are plotted as Mean±SEM with number per group (N); dpi TAM=days post-injection tamoxifen.

FIGS. 13A-13B show RV‘ area and RV’ VTI in mice. The mice were dosed with AAV vectors or formulation control (FB) and 4 weeks after they were treated with Tamoxifen, FIG. 13A show right ventricle area in millimeters squared (RV Area (mm²)) at baseline (left bars) and 28 days after Tamoxifen treatment. RV area was measured from echocardiograms taken at 28 days post-tamoxifen and 56 days following intravenous injection with FE, AAV9-hTnT-PKP2a (1E13 vg/kg), or AAVrh.74-hTnT-PKP2a (6E13 vg/kg). Statistical analyses were performed using a two-way ANOVA; (* *p 0,01, ***p K 0.001), Data are plotted as Mean±SEM with number per group (N); dpi TAM=days post-injection tamoxifen. FIG. 13B show right ventricle velocity time integral in millimeters per second (RV VTI (mm/sec)) at baseline (left bars) and 28 days after Tamoxifen treatment. RV-VTI was measured from echocardiograms taken at 28 days post-tamoxifen treatment and 56 days post-injection with FB (control), AAV9-hTnT-PKP2a (1E13 vg/kg), or A AVrh.74-hTnT-PKP2a (6E13 vg/kg). Statistical analyses were performed using two-way mixed effects ANOVA, followed by Tukey's post-hoc multiple comparison test (*p 51 0.05). Data are plotted as Mean±SEM with number per group (N); dpi TAM=days post-injection tamoxifen.

FIGS. 14A-14B illustrate the degree of fibrosis in left and right ventricles based on quantitation of the Percent Collagen following trichrome histological staining of the heart. Degree of fibrosis was assessed by trichrome staining in left and right ventricles of Control injected with FB and PKP2-cKO mice injected with FB, AAV9-hTnT-PKP2a (1E13 vg/kg), or AAVrh.74-hTnT-PKP2a (6E13 vg/kg) at 28 days post-tamoxifen and 56 days post-AAV injection. Quantitation of percent fibrotic heart tissue in left ventricle (FIG. 14A) and right ventricle (FIG. 14B) was performed. Statistical analyses were performed using one-way ANOVA. When appropriate, Tukey's post-hoc comparisons. The figures illustrate the degree of fibrosis in left and right ventricles based on quantitation of the Percent Collagen following trichrome histological staining of the heart. AAV-mediated overexpression of PKP2 resulted in robust attenuation of collagen, to varying degrees, in all AAV-injected groups [n=4 for all groups; p-values reflect results from One-way ANOVA with Bonferroni post-hoc analyses]. FIG. 14A is a bar graph of percent collagen in the left ventricle. FIG. 14B is a bar graph of percent collagen in the right ventricle.

FIGS. 15A-15B show PKP2 protein expression in mouse heart. FIG. 15A shows Western Blots (WB) of PKP2 (top panel) or loading control, GAPDH (bottom panel). PKP2 protein was assessed by Western blot in the heart from Control mice injected with FB or PKP2-cKO mice injected with FB, AAV9-hTnT-PKP2a (1E13 vg/kg), or AAVrh.74-hTnT-PKP2a (6E13 vg/kg) at 56 days post-AAV injection and 28 days post-tamoxifen injection. FIG. 15B show a bar graph of the Western Blot. Quantitation of PKP2 protein bands relative to GAPDH. Statistical analyses were performed using one-way ANOVA, followed by Tukey's post-hoc analyses (*p 0.05, * *p 0.01, ***p 5 0.001). Data are plotted as Mean SEM with number per group (N). The AAV vector serotype (AAV9 or rh.74 rh.74) and the dose (1E 13 vg/kg or 6E3.1 vg/kg) are noted.

FIGS. 16A-C show diagrams illustrating the Experimental Timeline for the experiments −28, +7 and +14 days. AAV was administered to the PKP2-cKO mice 7- or 14-days post-tamoxifen injection (FIG. 16C). Echocardiograms were performed at baseline (for AAV treated animals) and 28 days post-tamoxifen (for all animals) as well as at study endpoint at 5 months post-tamoxifen (FIG. 16A and FIG. 16B).

FIG. 17 shows a Kaplan Meier Survival Curve demonstrating the prevention of Disease-Related Mortality Following Intravenous AAVrh.74-hTnT-PKP2a in the PKP2-cKO Model of ACM. The Kaplan-Meier survival curve depicts the long-term survival of PKP2-cKO mice following intravenous infusion of AAVrh 74-hTnT-PKP2a when administered at +7-(6E13 vg/kg) or +14-(2E14 vg/kg) days post-tamoxifen. Also depicted are survival curves from animals that received intravenous AAV9-hTnT-PKP2a (3E13 vg/kg) ±7-days post-tamoxifen and FE-injected PKP2-cKO (PKP2-eKO-FE) and control (Control-FB) mice, Statistical Analyses using Log-rank (Mantel-Cox) test revealed significant differences between AAVrh.74-hTnT-PKP2a and AAV9-hTnT-PKP2a groups as well as both groups relative to controls (****p<0,001). The lines from top to bottom at 50 days correspond to AAVrh 74-hTnt-6E13+7D, AAVrh 74-hTnt-2E14+14D, and PKP2-cKO-FB. The lines from top to bottom at 150 days correspond to AAVrh.74-hTnt-6E13+7D, AAVrh.74-hTnt-2E14+14D, and AAV9-hTnt-3E13+7D.

FIGS. 18A-18B show left ventricle ejection fraction percentage (LVEF %) and left ventricle fractional shortening percentage (FS %) for AAV delayed injection paradigm (+7 and +14 days). FIG. 18A show left ventricle ejection fraction percentage (LVEF %) after 28 days and after 5 months of Tamoxifen treatment. LVEF was calculated from echocardiography performed 28 days and 5 months post-tamoxifen injection on FB injected control (Control-FB) or PKP2-cKO mice injected with FE (PKP2-cKO-FB), AAVrh 74-hTnT-PKP2a (6E 13 vg/kg, +7 days post-tamoxifen and 2E14 vg/kg,+14 days post-tamoxifen), and AA V9-hTnT-PKP2a (3E13 vg/kg, +7 days post-tamoxifen). Statistical analyses were performed using One way ANOVA followed by Dunnett's post—hoc comparisons. p values reflect significance relative to PKP2-cKO-FB at 28 days post-tamoxifen ***p<0.001. **** p 5 0.0001). Data are presented as Mean±SEM with number per group (N). FIG. 18B show left ventricle fractional shortening percentage (FS %) after 28 days and after 5 months of Tamoxifen treatment. FS was calculated from echocardiography performed 28 days and 5 months post-tamoxifen injection. Statistical analyses (One-way ANOVA) followed by Dunnett's post-hoc comparisons were performed. p values reflect significance relative to FEB injected PKP2-cKO (PKP2-cKO-FB) at 28 days post-tamoxifen (*p≤0.05, **p≤0.01, **** p≤0.000 1). Data are presented as Mean±SEM with number per group (N). The bars of the graphs from left to right correspond to the legend from top to bottom.

FIGS. 19A-19B: show right ventricle area (mm2) and right ventricle velocity time integral in millimeters per second (RV VTI (mm/sec)) for AAV delated injection paradigm (+7 and +14 days). FIG. 19A shows right ventricle area in millimeters squared (RN Area (mm²)) after 28 days and after 5 months of Tamoxifen treatment. RV Area was calculated from echocardiography performed 28 days and 5 months post-tamoxifen injection on FB injected control mice (Control-FB) or PKP2-cKO mice injected with FB (PKP2-cKO-FB), AAVrh.74-hTnT-PKP2a (6E13 vg/kg, +7 days post-tamoxifen), AAVrh.74-hTnT-PKP2a (2E14 vg/kg, +14 days post-tamoxifen), and AAV9-hTnT-PKP2a (3E13 vg/kg, +7 days post-tamoxifen). Statistical analyses were performed using One-way ANOVA followed by Dunnett's post-hoc comparisons. p values reflect significance relative to PKP2-cKO-FB mice-at 28 days post-tamoxifen (**** p<0.0001). Data are presented as Mean±SEM with number per group (N). FIG. 19B shows right ventricle velocity time integral in millimeters per second (RV VTI (mm/sec)) after 28 days and after 5 months of Tamoxifen treatment. RV-VTI was calculated from echocardiography performed 28 days and 5 months post-tamoxifen injection on Control-FB or PKP2-cKO mice injected with FB (PKP2-cKO-FB), AAVrh.74-hTnT-PKP2a (6E113 vg/kg, +7 days post-tamoxifen), AAVrh.74-hTnT-PKP2a (2E14 vg/kg, 14 days post-tamoxifen), and AAV9-hTnT-PKP2a (3E13 vg/kg, +7 days post-tamoxifen). Statistical analyses were performed using One-way ANOVA followed by Dunnett's post-hoc comparisons against PKP2-cKO-FB at 28 days post-tamoxifen (*p 0.05, **p 0.01 ***p 0.001). Data are presented as Mean±SEM with number per group (N). The bars of the graphs from left to right correspond to the legend from top to bottom.

FIGS. 20A-20C show the Mitigation of Arrhythmia following AAVrh.74 mediated overexpression of PKP2a in PKP2-cKO Mice using the AAV delayed injection paradigm (+−14 days) animal model. AAVrh.74-hTnT-PKP2a was injected 14 days following tamoxifen-induced knock-out of PKP2. Electrocardiography (ECG) data were collected at 21- and 28-days post-tamoxifen injection (7 and 14 days following AAV, respectively). FIG. 20A shows the proportion of cases with >1 00 premature ventricular contractions (PVCs) in animals treated with AAVrh.74-hTnT-PKP2a when compared to FB-treated PKP2-cKO controls. FIG. 20B shows levels of ectopic beats revealing alleviation of arrhythmia burden. (*p≤0.05, * *p≤0.01). Data are presented as Mean±SEM with number per group (N). For each treatment, the left bar is 21-days post-tamoxifen injection, and the right bar is 28-days post-tamoxifen injection. FIG. 20C shows an electrocardiogram at 21 days post-tamoxifen and treatment in a control mouse (FB) and a mouse treated with AAVrh.74-PKP2a.

FIGS. 21A-21B show the degree of fibrosis 5 months post-tamoxifen in left (FIG. 21A) and right ventricles (FIG. 21B) based on quantitation of the Percent Collagen following trichrome histological staining of the heart. AAV-mediated overexpression of PKP2 resulted in robust attenuation of collagen, to varying degrees, in all AAV-injected groups [n=4 or 8 per group; p-values reflect results from One-way ANOVA with Bonferroni post-hoc analyses]. Degree of fibrosis was assessed 5 months post-tamoxifen induction in the left and right ventricles of FB injected control mice (CON-FB), or PKP2-cKO mice treated with AAV9-hTnT-PKP2a (3E13 vg/kg, +7-days post-tamoxifen), AAVrh.74-hTnT-PKP2a (6E13 vg/kg, +7-days post-tamoxifen), and AAVrh.74-hTnT-PKP2a (2E114 vg/kg, +14-days post-tamoxifen). Quantitation of percent fibrotic heart tissue in left ventricle (FIG. 21A) and right ventricle (FIG. 21B). Statistical analyses were performed using One-way ANOVA followed by Dunnett's post-hoc comparisons against FB treated control mice at 28 day s post-tamoxifen (*p≤0,05, **p≤0.01). Data are presented as Mean±SEM with number per group (N).

FIG. 22 shows PKP2 protein expression in mouse heart 5 months post injection with A AV (+7 or +14 days post-tamoxifen paradigm). The bar graph shows the quantifications of the Western Blot. The AAV vector serotype AAV rh.74 rh.74 at the doses of 6E13 vg/kg (AAV 7 days post-tamoxifen) or 2E14 vg/kg (AAV injection 14 days post-tamoxifen). PKP2 protein was assessed by Western blot in the hearts from control mice administered FB (CON-FB) and AAVrh.74-hTnT-PKP2a (6E13 vg/kg and 2E314 vg/kg) 5 months post injection with AAV (+7-days post-tamoxifen). Quantitation of PKP2 protein bands is presented relative to GAPDH. Statistical analyses revealed no significant differences between groups. Data are presented as Mean±SEM with number per group (N).

FIGS. 23A-23C show dose-dependent detection of (FIG. 23A) vector genomes (ddPCR), (FIG. 23B) transgene PKP2a mRNA (RT-ddPCR) and (FIG. 23C) PKP2 protein expression (Western Blot) in cardiac tissue from AAVrh.74-PKP2a injected animals relative to controls. Red dotted line in FIG. 23C indicates endogenous expression level for PKP2 protein normalized to GAPDH in control-FB mice. Data are presented as Mean±SD. Left bars, control mice treated with Formulation Buffer (FB)(control); second from left bars, PKP2-cKO mice treated with FB (PKP2-cKO); third from left bars, PKP2-cKO mice treated with AAVrh.74-PKP2a 3×10¹³ vg/kg; right bars, PKP2-cKO mice treated with A AVrh.74-PKP2a 6×10¹³ vg/kg. Number of Mice studied noted in corresponding bars (N). Statistical analyses were performed using One-way ANOVA followed by Tukey's post-hoc analyses. <LoQ: Less than limit of quantitation; FE: Formulation Buffer.

FIGS. 24A-24C show representative images of immunofluorescence staining for plakophilin2 (PKP2; in red) and nuclei (DAPT; in blue) in hearts from (FIG. 24A) control mice treated with Formulation Buffer (FE), (FIG. 24B) PKP2-cK(mice treated with FB, and (FIG. 24C) PKP2-cKO mice treated with AAVrh.74-PKP2a 6×10¹³ vg/kg. Mice were treated with FE or AAVrh.74-PKP2a 28 days before Tamoxifen injection. White arrowheads in panels A and C highlight PKP2 localization at the intercalated disc. FE: Formulation Buffer. Scale bar panels on the left=150 μm, Scale bar panels on the right=50 μm.

FIGS. 25A-25C show representative images of left (LV) and right ventricular (RV) echocardiography to assess contractility in control and PKP2-cKO mice treated with Formulation Buffer (FB), or in PKP2-cKO mice treated with AAVrh.74-PKP2a 6×10¹³ vg/kg, 28 days before Tamoxifen injection (FIG. 25A). Measurements were collected 28 day s post-Tamoxifen injection. FIG. 25B shows quantification of left ventricular ejection fraction (LVEF) measured by long axis B-mode echocardiography, and FIG. 25C shows right ventricular (RV) area measured by modified long axis B-mode echocardiography. Red dotted lines indicate the average value recorded from PKP2-cKO mice injected with FB. Data are presented as Mean i SD. Left bars, control mice treated with FE; second from left bars, PKP2-cKO mice treated with FE; third from left bars, PKP2-cKO mice treated with AAVrh.74-PKP2a 3×10¹³ vg/kg; right bars, PKP2-cKO mice treated with AAVrh. 74-PKP2a 6×10¹³ va/kg. Statistical analyses were performed using One-way ANOVA followed by Tukey's post-hoc analyses. LVID: Left ventricular internal diameter, LVEF: Left ventricular ejection fraction, RV: Right ventricle, FE: Formulation Buffer.

FIGS. 26A-26C show cardiac fibrosis in AAVrh.74-PKP2a treated PKP2-cKO mice. The left panel of FIG. 26A shows representative images of Masson's trichrome staining of longitudinal heart sections of control and PKP2-cKO mice treated with Formulation Buffer (FB) and PKP2-cKO mice treated with AAVrh.74-PKP2a, 28 days before Tamoxifen injection. The right panel of FIG. 26A shows high contrast mask of the same sections emphasizing collagen deposition in blue. Scale bar=1 mm for all images. Hearts were extracted 28-days post-Tamoxifen injection. FIG. 26B shows quantification of the percentage of left ventricular fibrosis and FIG. 26C shows right ventricular fibrosis in hearts of the four different groups. Data are presented as Mean i SD. Left bars, control mice treated with FB; second from left bars, PKP2-cKO mice treated with FB; third from left bars, PKP2-cKO mice treated with AAVrh.74-PKP2a 3×10¹³ vg/kg; right bars, PKP2-cKO mice treated with AAVrh.74-PKP2a 6×10¹³ vg/kg. Statistical analyses were performed using One-way ANOVA followed by Tukey's post-hoc analyses. FB: Formulation Buffer.

FIG. 27 is a graph showing survival following AAVrh.74-PKP2a injection in PKP2-cKO mice at 7- or 14-days post-Tamoxifen. Kaplan Meier curve depicting the long-term survival of PKP2-cKO mice following AAVrh.74-PKP2a (6×10¹³ vg/kg, 7 days post-Tamoxifen or 2×10¹⁴ vg/kg, 14 days post-Tamoxifen) administration compared to control and PKP2-cKO mice injected with Formulation Buffer (FB).

FIGS. 28A-28D show disease progression in PKP2-cKO mice upon AAVrh.74-PKP2a treatment at 7- or 14-days post-Tamoxifen. The upper panel of FIG. 28A shows representative images of Masson's trichrome staining of longitudinal heart sections of control mice treated with Formulation Buffer (FB) and PKP2-cKO mice treated with AAVrh. 74-PKP2a, 2×10¹³ vg/kg 14 days post-Tamoxifen. Hearts were extracted 5 months post-Tamoxifen injection. The bottom panel of FIG. 28A shows high contrast mask of the same sections emphasizing collagen deposition in blue. Scale bar=1 mm for all images FIG. 28B shows quantification of the percentage of left ventricular fibrosis (left panel) and right ventricular fibrosis (right panel) by Masson's trichrome staining of longitudinal heart sections in control mice treated with FB and PKP2-cKO mice treated with AAVrh.74-PKP2a, 7 days (6×10¹³ vg/kg) or 14 days (2×10¹³ vg/kg) post-Tamoxifen. Hearts were extracted 5 months post-Tamoxifen. Red dotted line indicates fibrosis level in PKP2-cKO mice injected with Formulation Buffer (FB). FIG. 28C shows quantification of left ventricular ejection fraction (LVEF), and FIG. 28D shows quantification of right ventricular area (RV area) across time in PKP2-cKO mice treated with AAVrh.74-PKP2a, 7 or 14 days post-Tamoxifen with the dose indicated at the bottom of the bars. Echocardiography was performed at 28 days and 5 months post-Tamoxifen. Echocardiography for control and PKP2-cKO mice treated with FB was performed at 28 days post-Tamoxifen only. Data are presented as Mean±SD. Left bars are control mice treated with FB; second from left bars are PKP2-cKO mice treated with FB, third and fourth from left bars are PKP2-cKO mice treated with AAVrh.74-PKP2a 6×10¹³ vg/kg; two right bars are PKP2-cKO mice treated with A AVrh74-PKP2a 2×10¹⁴ vg/kg. Statistical analyses were performed using One-way ANOVA followed by Tukey's post-hoc analyses. FB: Formulation Buffer.

FIGS. 29A-29D show results of Isoproterenol-induced arrhythmias in PKP2-cKO hearts treated with AAVrh.74-PKP2a. FIG. 29A shows representative electrocardiogram (ECG) traces from PKP2-cKO mice treated with formulation buffer (FB), FIG. 29B shows representative ECG traces from PKP2-cKO mice treated with and AAVrh 74-PKP2a 6×10¹³ vg/kg, 14 days post-Tamoxifen injection. FIG. 29C shows the percentage of mice that presented with >100 premature ventricular contractions (PVCs) after isoproterenol (ISO).

FIG. 29D shows the number of ISO-induced ectopic beats in PKP2-cKO mice treated with FB or AAVrh.74-PKP2a. Data shown FIG. 29C and FIG. 29D were quantified over a period of 30 minutes after ISO injection, and ECGs were recorded 21 days post-Tamoxifen. Data presented as Mean±SD. Left bars, PKP2-cKO mice treated with FB; middle bars, PKP2-cKO mice treated with AAVrh.74-PKP2a 6×100¹³ vg/kg; and right bars, PKP2-cKO mice treated with AAVrh.74-PKP2a 2×10⁴ vg/kg. Statistical analyses were performed using One-way ANOVA followed by Dunns post-hoc analyses. PVC: Premature ventricular contraction, FB: Formulation Buffer.

FIGS. 30A-30D show transduction of PKP2 in the hearts of non-human primates. FIGS. 30A-B are photomicrographs following immunohistochemical labeling for PKP2 protein in sections through the nonhuman primate heart of either a control PB (FIG. 30A) or “Low Dose” treated animal (FIG. 30B). FIG. 30C and FIG. 30D show quantitation of PKP2 protein by Western blot and transgene mRNA (hPKP2a) in the ventricles of the nonhuman primate heart following intravenous infusion of either a “Low” (8×10¹³ vg/kg) or “High” (3×10¹⁴ vg/kg) dose AAVrh.74-PKP2a or FB (control).

FIGS. 31A-31D show cardiac expression of AnkyrinB in AAVrh.74-PKP2a-treated PKP2-cKO mice. FIG. 31A shows the method used for data acquisition and analysis of immunofluorescent (IF) AnkyrinB (AnkB) signals collected from paraffin-embedded tissue sections, Red line indicates the range of fluorescence intensities as plotted in the right panel. Peak depth was defined as the range between peak and valley. FIG. 311B shows representative images of IF staining for AnkB (in green) and nuclei (in blue) in hearts from control mice treated with Formulation Buffer (PB), PKP2-cKO mice treated with FB, and PKP2-cKO mice treated with AAVrh.74-PKP2a 6×10¹³ vg/kg 28 days before Tamoxifen injection. Scale bar:=100 μm. FIG. 31C shows quantification of AnkB peak depth (left panel) and mean intensity (right panel) in left ventricular sections of the three different groups. FIG. 31D shows quantification of AnkB peak depth (left panel) and mean intensity (right panel) in right ventricular sections of the three different groups. Data presented as Mean±SD. Left bars, control mice treated with FB; middle bars, PKP2-cKO mice treated with FB; right bars, PKP2-cKO mice treated with AAVrh.74-PKP2a 6×10¹³ vg/kg. Statistical significance was evaluated by One—was ANOVA followed by Tukey's post-hoc analyses.

FIGS. 32A-32C show cardiac expression of desmin in AAVrh.74-PKP2a-treated PKP2-cKO mice. FIG. 32A shows representative images of immunofluorescent staining; for desmin (in green) and nuclei (in blue) in hearts from control mice treated with Formulation Buffer (FB), PKP2-cKO mice treated with FB, and PKP2-cKO mice treated with AAVrh.74-PKP2a 6×10¹³ vg/kg 28 days before Tamoxifen injection. Scale bar=100 μm. FIG. 32B shows quantification of desmin peak depth (left panel) and mean intensity (right panel) in left ventricular sections of the three different groups. FIG. 32C shows quantification of desmin peak depth (left panel) and mean intensity (right panel) in right ventricular sections of the three different groups. Data presented as Mean±SD. Black bars, control mice treated with FB; red bars, PKP2-cKO mice treated with FB; purple bars, PKP2-cKO mice treated with AAVrh.74-PKP2a 6×10¹³ r vg/kg. Statistical significance was assessed by One-way ANOVA followed by Tukey's post-hoc test.

### DETAILED DESCRIPTION OF THE DISCLOSURE

The present disclosure provided gene therapy vectors for PKP2 that deliver a polynucleotide encoding a PKP2 polypeptide or a functional variant thereof, along with methods of use, and other compositions and methods,

In particular embodiments, the disclosure relates to a gene therapy vector comprising a promoter sequence operatively linked to a polynucleotide encoding a PKP2 polypeptide or a functional variant thereof. In some embodiments, the promoter is a Myosin Heavy-chain Creatine Kinase 7 (MHCK7) promoter. In some embodiment, the AAV vector is an AAV9 vector. In some embodiments, the promoter is an MHCK7 promoter and the AAV vector is an AAV9 vector. In some embodiments, the promoter is a hTNNT2 promoter. In some embodiment, the promoter is an hTNNT2 promoter and the AAV vector is an AAV9 vector. In some embodiments, the PKP2 is human PKP2a. In some embodiments, the PKP2 is human PKP2b. In some embodiment, the AAV vector is an AAVrh 74 vector. In some embodiments, the promoter is an MHCK7 promoter and the AAV vector is an AAVrh 74 vector. In some embodiments, the promoter is a hTNNT2 promoter. In some embodiment, the promoter is an hTNNT2 promoter and the AAV vector is an AAVrh.74 vector. In some embodiments, the PKP2 is human PKP2a. In some embodiments, the PKP2 is human PKP2b.

This disclosure further provides methods of treating a disorder or disorder in a subject by administering a gene therapy vector of the disclosure to the subject. In a certain embodiment, the disorder or disorder is arrhythmogenic cardiomyopathy (ACM).

In certain embodiments, the subject being treated is an ACM patient having one or more mutation in a PKP2 gene. More than half of ACM patients carry mutations in the desmosomal gene PKP2 encoding the protein Plakophilin-2 (PKP2). PKP2 is also associated with Brugada syndrome (BrS) and idiopathic ventricular fibrillation. It is a member of the armadillo repeat and plakophilin protein family. The protein contains nine central, conserved armadillo repeat domains flanked by N-terminal and C-terminal domains. It functions to link cadherins to intermediate filaments in the cytoskeleton.

Plakophilin 2 localizes to cell desmosomes and nuclei and binds plakoglobin, desmoplakin, and the desmosomal cadherins via an N-terminal head domain. PKP2 provides a lateral stabilizing force with the desmosomal-intermediate filament assembly facilitating cell-to-cell contact. It may also serve roles in intracellular signaling regulation, electrophysiologic and trafficking regulation, and control of transcription processes,

Intravenous injection of an AAV9 vector encoding a C-terminal deletion mutant of PKP2a (R735X) in the heart of wild-type mice accelerates the appearance of ACM when treated mice are subjected to exercise training. Cruz et al. J Am Col Cardiol. 65(14):1438-50 (2015). Mutant PKP2a causes a disease phenotype; and control AAV9 vector expressing the non-mutant PKP2a causes no phenotypic change in wild type mice. Heterologous expression of wild-type human PKP2a does not induce disease or altered function. These studies demonstrate that mutant PKP2a can cause a disease phenotype. They fail to demonstrate a curative role for PKP2, because heterologous expression of non-mutant PKP2 resulted in no phenotypic change in the wild-type mouse.

In accordance with the present disclosure, a polynucleotide encoding a PKP2 or functional variant thereof, wherein the PKP2 or functional variant thereof comprises at least 800 or at least 830 amino acids (e.g., no C terminal truncation at Arg-735), may be employed in generating a gene therapy vector. The resulting vector may be employed in treating diseases or disorders, e.g. a PKP2-related disease or disorder, e.g., ACM, Brugada syndrome (BrS), idiopathic ventricular fibrillation, hypertrophic cardiomyopathy (HCM), dilated cardiomyopathy (DCM), and others.

Unless otherwise defined, all technical and scientific terms used herein have the same meaning as commonly understood by one of ordinary skill in the art to which this disclosure pertains. Although methods and materials similar or equivalent to those described herein can be used in the practice of the present disclosure, suitable methods and materials are described below. In addition, the materials, methods, and examples described herein are illustrative only and are not intended to be limiting.

All publications and patents mentioned herein are hereby incorporated by reference in their entirety as if each individual publication or patent was specifically and individually indicated to be incorporated by reference, including but not limited to PCT Application Publication No. WO 2022/032226 and U.S. application Ser. Nos. 17/670,389, 17/670,390. In case of conflict, the present application, including any definitions herein, will control. However, mention of any reference, article, publication, patent, patent publication, and patent application cited herein is not, and should not be taken as an acknowledgment, or any form of suggestion, that they constitute valid prior art or form part of the common general knowledge in any country in the world.

In the present description, any concentration range, percentage range, ratio range, or integer range is to be understood to include the value of any integer within the recited range and, when appropriate, fractions thereof (such as one tenth and one hundredth of an integer), unless otherwise indicated. The term “about”, when immediately preceding a number or numeral, means that the number or numeral ranges plus or minus 10%. It should be understood that the terms “a” and “an” as used herein refer to “one or more” of the enumerated components unless otherwise indicated. The use of the alternative (e.g., “or”) should be understood to mean either one, both, or any combination thereof of the alternatives. The term “and/or” should be understood to mean either one, or both of the alternatives. As used herein, the terms “include” and “comprise” are used synonymously.

As used herein, the term “in a subject overtime” refers to an effect in a subject that occurs for about one day, for about one month, for about one year, for about one decade, and/or for about several decades.

As used herein, the terms “identity” and “identical” refer, with respect to a polypeptide or polynucleotide sequence, to the percentage of exact matching residues in an alignment of that “query” sequence to a “subject” sequence, such as an alignment generated by the BLAST algorithm. Identity is calculated, unless specified otherwise, across the full length of the subject sequence. Thus, a query sequence “shares at least x % identity to” a subject sequence if, when the query sequence is aligned to the subject sequence, at least x % (rounded down) of the residues in the subject sequence are aligned as an exact match to a corresponding residue in the query sequence. Where the subject sequence has variable positions (e.g., residues denoted X) an alignment to any residue in the query sequence is counted as a match. Sequence alignments may be performed using the NCBI Blast service (BLAST+version 2.12.0).

As used herein, the term “operatively linked” refers to a functional relationship between two or more nucleic acid (e.g., DNA) segments. Typically, it refers to the functional relationship of a transcriptional regulatory sequence to a transcribed sequence. For example, a promoter sequence is operatively linked to a coding sequence if it stimulates or modulates the transcription of the coding sequence in an appropriate host cell or other expression system. Generally, promoter transcriptional regulatory sequences that are operatively linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

As used herein, an “AAV vector” or “rAAV vector” refers to a recombinant vector comprising one or more polynucleotides of interest (or transgenes) that are flanked by AAV inverted terminal repeat sequences (ITRs). Such AAV vectors can be replicated and packaged into infectious viral particles when present in a host cell that has been transfected with a plasmid encoding and expressing rep and cap gene products. Alternatively, AAV vectors can be packaged into infectious particles using a host cell that has been stably engineered to express rep and cap genes.

As used herein, an “AAV virion” or “AAV viral particle” or “AAV vector particle” refers to a viral particle composed of at least one AAV capsid protein and an encapsidated polynucleotide AAV vector. As used herein, if the particle comprises a heterologous polynucleotide (i.e., a polynucleotide other than a wild-type AAV genome such as a transgene to be delivered to a mammalian cell), it is typically referred to as an “AAV vector particle” or simply an “AAV vector.” Thus, production of AAV vector particle necessarily includes production of AAV vector, as such a vector is contained within an AAV vector particle.

As used herein, “promoter” refers to a polynucleotide sequence capable of promoting initiation of RNA transcription from a polynucleotide in a eukaryotic cell.

As used herein, “vector genome” refers to the polynucleotide sequence packaged by the vector (e.g., an rAAV virion), including flanking sequences (in AAV, inverted terminal repeats). The terms “expression cassette” and “polynucleotide cassette” refer to the portion of the vector genome between the flanking ITR sequences. “Expression cassette” implies that the vector genome comprises at least one gene encoding a gene product operatively linked to an element that drives expression (e.g., a promoter).

As used herein, the term “patient in need” or “subject in need” refers to a patient or subject at risk of, or suffering from, a disease, disorder or condition that is amenable to treatment or amelioration with a recombinant gene therapy vector or gene editing system disclosed herein. A patient or subject in need may, for instance, be a patient or subject diagnosed with a disorder associated with heart. A subject may have a mutation in an PKP2 gene or deletion of all or a part of PKP2 gene, or of gene regulatory sequences, that causes aberrant expression of the PKP2 protein. “Subject” and “patient” are used interchangeably herein. The subject treated by the methods described herein may be an adult or a child. Subjects may range in age.

As used herein, the term “variant” refers to a protein that has one or more amino-acid substitution, insertion, or deletion as compared to a parental protein. As used herein, the term “functional variant” refers to a protein that has one or more amino-acid substitution, insertion, or deletion as compared to a parental protein, and which retains one or more desired activities of the parental protein.

As used herein, “treating” refers to ameliorating one or more symptoms of a disease or disorder. The term “preventing” refers to delaying or interrupting the onset of one or more symptoms of a disease or disorder or slowing the progression of PKP2-related disease or disorder, e.g., arrhythmogenic cardiomyopathy (ACM).

Adeno-associated virus (AAV) is a replication-deficient parvovirus, the single-stranded DNA genome of which is about 4.7 kb in length including two ~145-nucleotide inverted terminal repeat (ITRs). There are multiple known variants of AAV. also sometimes called serotypes when classified by antigenic epitopes. The nucleotide sequences of the genomes of the AAV serotypes are known. For example, the complete genome of AAV-1 is provided in GenBank Accession No. NC_002077; the complete genome of AAV-2 is provided in GenBank Accession No. NC 001401 and Srivastava et al., J. Virol., 45: 555-564 (1983); the complete genome of AAV-3 is provided in GenBank Accession No. NC_1829: the complete genome of AAV-4 is provided in GenBank Accession No. NC_001829, the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos, AX753246 and AX753249, respectively; the AAV-9 genome is provided in Gao et al., J. Virol., 78: 6381-6388 (2004); the AAV-10 genome is provided in Mol. Ther., 13(1): 67-76 (2006); and the AAV-1I genome is provided in Virology, 330(2): 375-383 (2004). The sequence of the AAVrh.74 genome is provided in U.S. Pat. No. 9,434,928, incorporated herein by reference. Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep78, rep68, rep52, and rep40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA. To generate AAV vectors, the rep and cap proteins may be provided in trans. Another significant feature of AAV is that it is an extremely stable and hearty virus. It easily withstands the conditions used to inactivate adenovirus (56° C. to 65° C. for several hours), making cold preservation of AAV less critical. AAV may even be lyophilized. Finally, AAV-infected cells are not resistant to superinfection.

Gene delivery viral vectors useful in the practice of the present disclosure can be constructed utilizing methodologies well known in the art of molecular biology. Typically, viral vectors carrying transgenes are assembled from polynucleotides encoding the transgene, suitable regulatory elements and elements necessary for production of viral proteins, which mediate cell transduction. Such recombinant viruses may be produced by techniques known in the art, e.g. by transfecting packaging cells or by transient transfection with helper plasimids or viruses. Typical examples of virus packaging cells include but are not limited to HeLa cells, SF9 cells (optionally with a baculovirus helper vector), 293 cells, etc. A Herpesvirus-based system can be used to produce AAV vectors, as described in US20170218395A1. Detailed protocols for producing such replication-defective recombinant viruses may be found for instance in WO95/14785, WO96/22378, U.S. Pat. Nos. 5,882,877, 6,013,516, 4,861,719, 5,278,056 and WO94/19478, the complete contents of each of which is hereby incorporated by reference.

The present disclosure contemplates compositions and methods of use related to Plakophilin-2 (PKP2) proteins or polypeptides. Various mutations in PKP2 are known to be associated with cardiomyopathy and heart failure, including diseases like those described in van Tintelen et al. Circulation 113:1650-58 (2006); Novelli Front. Cardiovasc. Med. (2008); and in other sources. Viral vector-mediated delivery of the PKP2 gene may therefore serve as a viable therapeutic for PKP2-related human diseases such as cardiomyopathy and heart failure.

More than 230 mutations in the PKP2 gene have been identified in people with arrhythmogenic cardiomyopathy (A CM). (See “PKP2 gene,” MedlinePlus). This condition most commonly affects the myocardium surrounding the right ventricle, one of the two lower chambers of the heart. ACM increases the risk of an abnormal heartbeat (arrhythmia) and sudden death. Some PKP2 gene mutations lead to the production of an abnormally short version of plakophilin 2. Other mutations alter the structure of plakophilin 2 by adding, deleting, or changing one or more of its protein building blocks (amino acids). Studies suggest that the altered protein impairs the formation and function of desmosomes,

Without normal desmosomes, cells of the myocardium detach from one another and die, particularly when the heart muscle is placed under stress (such as during vigorous exercise). The damaged myocardium is gradually replaced by fat and scar tissue. As this abnormal tissue builds up, the walls of the right ventricle become stretched out, preventing the heart from pumping blood effectively. These changes also disrupt the electrical signals that control the heartbeat, which can lead to arrhythmia. Description of PKP2-related disease may be found in the following references: Bonne et al. Genomics 51:452-454 (1998) [PubMed: 9721216]; Bonne et al. Cytogenet. Cell Genet. 88:286-287 (2000) [PubMed: 10828611]; Dalal et al., Circulation 113:1641-1649 (2006) [Pub Med: 16549640]; Gerull et al. Nature Genet. 36:1162-1164 (2004) [PubMed: 15489853]; Grossmann et al. J Cell Biol. 167:149-160 (2004) [PubMed: 15479741]; Marcus et al. Circulation 65:384-398 (1982) [PubMed: 7053899]; and Mertens et al. J. Cell/Biol. 135:1009-1025 (1996) [PubMed: 8922383]. See also OMIM.org entry 602861 (“PLAKOPHILIN 2; PKP2”).

The native sequences of human PKP2a and its isoform PKP2b are shown below, with Arg-735 underlined:

PKP2a-837 amino acids
(SEQ ID NO: 1)

| | |
|---|---|
| 1 | MAAPGAPAEY GYIRTVLGQQ ILGQLDSSSL ALPSEAKLKL |
| 41 | AGSSGRGGQT VKSLRIQEQV QQTLARKGRS SVGNGNLHRT |
| 81 | SSVPEYVYNL HLVENDFVGG RSPVPKTYDM LKAGTTATYE |
| 121 | GRWGRGTAQY SSQKSVEERS LRHPLRRLEI SPDSSPERAH |
| 161 | YTHSDYQYSQ RSQAGHTLHH QESRRAALLV PPRYARSEIV |
| 201 | GVSRAGTTSR QRHFDTYHRQ YQHGSVSDTV FDSIPANPAL |
| 241 | LTYPRPGTSR SMGNLLEKEN YLTAGLTVGQ VRPLVPLQPV |
| 281 | TQNRASRSSW HQSSFHSTRT LREAGPSVAV DSSGRRAHLT |
| 321 | VGQAAAGGSG NLLTERSTFT DSQLGNADME MTLERAVSML |
| 361 | EADHMLPSRI SAAATFIQHE CFQKSEARKR VNQLRGILKL |
| 401 | LQLLKVQNED VQRAVCGALR NLVFEDNDNK LEVAELNGVP |
| 441 | RLLQVLKQTR DLETKKQITG LLWNLSSNDK LKNLMITEAL |
| 481 | LTLTENIIIP FSGWPEGDYP KANGLLDFDI FYNVTGCLRN |
| 521 | MSSAGADGRK AMRRCDGLID SLVHYVRGTI ADYQPDDKAT |
| 561 | ENCVCILHNL SYQLEAELPE KYSQNIYIQN RNIQTDNNKS |
| 601 | IGCFGSRSRK VKEQYQDVPM PEEKSNPKGV EWLWHSIVIR |
| 641 | MYLSLIAKSV RNYTQEASLG ALQNLTAGSG PMPTSVAQTV |
| 681 | VQKESGLQHT RKMLHVGDPS VKKTAISLLR NLSRNLSLQN |
| 721 | EIAKETLPDL VSIIPDTVPS TDLLIETTAS ACYTLNNIIQ |
| 761 | NSYQNARDLL NTGGIQKIMA ISAGDAYASN KASKAASVLL |
| 801 | YSLWAHTELH HAYKKAQFKK TDFVNSRTAK AYHSLKD |

PKP2b-881 amino acids
(SEQ ID NO: 2)

| | |
|---|---|
| 1 | MAAPGAPAEY GYIRTVLGQQ ILGQLDSSSL ALPSEAKLKL |
| 41 | AGSSGRGGQT VKSLRIQEQV QQTLARKGRS SVGNGNLHRT |
| 81 | SSVPEYVYNL HLVENDFVGG RSPVPKTYDM LKAGTTATYE |
| 121 | GRWGRGTAQY SSQKSVEERS LRHPLRRLEI SPDSSPERAH |
| 161 | YTHSDYQYSQ RSQAGHTLHH QESRRAALLV PPRYARSEIV |
| 201 | GVSRAGTTSR QRHFDTYHRQ YQHGSVSDTV FDSIPANPAL |
| 241 | LTYPRPGTSR SMGNLLEKEN YLTAGLTVGQ VRPLVPLQPV |
| 281 | TQNRASRSSW HQSSFHSTRT LREAGPSVAV DSSGRRAHLT |
| 321 | VGQAAAGGSG NLLTERSTFT DSQLGNADME MTLERAVSML |
| 361 | EADHMLPSRI SAAATFIQHE CFQKSEARKR VNQLRGILKL |
| 401 | LQLLKVQNED VQRAVCGALR NLVFEDNDNK LEVAELNGVP |
| 441 | RLLQVLKQTR DLETKKQITD HTVNLRSRNG WPGAVAHACN |
| 481 | PSTLGGQGGR ITRSGVRDQP DQHGLLWNLS SNDKLKNLMI |
| 521 | TEALLTLTEN IIIPFSGWPE GDYPKANGLL DFDIFYNVTG |
| 561 | CLRNMSSAGA DGRKAMRRCD GLIDSLVHYV RGTIADYQPD |
| 601 | DKATENCVCI LHNLSYQLEA ELPEKYSQNI YIQNRNIQTD |
| 641 | NNKSIGCFGS RSRKVKEQYQ DVPMPEEKSN PKGVEWLWHS |
| 681 | IVIRMYLSLI AKSVRNYTQE ASLGALQNLT AGSGPMPTSV |
| 721 | AQTVVQKESG LQHTRKMLHV GDPSVKKTAI SLLRNLSRNL |
| 761 | SLQNEIAKET LPDLVSIIPD TVPSTDLLIE TTASACYTLN |
| 801 | NIIQNSYQNA RDLLNTGGIQ KIMAISAGDA YASNKASKAA |
| 841 | SVLLYSLWAH TELHHAYKKA QFKKTDFVNS RTAKAYHSLK |
| 881 | D |

In some embodiments, the PKP2 protein comprises a polypeptide sequence at least or about 75%, at least 80%, at least or about 85%, at least or about 90%, at least or about 92%, at least or about 93%, at least or about 94%, at least or about 95%, at least or about 96%, at least or about 97%, at least or about 98%, at least or about 99%, or 100% identical to any one of SEQ ID NO: I or SEQ ID NO: 2. In some embodiments, the PKP2 protein is a wild-type or native PKP2 protein, e.g., human PKP2a or human PKP2b.

In some embodiments, the disclosure provides a recombinant adeno-associated virus (rAAV) virion, comprising a capsid and a vector genome, wherein the vector genome comprises a polynucleotide sequence encoding a PKP2 or a functional variant thereof, operatively linked to a promoter. In some embodiments, the disclosure provides a recombinant adeno-associated virus (rAAV) virion, comprising a capsid and a vector genome, wherein the vector genome comprises a polynucleotide sequence encoding an PKP2, operatively linked to a promoter.

In certain embodiments, the polynucleotide encoding the PKP2a may comprise a polynucleotide sequence at least or about 75%, at least or about 80%, at least or about 85%, at least or about 90%, at least or about 92%, at least or about 93%, at least 94%, at least or about 95%, at least or about 96%, at least or about 97%, at least or about 98%, at least or about 99%, or 100% identical to SEQ ID NO: 3.

In certain embodiments, the polynucleotide encoding the PKP2b may comprise a polynucleotide sequence at least or about 75%, at least or about 80%, at least or about 85%, at least or about 90%, at least or about 92%, at least or about 93%, at least or about 94%, at least or about 95%, at least or about 96%, at least or about 97%, at least or about 98%, 9 at least or about 9%, or 100% identical to SEQ ID NO: 4.

Optionally, the polynucleotide sequence encoding the vector genome may comprise a Kozak sequence, including but not limited to GCCACCATGG (SEQ ID NO: 5). Kozak sequence may overlap the polynucleotide sequence encoding an PKP2a protein or a functional variant thereof. For example, the vector genome may comprise a polynucleotide sequence (with first ten nucleotides constituting the Kozak sequence) at least or about 75%, at least or about 80%, at least or about 85%, at least or about 90%, at least or about 92%, at least or about 93%, at least or about 94%, at least or about 95%, at least or about 96%, at least or about 97%, at least or about 98%, at least or about 99%, or 100% identical to SEQ ID NO: 6.

In certain embodiment, a Kozak sequence may overlap the polynucleotide sequence encoding a PKP2 (e.g., PKP2a or PKP2b) protein or a functional variant thereof. For example, the vector genome may comprise a polynucleotide sequence (with first ten nucleotides constituting the Kozak sequence) at least or about 75%, at least or about 80%, at least or about 85% at least or about 90%, at least or about 92%, at least or about 93%, at least or about 94%, at least or about 95%, at least or about 96%, at least or about 97%, at least or about 98%, at least or about 99%, or 100% identical to SEQ ID NO: 7.

In some embodiments, the Kozak sequence is an alternative Kozak sequence comprising or consisting of any one of:

(SEQ ID NO: 16)
(gcc)gccRccAUGG;
AGNNAUGN;
ANNAUGG;
ANNAUGC;
ACCAUGG;
and
(SEQ ID NO: 18)
GACACCAUGG.

In some embodiments, the vector genome comprises no Kozak sequence.

The polynucleotide sequence may be codon-optimized. For example, the vector genome may comprise a polynucleotide sequence encoding a PKP2a that shares at least or about 75%, at least or about 80%, at least or about 85%, at least or about 90%, at least or about 92%, at least or about 93%, at least or about 94%, at least or about 95%, at least or about 96%, at least or about 97%, at least or about 98%, at least or about 99%, or 100% identity to SEQ ID NO: 87. The vector genome may comprise a polynucleotide sequence encoding a PKP2b that shares at least or about 75%, at least or about 80%, at least or about 85%, at least or about 90%, at least or about 92%, at least or about 93%, at least or about 94%, at least or about 95%, at least or about 96%, at least or about 97%, at least or about 98%, at least or about 99%, or 100% identity to SEQ ID NO: 88.

The AAV virions of the disclosure comprise a vector genome. The vector genome may comprise an expression cassette (or a polynucleotide cassette for gene-editing applications not requiring expression of the polynucleotide sequence). Any suitable inverted terminal repeats (ITRs) may be used. The ITRs may be AAV IRs from the same serotype as the capsid present in the AAV virion, or a different serotype from the capsid (e.g., AAV2 ITRs may be used with an AAV virion having an AAV9 capsid or an AAVrh.74 capsid). In each case, the serotype of the capsid determines the name applied to the virion. The ITR are generally the most 5′ and most 3′ elements of the vector genome. The vector genome will also generally contain, in 5′ to 3′ order, a promoter, a transgene, 3′ untranslated region (UTR) sequences (e.g., a WPRE element), and a polyadenylation sequence. In variations, the vector genome includes an enhancer element (generally 5′ to the promoter) and/or an exon (generally 3′ to the promoter). In variations, the vector genomes of the disclosure encode a partial or complete transgene sequence used as a repair template in a gene editing system. In such variations, the vector genome may comprise an exogenous promoter, or the gene editing system may insert the transgene into a locus in the genome having an endogenous promoter, such as a cardiac- or myocyte-specific promoter.

In some embodiments, the 5′ ITR comprises a polynucleotide sequence at least or about 75%, at least or about 80%, at least or about 85%, at least or about 90%, at least or about 92%, at least or about 93%, at least or about 94%, at least or about 95%, at least or about 96%, at least or about 97%, at least or about 98%, at least or about 99%, or 100% identical to SEQ ID NO: 20.

In some embodiments, the 5′ ITR comprises a polynucleotide sequence at least or about 75%, at least or about 80%, at least or about 85%, at least or about 90%, at least or about 92%, at least or about 93%, at least or about 94%, at least or about 95%, at least or about 96%, at least or about 97% at least or about 98%, at least or about 99%, or 100% identical to SEQ ID NO: 21

In some embodiments, the 5′ ITR comprises a polynucleotide sequence at least or about 75%, at least or about 80%, at least or about 85%, at least or about 90%, at least or about 92%, at least or about 93%, at least or about 94%, at least or about 95%, at least or about 96%, at least or about 97% at least or about 98%, at least or about 99%, or 100% identical to SEQ ID NO: 22)

In some embodiments, the 5′ ITR comprises a polynucleotide sequence at least or about 75%, at least or about 80%, at least or about 85%, at least or about 90%, at least or about 92%, at least or about 93%, at least or about 94%, at least or about 95%, at least or about 96%, at least or about 97% at least or about 98%, at least or about 99%, or 100% identical to SEQ ID NO: 23

In some embodiments, the 3′ ITR comprises a polynucleotide sequence at least or about 75%, at least or about 80%, at least or about 85%, at least or about 90%, at least or about 92%, at least or about 93%, at least or about 94%, at least or about 95%, at least or about 96%, at least or about 97% at least or about 98%, at least or about 99%, or 100% identical to SEQ ID NO: 24,

In some embodiments, the 3 ITR comprises a polynucleotide sequence at least or about 75%, at least or about 80%, at least or about 85%, at least or about 90%, at least or about 92%, at least or about 93%, at least or about 94%, at least or about 95%, at least or about 96%, at least or about 97%, at least or about 98%, at least or about 99%, or 100% identical to SEQ ID NO: 25.

In some embodiments, the 3 ITR comprises a polynucleotide sequence at least or about 75%, at least or about 80%, at least or about 85%, at least or about 90%, at least or about 92%, at least or about 93%, at least or about 94%, at least or about 95%, at least or about 96%, at least or about 97%, at least or about 98%, at least or about 99%, or 100% identical to SEQ ID NO: 26.

In some embodiments the vector genome comprises one or more filler sequences, e.g., at least or about 75%, at least or about 80%, at least or about 85%, at least or about 90%, at least or about 92%, at least or about 93%, at least or about 94%, at least or about 95%, at least or about 96%, at least or about 97%, at least or about 98%, at least or about 99%, or 100% identical to SEQ ID NO: 27; SEQ ID NO: 28; or SEQ ID NO: 29.

In some embodiments, the polynucleotide sequence encoding an PKP2 protein or functional variant thereof is operatively linked to a promoter. In certain embodiments, the promoter is an MHCK7 promoter. In certain embodiments, the promoter is an TNNT2 promoter.

The present disclosure contemplates use of various promoters. Promoters useful in embodiments of the present disclosure include, without limitation, a cytomegalovirus (CMV) promoter, phosphoglycerate kinase (PGK) promoter, or a promoter sequence comprised of the CMV enhancer and portions of the chicken beta-actin promoter and the rabbit beta-globin gene (CAG). In some cases, the promoter may be a synthetic promoter. Exemplary synthetic promoters are provided by Schlabach et al. PNAS USA. 107(6):2538-43 (2010). In some embodiments, the promoter comprises a polynucleotide sequence at least or about 75%, at least or about 80%, at least or about 85%, at least or about 90%, at least or about 92%, at least or about 93%, at least or about 94%, at least or about 95%, at least or about 96%, at least or about 97%, at least or about 98%, at least or about 99%, or 100% identical to SEQ ID NO: 30.

In some embodiments, a polynucleotide sequence encoding an PKP2 protein or functional variant thereof is operatively linked to an inducible promoter. An inducible promoter may be configured to cause the polynucleotide sequence to be transcriptionally expressed or not transcriptionally expressed in response to addition or accumulation of an agent or in response to removal, degradation, or dilution of an agent. The agent may be a drug. The agent may be tetracycline or one of its derivatives, including, without limitation, doxycycline. In some cases, the inducible promoter is a tert-on promoter, a tet-off promoter, a chemically-regulated promoter, a physically-regulated promoter (i.e., a promoter that responds to presence or absence of light or to low or high temperature). Inducible promoters include heavy metal ion inducible promoters (such as the mouse mammary tumor virus (mMTV) promoter or various growth hormone promoters), and the promoters from T7 phage which are active in the presence of T7 RNA polymerase. This list of inducible promoters is non-limiting.

In some cases, the promoter is a tissue-specific promoter, such as a promoter capable of driving expression in a cardiac cell to a greater extent than in a non-cardiac cell. In some embodiments, tissue-specific promoter is a selected from any various cardiac cell-specific promoters including but not limited to, desmin (Des), alpha-myosin heavy chain (α-MHC), myosin light chain 2 (MLC-2), cardiac troponin C (cTnC), cardiac troponin T (hTNNT2), muscle creatine kinase (CK) and combinations of promoter/enhancer regions thereof, such as MHCK7. In some cases, the promoter is a ubiquitous promoter. A “ubiquitous promoter” refers to a promoter that is not tissue-specific under experimental or clinical conditions. In some cases, the ubiquitous promoter is any one of Cytomegalovirus (CMV), Cytomegalovirus early enhancer element chicken beta-Actin gene intron with the splice acceptor of the rabbit beta-Globin gene (CAG), ubiquitin C (UBC), Phosphoglycerate Kinase (PGK), Eukaryotic translation elongation factor I alpha I (EF1-alpha), Glyceraldehyde 3-phosphate dehydrogenase (GAPDH), simian virus 40 (SV40). Hepatitis B virus (HBV), chicken beta-actin, and human beta-actin promoters.

In some embodiments, the promoter sequence is selected from Table 3. In some embodiments, the promoter comprises a polynucleotide sequence at least or about 75%, at least or about 80%, at least or about 85%, at least or about 90%, at least or about 92%, at least or about 93%, at least or about 94%, at least or about 95%, at least or about 96%, at least or about 97%, at least or about 98%, at least or about 99%, or 100% identical to any one of SEQ ID NOS: 31-51. In some embodiments, the promoter comprises a fragment of a polynucleotide sequence of any one of SEQ ID NOs: 31-48, e.g., a fragment comprising at least 25% at least 50%, at least 75%, 80%, 85%, 90%, 92%, 93%, 94%, 95%, 96%, 97%, 98%, 99%, or 100%, of any one of SEQ ID NOs: 31-48.

TABLE 3

| | | SEQ ID |
|---|---|---|
| PROMOTER | SEQUENCE | NO: |
| MHCK7 | ACCCTTCAGATTAAAAATAACTGAGGTAAGGGCCTGGGTAG | 31 |
| | GGGAGGTGGTGTGAGACGCTCCTGTCTCTCCTCTATCTGCCC | |
| | ATCGGCCCTTTGGGGAGGAGGAATGTGCCCAAGGACTAAAA | |
| | AAAGGCCATGGAGCCAGAGGGGCGAGGGCAACAGACCTTTC | |
| | ATGGGCAAACCTTGGGGCCCTGCTGTCTAGCATGCCCCACTA | |
| | CGGGTCTAGGCTGCCCATGTAAGGAGGCAAGGCCTGGGGAC | |
| | ACCCGAGATGCCTGGTTATAATTAACCCAGACATGTGGCTGC | |
| | CCCCCCCCCCCCAACACCTGCTGCCTCTAAAAATAACCCTGT | |
| | CCCTGGTGGATCCCCTGCATGCGAAGATCTTCGAACAAGGCT | |
| | GTGGGGGACTGAGGGCAGGCTGTAACAGGCTTGGGGGCCAG | |
| | GGCTTATACGTGCCTGGGACTCCCAAAGTATTACTGTTCCAT | |
| | GTTCCCGGCGAAGGGCCAGCTGTCCCCCGCCAGCTAGACTCA | |
| | GCACTTAGTTTAGGAACCAGTGAGCAAGTCAGCCCTTGGGGC | |
| | AGCCCATACAAGGCCATGGGGCTGGGCAAGCTGCACGCCTG | |
| | GGTCCGGGGTGGGCACGGTGCCCGGGCAACGAGCTGAAAGC | |
| | TCATCTGCTCTCAGGGGCCCCTCCCTGGGGACAGCCCCTCCT | |
| | GGCTAGTCACACCCTGTAGGCTCCTCTATATAACCCAGGGGC | |
| | ACAGGGGCTGCCCTCATTCTACCACCACCTCCACAGCACAGA | |
| | CAGACACTCAGGAGCCAGCCAG | |
| Human cardiac | CTCAGTCCATTAGGAGCCAGTAGCCTGGAAGATGTCTTTACC | 33 |
| troponin T | CCCAGCATCAGTTCAAGTGGAGCAGCACATAACTCTTGCCCT | |
| promoter | CTGCCTTCCAAGATTCTGGTGCTGAGACTTATGGAGTGTCTT | |
| (without exon 1) | GGAGGTTGCCTTCTGCCCCCCAACCCTGCTCCCAGCTGGCCC | |
| hTnnT2/HTNNT2 | TCCCAGGCCTGGGTTGCTGGCCTCTGCTTTATCAGGATTCTCA | |
| | AGAGGGACAGCTGGTTTATGTTGCATGACTGTTCCCTGCATA | |
| | TCTGCTCTGGTTTTAAATAGCTTATCTGAGCAGCTGGAGGAC | |
| | CACATGGGCTTATATGGCGTGGGGTACATGTTCCTGTAGCCT | |
| | TGTCCCTGGCACCTGCCAAAATAGCAGCCAACACCCCCCACC | |
| | CCCACCGCCATCCCCCTGCCCCACCCGTCCCCTGTCGCACAT | |
| | TCCTCCCTCCGCAGGGCTGGCTCACCAGGCCCCAGCCCACAT | |
| | GCCTGCTTAAAGCCCTCTCCATCCTCTGCCTCACCCAGT | |
| Human cardiac | CTCAGTCCATTAGGAGCCAGTAGCCTGGAAGATGTCTTTACC | 32 |
| troponin T | CCCAGCATCAGTTCAAGTGGAGCAGCACATAACTCTTGCCCT | |
| promoter (with | CTGCCTTCCAAGATTCTGGTGCTGAGACTTATGGAGTGTCTT | |
| exon 1, | GGAGGTTGCCTTCTGCCCCCCAACCCTGCTCCCAGCTGGCCC | |
| underlined) | TCCCAGGCCTGGGTTGCTGGCCTCTGCTTTATCAGGATTCTCA | |
| hTnnT2/HTNNT2 | AGAGGGACAGCTGGTTTATGTTGCATGACTGTTCCCTGCATA | |
| | TCTGCTCTGGTTTTAAATAGCTTATCTGAGCAGCTGGAGGAC | |
| | CACATGGGCTTATATGGCGTGGGGTACATGTTCCTGTAGCCT | |
| | TGTCCCTGGCACCTGCCAAAATAGCAGCCAACACCCCCCACC | |
| | CCCACCGCCATCCCCCTGCCCCACCCGTCCCCTGTCGCACAT | |
| | TCCTCCCTCCGCAGGGCTGGCTCACCAGGCCCCAGCCCACAT | |
| | GCCTGCTTAAAGCCCTCTCCATCCTCTGCCTCACCCAGTCCCC | |
| | GCTGAGACTGAGCAGACGCCTCCAGGATCTGTCGGCAG | |
| Mouse α- | GGTACCGGATCCTGCAAGGTCACACAAGGGTCTCCACCCACC | 34 |
| cardiac myosin | AGGTGCCCTAGTCTCAATTTCAGTTTCCATGCCTTGTTCTCAC | |
| heavy chain | AATGCTGGCCTCCCCAGAGCTAATTTGGACTTTGTTTTTATTT | |
| promoter | CAAAAGGGCCTGAATGAGGAGTAGATCTTGTGCTACCCAGC | |
| (αMHC) | TCTAAGGGTGCCCGTGAAGCCCTCAGACCTGGAGCCTTTGCA | |
| | ACAGCCCTTTAGGTGGAAGCAGAATAAAGCAATTTTCCTTAA | |
| | AGCCAAAATCCTGCCTCTAGACTCTTCTTCTCTGACCTCGGTC | |
| | CCTGGGCTCTAGGGTGGGGAGGTGGGGCTTGGAAGAAGAAG | |
| | GTGGGGAAGTGGCAAAAGCCGATCCCTAGGGCCCTGTGAAG | |
| | TTCGGAGCCTTCCCTGTACAGCACTGGCTCATAGATCCTCCT | |
| | CCAGCCAAACATAGCAAGAAGTGATACCTCCTTTGTGACTTC | |
| | CCCAGGCCCAGTACCTGTCAGGTTGAAACAGGATTTAGAGA | |
| | AGCCTCTGAACTCACCTGAACTCTGAAGCTCATCCACCAAGC | |
| | AAGCACCTAGGTGCCACTGCTAGTTAGTATCCTACGCTGATA | |
| | ATATGCAGAGCTGGGCCACAGAAGTCCTGGGGTGTAGGAAC | |
| | TGACCAGTGACTTTTCAGTCGGCAAAGGTATGACCCCCTCAG | |
| | CAGATGTAGTAATGTCCCCTTAGATCCCATCCCAGGCAGGTC | |
| | TCTAAGAGGACATGGGATGAGAGATGTAGTCATGTGGCATT | |
| | CCAAACACAGCTATCCACAGTGTCCCTTGCCCCTTCCACTTA | |
| | GCCAGGAGGACAGTAACCTTAGCCTATCTTTCTTCCTCCCCA | |
| | TCCTCCCAGGACACACCCCCTGGTCTGCAGTATTCATTTCTTC | |
| | CTTCACGTCCCCTCTGTGACTTCCATTTGCAAGGCTTTTGACC | |
| | TCTGCAGCTGCTGGAAGATAGAGTTTGGCCCTAGGTGTGGCA | |
| | AGCCATCTCAAGAGAAAGCAGACAACAGGGGGACCAGATTT | |
| | TGGAAGGATCAGGAACTAAATCACTGGCGGGCCTGGGGGTA | |
| | GAAAAAAGAGTGAGTGAGTCCGCTCCAGCTAAGCCAAGCTA | |
| | GTCCCCGAGATACTCTGCCACAGCTGGGCTGCTCGGGGTAGC | |
| | TTTAGGAATGTGGGTCTGAAAGACAATGGGATTGGAAGACA | |
| | TCTCTTTGAGTCTCCCCTCAACCCCACCTACAGACACACTCGT | |
| | GTGTGGCCAGACTCCTGTTCAACAGCCCTCTGTGTTCTGACC | |
| | ACTGAGCTAGGCAACCAGAGCATGGGCCCTGTGCTGAGGAT | |
| | GAAGAGTTGGTTACCAATAGCAAAAACAGCAGGGGAGGGAG | |
| | AACAGAGAACGAAATAAGGAAGGAAGAAGGAAAGGCCAGT | |
| | CAATCAGATGCAGTCAGAAGAGATGGGAAGCCAACACACAG | |
| | CTTGAGCAGAGGAAACAGAAAAGGGAGAGATTCTGGGCATA | |
| | AGGAGGCCACAGAAAGAAGAGCCCAGGCCCCCCAAGTCTCC | |
| | TCTTTATACCCTCATCCCGTCTCCCAATTAAGCCCACTCTTCT | |
| | TCCTAGATCAGACCTGAGCTGCAGCGAAGAGACCCGTAGGG | |
| | AGGATCACACTGGATGAAGGAGATGTGTGGAGAAGTCCAGG | |
| | GAACCTAAGAGCCAGAGCCTAAAAGAGCAAGAGATAAAGGT | |
| | GCTTCAAAGGTGGCCAGGCTGTGCACACAGAGGGTCGAGGA | |
| | CTGGTGGTAGAGCCTCAAGATAAGGATGATGCTCAGAATGG | |
| | GCGGGGGGGGGGATTCTGGGGGGGGGAGAGAGAAGGTGAG | |
| | AAGGAGCCTGGAACAGAGAATCTGGAAGCGCTGGAAACGAT | |
| | ACCATAAAGGGAAGAACCCAGGCTACCTTTAGATGTAAATC | |
| | ATGAAAGACAGGGAGAAGGGAAGCTGGAGAGAGTAGAAGG | |
| | ACCCCGGGGCAAGACATTGAAGCAAGGACAAGCCAGGTTGA | |
| | GCGCTCCGTGAAATCAGCCTGCTGAAGGCAGAGCCCTGGTAT | |
| | GAGCACCAGAACAGCAGAGGCTAGGGTTAATGTCGAGACAG | |
| | GGAACAGAAGGTAGACACAGGAACAGACAGAGACGGGGGA | |
| | GCCAGGTAACAAAGGAATGGTCCTTCTCACCTGTGGCCAGA | |
| | GCGTCCATCTGTGTCCACATACTCTAGAATGTTCATCAGACT | |
| | GCAGGGCTGGCTTGGGAGGCAGCTGGAAAGAGTATGTGAGA | |
| | GCCAGGGGAGACAAGGGGGCCTAGGAAAGGAAGAAGAGGG | |
| | CAAACCAGGCCACACAAGAGGGCAGAGCCCAGAACTGAGTT | |
| | AACTCCTTCCTTGTTGCATCTTCCATAGGAGGCAGTGGGAAC | |
| | TCTGTGACCACCATCCCCCATGAGCCCCCACTACCCATACCA | |
| | AGTTTGGCCTGAGTGGCATTCTAGGTTCCCTGAGGACAGAGC | |
| | CTGGCCTTTGTCTCTTGGACCTGACCCAAGCTGACCCAATGT | |
| | TCTCAGTACCTTATCATGCCCTCAAGAGCTTGAGAACCAGGC | |
| | AGTGACATATTAGGCCATGGGCTAACCCTGGAGCTTGCACAC | |
| | AGGAGCCTCAAGTGACCTCCAGGGACACAGCTGCAGACAGG | |
| | TGGCCTTTATCCCCAAAGAGCAACCATTIGGCATAGGTGGCT | |
| | GCAAATGGGAATGCAAGGTTGAATCAGGTCCCTTCAAGAAT | |
| | ACTGCATGCAAGACCTAAGACCCCTGGAGAGAGGGGTATGC | |
| | TCCTGCCCCCACCCACCATAAGGGGAGTGAACTATCCTAGGG | |
| | GGCTGGCGACCTTGGGGAGACACCACATTACTGAGAGTGCT | |
| | GAGCCCAGAAAAACTGACCGCCCTGTGTCCTGCCCACCTCCA | |
| | CACTCTAGAGCTATATTGAGAGGTGACAGTAGATAGGGTGG | |
| | GAGCTGGTAGCAGGGAGAGTGTTCCTGGGTGTGAGGGTGTA | |
| | GGGGAAAGCCAGAGCAGGGGAGTCTGGCTTTGTCTCCTGAA | |
| | CACAATGTCTACTTAGTTATAACAGGCATGACCTGCTAAAGA | |
| | CCCAACATCTACGACCTCTGAAAAGACAGCAGCCCTGGAGG | |
| | ACAGGGGTTGTCTCTGAGCCTTGGGTGCTTGATGGTGCCACA | |
| | AAGGAGGGCATGAGTGTGAGTATAAGGCCCCAGGAGCGTTA | |
| | GAGAAGGGCACTTGGGAAGGGGTCAGTCTGCAGAGCCCCTA | |
| | TCCATGGAATCTGGAGCCTGGGGCCAACTGGTGTAAATCTCT | |
| | GGGCCTGCCAGGCATTCAAAGCAGCACCTGCATCCTCTGGCA | |
| | GCCTGGGGAGGCGGAAGGGAGCAACCCCCCACTTATACCCT | |
| | TTCTCCCTCAGCCCCAGGATTAACACCTCTGGCCTTCCCCCTT | |
| | CCCACCTCCCATCAGGAGTGGAGGGTTGCAGAGGGAGGGTA | |
| | AAAACCTACATGTCCAAACATCATGGTGCACGATATATGGAT | |
| | CAGTATGTGTAGAGGCAAGAAAGGAAATCTGCAGGCTTAAC | |
| | TGGGTTAATGTGTAAAGTCTGTGTGCATGTGTGTGTGTCTGA | |
| | CTGAAAACGGGCATGGCTGTGCAGCTGTTCAGTTCTGTGCGT | |
| | GAGGTTACCAGACTGCAGGTTTGTGTGTAAATTGCCCAAGGC | |
| | AAAGTGGGTGAATCCCTTCCATGGTTTAAAGAGATTGGATGA | |
| | TGGCCTGCATCTCAAGGACCATGGAAAATAGAATGGACACT | |
| | CTATATGTGTCTCTAAGCTAAGGTAGCAAGGTCTTTGGAGGA | |
| | CACCTGTCTAGAGATGTGGGCAACAGAGACTACAGACAGTA | |
| | TCTGTACAGAGTAAGGAGAGAGAGGAGGGGGTGTAGAATTC | |
| | TCTTACTATCAAAGGGAAACTGAGTCGTGCACCTGCAAAGTG | |
| | GATGCTCTCCCTAGACATCATGACTTTGTCTCTGGGGAGCCA | |
| | GCACTGTGGAACTTCAGGTCTGAGAGAGTAGGAGGCTCCCCT | |
| | CAGCCTGAAGCTATGCAGATAGCCAGGGTTGAAAGGGGGAA | |
| | GGGAGAGCCTGGGATGGGAGCTTGTGTGTTGGAGGCAGGGG | |
| | ACAGATATTAAGCCTGGAAGAGAAGGTGACCCTTACCCAGT | |
| | TGTTCAACTCACCCTTCAGATTAAAAATAACTGAGGTAAGGG | |
| | CCTGGGTAGGGGAGGTGGTGTGAGACGCTCCTGTCTCTCCTC | |
| | TATCTGCCCATCGGCCCTTTGGGGAGGAGGAATGTGCCCAAG | |
| | GACTAAAAAAAGGCCATGGAGCCAGAGGGGCGAGGGCAAC | |
| | AGACCTTTCATGGGCAAACCTTGGGGCCCTGCTGTCCTCCTG | |
| | TCACCTCCAGAGCCAAGGGATCAAAGGAGGAGGAGCCAGGA | |
| | CAGGAGGGAAGTGGGAGGGAGGGTCCCAGCAGAGGACTCC | |
| | AAATTTAGGCAGCAGGCATATGGGATGGGATATAAAGGGGC | |
| | TGGAGCACTGAGAGCTGTCAGAGATTTCTCCAACCCAGGTAA | |
| | GAGGGAGTTTCGGGTGGGGGCTCTTCACCCACACCAGACCTC | |
| | TCCCCACCTAGAAGGAAACTGCCTTTCCTGGAAGTGGGGTTC | |
| | AGGCCGGTCAGAGATCTGACAGGGTGGCCTTCCACCAGCCT | |
| | GGGAAGTTCTCAGTGGCAGGAGGTTTCCACAAGAAACACTG | |
| | GATGCCCCTTCCCTTACGCTGTCTTCTCCATCTTCCTCCTGGG | |
| | GATGCTCCTCCCCGTCTTGGTTTATCTTGGCTCTTCGTCTTCA | |
| | GCAAGATTTGCCCTGTGCTGTCCACTCCATCTTTCTCTACTGT | |
| | CTCCGTGCCTTGCCTTGCCTTCTTGCGTGTCCTTCCTTTCCAC | |
| | CCATTTCTCACTTCACCTTTTCTCCCCTTCTCATTTGTATTCAT | |
| | CCTTCCTTCCTTCCTTCCTTCCTTCCTTCCTTCCTTCCTTCCTTC | |
| | CTTTCTCCCTTCCTTCCTTCCTTCCTTCCTTCCTTCCTTCCTTCC | |
| | TTCCTGTGTCAGAGTGCTGAGAATCACACCTGGGGTTCCCAC | |
| | CCTTATGTAAACAATCTTCCAGTGAGCCACAGCTTCAGTGCT | |
| | GCTGGGTGCTCTCTTACCTTCCTCACCCCCTGGCTTGTCCTGT | |
| | TCCATCCTGGTCAGGATCTCTAGATTGGTCTCCCAGCCTCTGC | |
| | TACTCCTCTTCCTGCCTGTTCCTCTCTCTGTCCAGCTGCGCCA | |
| | CTGTGGTGCCTCGTTCCAGCTGTGGTCCACATTCTTCAGGATT | |
| | CTCTGAAAAGTTAACCAGGTGAGAATGTTTCCCCTGTAGACA | |
| | GCAGATCACGATTCTCCCGGAAGTCAGGCTTCCAGCCCTCTC | |
| | TTTCTCTGCCCAGCTGCCCGGCACTCTTAGCAAACCTCAGGC | |
| | ACCCTTACCCCACATAGACCTCTGACAGAGAAGCAGGCACTT | |
| | TACATGGAGTCCTGGTGGGAGAGCCATAGGCTACGGTGTAA | |
| | AAGAGGCAGGGAAGTGGTGGTGTAGGAAAGTCAGGACTTCA | |
| | CATAGAAGCCTAGCCCACACCAGAAATGACAGACAGATCCC | |
| | TCCTATCTCCCCCATAAGAGTTTGAGTCGACCCGCGGCCCCG | |
| | AATTG | |
| Chicken cardiac | GGGATAAAAGCAGTCTGGGCTTTCACATGACAGCATCTGGG | 35 |
| troponin T | GCTGCGGCAGAGGGTCGGGTCCGAAGCGCTGCCTTATCAGC | |
| promoter | GTCCCCAGCCCTGGGAGGTGACAGCTGGCTGGCTTGTGTCAG | |
| (cTnT) | CCCCTCGGGCACTCACGTATCTCCGTCCGACGGGTTTAAAAT | |
| | AGCAAAACTCTGAGGCCACACAATAGCTTGGGCTTATATGG | |
| | GCTCCTGTGGGGGAAGGGGGAGCACGGAGGGGGCCGGGGCC | |
| | GCTGCTGCCAAAATAGCAGCTCACAAGTGTTGCATTCCTCTC | |
| | TGGGCGCCGGGCACATTCCTGCTGGCTCTGCCCGCCCCGGGG | |
| | TGGGCGCCGGGGGGACCTTAAAGCCTCTGCCCCCCAAGGAG | |
| | CCCTTCCCAGACAGCCGCCGGCACCCACCGCTCCGTGGGA | |
| Human Creatine | CTCTCAGCCCTGGAAGTCCTTGCTCACAGCCGAGGCGCCGAG | 36 |
| Kinase M | AGCGCTTGCTCTGCCCAGATCTGCGCGAGTCTGGCGCCCGCG | |
| (hCKM) | CTCTGAACGGCGTCGCTGCCCAGCCCCCTTCCCCGGGAGGTG | |
| | GGAGCGGCCACCCAGGGCCCCGTGGCTGCCCTTGTAAGGAG | |
| | GCGAGGCCCGAGGACACCCGAGACGCCCGGTTATAATTAAC | |
| | CAGGACACGTGGCGAACCCCCCTCCAACACCTGCCCCCGAA | |
| | CCCCCCCATACCCAGCGCCTCGGGTCTCGGCCTTTGCGGCAG | |
| | AGGAGACAGCAAAGCGCCCTCTAAAAATAACTCCTTTCCCG | |
| | GCGACCGAGACCCTCCCTGTCCCCCGCACAGCGGAAATCTCC | |
| | CAGTGGCACCGAGGGGGCGAGGGTTAAGTGGGGGGGAGGGT | |
| | GACCACCGCCTCCCACCCTTGCCCTGAGTTTGAATCTCTCCA | |
| | ACTCAGCCAGCCTCAGTTTCCCCTCCACTCAGTCCCTAGGAG | |
| | GAAGGGGCGCCCAAGCGCGGGTTTCTGGGGTTAGACTGCCC | |
| | TCCATTGCAATTGGTCCTTCTCCCGGCCTCTGCTTCCTCCAGC | |
| | TCACAGGGTATCTGCTCCTCCTGGAGCCACACCTTGGTTCCC | |
| | CGAGGTGCCGCTGGGACTCGGGTAGGGGTGAGGGCCCAGGG | |
| | GGCACAGGGGGAGCCGAGGGCCACAGGAAGGGCTGGTGGCT | |
| | GAAGGAGACTCAGGGGCCAGGGGACGGTGGCTTCTACGTGC | |
| | TTGGGACGTTCCCAGCCACCGTCCCATGTTCCCGGGGGGGG | |
| | CCAGCTGTCCCCACCGCCAGCCCAACTCAGCACTTGGTCAGG | |
| | GTATCAGCTTGGTGGGGGGGCGTGAGCCCAGCCCCTGGGGC | |
| | GGCTCAGCCCATACAAGGCCATGGGGCTGGGCGCAAAGCAT | |
| | GCCTGGGTTCAGGGTGGGTATGGTGCGGGAGCAGGGAGGTG | |
| | AGAGGCTCAGCTGCCCTCCAGAACTCCTCCCTGGGGACAACC | |
| | CCTCCCAGCCAATAGCACAGCCTAGGTCCCCCTATATAAGGC | |
| | CACGGCTGCTGGCCCTTCCTTTGGGTCAGTGTCACCTCCAGG | |
| | ATACAGACA | |
| Human beta- | GCCCAGCACCCCAAGGCGGCCAACGCCAAAACTCTCCCTCCT | 37 |
| actin (HuBa) | CCTCTTCCTCAATCTCGCTCTCGCTCTTTTTTTTTTTCGCAAAA | |
| | GGAGGGGAGAGGGGGTAAAAAAATGCTGCACTGTGCGGCGA | |
| | AGCCGGTGAGTGAGCGGCGCGGGGCCAATCAGCGTGCGCCG | |
| | TTCCGAAAGTTGCCTTTTATGGCTCGAGCGGCCGCGGCGGCG | |
| | CCCTATAAAACCCAGCGGCGCGACGCGCCACCACCGCCGAG | |
| | TC | |
| Chicken beta- | GGTCGAGGTGAGCCCCACGTTCTGCTTCACTCTCCCCATCTC | 38 |
| actin (CBA) | CCCCCCCTCCCCACCCCCAATTTTGTATTTATTTATTTTTTAAT | |
| | TATTTTGTGCAGCGATGGGGGCGGGGGGGGGGGGGGGCGCGC | |
| | GCCAGGCGGGGCGGGGCGGGGCGAGGGCGGGGGGCGGGGCG | |
| | AGGCGGAGAGGTGCGGCGGCAGCCAATCAGAGCGGCGCGCT | |
| | CCGAAAGTTTCCTTTTATGGCGAGGCGGCGGCGGCGGCGGCC | |
| | CTATAAAAAGCGAAGCGCGCGGCGGGCGGGGA | |
| Cytomegalovirus | TGGTGATGCGGTTTTGGCAGTACACCAATGGGCGTGGATAGC | 39 |
| (CMV) | GGTTTGACTCACGGGGATTTCCAAGTCTCCACCCCATTGACG | |
| | TCAATGGGAGTTTGTTTTGGCACCAAAATCAACGGGACTTTC | |
| | CAAAATGTCGTAATAACCCCGCCCCGTTGACGCAAATGGGC | |
| | GGTAGGCGTGTACGGTGGGAGGTCTATATAAGCAGAGCTCG | |
| | TTTAGTGAACCG | |
| Cytomegalovirus | TAGTTATTAATAGTAATCAATTACGGGGTCATTAGTTCATAG | 40 |
| (CMV) | CCCATATATGGAGTTCCGCGTTACATAACTTACGGTAAATGG | |
| (second version) | CCCGCCTGGCTGACCGCCCAACGACCCCCGCCCATTGACGTC | |
| | AATAATGACGTATGTTCCCATAGTAACGCCAATAGGGACTTT | |
| | CCATTGACGTCAATGGGTGGAGTATTTACGGTAAACTGCCCA | |
| | CTTGGCAGTACATCAAGTGTATCATATGCCAAGTACGCCCCC | |
| | TATTGACGTCAATGACGGTAAATGGCCCGCCTGGCATTATGC | |
| | CCAGTACATGACCTTATGGGACTTTCCTACTTGGCAGTACAT | |
| | CTACGTATTAGTCATCGCTATTACCATGGTGATGCGGTTTTG | |
| | GCAGTACATCAATGGGCGTGGATAGCGGTTTGACTCACGGG | |
| | GATTTCCAAGTCTCCACCCCATTGACGTCAATGGGAGTTTGT | |
| | TTTGGCACCAAAATCAACGGGACTTTCCAAAATGTCGTAACA | |
| | ACTCCGCCCCATTGACGCAAATGGGCGGTAGGCGTGTACGGT | |
| | GGGAGGTCTATATAAGCAGAGCTGGTTTAGTGAACCGT | |
| Cytomegalovinus | CGTTACATAACTTACGGTAAATGGCCCGCCTGGCTGACCGCC | 41 |
| (CMV) (third | CAACGACCCCCGCCCATTGACGTCAATAATGACGTATGTTCC | |
| version) | CATAGTAACGCCAATAGGGACTTTCCATTGACGTCAATGGGT | |
| | GGAGTATTTACGGTAAACTGCCCACTTGGCAGTACATCAAGT | |
| | GTATCATATGCCAAGTACGCCCCCTATTGACGTCAATGACGG | |
| | TAAATGGCCCGCCTGGCATTATGCCCAGTACATGACCTTATG | |
| | GGACTTTCCTACTTGGCAGTACATCTACGTATTAGTCATCGCT | |
| | ATTACCATGGTGATGCGGTTTTGGCAGTACATCAATGGGCGT | |
| | GGATAGCGGTTTGACTCACGGGGATTTCCAAGTCTCCACCCC | |
| | ATTGACGTCAATGGGAGTTTGTTTTGGCACCAAAATCAACGG | |
| | GACTTTCCAAAATGTCGTAACAACTCCGCCCCATTGACGCAA | |
| | ATGGGCGGTAGGCGTGTACGGTGGGAGGTCTATATAAGCAG | |
| | AGCT | |
| CAG promoter | ACTTACGGTAAATGGCCCGCCTGGCTGACCGCCCAACGACCC | 42 |
| (first version) | CCGCCCATTGACGTCAATAATGACGTATGTTCCCATAGTAAC | |
| | GCCAATAGGGACTTTCCATTGACGTCAATGGGTGGAGTATTT | |
| | ACGGTAAACTGCCCACTTGGCAGTACATCAAGTGTATCATAT | |
| | GCCAAGTACGCCCCCTATTGACGTCAATGACGGTAAATGGCC | |
| | CGCCTGGCATTATGCCCAGTACATGACCTTATGGGACTTTCC | |
| | TACTTGGCAGTACATCTACGTATTAGTCATCGCTATTACCAT | |
| | GGTCGAGGTGAGCCCCACGTTCTGCTTCACTCTCCCCATCTC | |
| | CCCCCCCTCCCCACCCCCAATTTTGTATTTATTTATTTTTTAAT | |
| | TATTTTGTGCAGCGATGGGGGCGGGGGGGGGGGGGGGCGCGC | |
| | GCCAGGCGGGGCGGGGCGGGGCGAGGGGCGGGGCGGGGCG | |
| | AGGCGGAGAGGTGCGGCGGCAGCCAATCAGAGCGGCGCGCT | |
| | CCGAAAGTTTCCTTTTATGGCGAGGCGGCGGCGGCGGCGGCC | |
| | CTATAAAAAGCGAAGCGCGCGGCGGGGGG | |
| CAG promoter | CGTTACATAACTTACGGTAAATGGCCCGCCTGGCTGACCGCC | 43 |
| (second version) | CAACGACCCCCGCCCATTGACGTCAATAATGACGTATGTTCC | |
| | CATAGTAACGCCAATAGGGACTTTCCATTGACGTCAATGGGT | |
| | GGAGTATTTACGGTAAACTGCCCACTTGGCAGTACATCAAGT | |
| | GTATCATATGCCAAGTACGCCCCCTATTGACGTCAATGACGG | |
| | TAAATGGCCCGCCTGGCATTATGCCCAGTACATGACCTTATG | |
| | GGACTTTCCTACTTGGCAGTACATCTACGTATTAGTCATCGCT | |
| | ATTACCATGTCGAGGTGAGCCCCACGTTCTGCTTCACTCTCC | |
| | CCATCTCCCCCCCCTCCCCACCCCCAATTTTGTATTTATTTAT | |
| | TTTTTAATTATTTTGTGCAGCGATGGGGGCGGGGGGGGGGGG | |
| | GGCGCGCGCCAGGCGGGGCGGGGCGGGGCGAGGGGCGGGG | |
| | CGGGGCGAGGCGGAGAGGTGCGGCGGCAGCCAATCAGAGC | |
| | GGCGCGCTCCGAAAGTTTCCTTTTATGGCGAGGCGGCGGCGG | |
| | CGGCGGCCCTATAAAAAGCGAAGCGCGCGGCGGGCG | |
| Human EF1- | CAACCTTTGGAGCTAAGCCAGCAATGGTAGAGGGAAGATTC | 44 |
| alpha (EF1-α) | TGCACGTCCCTTCCAGGCGGCCTCCCCGTCACCACCCCCCCC | |
| | AACCCGCCCCGACCGGAGCTGAGAGTAATTCATACAAAAGG | |
| | ACTCGCCCCTGCCTTGGGGAATCCCAGGGACCGTCGTTAAAC | |
| | TCCCACTAACGTAGAACCCAGAGATCGCTGCGTTCCCGCCCC | |
| | CTCACCCGCCCGCTCTCGTCATCACTGAGGTGGAGAATAGCA | |
| | TGCGTGAGGCTCCGGTGCCCGTCAGTGGGCAGAGCGCACAT | |
| | CGCCCACAGTCCCCGAGAAGTTGGGGGGAGGGGTCGGCAAT | |
| | TGAACGGGTGCCTAGAGAAGGTGGCGCGGGGTAAACTGGGA | |
| | AAGTGATGTCGTGTACTGGCTCCGCCTTTTTCCCGAGGGTGG | |
| | GGGAGAACCGTATATAAGTGCAGTAGTCGCCGTGAACGTT | |
| Human | ACTTGTGGACAAAGTTTGCTCTATTCCACCTCCTCCAGGCCCT | 48 |
| CamKIIa | CCTTGGGTCCATCACCCCAGGGGTGCTGGGTCCATCCCACCC | |
| (CaMKIIa) | CCAGGCCCACACAGGCTTGCAGTATTGTGTGCGGTATGGTCA | |
| | GGGCGTCCGAGAGCAGGTTTCGCAGTGGAAGGCAGGCAGGT | |
| | GTTGGGGAGGCAGTTACCGGGGCAACGGGAACAGGGCGTTT | |
| | TGGAGGTGGTTGCCATGGGGACCTGGATGCTGACGAAGGCT | |
| | CGCGAGGCTGTGAGCAGCCACAGTGCCCTGC | |

In a certain embodiment, the vector genome comprises a polynucleotide sequence at least or about 75%, at least or about 80%, at least or about 85%, at least or about 90%, at least or about 92%, at least or about 93%, at least or about 94%, at least or about 95%, at least or about 96%, at least or about 97%, at least or about 98%, at least or about 99%, or 100% identical to SEQ ID NO: 31. In a certain embodiment, the vector genome comprises a polynucleotide sequence at least or about 75%, at least or about 80%, at least or about 85%, at least or about 90%, at least or about 92%, at least or about 93%, at least or about 94%, at least or about 95%, at least or about 96%, at least or about 97%, at least or about 98%, at least or about 99%, or 100% identical to SEQ ID NO: 32. In a certain embodiment, the vector genome comprises a polynucleotide sequence at least or about 75%, at least or about 80%, at least or about 85%, at least or about 90%, at least or about 92%, at least or about 93%, at least or about 94%, at least or about 95%, at least or about 96%, at least or about 97%, at least or about 98%, at least or about 99%, or 10% identical to SEQ ID NO: 33.

Further illustrative examples of promoters are the SV40 late promoter from simian virus 40, the Baculovirus polyhedron enhancer/promoter element, Herpes Simplex Virus thymidine kinase (HSV tk), the immediate early promoter from cytomegalovirus (CMV) and various retroviral promoters including LTR elements. A large variety of other promoters are known and generally available in the art, and the sequences of many such promoters are available in sequence databases such as the GenBank database.

In some cases, vectors of the present disclosure further comprise one or more regulatory elements selected from the group consisting of an enhancer, an intron, a poly-A signal, a 2A peptide encoding sequence, a WPRE (Woodchuck hepatitis virus posttranscriptional regulatory element), and a HPRE (Hepatitis B posttranscriptional regulatory element).

In some embodiments, the vector comprises a CMV enhancer.

In certain embodiments, the vectors comprise one or more enhancers. In particular embodiments, the enhancer is a CMV enhancer sequence. a GAPDH enhancer sequence. a β-actin enhancer sequence, or an EF1-α enhancer sequence. Sequences of the foregoing are known in the art. For example, the sequence of the CMV immediate early (IE) enhancer is SEQ ID NO: 50.

In certain embodiments, the vectors comprise one or more introns. In particular embodiments, the intron is a rabbit globin intron sequence, a chicken β-actin intron sequence, a synthetic intron sequence, an SV40 intron, or an EF1-α intron sequence.

In certain embodiments, the vectors comprise a polyA sequence. In particular embodiments, the polyA sequence is a rabbit globin polyA sequence, a human growth hormone polyA sequence, a bovine growth hormone polyA sequence, a PGK polyA sequence, an SV40 polyA sequence, or a TK polyA sequence. In some embodiments, the poly-A signal may be a bovine growth hormone polyadenylation signal (bGHpA).

In certain embodiments, the vectors comprise one or more transcript stabilizing element. In particular embodiments, the transcript stabilizing element is a WPRE sequence, a HPRE sequence, a scaffold-attachment region, a 3′ UTR, or a 5′ UTR. In particular embodiments, the vectors comprise both a 5′ U TR and a 3′ UTR.

In some embodiments, the vector comprises a 5′ untranslated region (UTR) selected from Table 4. In some embodiments, the vector genome comprises a polynucleotide sequence at least or about 75%, at least or about 80%, at least or about 85%, at least or about 90%, at least or about 92%, at least or about 93%, at least or about 94%, at least or about 95%, at least or about 96%, at least or about 97%, at least or about 98%, at least or about 99%, or 100% identical to any one of SEQ ID NOS 51-61.

TABLE 4

| 5′ UNTRANSLATED | | SEQ ID |
|---|---|---|
| REGION | SEQUENCE | NO: |
| Human beta-actin | CGCGTCCGCCCGCGAGCACAGAGCCTCGCCTTTGCCGAT | 51 |
| exon/intron | CCGCCGCCCGTCCACACCCGCCGCCAGGTAAGCCCGGCC | |
| | AGCCGACCGGGGCATGCGGCCGCGGCCCTTCGCCCGTG | |
| | CAGAGCCGCCGTCTGGGCCGCAGCGGGGGGCGCATGGG | |
| | GCGGAACCGGACCGCCGTGGGGGGCGCGGGAGAAGCCC | |
| | CTGGGCCTCCGGAGATGGGGGACACCCCACGCCAGTTC | |
| | GCAGGCGCGAGGCCGCGCTCGGGCGGGCGCGCTCCGGG | |
| | GGTGCCGCTCTCGGGGCGGGGGCAACCGGCGGGGTCTT | |
| | TGTCTGAGCCGGGCTCTTGCCAATGGGGATCGCACGGTG | |
| | GGCGCGGCGTAGCCCCCGTCAGGCCCGGTGGGGGCTGG | 52 |
| | GGCGCCATGCGCGTGCGCGCTGGTCCTTTGGGCGCTAAC | |
| | TGCGTGCGCGCTGGGAATTGGCGCTAATTGCGCGTGCGC | |
| | GCTGGGACTCAATGGCGCTAATCGCGCGTGCGTTCTGGG | |
| | GCCCGGGCGCTTGCGCCACTTCCTGCCCGAGCCGCTGGC | |
| | GCCCGAGGGTGTGGCCGCTGCGTGCGCGCGCGCGACCC | |
| | GGTCGCTGTTTGAACCGGGCGGAGGCGGGGCTGGCGCC | |
| | CGGTTGGGAGGGGGTTGGGGCCTGGCTTCCTGCCGCGCG | |
| | CCGCGGGGACGCCTCCGACCAGTGTTTGCCTTTTATGGT | |
| | AATAACGCGGCCGGCCCGGCTTCCTTTGTCCCCAATCTG | |
| | GGCGCGCGCCGGCGCCCCCTGGCGGCCTAAGGACTCGG | |
| | CGCGCCGGAAGTGGCCAGGGCGGCAGCGGCTGCTCTTG | |
| | GCGGCCCCGAGGTGACTATAGCCTTCTTTTGTGTCTTGA | |
| | TAGTTCGCCAGCCTCTGCTAACCATGTTCATGCCTTCTTC | |
| | TTTTTCCTACAGCTCCTGGGCAACGTGCTGGTTATTGTGC | |
| | TGTCTCATCATTTTGGCAAAGAATTC | |
| Chicken beta-actin | GTCGCTGCGCGCTGCCTTCGCCCCGTGCCCCGCTCCGCC | |
| exon/intron + rabbit | GCCGCCTCGCGCCGCCCGCCCCGGCTCTGACTGACCGCG | |
| globin intron | TTACTCCCACAGGTGAGCGGGCGGGACGGCCCTTCTCCT | |
| | CCGGGCTGTAATTAGCGCTTGGTTTAATGACGGCTTGTT | |
| | TCTTTTCTGTGGCTGCGTGAAAGCCTTGAGGGGCTCCGG | |
| | GAGGGCCCTTTGTGCGGGGGGAGCGGCTCGGGGGGTGC | |
| | GTGCGTGTGTGTGTGCGTGGGGAGCGCCGCGTGCGGCTC | |
| | CGCGCTGCCCGGCGGCTGTGAGCGCTGCGGGCGCGGCG | |
| | CGGGGCTTTGTGCGCTCCGCAGTGTGCGCGAGGGGAGC | |
| | GCGGCCGGGGGCGGTGCCCCGCGGTGCGGGGGGGGCTG | |
| | CGAGGGGAACAAAGGCTGCGTGCGGGGTGTGTGCGTGG | |
| | GGGGGTGAGCAGGGGGTGTGGGCGCGTCGGTCGGGCTG | |
| | CAACCCCCCCTGCACCCCCCTCCCCGAGTTGCTGAGCAC | |
| | GGCCCGGCTTCGGGTGCGGGGCTCCGTACGGGGCGTGG | |
| | CGCGGGGCTCGCCGTGCCGGGGGGGGGGGGCGGCAGG | |
| | TGGGGGTGCCGGGGGGGGGGGGGCCGCCTCGGGCCGGG | |
| | GAGGGCTCGGGGGAGGGGCGCGGCGGCCCCCGGAGCGC | |
| | CGGCGGCTGTCGAGGCGCGGCGAGCCGCAGCCATTGCC | |
| | TTTTATGGTAATCGTGCGAGAGGGCGCAGGGACTTCCTT | |
| | TGTCCCAAATCTGTGCGGAGCCGAAATCTGGGAGGCGC | |
| | CGCCGCACCCCCTCTAGCGGGCGCGGGGCGAAGCGGTG | |
| | CGGCGCCGGCAGGAAGGAAATGGGGGGGGAGGGCCTTC | |
| | GTGCGTCGCCGCGCCGCCGTCCCCTTCTCCCTCTCCAGC | |
| | CTCGGGGCTGTCCGCGGGGGGACGGCTGCCTTCGGGGG | |
| | GGACGGGGCAGGGGGGGGTTCGGCTTCTGGCGTGTGAC | |
| | CGGCGGCTCTAGAGCCTCTGCTAACCATGTTCATGCCTT | |
| | CTTCTTTTTCCTACAGCTCCTGGGCAACGTGCTGGTTATT | |
| | GTGCTGTCTCATCATTTTGGCAAAGAATTC | |
| SV40 intron | GGTAAGTTTAGTCTTTTTGTCTTTTATTTCAGGTCCCGGA | 53 |
| (Chimeric intron | TCCGGTGGTGGTGCAAATCAAAGAACTGCTCCTCAGTGG | |
| sequence) | ATGTTGCCTTTACTTCTAGGCCTGTACGGAAGTGTTACTT | |
| | CTGCTCTAAAAGCTGCGGAATTGTACCCGC | |
| 5′ UTR-Syn1 Hs | AGTCTGCGGTGGGCAGCGGAGGAGTCGTGTCGTGCCTG | 54 |
| | AGAGCGCAGCTGTGCTCCTGGGCACCGCGCAGTCCGCCC | |
| | CCGCGGCTCCTGGCCAGACCACCCCTAGGACCCCCTGCC | |
| | CCAAGTCGCA | |
| CMV IE exon | TCAGATCGCCTGGAGAGGCCATCCACGCTGTTTTGACCT | 55 |
| | CCATAGTGGACACCGGGACCGATCCAGCCTCCGCGGCC | |
| | GGGAACGGTGCATTGGAACGCGGATTCCCCGTGCCAAG | |
| | AGTGAC | |
| TPL-ePKP2 (adenovirus | CTCACTCTCTTCCGCATCGCTGTCTGCGAGGGCCAGCTG | 56 |
| derived enhancer | TTGGGCTCGCGGTTGAGGACAAACTCTTCGCGGTCTTTC | |
| element) | CAGTACTCTTGGATCGGAAACCCGTCGGCCTCCGAACGG | |
| | TACTCCGCCACCGAGGGACCTGAGCGAGTCCGCATCGA | |
| | CCGGATCGGAAAACCTCTCGAGAAAGGCGTCTAACCAG | |
| | TCACAGTCGCAAGGTAGGCTGAGCACCGTGGCGGGCGG | |
| | CAGCGGGTGGCGGTCGGGGTTGTTTCTGGCGGAGGTGCT | |
| | GCTGATGATGTAATTAAAGTAGGCGGTCTTGAGACGGC | |
| | GGATGGTCGAGGTGAGGTGTGGCAGGCTTGAGATCCAG | |
| | CTGTTGGGGTGAGTACTCCCTCTCAAAAGCGGGCATTAC | |
| | TTCTGCGCTAAGATTGTCAGTTTCCAAAAACGAGGAGGA | |
| | TTTGATATTCACCTGGCCCGATCTGGCCATACACTTGAG | |
| | TGACAATGACATCCACTTTGCCTTTCTCTCCACAGGTGTC | |
| | CACTCCCAG | |
| Human EF1-α | CTTTTTCGCAACGGGTTTGCCGCCAGAACACAGGTAAGT | 57 |
| intron/exon | GCCGTGTGTGGTTCCCGCGGGCCTGGCCTCTTTACGGGT | |
| | TATGGCCCTTGCGTGCCTTGAATTACTTCCACCTGGCTCC | |
| | AGTACGTGATTCTTGATCCCGAGCTGGAGCCAGGGGCG | |
| | GGCCTTGCGCTTTAGGAGCCCCTTCGCCTCGTGCTTGAG | |
| | TTGAGGCCTGGCCTGGGCGCTGGGGCCGCCGCGTGCGA | |
| | ATCTGGTGGCACCTTCGCGCCTGTCTCGCTGCTTTCGATA | |
| | AGTCTCTAGCCATTTAAAATTTTTGATGACGTGCTGCGA | |
| | CGCTTTTTTTCTGGCAAGATAGTCTTGTAAATGCGGGCC | |
| | AGGATCTGCACACTGGTATTTCGGTTTTTGGGCCCGCGG | |
| | CCGGCGACGGGGCCCGTGCGTCCCAGCGCACATGTTCG | |
| | GCGAGGCGGGGCCTGCGAGCGCGGCCACCGAGAATCGG | |
| | ACGGGGGTAGTCTCAAGCTGGCCGGCCTGCTCTGGTGCC | |
| | TGGCCTCGCGCCGCCGTGTATCGCCCCGCCCTGGGCGGC | |
| | AAGGCTGGCCCGGTCGGCACCAGTTGCGTGAGCGGAAA | |
| | GATGGCCGCTTCCCGGCCCTGCTCCAGGGGGCTCAAAAT | |
| | GGAGGACGCGGCGCTCGGGAGAGCGGGGGGGTGAGTCA | |
| | CCCACACAAAGGAAAAGGGCCTTTCCGTCCTCAGCCGTC | |
| | GCTTCATGTGACTCCACGGAGTACCGGGCGCCGTCCAGG | |
| | CACCTCGATTAGTTCTGGAGCTTTTGGAGTACGTCGTCTT | |
| | TAGGTTGGGGGGAGGGGTTTTATGCGATGGAGTTTCCCC | |
| | ACACTGAGTGGGTGGAGACTGAAGTTAGGCCAGCTTGG | |
| | CACTTGATGTAATTCTCCTTGGAATTTGGCCTTTTTGAGT | |
| | TTGGATCTTGGTTCATTCTCAAGCCTCAGACAGTGGTTC | |
| | AAAGTTTTTTTCTTCCATTTCAG | |
| Human EF1-α, intron A | GTAAGTGCCGTGTGTGGTTCCCGCGGGCCTGGCCTCTTT | 58 |
| | ACGGGTTATGGCCCTTGCGTGCCTTGAATTACTTCCACC | |
| | TGGCTGCAGTACGTGATTCTTGATCCCGAGCTTCGGGTT | |
| | GGAAGTGGGTGGGAGAGTTCGAGGCCTTGCGCTTAAGG | |
| | AGCCCCTTCGCCTCGTGCTTGAGTTGAGGCCTGGCCTGG | |
| | GCGCTGGGGCCGCCGCGTGCGAATCTGGTGGCACCTTCG | |
| | CGCCTGTCTCGCTGCTTTCGATAAGTCTCTAGCCATTTAA | |
| | AATTTTTGATGACCTGCTGCGACGCTTTTTTTCTGGCAAG | |
| | ATAGTCTTGTAAATGCGGGCCAAGATCTGCACACTGGTA | |
| | TTTCGGTTTTTGGGGCCGCGGGCGGCGACGGGGCCCGTG | |
| | CGTCCCAGCGCACATGTTCGGCGAGGCGGGGCCTGCGA | |
| | GCGCGGCCACCGAGAATCGGACGGGGGTAGTCTCAAGC | |
| | TGGCCGGCCTGCTCTGGTGCCTGGCCTCGCGCCGCCGTG | |
| | TATCGCCCCGCCCTGGGCGGCAAGGCTGGCCCGGTCGGC | |
| | ACCAGTTGCGTGAGCGGAAAGATGGCCGCTTCCCGGCC | |
| | CTGCTGCAGGGAGCTCAAAATGGAGGACGCGGCGCTCG | |
| | GGAGAGCGGGCGGGTGAGTCACCCACACAAAGGAAAA | |
| | GGGCCTTTCCGTCCTCAGCCGTCGCTTCATGTGACTCCA | |
| | CGGAGTACCGGGCGCCGTCCAGGCACCTCGATTAGTTCT | |
| | CGAGCTTTTGGAGTACGTCGTCTTTAGGTTGGGGGGAGG | |
| | GGTTTTATGCGATGGAGTTTCCCCACACTGAGTGGGTGG | |
| | AGACTGAAGTTAGGCCAGCTTGGCACTTGATGTAATTCT | |
| | CCTTGGAATTTGCCCTTTTTGAGTTTGGATCTTGGTTCAT | |
| | TCTCAAGCCTCAGACAGTGGTTCAAAGTTTTTTTCTTCCA | |
| | TTTCAG | |
| 5′ UTR human | TCAGAAGCCCCGGGCTCGTCAGTCAAACCGGTTCTCTGT | 59 |
| CamKIIa | TTGCACTCGGCAGCACGGGCAGGCAAGTGGTCCCTAGG | |
| | TTCGGG | |
| B-globin intron | GTGAGTCTATGGGACCCTTGATGTTTTCTTTCCCCTTCTT | 60 |
| | TTCTATGGTTAAGTTCATGTCATAGGAAGGGGAGAAGTA | |
| | ACAGGGTACACATATTGACCAAATCAGGGTAATTTTGCA | |
| | TTTGTAATTTTAAAAAATGCTTTCTTCTTTTAATATACTT | |
| | TTTTGTTTATCTTATTTCTAATACTTTCCCTAATCTCTTTC | |
| | TTTCAGGGCAATAATGATACAATGTATCATGCCTCTTTG | |
| | CACCATTCTAAAGAATAACAGTGATAATTTCTGGGTTAA | |
| | GGCAATAGCAATATTTCTGCATATAAATATTTCTGCATA | |
| | TAAATTGTAACTGATGTAAGAGGTTTCATATTGCTAATA | |
| | GCAGCTACAATCCAGCTACCATTCTGCTTTTATTTTATGG | |
| | TTGGGATAAGGCTGGATTATTCTGAGTCCAAGCTAGGCC | |
| | CTTTTGCTAATCATGTTCATACCTCTTATCTTCCTCCCAC | |
| | AG | |
| SV40 intron (long form; | TCTAGAGGATCCGGTACTCGAGGAACTGAAAAACCAGA | 61 |
| underlined 5′ and 3′ | AAGTTAACTGGTAAGTTTAGTCTTTTTGTCTTTTATTTCA | |
| extensions) | GGTCCCGGATCCGGTGGTGGTGCAAATCAAAGAACTGC | |
| | TCCTCAGTGGATGTTGCCTTTACTTCTAGGCCTGTACGG | |
| | AAGTGTTACTTCTGCTCTAAAAGCTGCGGAATTGTACCC | |
| | GC | |

In some embodiments, the vector comprises a 3′ untranslated region selected from Table 5. In some embodiments, the vector genome comprises a polynucleotide sequence at least or about 75 at least or about 80%, at least or about 85%, at least or about 90%, at least or about. 92%, at. least or about 93%, at least, or about 94%, at least or about 95%, at least or about 96%, at. least or about. 97%, at, least or about 98%, at least or about 99% or 100% identical to any one of SEQ ID NOS 62-70.

TABLE 5

| 3′ UNTRANSLATED | | SEQ ID |
|---|---|---|
| REGION | SEQUENCE | NO: |
| WPRE(x) (mutated | AATCAACCTCTGGATTACAAAATTTGTGAAAGATTGACT | 62 |
| woodchuck hepatitis | GGTATTCTTAACTATGTTGCTCCTTTTACGCTATGTGGAT | |
| regulatory element- | ACGCTGCTTTAATGCCTTTGTATCATGCTATTGCTTCCCGT | |
| version 1) | ATGGCTTTCATTTTCTCCTCCTTGTATAAATCCTGGTTGCT | |
| | GTCTCTTTATGAGGAGTTGTGGCCCGTTGTCAGGCAACGT | |
| | GGCGTGGTGTGCACTGTGTTTGCTGACGCAACCCCCACTG | |
| | GTTGGGGCATTGCCACCACCTGTCAGCTCCTTTCCGGGAC | |
| | TTTCGCTTTCCCCCTCCCTATTGCCACGGCGGAACTCATC | |
| | GCCGCCTGCCTTGCCCGCTGCTGGACAGGGGCTCGGCTGT | |
| | TGGGCACTGACAATTCCGTGGTGTTGTCGGGGAAATCAT | |
| | CGTCCTTTCCTTGGCTGCTCGCCTGTGTTGCCACCTGGATT | |
| | CTGCGCGGGACGTCCTTCTGCTACGTCCCTTCGGCCCTCA | |
| | ATCCAGCGGACCTTCCTTCCCGCGGCCTGCTGCCGGCTCT | |
| | GCGGCCTCTTCCGCGTCTTCGCCTTCGCCCTCAGACGAGT | |
| | CGGATCTCCCTTTGGGCCGCCTCCCCGC | |
| WPRE(x) (mutated | TCAACCTCTGGATTACAAAATTTGTGAAAGATTGACTGGT | 63 |
| woodchuck hepatitis | ATTCTTAACTATGTTGCTCCTTTTACGCTATGTGGATACG | |
| regulatory element- | CTGCTTTAATGCCTTTGTATCATGCTATTGCTTCCCGTATG | |
| version 2) | GCTTTCATTTTCTCCTCCTTGTATAAATCCTGGTTGCTGTC | |
| | TCTTTATGAGGAGTTGTGGCCCGTTGTCAGGCAACGTGGC | |
| | GTGGTGTGCACTGTGTTTGCTGACGCAACCCCCACTGGTT | |
| | GGGGCATTGCCACCACCTGTCAGCTCCTTTCCGGGACTTT | |
| | CGCTTTCCCCCTCCCTATTGCCACGGCGGAACTCATCGCC | |
| | GCCTGCCTTGCCCGCTGCTGGACAGGGGCTCGGCTGTTGG | |
| | GCACTGACAATTCCGTGGTGTTGTCGGGGAAATCATCGTC | |
| | CTTTCCTTGGCTGCTCGCCTGTGTTGCCACCTGGATTCTGC | |
| | GCGGGACGTCCTTCTGCTACGTCCCTTCGGCCCTCAATCC | |
| | AGCGGACCTTCCTTCCCGCGGCCTGCTGCCGGCTCTGCGG | |
| | CCTCTTCCGCGTCTTCGCCTTCGCCCTCAGACGAGTCGGA | |
| | TCTCCCTTTGGGCCGCCTCCCCGCA | |
| WPRE(x) (mutated | TTCCTGTTAATCAACCTCTGGATTACAAAATTTGTGAAAG | 64 |
| woodchuck hepatitis | ATTGACTGGTATTCTTAACTATGTTGCTCCTTTTACGCTAT | |
| regulatory element- | GTGGATACGCTGCTTTAATGCCTTTGTATCATGCTATTGC | |
| version 3) | TTCCCGTATGGCTTTCATTTTCTCCTCCTTGTATAAATCCT | |
| | GGTTGCTGTCTCTTTATGAGGAGTTGTGGCCCGTTGTCAG | |
| | GCAACGTGGCGTGGTGTGCACTGTGTTTGCTGACGCAAC | |
| | CCCCACTGGTTGGGGCATTGCCACCACCTGTCAGCTCCTT | |
| | TCCGGGACTTTCGCTTTCCCCCTCCCTATTGCCACGGCGG | |
| | AACTCATCGCCGCCTGCCTTGCCCGCTGCTGGACAGGGG | |
| | CTCGGCTGTTGGGCACTGACAATTCCGTGGTGTTGTCGGG | |
| | GAAGCTGACGTCCTTTCCGCGGCTGCTCGCCTGTGTTGCC | |
| | ACCTGGATTCTGCGCGGGACGTCCTTCTGCTACGTCCCTT | |
| | CGGCCCTCAATCCAGCGGACCTTCCTTCCCGCGGCCTGCT | |
| | GCCGGCTCTGCGGCCTCTTCCGCCTCTTCGCCTTCGCCCT | |
| | CAGACGAGTCGGATCTCCCTTTGGGCCGCCTCCCCGCCCA | |
| | TGTATCTTTTTCACCTGTGCCTTGTTTTTGCCTGTGTTCCG | |
| | CGTCCTACTTTTCAAGCCTCCAAGCTGTGCCTTGGGCGGC | |
| | TTTGGGGCATGGACATAGATCCCTATAAAGAATTTGGTTC | |
| | ATCTTATCAGTTGTTGAATTTTCTTCCTTTGGAC | |
| CAAX | TGTGTGATAATG | 65 |
| EES | CTGTTCTCATCACATCATATCAAGGTTATATACCATCAAT | 66 |
| HPRE | ATTGCCACAGATGTTACTTAGCCTTTTAATATTTCTCTAAT | 67 |
| | TTAGTGTATATGCAATGATAGTTCTCTGATTTCTGAGATT | |
| | GAGTTTCTCATGTGTAATGATTATTTAGAGTTTCTCTTTCA | |
| | TCTGTTCAAATTTTTGTCTAGTTTTATTTTTTACTGATTTG | |
| | TAAGACTTCTTTTTATAATCTGCATATTACAATTCTCTTTA | |
| | CTGGGGTGTTGCAAATATTTTCTGTCATTCTATGGCCTGA | |
| | CTTTTCTTAATGGTTTTTTAATTTTAAAAATAAGTCTTAAT | |
| | ATTCATGCAATCTAATTAACAATCTTTTCTTTGTGGTTAG | |
| | GACTTTGAGTCATAAGAAATTTTTCTCTACACTGAAGTCA | |
| | TGATGGCATGCTTCTATATTATTTTCTAAAAGATTTAAAG | |
| | TTTTGCCTTCTCCATTTAGACTTATAATTCACTGGAATTTT | |
| | TTTGTGTGTATGGTATGACATATGGGTTCCCTTTTATTTTT | |
| | TACATATAAATATATTTCCCTGTTTTTCTAAAAAAGAAAA | |
| | AGATCATCATTTTCCCATTGTAAAATGCCATATTTTTTTCA | |
| | TAGGTCACTTACATATATCAATGGGTCTGTTTCTGAGCTC | |
| | TACTCTATTTTATCAGCCTCACTGTCTATCCCCACACATCT | |
| | CATGCTTTGCTCTAAATCTTGATATTTAGTGGAACATTCT | |
| | TTCCCATTTTGTTCTACAAGAATATTTTTGTTATTGTCTTT | |
| | GGGCTTTCTATATACATTTTGAAATGAGGTTGACAAGTTA | |
| | ATAACAGGCCTATTGATTGGAAAGTTTGTCAACGAATTGT | |
| | GGGTCTTTTGGGGTTTGCTGCCCCTTTTACGCAATGTGGA | |
| | TATCCTGCTTTAATGCCTTTATATGCATGTATACAAGCAA | |
| | AACAGGCTTTTACTTTCTCGCCAACTTACAAGGCCTTTCT | |
| | CAGTAAACAGTATATGACCCTTTACCCCGTTGCTCGGCAA | |
| | CGGCCTGGTCTGTGCCAAGTGTTTGCTGACGCAACCCCCA | |
| | CTGGTTGGGGCTTGGCCATAGGCCATCAGCGCATGCGTG | |
| | GAACCTTTGTGTCTCCTCTGCCGATCCATACTGCGGAACT | |
| | CCTAGCCGCTTGTTTTGCTCGCAGCAGGTCTGGAGCAAAC | |
| | CTCATCGGGACCGACAATTCTGTCGTACTCTCCCGCAAGT | |
| | ATACATCGTTTCCATGGCTGCTAGGCTGTGCTGCCAACTG | |
| | GATCCTGCGCGGGACGTCCTTTGTTTACGTCCCGTCGGCG | |
| | CTGAATCCCGCGGACGACCCCTCCCGGGGCCGCTTGGGG | |
| | CTCTACCGCCCGCTTCTCCGTCTGCCGTACCGTCCGACCA | |
| | CGGGGCGCACCTCTCTTTACGCGGACTCCCCGTCTGTGCC | |
| | TTCTCATCTGCCGGACCGTGTGCACTTCGCTTCACCTCTG | |
| | CACGTCGCATGGAGGCCACCGTGAACGCCCACCGGAACC | |
| | TGCCCAAGGTCTTGCATAAGAGGACTCTTGGACTTTCAGC | |
| | AATGTCATC | |
| R2V17 (HepB derived | TTCCTGTAAACAGGCCTATTGATTGGAAAGTTTGTCAACG | 68 |
| enhancer element) | AATTGTGGGTCTTTTGGGGTTTGCTGCCCCTTTTACGCAA | |
| | TGTGGATATCCTGCTTTAATGCCTTTATATGCATGTATAC | |
| | AAGCAAAACAGGCTTTTACTTTCTCGCCAACTTACAAGGC | |
| | CTTTCTCAGTAAACAGTATATGACCCTTTACCCCGTTGCT | |
| | CGGCAACGGCCTGGTCTGTGCCAAGTGTTTGCTGACGCA | |
| | ACCCCCACTGGTTGGGGCTTGGCCATAGGCCATCAGCGC | |
| | ATGCGTGGAACCTTTGTGTCTCCTCTGCCGATCCATACTG | |
| | CGGAACTCCTAGCCGCTTGTTTTGCTCGCAGCTGGACTGG | |
| | AGCAAACCTCATCGGGACCGACAATTCTGTCGTACTCTCC | |
| | CGCAAGCACTCACCGTTTCCGCGGCTGCTCGCCTGTGTTG | |
| | CCACCTGGATTCTGCGCGGGACGTCCTTCTGCTACGTCCC | |
| | TTCGGCCCTCAATCCAGCGGACCTTCCTTCCCGCGGCCTG | |
| | CTGCCGGCTCTGCGGCCTCTTCCGCCTCTTCGCCTTCGCC | |
| | CTCAGACGAGTCGGATCTCCCTTTGGGCCGCCTCCCCGCC | |
| | CATGTATCTTTTTCACCTGTGCCTTGTTTTTGCCTGTGTTC | |
| | CGCGTCCTACTTTTCAAGCCTCCAAGCTGTGCCTTGGGCG | |
| | GCTTTGGGGCATGGACATAGATCCCTATAAAGAATTTGG | |
| | TTCATCTTATCAGTTGTTGAATTTTCTTCCTTTGGAC | |
| 3′UTR(globin) | GCTGGAGCCTCGGTAGCCGTTCCTCCTGCCCGCTGGGCCT | 69 |
| | CCCAACGGGCCCTCCTCCCCTCCTTGCACCGGCCCTTCCT | |
| | GGTCTTTGAATAAA | |
| WPRE(r) | ATTCGAGCATCTTACCGCCATTTATTCCCATATTTGTTCTG | 70 |
| | TTTTTCTTGATTTGGGTATACATTTAAATGTTAATAAAAC | |
| | AAAATGGTGGGGCAATCATTTACATTTTTAGGGATATGTA | |
| | ATTACTAGTTCAGGTGTATTGCCACAAGACAAACATGTTA | |
| | AGAAACTTTCCCGTTATTTACGCTCTGTTCCTGTTAATCA | |
| | ACCTCTGGATTACAAAATTTGTGAAAGATTGACTGATATT | |
| | CTTAACTATGTTGCTCCTTTTACGCTGTGTGGATATGCTG | |
| | CTTTAATGCCTCTGTATCATGCTATTGCTTCCCGTACGGCT | |
| | TTCGTTTTCTCCTCCTTGTATAAATCCTGGTTGCTGTCTCT | |
| | TTATGAGGAGTTGTGGCCCGTTGTCCGTCAACGTGGCGTG | |
| | GTGTGCTCTGTGTTTGCTGACGCAACCCCCACTGGCTGGG | |
| | GCATTGCCACCACCTGTCAACTCCTTTCTGGGACTTTCGC | |
| | TTTCCCCCTCCCGATCGCCACGGCAGAACTCATCGCCGCC | |
| | TGCCTTGCCCGCTGCTGGACAGGGGCTAGGTTGCTGGGC | |
| | ACTGATAATTCCGTGGTGTTGTCGGGGAAGGGCC | |

In some embodiments, the vector comprises a polyadenylation (polyA) signal selected from Table 6, In some embodiments, the polyA signal comprises a polynucleotide sequence at least 75%, 80%, 85%, 90%, 92%, 93%, 94%, 95%, 96%, 97%, 98%, 99% or 100% identical to any one of SEQ ID NOS 71-75.

TABLE 6

| POLY- | | |
|---|---|---|
| ADENYLATION | | SEQ ID |
| SITE | SEQUENCE | NO: |
| Rabbit globin | TGGCTAATAAAGGAAATTTATTTTCATTGCAATAGTGTG | 71 |
| (pAGlobin-Oc) | TTGGAATTTTTTGTGTCTCTCACTCGGAAGAACATATGG | |
| | GAGGGCAAATCATTTAAAACATCAGAATGAGTATTTGGT | |
| | TTAGAGTTTGGCAACATATGCCCATATGCTGGCTGCCAT | |
| | GAACAAAGGTTGGCTATAAAGAGGTCATCAGTATATGA | |
| | AACAGCCCCCTGCTGTCCATTCCTTATTCCATAGAAAAG | |
| | CCTTGACTTGAGGTTAGATTTTTTTTATATTTTGTTTTGTG | |
| | TTATTTTTTTCTTTAACATCCCTAAAATTTTCCTTACATGT | |
| | TTTACTAGCCAGATTTTTCCTCCTCTCCTGACTACTCCCA | |
| | GTCATAGCTGTCCCTCTTCTCTTATGGAGATC | |
| Bovine growth | TTGCCAGCCATCTGTTGTTTGCCCCTCCCCCGTGCCTTCC | 72 |
| hormone (pAGH-Bt- | TTGACCCTGGAAGGTGCCACTCCCACTGTCCTTTCCTAA | |
| version 1) | TAAAATGAGGAAATTGCATCGCATTGTCTGAGTAGGTGT | |
| | CATTCTATTCTGGGGGGTGGGGTGGGGCAGGACAGCAA | |
| | GGGGGAGGATTGGGAATACAATAGCAGGCATGCTGGGG | |
| | ATGCGGTGGGCTCTATGGGTACCCAGGTGCTGAAGAATT | |
| | GACCCGGTTCCTCCTGGG | |
| Bovine growth | TTGCCAGCCATCTGTTGTTTGCCCCTCCCCCGTGCCTTCC | 73 |
| hormone (pAGH-Bt- | TTGACCCTGGAAGGTGCCACTCCCACTGTCCTTTCCTAA | |
| version 2) | TAAAATGAGGAAATTGCATCGCATTGTCTGAGTAGGTGT | |
| | CATTCTATTCTGGGGGGTGGGGTGGGGCAGGACAGCAA | |
| | GGGGGAGGATTGGGAAGACAATAGCAGGCATGCTGGGG | |
| | ATGCGGTGGGCTCTATGGGTACCCAGGTGCTGAAGAATT | |
| | GACCCGGTTCCTCCTGGG | |
| Bovine growth | CTGTGCCTTCTAGTTGCCAGCCATCTGTTGTTTGCCCCTC | 74 |
| hormone (pAGH-Bt- | CCCCGTGCCTTCCTTGACCCTGGAAGGTGCCACTCCCAC | |
| version 3) | TGTCCTTTCCTAATAAAATGAGGAAATTGCATCGCATTG | |
| | TCTGAGTAGGTGTCATTCTATTCTGGGGGGTGGGGTGGG | |
| | GCAGGACAGCAAGGGGGAGGATTGGGAAGACAATAGC | |
| | AGGCATGCTGGGGATGCGGTGGGCTCTATGG | |
| Human growth | CTGCCCGGGTGGCATCCCTGTGACCCCTCCCCAGTGCCT | 75 |
| hormone (pAGH-Hs) | CTCCTGGCCCTGGAAGTTGCCACTCCAGTGCCCACCAGC | |
| | CTTGTCCTAATAAAATTAAGTTGCATCATTTTGTCTGACT | |
| | AGGTGTCCTTCTATAATATTATGGGGTGGAGGGGGGTGG | |
| | TATGGAGCAAGGGGCCCAAGTTGGGAAGAAACCTGTAG | |
| | GGCCTGC | |

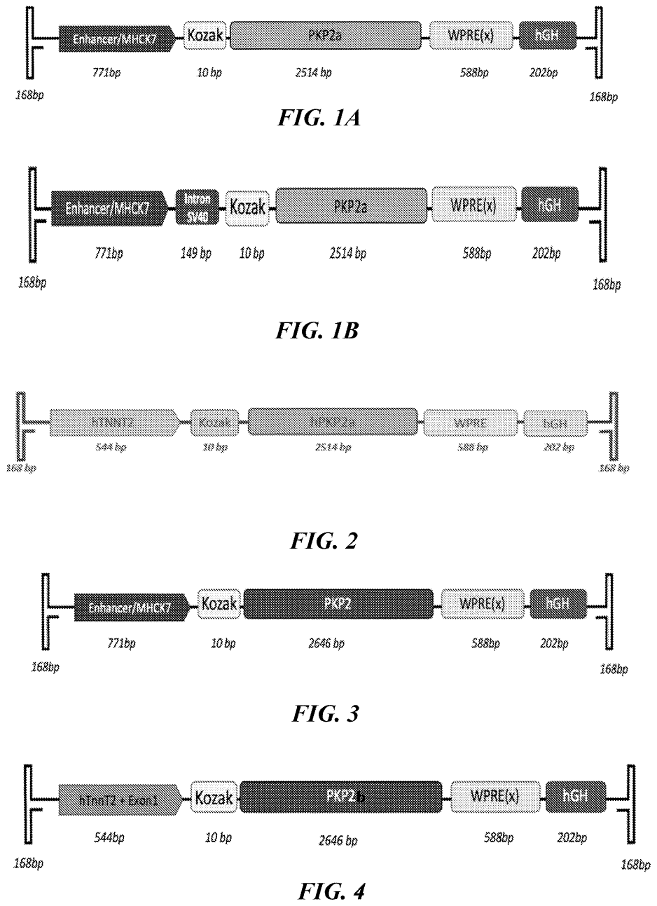

Illustrative vector genomes are depicted in FIGS. 1-4; and provided as SEQ ID NOs: 12-15, 89-92, and 97-99. The-expression cassette of each vector genome sequence is SEQ ID NOs. 8-11, 93-96, and 100-102, respectively. In some embodiments, the vector genome comprises, consists essentially of. or consists of a polynucleotide sequence that shares at least or about 90%, at least or about 91%, at least or about 92%, at least or about. 93% at least or about 94%, at least or about 95%, at least or about 96%, at least or about 97%, at least or about 98%, at least or about 99%, or 100% identity to any one of SEQ ID NOs: 12-15. 89-92, or 97-99, optionally with or without the ITR sequences. In some embodiments, the vector genomic comprises, consists essentially of, or consists of a polynucleotide sequence, that shares at least or about 90%, at least or about 91% at least or about 92%, at least or about 93%, at least or about 94%, at least or about 95%, at least or about 96%, at least or about 97%, at least or about 9P/%, at least or about 99%, or 1 00% identity to any one of SEQ ID NOs: 8-11, 93-96, or 100-102. In certain embodiments, vectors and expression cassettes disclosed herein demonstrate advantageous properties as compared to alternatives, such as, e.g., higher expression in mammalian cells (including target cells), more selective expression in target tissue (e.g., cells of the heart, such as cardiomyocytes), higher infection rate, and/or increased manufacturability, e.g., higher manufacturing yield. In particular embodiments, the target tissue is heart tissue or cardiac tissue.

In a certain embodiment, the vector genome comprises, in 5′ to 3′ order, a 5′ ITR; an MHCK7 promoter; a PKPa transgene; an WPRE(x) element; an pAGH-HS sequence; and a 3′ ITR, The vector genome may comprise, in 5′ to 3′ order, the polynucleotide sequences SEQ ID NO: 31; any one of SEQ ID NOs: 3, 6, and 87; SEQ ID NO: 63; and SEQ ID NO: 75; or polynucleotide sequences sharing at least or about 90%, at least or about 91%, at least or about 92%, at least or about 93%, at least or about 94%, at least or about 95%, at least or about 96%, at least or about 97%, at least or about 98%, at least or about 99%, or 100% identity to each of the foregoing. In certain embodiments, this vector genome is packaged in an AAV9 or AAVrh.74 vector. The PKP2a transgene of this embodiment is a full length or substantially full length transgene, i.e., a transgene encoding a PKPa of at least 800, at least 837, or at least 830 amino acids. In particular embodiments, the PKP2a polypeptide includes 837 amino acids. In related embodiments, instead of a PKP2a transgene, the transgene is a full length or substantially full length transgene, i.e., a transgene encoding a PKP2b of at least 800, at least 830, at least 837, or at least 881 amino acids. In particular embodiments, the PKP2b polypeptide includes 881 amino acids

In a certain embodiment, the vector genome comprises, in 5′ to 3′ order, a 5′ ITR; a hTnnT2 promoter; a PKPa transgene; an WPRE(x) element; an pAGH-HS sequence; and a 3′ IR. The vector genome may comprise, in 5′ to 3′ order, the polynucleotide sequences SEQ ID NO: 32 or 33; any one of SEQ ID NOs: 3, 6, and 87; SEQ ID NO: 63; and SEQ ID NO: 75; or polynucleotide sequences sharing at least or about 90%, at least or about 91%, at least or about 92%, at least or about 93%, at least or about 94%, at least or about 95%, at least or about 96%, at least or about 97%, at least or about 98%, at least or about 99%, or 100% identity to each of the foregoing. In certain embodiments, this vector genome is packaged in an AAV9 or AAVrh.74 vector. The PKP2a transgene of this embodiment is a full length or substantially full length transgene, i.e., a transgene encoding a PIKPa of at least 800, at least 837, or at least 830 amino acids. In particular embodiments, the PKP2a polypeptide includes 837 amino acids. In related embodiments, instead of a PKP2a transgene, the transgene is a full length or substantially fill length transgene, i.e., a transgene encoding a PKP2b of at least 800, at least 830, at least 837, or at least 881 amino acids. In particular embodiments, the PKP2b polypeptide includes 881 amino acids.

In a certain embodiment, the vector genome comprises, in 5′ to 3′ order, a 5′ ITR; an MHCK7 promoter; a PKPb transgene; an WPRE(x) element; an pAGH-HS sequence; and a 3′ ITR. The vector genome may comprise, in 5′ to 3′ order, the polynucleotide sequences SEQ ID NO: 31; any one of SEQ I D NOs: 4, 7, and 88; SEQ ID NO: 63; and SEQ ID NO: 75; or polynucleotide sequences sharing at least or about 90%, at least or about 91%, at least or about 92%, at least or about 93%, at least or about 94%, at least or about 95%, at least or about 96%, at least or about 97%, at least or about 98%, at least or about 99%, or 100% identity to each of the foregoing. In certain embodiments, this vector genome is packaged in an AAV9 or AAVrh.74 vector. The PKP2a transgene of this embodiment is a full length or substantially full length transgene, i.e., a transgene encoding a PKPa of at least 800, at least 837, or at least 830 amino acids. In particular embodiments, the PKP2a polypeptide includes 837 amino acids. In related embodiments, instead of a PKP2a transgene, the transgene is a full length or substantially full length transgene, i.e., a transgene encoding a PKP2b of at least 800, at least 830, at least 837, or at least 881 amino acids. In particular embodiments, the PKP2b polypeptide includes 881 amino acids.

In a certain embodiment, the vector genome comprises, in 5′ to 3′ order, a 5′ ITR; a hTnnT2 promoter: a PKPb transgene; an WPRE(x) element; an pAGH-HS sequence; and a 3′ ITR. The vector genome may comprise, in 5′ to 3′ order, the polynucleotide sequences SEQ ID NO: 32 or 33; any one of SEQ ID NOs: 4, 7, and 88; SEQ ID NO: 63; and SEQ ID NO: 75; or polynucleotide sequences sharing at least or about 90%, at least or about 91%, at least or about 92%, at least or about 93%, at least or about 94%, at least or about 95%, at least or about 96%, at least or about 97%, at least or about 98%, at least or about 99%, or 100% identity to each of the foregoing. In certain embodiments, this vector genome is packaged in an AAV9 or AAVrh.74 vector. The PKP2a transgene of this embodiment is a fill length or substantially full length transgene, i.e., a transgene encoding; a PKPa of at least 800, at least 837, or at least 830 amino acids. In particular embodiments, the PKP2a polypeptide includes 837 amino acids. In related embodiments, instead of a PKP2a transgene, the transgene is a full length or substantially full length transgene, i.e., a transgene encoding a PKP2b of at least 800, at least 830, at least 837, or at least 881 amino acids. In particular embodiments, the PKP2b polypeptide includes 881 amino acids.

In a certain embodiment, the vector genome comprises, in 5′ to 3′ order, a 5′ ITR; an MHCK7 promoter; a PKPa transgene; optionally a WPRE element; a polyadenylation sequence; and a 3′ ITR. The vector genome may comprise, in 5′ to 3′ order, the polynucleotide sequences SEQ ID NO: 31; any one of SEQ ID NOs: 3, 6, and 87; or polynucleotide sequences sharing at least or about 90%, at least or about 91%, at least or about 92%, at least or about 93%, at least or about 94%, at least or about 95%, at least or about 96%, at least or about 97%, at least or about 98%, at least or about 99%, or 100% identity to each of the foregoing. In certain embodiments. this vector genome is packaged in an AAV9 or AAVrh.74 vector. The PKP2a transgene of this embodiment is a full length or substantially full length transgene, i.e., a transgene encoding a PKPa of at least 800, at least 837, or at least 830 amino acids. In particular embodiments, the PKP2a polypeptide includes 837 amino acids. In related embodiments, instead of a PKP2a transgene, the transgene is a full length or substantially full length transgene, i.e., a transgene encoding a PKP2b of at least 800, at least 830, at least 837, or at least 881 amino acids. In particular embodiments, the PKP2b polypeptide includes 881 amino acids.

In a certain embodiment, the vector genome comprises, in 5′ to 3′ order, a 5′ ITR; a hTnnT2 promoter: a PKPa transgene; optionally a WPRE element; a polyadenylation sequence; and a 3′ IR. The vector genome may comprise, in 5′ to 3′ order, the polynucleotide sequences SEQ ID NO: 32 or 33; any one of SEQ ID NOs: 3, 6, and 87: or polynucleotide sequences sharing at least or about 90%, at least or about 91%, at least or about 92%, at least or about 93%, at least or about 94%, at least or about 95%, at least or about 96%, at least or about 97%, at least or about 98%, at least or about 99%, or 100% identity to each of the foregoing. In certain embodiments, this vector genome is packaged in an AAV9 or AAVrh.74 vector. The PKP2a transgene of this embodiment is a full length or substantially full length transgene, i.e., a transgene encoding a PKPa of at least 800, at least 837, or at least 830 amino acids. In particular embodiments, the PKP2a polypeptide includes 837 amino acids. In related embodiments, instead of a PKP2a transgene, the transgene is a full length or substantially full length transgene, i.e., a transgene encoding a PKP2b of at least 800, at least 830, at least 837, or at least 881 amino acids. In particular embodiments, the PKP2b polypeptide includes 881 amino acids.

In a certain embodiment, the vector genome comprises, in 5′ to 3′ order, a 5′ ITR; an MHCK7 promoter; a PKPb transgene; optionally a WPRE element; a polyadenylation sequence; and a 3′ ITR. The vector genome may comprise, in 5′ to 3′ order, the polynucleotide sequences SEQ ID NO: 31; any one of SEQ ID NOs: 4, 7, and 88; or polynucleotide sequences sharing at least or about 90%, at least or about 91%, at least or about 92%, at least or about 93%, at least or about 94%, at least or about 95%, at least or about 96%, at least or about 97%, at least or about 98%, at least or about 99%, or 100% identity to each of the foregoing. In certain embodiments, this vector genome is packaged in an AAV9 or AAVrh.74 vector. The PKP2a transgene of this embodiment is a full length or substantially full length transgene, i.e., a transgene encoding; a PKPa of at least 800, at least 837, or at least 830 amino acids. In particular embodiments, the PKP2a polypeptide includes 837 amino acids. In related embodiments, instead of a PKP2a transgene, the transgene is a full length or substantially full length transgene, i.e., a transgene encoding a PKP2b of at least 800, at least 830, at least 837, or at least 881 amino acids. In particular embodiments, the PKP2b polypeptide includes 881 amino acids.

In a certain embodiment, the vector genome comprises, in 5′ to 3′ order, a 5′ ITR; a hTnnT2 promoter; a PKPb transgene; optionally a WPRE element; a polyadenylation sequence; and a 3′ ITR. The vector genome may comprise, in 5′ to 3′ order, the polynucleotide sequences SEQ ID NO: 32 or 33; any one of SEQ ID NOs: 4, 7, and 88; or polynucleotide sequences sharing at least or about 90%, at least or about 91%, at least or about 92%, at least or about 93%, at least or about 94%, at least or about 95%, at least or about 96%, at least or about 97% at least or about 98%, at least or about 99%, or 100% identity to each of the foregoing. In certain embodiments, this vector genome is packaged in an AAV9 or AAVrh.74 vector. The PKP2a transgene of this embodiment is a full length or substantially full length transgene, i.e., a transgene encoding a PKPa of at least 800, at least 837, or at least 830 amino acids. In particular embodiments, the PKP2a polypeptide includes 837 amino acids. In related embodiments, instead of a PKP2a transgene, the transgene is a full length or substantially full length transgene, i.e., a transgene encoding a PKP2b of at least 800, at least 830, at least 837, or at least 881 amino acids. In particular embodiments, the PKP2b polypeptide includes 881 amino acids.

In a certain embodiment, the vector genome comprises, in 5′ to 3′ order, a 5′ ITR; an MHCK7 promoter; SV40 intron; a PKPa transgene; optionally a WPRE element; a polyadenylation sequence; and a 3′ ITR. The vector genome may comprise, in 5′ to 3′ order, the polynucleotide sequences SEQ ID NO: 31; SEQ ID NO: 53 or 61; any one of SEQ ID NOs: 3, 6, and 87; or polynucleotide sequences sharing at least or about 90%, at least or about 91%, at least or about 92%, at least or about 93%. at least or about 94%, at least or about 95%, at least or about 96%, at least or about 97%, at least or about 98%, at least or about 99%, or 100% identity to each of the foregoing. In certain embodiments, this vector genome is packaged in an AAV9 or A AVrh.74 vector. The PKP2a transgene of this embodiment is a full length or substantially full length transgene, i.e., a transgene encoding a PKPa of at least 800, at least 837, or at least 830 amino acids. In particular embodiments, the PKP2a polypeptide includes 837 amino acids. In related embodiments, instead of a PKP2a transgene, the transgene is a full length or substantially full length transgene, i.e., a transgene encoding a PKP2b of at least 800, at least 830, at least 837, or at least 881 amino acids. In particular embodiments, the PKP2b polypeptide includes 881 amino acids.

In a certain embodiment, the vector genome comprises, in 5′ to 3′ order, a 5′ ITR; a hTnnT2 promoter; SV40 intron; a PKPa transgene; optionally a WPRE element; a polyadenylation sequence; and a 3′ ITR. The vector genome may comprise, in 5′ to 3′ order, the polynucleotide sequences SEQ ID NO: 32 or 33; SEQ ID NO: 53 or 61; any one of SEQ ID NOs: 3, 6, and 87; or polynucleotide sequences sharing at least or about 90%, at least or about 91%, at least or about 92%, at least or about 93%, at least or about 94%, at least or about 95%, at least or about 96%, at least or about 97%, at least or about 98%, at least or about 99%, or 10% identity to each of the foregoing. In certain embodiments, this vector genome is packaged in an AAV9 or AAVrh.74 vector. The PKP2a transgene of this embodiment is a full length or substantially full length transgene, i.e., a transgene encoding a PKPa of at least 800, at least 837, or at least 830 amino acids. In particular embodiments, the PKP2a polypeptide includes 837 amino acids. In related embodiments, instead of a PKP2a transgene, the transgene is a full length or substantially full length transgene, i.e., a transgene encoding a PKP2b of at least 800, at least 830, at least 837, or at least 881 amino acids. In particular embodiments, the PKP2b polypeptide includes 881 amino acids.

In a certain embodiment, the vector genome comprises, in 5′ to 3′ order, a 5′ ITR; an MHCK7 promoter; SV40 intron; a PKPb transgene; optionally a WPRE element; a polyadenylation sequence; and a 3′ ITR. The vector genome may comprise, in 5′ to 3′ order, the polynucleotide sequences SEQ TD NO: 31; SEQ ID NO: 53 or 61; any one of SEQ ID NOs: 4, 7, and 88; or polynucleotide sequences sharing at least or about 90%, at least or about 91%, at least or about 92%, at least or about 93%, at least or about 94%, at least or about 95%, at least or about 96%, at least or about 97%, at least or about 98%, at least or about 99%, or 100% identity to each of the foregoing. In certain embodiments, this vector genome is packaged in an AAV9 or AAVrh.74 vector. The PKP2a transgene of this embodiment is a full length or substantially full length transgene, i.e., a transgene encoding a PKPa of at least 800, at least 837, or at least 830 amino acids. In particular embodiments, the PKP2a polypeptide includes 837 amino acids. In related embodiments, instead of a PKP2a transgene, the transgene is a full length or substantially full length transgene, i.e., a transgene encoding a PKP2b of at least 800, at least 830, at least 837, or at least 881 amino acids. In particular embodiments, the PKP2b polypeptide includes 881 amino acids.

In a certain embodiment, the vector genome comprises, in 5′ to 3′ order, a 5′ ITR; a hTnnT2 promoter; SV40 intron; a PKPb transgene; optionally a WPRE element; a polyadenylation sequence; and a 3′ ITR. The vector genome may comprise, in 5′ to 3′ order, the polynucleotide sequences SEQ ID NO: 32 or 33; SEQ ID NO: 53 or 61; any one of SEQ ID NOs: 4, 7, and 88: or polynucleotide sequences sharing at least or about 90%, at least or about 91%, at least or about 92%, at least or about 93%. at least or about 94%, at least or about 95%, at least or about 96%, at least or about 97%, at least or about 98%, at least or about 99%, or 100% identity to each of the foregoing. In certain embodiments, this vector genome is packaged in an AAV9 or AAVrh.74 vector. The PKP2a transgene of this embodiment is a full length or substantially frill length transgene, i.e., a transgene encoding a PKPa of at least 800, at least 837, or at least 830 amino acids. In particular embodiments, the PKP2a polypeptide includes 837 amino acids. In related embodiments, instead of a PKP2a transgene, the transgene is a full length or substantially full length transgene, i.e., a transgene encoding a PKP2b of at least 800, at least 830, at least 837, or at least 881 amino acids. In particular embodiments, the PKP2b polypeptide includes 881 amino acids.

In each case the optionally WPRE element may be present or absent.

In certain embodiments, the vector is an AAVrh.74, and the vector genome encodes PKP2-A (shorter isoform, predominant expression in the heart), either with or without codon optimization (e.g., optimized for human expression and CpG island reduction) operably linked to a cardiac specific promoter. The AAVrh.74 vector is administered intravenously (IV) at a mid E13 vg/kg to mid E14 vg/kg dose range, e.g., for a patient population requiring high vector load, e.g., adults, who present on average at age 35.

### Adeno-Associated Virus Vector

AAV vectors useful in the practice of the present disclosure can be packaged into AAV virions (viral particles) using various systems including adenovirus-based and helper-free systems. Standard methods in AAV biology include but are not limited to those described in Kwon and Schaffer. Pharm Res. (2008) 25(3):489-99; Wu et al. Mol. Ther. (2006) 14(3)316-27. Burger et al. Mo. Ther. (2004) 10(2):302-17; Grimm et al. Curr Gene Ther. (2003) 3(4):281-304; Deyle D R, Russell D W. Curr Opin. Mol Ther. (2009) 11(4):442-447; McCarty et al. Gene Ther. (2001) 8(16):1248-54; and Duan et al. Mol Ther. (2001) 4(4):383-91. Illustrative helper-free systems include but are not limited to those described in U.S. Pat. Nos. 6,004,797; 7,588,772; and 7,094,604.

AAV DNA in the rAAV genomes may be from any AAV variant or AAV serotype for which a recombinant virus can be derived including, but not limited to, AAV serotypes AAV-1. AAV-2. A AV-3, AAV-4. AAV-5, AAV-6, AAV-7, A AV-8, A AV-9. AAV-10, AAV-11, AAV-12, AAV-13, and AAVrh.10, including wild-type and variants of any of these serotypes. Production of pseudotyped rAAV is disclosed in, for example, WO 01/83692. Other types of rAAV variants, for example rAAV with capsid mutations, are also contemplated See, for example. Marsic et al., Molecular Therapy, 22(11): 1900-1909 (2014). The nucleotide sequences of the genomes of various AAV serotypes are known in the art.

In some cases, the rAAV comprises a self-complementary genome. As defined herein, an rAAV comprising a “self-complementary” or “double stranded” genome refers to an rAAV which has been engineered such that the coding region of the rAAV is configured to form an intra-molecular double-stranded DNA template, as described in McCarty et al. Self-complementary recombinant adeno-associated virus (scAAV) vectors promoter efficient transduction independently of DNA synthesis. Gene Therapy. 8 (16): 1248-54 (2001). The present disclosure contemplates the use, in some cases, of an rAAV comprising a self-complementary genome because upon infection (such transduction), rather than waiting for cell mediated synthesis of the second strand of the rAAV genome, the two complementary halves of scAAV will associate to form one double stranded DNA (dsDNA) unit that is ready for immediate replication and transcription. It will be understood that instead of the full coding capacity found in rAAV (4.7-6 kb), rAAV comprising a self-complementary genome can only hold about half of that amount (≈2.4 kb).

In other cases, the rAAV vector comprises a single stranded genome. As defined herein, a “single standard” genome refers to a genome that is not self-complementary. In most cases, non-recombinant AAVs have singled stranded DNA genomes. There have been some indications that rAAVs should be scAAVs to achieve efficient transduction of cells. The present disclosure contemplates, however, rAAV vectors that may be have singled stranded genomes, rather than self-complementary genomes, with the understanding that other genetic modifications of the rAAV vector may be beneficial to obtain optimal gene transcription in target cells. In some cases, the present disclosure relates to single-stranded rAAV vectors capable of achieving efficient gene transfer to anterior segment in the mouse eye. See Wang et al. Single stranded adeno-associated virus achieves efficient gene transfer to anterior segment in the mouse eye. PLoS ONE 12(8): e0182473 (2017).

In some cases, the rAAV vector is of the serotype A AV1, AAV2, AAV4, AAV5, AAV6, AAV7, AAV8, AAV9, AAV10, AAV11, AAV12, AAV13, AAVrh.10, or AAVrh.74. Production of pseudotyped rAAV is disclosed in, for example, WO 01/83692. Other types of rAAV variants, for example rAAV with capsid mutations, are also contemplated. See, for example, Marsic et al., Molecular Therapy, 22(11): 1900-1909 (2014). In some cases, the rAAV vector is of the serotype AAV9. In some embodiments, said rAAV vector is of serotype AAV9 and comprises a single stranded genome. In some embodiments, said rAAV vector is of serotype AAV9 and comprises a self-complementary genome. In some embodiments, a rAAV vector comprises the inverted terminal repeat (ITR) sequences of AAV2. In some embodiments, the rAAV vector comprises an AAV2 genome, such that the rAAV vector is an AAV-2/9 vector, an AAV-2/6 vector, or an AAV-2/8 vector.

Full-length sequences and sequences for capsid genes for most known AAVs are provided in U.S. Pat. No. 8,524,446. which is incorporated herein in its entirety.

AAV vectors may comprise wild-type AAV sequences or they may comprise one or more modifications to a wild-type AAV sequence. In certain embodiments, an A AV vector comprises one or more amino acid modifications, optionally substitutions, deletions, or insertions, within a capsid protein, optionally VP1, VP2 and/or P3. In particular embodiments, the modification provides for reduced immunogenicity when the AAV vector is provided to a subject.

Capsid proteins of a rAAV may be modified so that the rAAV is targeted to a particular target tissue of interest such as cardiomyocytes. In some embodiments, the rAAV is directly injected into the intracerebroventricular space of the subject,

In some embodiments, the rAAV virion is an AAV2 rAAV virion. The capsid may be an A AV2 capsid or functional variant thereof. In some embodiments, the AAV2 capsid comprises a capsid protein that shares at least or about 90%, at least or about 95%, at least or about 98%, at least or about 99%, or 100% identity to a reference AAV2 capsid protein, e.g., SEQ ID NO: 76.

In some embodiments, the rAAV virion is an AAV9 rAAV virion. The capsid may be an AAV9 capsid or functional variant thereof. In some embodiments, the AAV9 capsid comprises a capsid protein that shares at least or about 90%, at least or about 95%, at least or about 98%, at least or about 99%, or 100% identity to a reference AAV9 capsid protein, e.g., SEQ ID NO: 77.

In some embodiments, the rAAV virion is an AAV6 rAAV virion. The capsid may be an AAV9 capsid or functional variant thereof. In some embodiments, the AAV6 capsid comprises a capsid protein that shares at least or about 90%, at least or about 95%, at least or about 98%, at least or about 99%, or 100% identity to a reference AAV6 capsid protein, e.g., SEQ ID NO: 78.

In some embodiments, the rAAV virion is an AAVrh.10 rAAV virion. The capsid may be an AAV9 capsid or functional variant thereof. In some embodiments, the AAVrh 10 capsid comprises a capsid protein that shares at least or about 90%, at least or about 95%, at least or about 98%, at least or about 99%, or 100% identity to a reference AAVrh.10 capsid protein, e.g., SEQ ID NO: 79.

In some embodiments, the rAAV virion is an AAVrh.74 rAAV virion. The capsid may be an A AVrh.74 capsid or functional variant thereof. In some embodiments, the AAVrh.74 capsid comprises a capsid protein that shares at least or about 90%, at least or about 95%, at least or about 98%, at least or about 99%, or 100% identity to a reference AAVrh.74 capsid protein. e.g., SEQ ID NOs: 81-83.

The polynucleotide sequence of wild-type AAVrh.74 cap is provided as SEQ ID NO: 80. The disclosure further provides protein sequences for AAVrh.74 VP1, VP2, and VP3, including SEQ ID NOs: 81-83, and homologs or functional variants thereof.

(AAVrh.74 VP1; SEQ ID NO: 81)
MAADGYLPDWLEDNLSEGIREWWDLKPGAPKPKANQQKQDNGRGLVLPGYKYLG
PFNGLDKGEPVNAADAAALEHDKAYDQQLQAGDNPYLRYNHADAEFQERLQEDTS
FGGNLGRAVFQAKKRVLEPLGLVESPVKTAPGKKRPVEPSPQRSPDSSTGIGKKGQQ
PAKKRLNFGQTGDSESVPDPQPIGEPPAGPSGLGSGTMAAGGGAPMADNNEGADGV
GSSSGNWHCDSTWLGDRVITTSTRTWALPTYNNHLYKQISNGTSGGSTNDNTYFGY
STPWGYFDFNRFHCHFSPRDWQRLINNNWGFRPKRLNFKLFNIQVKEVTQNEGTKTI
ANNLTSTIQVFTDSEYQLPYVLGSAHQGCLPPFPADVFMIPQYGYLTLNNGSQAVGR
SSFYCLEYFPSQMLRTGNNFEFSYNFEDVPFHSSYAHSQSLDRLMNPLIDQYLYYLSR
TQSTGGTAGTQQLLFSQAGPNNMSAQAKNWLPGPCYRQQRVSTTLSQNNNSNFAW
TGATKYHLNGRDSLVNPGVAMATHKDDEERFFPSSGVLMFGKQGAGKDNVDYSSV
MLTSEEEIKTTNPVATEQYGVVADNLQQQNAAPIVGAVNSQGALPGMVWQNRDVY
LQGPIWAKIPHTDGNFHPSPLMGGFGLKHPPPQILIKNTPVPADPPTTFNQAKLASFIT
QYSTGQVSVEIEWELQKENSKRWNPEIQYTSNYYKSTNVDFAVNTEGTYSEPRPIGT
RYLTRNL
(AAVrb.74 VP2; SEQ ID NO: 82)
TAPGKKRPVEPSPQRSPDSSTGIGKKGQQPAKKRLNFGQTGDSESVPDPQPIGEPPAG
PSGLGSGTMAAGGGAPMADNNEGADGVGSSSGNWHCDSTWLGDRVITTSTRTWAL
PTYNNHLYKQISNGTSGGSTNDNTYFGYSTPWGYFDFNRFHCHFSPRDWQRLINNN
WGFRPKRLNFKLFNIQVKEVTQNEGTKTIANNLTSTIQVFTDSEYQLPYVLGSAHQG
CLPPFPADVFMIPQYGYLTLNNGSQAVGRSSFYCLEYFPSQMLRTGNNFEFSYNFED
VPFHSSYAHSQSLDRLMNPLIDQYLYYLSRTQSTGGTAGTQQLLFSQAGPNNMSAQ
AKNWLPGPCYRQQRVSTTLSQNNNSNFAWTGATKYHLNGRDSLVNPGVAMATHK
DDEERFFPSSGVLMFGKQGAGKDNVDYSSVMLTSEEEIKTTNPVATEQYGVVADNL
QQQNAAPIVGAVNSQGALPGMVWQNRDVYLQGPIWAKIPHTDGNFHPSPLMGGFG
LKHPPPQILIKNTPVPADPPTTFNQAKLASFITQYSTGQVSVEIEWELQKENSKRWNPE
IQYTSNYYKSTNVDFAVNTEGTYSEPRPIGTRYLTRNL
(AAVrh.74 VP3; SEQ ID NO: 83)
MAAGGGAPMADNNEGADGVGSSSGNWHCDSTWLGDRVITTSTRTWALPTYNNHL
YKQISNGTSGGSTNDNTYFGYSTPWGYFDFNRFHCHFSPRDWQRLINNNWGFRPKR
LNFKLFNIQVKEVTQNEGTKTIANNLTSTIQVFTDSEYQLPYVLGSAHQGCLPPFPAD
VFMIPQYGYLTLNNGSQAVGRSSFYCLEYFPSQMLRTGNNFEFSYNFEDVPFHSSYA
HSQSLDRLMNPLIDQYLYYLSRTQSTGGTAGTQQLLFSQAGPNNMSAQAKNWLPGP
CYRQQRVSTTLSQNNNSNFAWTGATKYHLNGRDSLVNPGVAMATHKDDEERFFPS
SGVLMFGKQGAGKDNVDYSSVMLTSEEEIKTTNPVATEQYGVVADNLQQQNAAPI
VGAVNSQGALPGMVWQNRDVYLQGPIWAKIPHTDGNFHPSPLMGGFGLKHPPPQIL
IKNTPVPADPPTTFNQAKLASFITQYSTGQVSVEIEWELQKENSKRWNPEIQYTSNYY
KSTNVDFAVNTEGTYSEPRPIGTRYLTRNL

In certain cases, the AAVrh.74 capsid comprises the amino acid sequence set forth in SEQ ID NO: 81. In some embodiments, the rAAV vector comprises a polypeptide that comprises, or consists essentially of, or yet further consists of a sequence, e.g., at least 65%, at least 70%, at least 75%, at least 80%, 81%, 82%, 83%, 84%, 85%, 86%, 87%, 88%, or 89%, more typically 90%, 91q %, 92%, 93%, 94%, 95%, 96%, 97%, 98%, 99% or more identical to amino acid sequence of AAVrh.74 VP1 which is set forth in SEQ ID NO: 81. In some embodiments, the rAAV vector comprises a polypeptide that comprises, or consists essentially of, or yet further consists of a sequence, e.g. at least 65%, at least 70%, at least 75%, at least 80%, 81%, 82%, 83%, 84%, 85%, 86%, 87%, 88%, or 89%, more typically 90%, 91%, 92%, 93%, 94%, 95%, 96%, 97%, 98%, 99% or more identical to amino acid sequence of AAVrh.74 VP2 which is set forth in SEQ ID NO: 82. In some embodiments, the rAAV vector comprises a polypeptide that comprises, or consists essentially of, or yet further consists of a sequence, e.g., at least 65%, at least 70%, at least 75%, at least 80%, 81%, 82%, 83%, 84%, 85%, 86%, 87%, 88%, or 89%, more typically 90%, 91%, 92%, 93%, 94%, 95%, 96%, 97%, 98%, 99% or more identical to amino acid sequence of AAVrh.74 VP3 which is set forth in SEQ ID NO: 83.

In some embodiments, the rAAV virion is an A AV-PHP, B rAAV virion or a neurotrophic variant thereof, such as, without limitation, those disclosed in Int'l Pat. Pub. Nos. WO 2015/038958 A1 and WO 2017/100671 A1. For example, the AAV capsid protein may comprise at least 4 contiguous amino acids from the sequence TLAVPFK (SEQ ID N0:85) or KFPVALT (SEQ ID NO:86), e.g., inserted between a sequence encoding for amino acids 588 and 589 of AAV9 capsid protein.

The capsid may be an AAV-PHP.B capsid or functional variant thereof. In some embodiments, the AAV-PHP.B capsid comprises a capsid protein that shares at least 98%, 99%, or 100% identity to a reference AAV-PHP.B capsid protein. e.g., SEQ ID NO: 84.

Further AAV capsids used in the rAAV virions of the disclosure include those disclosed in Pat. Pub. Nos. WO 2009/012176 A2 and WO 2015/168666 A2.

Without being bound by theory, the present inventors have determined that an AAV9 vector, e.g., an AAVrh.74 or an AAVrh.10 vector, will confer desirable cardiac tropism on the vector. Without being bound by theory, the present inventors have further determined that an AAV9 vector, e.g., an AAVrh.74 or an AAVrh.10 vector, will provide desired specificity to cardiac cells.

In an aspect, the disclosure provides pharmaceutical compositions comprising the rAAV virion of the disclosure and one or more pharmaceutically acceptable carriers, diluents, or excipients.

For purposes of administration, optionally by injection, various solutions can be employed, such as sterile aqueous solutions, Such aqueous solutions can be buffered, if desired, and the liquid diluent first rendered isotonic with saline or glucose. Solutions of rAAV as a free acid (DNA contains acidic phosphate groups) or a pharmacologically acceptable salt can be prepared in water suitably mixed with a surfactant such as Poloxamer 188, e.g., at 0.001% or 0.01%. A dispersion of rAAV can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical forms suitable for injectable use include but are not limited to sterile aqueous solutions or dispersions and sterile powders for the extemporaneous preparation of sterile injectable solutions or dispersions. In all cases the form is sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example. glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions may be prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the certain methods of preparation are vacuum drying and the freeze-drying technique that yield a powder of the active ingredient plus any additional desired ingredient from the previously sterile-filtered solution thereof.

In another aspect, the disclosure comprises a kit comprising an rAAV virion of the disclosure and instructions for use.

In an aspect, the disclosure provides a method of increasing PKP2 activity in a cell, comprising contacting the cell with an rAAV of the disclosure. In another aspect, the disclosure provides a method of increasing PKP2 activity in a subject, comprising administering to the subject an rAAV of the disclosure. In some embodiments, the cell and/or subject is deficient in PKP2 messenger RNA or PKP2 protein expression levels and/or activity and/or comprises a loss-of-function mutation in PKP2. The cell may be a cardiac cell, e.g., a cardiomyocyte cell. In particular embodiments, the subject is a mammal, e.g., a human.

In some embodiments, the method promotes survival of cardiac cell, e.g. a cardiomyocyte cell, in cell culture and/or in vivo. In some embodiments, the method promotes and/or restores function of the heart.

In another aspect, the disclosure provides a method of treating a disease or disorder in a subject in need thereof, comprising administering to the subject an effective amount of an rAAV virion of the disclosure. In some embodiments, the disease or disorder is a cardiac disease or disorder, Illustrative cardiac disorders include heart failure, arrhythmogenic right ventricular cardiomyopathy (ACM), Brugada syndrome (BrS) and idiopathic ventricular fibrillation. In certain embodiments, the subject suffers from or is at risk for arrhythmogenic right ventricular cardiomyopathy (ACM). In particular embodiments, the subject is a mammal, e.g., a human, having a loss-of-function mutation in a PKP2?gene. In particular methods, treatment with the rAAV virion results in expression of the PKP2 protein encoded by the rAAV virion in the subject, e.g., in the subject's heart or cardiac tissue. In certain embodiments, treatment with the rAAV virion results in at least two-fold, at least five-fold, at least ten-fold, or more PKP2 protein levels detectable in the subject's heart.

High-risk PKP2 patients include, e.g., 1) carriers of protein-truncating PKP2 variants including nonsense, frameshift, deletions, stop-gain mutations, 2) patients with ICD implant or 3) patients who meet HRS class I or Ila criteria for ICD placement (nay have LCD in place). In particular embodiments, methods of the disclosure may be used to treat any of the disclosed patient populations.

STOP-GAIN variants are a type of nonsense mutation, e.g., a substitution which causes the gain of a stop codon and truncated protein with loss of function (or due to nonsense-mediated decay, degradation of the truncated transcript and significant decrease in the amount of protein). Non-limiting examples of STOP-GAIN variant mutations include, e.g., Arg79X, Tyr86X, Gin133X, Val406SerfsX3, Tyr616X, Trp676X, Tyr807X, Trp848X, Y86X, V406X, Y616X, W848X, and Y807X, relative to a human PKP2 gene encoding a human PKP2 having the sequence of SEQ ID NO: 2, wherein X indicates a premature translation termination codon (also designated * or Ter) caused by a nonsense mutation or a frameshift and early stop. High-end PKP2 prevalence (163k) * PKP2 stop-gain frequency (0.28) * PKP2 ICD frequency (0.41)=18.7k pts. High-end PKP2 prevalence (153k) * PKP2 stop-gain frequency (0.28) * PKP2 ICD frequency (0.41) 17.5k pts.

In particular embodiments, subjects include PKP2-ACM patients with a pathogenic PKP2 variant and clinical diagnosis. ACM prevalence ranges from 1:1000 to 1:5000 (Peters S, Trümmel M, Meyners W. Prevalence of right ventricular dysplasia-cardiomyopathy in a non-referral hospital. Int I Cardiol. 2004; 97(3):499-501; and McKenna W J, Judge D P. Epidemiology of the inherited cardiomyopathies. Nat Rev Cardiol. 2021; 18(1):22-36). In 2,572 ACM patients assessed from 13 publications an aggregated mean of 32.9% had PKP2 mutations.

| Study | Geography | ARVC N | PKP2N | % | DOI |
|---|---|---|---|---|---|
| Christensen et al., | | 230 | 94 | 40.9 | 10.1136/jmedgenet-2021-107911 |
| 2021 | | | | | |
| Protonotarios et al., | WW | 554 | 118 | 21.3 | 10.1093/eurheartj/ehac235 |
| Eur Heart J, 2022 | | | | | |
| Jorda et al., Eur Heart | North America | 429 | 111 | 25.9 | 10.1093/eurheartj/ehac289 |
| J, 2022 | & Europe | | | | |
| Paldino et al., 2022 | US, Italy | 70 | 24 | 34.3 | 10.1016/j.jacc.2022.08.804 |
| Kato et al., 2015 | Japan | 57 | 11 | 19.3 | 10.1016/j.jjcc.2015.10.013 |
| Groeneweg et al., Circ | US, | 439 | 202 | 46 | 10.1161/CIRCGENETICS.114.001003 |
| Genetics, 2015 | Netherlands | | | | |
| Alcalde et al., 2014 | Spain | 30 | 13 | 43.3 | 10.1371/journal.pone.0100560 |
| Cox et al., Circulation | | 147 | 76 | 51.7 | 10.1161/circulationaha.110.988287 |
| 2011 | | | | | |
| Kapplinger et al., | | 175 | 87 | 49.7 | 10.1016/j.jacc.2010.12.036 |
| Circulation, 2011 | | | | | |
| Klauke et al., 2010 | Germany | 23 | 6 | 26.1 | 10.1093/hmg/ddq387 |
| Fressart et al., 2010 | France | 135 | 42 | 31.1 | 10.1093/europace/euq104 |
| Xu et al., 2010 | North America, | 198 | 38 | 29.6 | 10.1016/j.jacc.2009.11.020 |
| | Italy | | | | |
| Bhuiyan et al., 2009 | Netherlands | 85 | 24 | 29.6 | 10.1161/circgenetics.108.839829 |
| Total | | 2572 | 846 | 32.9 | |

Utilizing the conservative ACM prevalence (1:5000) and the 32.9% PKP2 mutation frequency in ACM, the prevalence of PKP2-ACM patients across the US and EU is approximately 50,000 patients.

In particular embodiments, the subject comprises a PKP2 STOP-GAIN VARIANT mutation, including but not limited to any specifically disclosed herein, while in other embodiments, the subject may comprise a different type of PKP2 mutation.

In particular embodiments, the subject may comprise an implantable cardioverter-defibrillator (ICD), while in other embodiments, the subject may not comprise an ICD

In particular embodiments, the subject comprises a PKP2 STOP-GAIN VARIANT mutation, including but not limited to any disclosed herein, while in other embodiments, the subject may comprise a different type of PKP2 mutation and an implantable cardioverter-defibrillator (ICD).

In particular embodiments, a subject treated with the disclosed vectors or according to the disclosed methods comprises a PKP2 STOP-GAIN VARIANT mutation and/or has an implantable cardioverter-defibrillator (ICD).https://www.mayoclinic.org/tests-procedures/implantable-cardioverter-defibrillators/about/pac-20384692

The AAV-mediated delivery of PKP2 protein to the heart may increase life span, prevent or attenuate cardiac cell degeneration, heart failure, scarring, reduced ejection fraction, arrythmia, angina, exercise intolerance, angina (chest pain), sudden cardiac death, exertional myalgias and cramps. The AAV-mediated delivery of PKP2 protein to the heart may show improvement from or prevent normal disease course detected by use of pathological electrocardiogram, cardiac MRI, heart biopsy, decrease in paroxysmal ventricular arrhythmias, decrease in sudden cardiac death, and/or decrease in or lack of further development of fibro-fatty deposits in right ventricular myocardium. The methods of the disclosure may prevent a decrease in, restore, and/or increase right ventricular ejection fraction (RVEF).

The methods disclosed herein may provide efficient biodistribution in the heart. They may result in sustained in expression in all, or a substantial fraction of, cardiac cells, e.g., cardiomyocytes. Notably, the methods disclosed herein may provide long-lasting expression of PKP2 protein throughout the life of the subject following AAV vector administration. In some embodiments, PKP2 protein expression in response to treatment lasts at least 1, 2, 3, 4, 5, C, 7, 8, 9, 10, 15, 20, 25, 30, 35, or 40 years.

Combination therapies are also contemplated by the disclosure. Combinations of methods of the disclosure with standard medical treatments (e.g., corticosteroids or topical pressure reducing medications) are specifically contemplated, as are combinations with novel therapies. In some cases, a subject may be treated with a steroid and/or combination of immune suppressing agents to prevent or to reduce an immune response to administration of a rAAV described herein.

In some embodiments, the AAV vector is administered at a dose of between about 1×10¹² and 5×10¹⁴ vector genomes (vg) or between about 1×10¹² and 6×10¹⁴ vg of the AAV vector per kilogram (kg) of total body mass of the subject (vg/kg). In some embodiments, the AAV vector is administered at a dose of between about 1×10¹³ and 5×10¹⁴ vg/kg. In some embodiments, the AAV vector is administered at a dose of between about 1×10¹³ and 1×10¹⁴ vg/kg. In some embodiments, the AAV vector is administered at a dose of between about 3×10¹³ and 3×10¹⁴ vg/kg. In some embodiments, the AAV vector is administered at a dose of between about 5×10¹³ and 3×10¹⁴ vg/kg. In some embodiments, the AAV vector is administered at a dose of between about 5×10¹³ and 5×10¹⁴ vg/kg. In some embodiments, the AAV vector is administered at a dose of between about 5×10¹³ and 1×10¹⁴ vg/kg. In some embodiments, the AAV vector is administered at a dose of less than about 1×10¹² vg/kg, less than about 3×10¹² vg/kg, less than about 5×10¹² vg/kg, less than about 7×10¹² vg/kg, less than about 1×10¹³ vg/kg, less than about 3×10¹³ vg/kg, less than about 5×10¹³ vg/kg, less than about 7×10¹³ vg/kg, less than about 1×10¹⁴ vg/kg, less than about 3×10¹³ vg/kg, less than about 5×10¹⁴ vg/kg, less than about 7×10¹⁴ vg/kg, less than about 1×10¹³ vg/kg, less than about 3×10¹⁵ vg/kg, less than about 5×10¹³ vg/kg, or less than about 7×10¹⁵ vg/kg. In certain embodiments, the AAV vector delivered at any of these doses is an AAV9 vector or an AAV rh. 74 vector, e.g., a vector comprising a sequence disclosed in any of SEQ ID NOs: 8-15 or 89-96. In some cases, it may be advantageous to use a higher dose for an AAV rh.74 vector than for an AAV9 vector. In certain embodiments, an AAV9 vector is administered at a dose of between about 1×10¹³ and about 1×10¹⁴ vg/kg, e.g., about 1×¹³, about 2×10¹³, about 3×10¹³, about 4×10¹³, or about 5×10¹³ vg/kg. In certain embodiments, an AAVrh.74 vector is administered at a dose of between about 5×10¹³ and about 5×10¹⁴ vg/kg, e.g., about 5×10¹³ about 6×10¹³, about 7×10¹³, about 8×10¹³, about 9×10¹³, about 1×10¹³ about 2×10¹³, about 3×10¹⁴ about 4×10¹⁴, or about 5×10¹³ vg/kg. In some embodiments, the AAV vector, e.g., an AVrh.74 vector, is administered at a dosage of at least about 5×10¹³ vg/kg, at least about 6×10¹³ vg/kg, at least about 7×10¹³ vg/kg, at least about 8/10¹³ vg/kg, at least about 9×10¹³ vg/kg, at least about 1×10¹⁴ vg/kg, at least about 2×10¹³ vg/kg, at least about 3 10¹⁴ vg/kg, at least about 4×10¹⁴ vg/kg, at least about 5×10¹⁴ vg/kg, at least about 6×10¹⁴ vg/kg, or at least about 7×10¹⁴ vg/kg.

In some embodiments, the AAV vector is administered at a dose of about 1×10¹² vg/kg, about 3×10¹³ vg/kg, about 5×10¹³ vg/kg, about 7×10¹³ vg/kg, about 1×10¹³ vg/kg, about 2×10¹³ vg/kg, about 3×10¹³ vg/kg, about 4×10¹³ vg/kg. about 5×10¹³ vg/kg. about 6×10¹³ vg/kg, about 7×10¹³ vg/kg, about 8×10¹³ vg/kg, about 9×10¹³ vg/kg, about 1×10¹⁴ vg/kg, about 2×10¹⁴ vg/kg, about 3×10¹⁴ vg/lg, about 4×10¹³ vg/kg, about 5×10¹³ vg/kg, about 6×10¹⁴ vg/kg, about 7×10¹³ vg/kg, about 8×10¹³ vg/kg, about 9×10¹⁴ vg/kg, about 1×10¹⁵ vg/kg, about 3×10¹⁵ vg/kg, about 5×10¹³ vg/kg, or about 7×10¹³ vg/kg. In certain embodiments, the AAV vector delivered at any of these doses is an AAV9 vector or an AAV rh.74 vector, e.g., a vector comprising a sequence disclosed in any of SEQ ID NOs: 8-15 or 89-96. In certain embodiments, an AAV9 vector is administered at a dose of between about 1×10¹³ and about 1×10¹⁴ vg/kg. e.g., about 1×10¹³, about 2×10¹³, about 3×10¹³. about 4×10¹³, or about 5 ×¹³ vg/kg. In certain embodiments, an AAVrh.74 vector is administered at a dose of between about 5×10¹³ and about 5×10¹⁴ vg/kg, e.g., about 5×10¹³, about 6×10¹³, about 7×10¹³, about 8×10¹³ about 9×10¹³ about 1×10¹⁴ about 2×10¹⁴, about 3×10¹⁴, about 4×10¹⁴ or about 5×10¹³ vg/kg. In some embodiments, the AAV vector, e.g., an AVrh.74 vector, is administered at a dosage of at least about 5×10¹³ vg/kg, at least about 6×10¹³ vg/kg, at least about 7×10¹³ vg/kg, at least about 8/10¹³ vg/kg, at least about 9/10¹³ vg/kg, at least about 1×10¹⁴ vg/kg, at least about 2×10¹⁴ vg/kg, at least about 3×10¹⁴ vg/kg, at least about 4×10¹⁴ vg/kg, at least about 5×10¹⁴ vg/kg, at least about 6×10¹⁴ vg/kg, or at least about 7×10¹⁴ vg/kg.

In some embodiments, the AAV vector is administered at a dose of 1×10¹² vg/kg, 3×10¹³ vg/kg, 5×10¹³ vg/kg, 7×10¹² vg/kg, 1×10¹³ vg/kg 2×10¹³ vg/kg 3×10¹⁵ vg/kg, 4×10¹³ vg/kg, 5×10¹³ vg/kg, 6×10¹³ vg/kg, 7×10¹³ vg/kg , 8×10¹³ vg/kg, 9×10¹³ vg/kg, 1×10¹⁴ vg/kg, 2×10¹⁴ vg/kg, 3×10¹⁴ vg/kg, 4×10¹⁴ vg/kg, 5×10¹³ vg/kg, 6×10¹³ vg/kg, 7×10¹³ vg/kg, 8×10¹⁴ vg/kg, 9×10¹⁴ vg/kg, 1×10¹³ vg/kg 3×10¹³ kg, 5×10¹³ vg/k, or 7×10¹³ vg/kg. In certain embodiments, the AAV vector delivered at any of these doses is an AAV9 vector or an AAV rh.74 vector, e.g., a vector comprising a sequence disclosed in any of SEQ ID NOS: 8-15 or 89-96. In certain embodiments, an AAV9 vector is administered at a dose of between about 1×10¹³ and about 1×10¹³ vg/kg, e.g., about 1×10¹³ , about 2×10¹³, about 3×10¹³ about 4×10¹³ or about 5×10¹³ vg/kg. In certain embodiments, an AAVrh74 vector is administered at a dose of between about 5×10¹³ and about 5×10¹³ vg/kg, e.g., about 5×10¹³, about 6×10¹³, about 7×10¹³ about 8×10¹³ about 9×10¹³ about 1×10¹⁴ about 2×10¹³, about 3×10¹³ about 4×10¹⁴, or about 5×10¹⁴ vg/kg. In some embodiments, the AAV vector, e.g., an AVrb.74 vector, is administered at a dosage of at least about 5×10¹³ vg/kg, at least about 6×10¹³ vg/kg, at least about 7×10¹³ vg/kg, at least about 8×10¹³ vg/kg, at least about 9×10¹³ vg/kg, at least about 1×10¹³ vg/kg, at least about 2×10¹³ vg/kg, at least about 3×10¹⁴ vg/kg, at least about 4×10¹³ vg/kg, at least about 5×10¹⁴ vg/kg, at least about 6×10¹⁴ vg/kg, or at least about 7×10¹⁴ vg/kg.

In some embodiments, the AAV vector is administered systemically at a dose of between about 1×10¹² and 5×10¹⁴ vector genomes (vg) of the AAV vector per kilogram (kg) of total body mass of the subject (vg/kg). In some embodiments, the AAV vector is administered systemically at a dose of between about 1×10¹³ and 5×10¹³ vg/kg. In some embodiments, the A AV vector is administered systemically at a dose of between about 5×10¹³ and 3×10¹³ vg/kg. In some embodiments, the AAV vector is administered systemically at a dose of between about 5×10¹³ and 1×10¹⁴ vg/kg. In some embodiments, the AAV vector is administered systemically at a dose of less than about 1×10¹² vg/kg, less than about 3×10¹² vg/kg, less than about 5×10¹² vg/kg, less than about 7×10¹² vg/kg, less than about 1×10¹³ vg/kg, less than about 3×10¹³ vg/kg, less than about 5 10¹² vg/kg, less than about 7×10¹³ vg/kg, less than about 1×10¹⁴ vg/kg, less than about 3×10¹³ vg/kg, less than about 5×10¹³ vg/kg, less than about 7×10¹³ vg/kg, less than about 1×10¹³ vg/kg, less than about 3×10¹³ vg/kg, less than about 5×10¹⁵ vg/kg, or less than about 7×10¹⁵ vg/kg. In certain embodiments, the AAV vector delivered at any of these doses is an AAV9 vector or an AAV rh. 74 vector, e.g., a vector comprising a sequence disclosed in any of SEQ ID NOs: 8-15 or 89-96. In certain embodiments, an AAV9 vector is administered at a dose of between about 1×10¹³ and about 1. ×10¹³ vg/kg, e.g., about 1×10¹³, about 2×10¹³, about 3×10¹³, about 4×10¹³, or about 5×10¹³ vg/kg. In certain embodiments, an AAVrh 74 vector is administered at a dose of between about 5×10¹⁴ and about 5×10¹³ vg/kg, e.g., about 5×10¹³, about 6×10¹³, about 7×10¹³, about 8×10¹³, about 9×10¹³, about 2×10¹³, about 2×10¹³: about 3×10¹³, about 4×10¹⁴, or about 5×10¹³ vg/kg. In some embodiments, the AAV vector, e.g., an AVrh.74 vector, is administered at a dosage of at least about 5×10¹³ vg/kg, at least about 6×10¹³ vg/kg, at least about 7×10¹³ vg/kg, at least about 8×10¹³ vg/kg, at least about 9×10¹³ vg/kg, at least about 1×10¹³ vg/kg, at least about 2×10¹⁴ vg/kg, at least about 3×10¹⁴ vg/kg, at least about 4×10¹⁴ vg/kg, at least about 5×10¹⁴ vg/kg, at least about 6×10¹³ vg/kg, or at least about 7×10¹⁴ vg/kg.

In some embodiments, the AAV vector is administered systemically at a dose of about 1×10¹² vg/kg, about 3×10¹² vg/kg, about 5×10¹² vg/kg, about 7×10¹² vg/kg, about 1×10¹³ vg/kg, about 2×10¹³ vg/kg, about 3×10¹³ vg/kg, about 4×10¹³ vg/kg, about 5×10¹³ vg/kg, about 6×10¹³ vg/kg, about 7×10¹³ vg/kg, about 8×10¹³ vg/kg, about 9×10¹³ vg/kg, about 1×10¹³ vg/kg, about 2×10¹⁴ vg/kg, about 3×10¹⁴ vg/kg, about 4×10¹⁴ vg/kg, about 5×10¹³ vg/kg, about 6×10¹³ vg/kg, about 7×10¹³ vg/kg, about 8×10¹³ vg/kg, about 9×10¹⁴ vg/kg, about 1×10¹⁵ vg/kg, about 3×10¹⁵ vg/kg, about 5×10¹³ vg/kg, or about 7×10¹³ vg/kg. In certain embodiments, the AAV vector delivered at any of these doses is an AAV9 vector or an AAV rh.74 vector, e.g., a vector comprising a sequence disclosed in any of SEQ ID NOs: 8-15 or 89-96. In certain embodiments, an AAV9 vector is administered at a dose of between about 1×10¹³ and about 1×10¹⁴ vg/kg, e.g., about 1×10¹³, about 2×10¹³, about 3×10¹³. about 4×10¹³, or about 5×10¹³ vg/kg. In certain embodiments, an AAVrh 74 vector is administered at a dose of between about 5×10¹³ and about 5×10¹⁴ vg/kg, e.g., about 5×10¹³ about 6>×10¹³ about 7×10¹³ about 8×10¹³ about 9×10¹⁴, about 1×10¹³ about 2×10¹⁴, about 3×10¹⁴, about 4×10¹⁴, or about 5×10¹³ vg/kg. In some embodiments, the AAV vector, e.g., an AVrh.74 vector, is administered at a dosage of at least about 5×10¹³ vg/kg, at least about 6×10¹⁴ vg/kg, at least about 7×10¹³ vg/kg, at least about 8>10¹³ vg/kg, at least about 9×10¹³ vg/kg, at least about 1×10¹⁴ vg/kg, at least about 2×10¹³ vg/kg, at least about 3×10¹⁴ vg/kg, at least about 4×10¹⁴ vg/kg, at least about 5×10¹⁴ vg/kg, at least about 6×10¹⁴ vg/kg, or at least about 7×10¹⁴ vg/kg.

In some embodiments, the AAV vector is administered systemically at a dose of 1×10¹³ vg/kg, 3×10¹² vg/kg, 5×10¹³ vg/kg, 7×10¹² vg/kg, 1×10¹³ vg/kg, 2×10¹³ vg/kg, 3×10¹³ vg/kg, 4×10¹³ vg/kg, 5×10¹⁴ vg/kg, 6×10¹³ vg/kg, 7×10¹³ vg/kg, 8>×10¹³ vg/kg, 9×10¹³ vg/kg, 1×10¹⁴ vg/kg, 2×10¹⁴ vg/kg, 3×10¹⁴ vg/kg, 4×10¹⁴ vg/kg, 5×10⁴ vg/kg, 6×10¹⁴ vg/kg, 7×10¹⁴ vg/kg, 8×10¹³ vg/kg, 9×10¹² vg/kg. 1×10¹³ vg/kg 3×10¹³ vg/kg, 5×10¹³ vg/kg, or 7×10¹⁴ vg/kg. In certain embodiments, the AAV vector delivered at any of these doses is an AAV9 vector or an A AV rh.74 vector, e.g., a vector comprising a sequence disclosed in any of SEQ ID NOs: 8-15 or 89-96. In certain embodiments, an AAV9 vector is administered at a dose of between about 1×10¹³ and about 1×10¹⁴ vg/kg, e.g., about 1×10¹³ about 2×10¹³ about 3:×10¹³ about 4×10¹³. or about 5×10¹³, In certain embodiments, an AAVrh.74 vector is administered at a dose of between about 5×10¹³ and about 5×10¹⁴ vg/kg, e.g., about 5×10¹³, about 6×10¹³, about 7×10¹³, about 8×10¹³, about 9×10¹³, about 1×10¹³ , about 2×10¹⁴, about 3×10¹³ , about 4×10¹⁴ or about 5×10¹⁴ vg/kg. In some embodiments, the AAV vector, e.g., an AVrh.74 vector, is administered at a dosage of at least about 5×10¹³ vg/kg, at least about 6×10¹³ vg/kg, at least about 7×10¹³ vg/kg, at least about 8×10¹³ vg/kg, at least about 9×10¹³ vg/kg, at least about 1×10¹³ vg/kg, at least about 2×10¹³ vg/kg, at least about 3×10¹³ vg/kg, at least about 4×10¹³ vg/kg, at least about 5×10¹⁴ vg/kg, at least about 6×10¹⁴ vg/kg, or at least about 7×10¹⁴ vg/kg.

In some embodiments, the AAV vector is administered intravenously at a dose of between about 1×10¹² and 5×10¹⁴ vector genomes (vg) of the AAV vector per kilogram (kg) of total body mass of the subject (vg/kg). In some embodiments, the AAV vector is administered intravenously at a dose of between about 1×10¹³ and 5×10¹⁴ vg/kg. In some embodiments, the AAV vector is administered intravenously at a dose of between about 5×10¹³ and 3×10¹⁴ vg/kg. In some embodiments, the A AV vector is administered intravenously at a dose of between about 5×10¹³ and 1×10¹⁴ vg/kg. In some embodiments, the AAV vector is administered intravenously at a dose of less than about 1×10¹² vg/kg, less than about 3×10¹² vg/kg, less than about 5×10¹² vg/kg, less than about 7×10¹³ vg/kg, less than about 1×10¹³ vg/kg, less than about 3×10¹³ vg/kg, less than about 5×10¹³ vg/kg, less than about 7×10¹³ vg/kg, less than about 1×10¹³ vg/kg, less than about 3×10¹³ vg/kg, less than about 5×10¹³ vg/kg, less than about 7×10¹³ vg/kg, less than about 1×10¹⁴ vg/kg, less than about 3×10¹⁵ vg/kg, less than about 5×10¹⁵ vg/kg, or less than about 7×10¹⁵ vg/kg. In certain embodiments, the AAV vector delivered at any of these doses is an AAV9 vector or an AAV rh.74 vector, e.g., a vector comprising a sequence disclosed in any of SEQ ID NOs: 8-15 or 89-96. In certain embodiments, an AAV9 vector is administered at a dose of between about 1×10¹³ and about 1×10¹⁴ vg/kg, e.g., about 1×10¹³ , about 2×10¹³, about 3 ×10¹³, about 4×10¹³, or about 5×10¹³. In certain embodiments, an AAVrh.74 vector is administered at a dose of between about 5×10¹³ and about 5×10¹³ vg/kg, e.g., about 5×10¹³, about 6×10¹³, about 7×10¹³ about 8×10¹³, about 9×10¹³ about 1×10¹³ about 2×10¹⁴, about 3×10¹³ about 4×10¹⁴ or about 5×10¹⁴ vg/kg. In some embodiments, the AAV vector, e.g., an AVrh.74 vector, is administered at a dosage of at least about 5×10¹³ vg/kg, at least about 6×10¹³ vg/kg, at least about 7×10¹³ vg/kg, at least about 8×10¹³ vg/kg, at least about 9×10¹³ vg/kg, at least about 1×10¹³ vg/kg, at least about 2×10¹⁴ vg/kg, at least about 3/10¹³ vg/kg, at least about 4×10¹³ vg/kg, at least about 5×10¹⁴ vg/kg, at least about 6×10¹³ vg/kg, or at least about 7×10¹³ vg/kg.

In sone embodiments, the AAV vector is administered intravenously at a dose of about 1×10¹² vg/kg, about 3×10¹⁵ vg/kg, about 5×10¹⁵ vg/kg, about 7×10¹³ vg/kg, about 1×10¹³ vg/kg, about 2×10¹³ vg/kg, about 3×10¹³ vg/kg, about 4×10¹³ vg/kg, about 5×10¹³ vg/kg, about 6×10¹³ vg/kg, about 7×10¹³ vg/kg, about 8×10¹³ vg/kg about 9×10¹³ vg/kg, about 1×10¹³ vg/kg, about 2×10¹³ vg/kg, about 3×10¹³ vg/kg, about 4×10¹³ vg/kg, about 5×10¹⁴ vg/kg, about 6×10¹³ vg/kg, about 7×10¹⁴ vg/kg, about 8×10¹⁴ vg/kg, about 9/10¹⁴ vg/kg, about 1×10¹⁵ vg/kg, about 3×10¹⁵ vg/kg, about 5×10¹⁵ vg/kg, or about 7×10¹⁵ vg/kg. In certain embodiments, an AAV9 vector is administered at a dose of between about 1×10¹³ and about 1×10¹³ vg/kg, e.g., about 1×10), about 2×10¹³, about 3×10¹³. about 4×10, or about 5×10¹³ vg/kg. In certain embodiments, an AAVrh.74 vector is administered at a dose of between about 5×10¹³ and about 5×10¹² vg/kg, e.g., about 5×10¹³ about 6×10¹³, about 7×10¹³, about 8×10¹³ , about 9×100, about 1×10¹³ , about 2×10¹⁴, about 3×10¹⁴ about 4×10¹³ , or about 5×10¹⁴ vg/kg. In some embodiments, the A AV vector, e.g., an AVrh.74 vector, is administered at a dosage of at least about 5×10¹³ vg/kg, at least about 6×10¹³ vg/kg, at least about 7×10¹³ vg/kg, at least about 8×10¹³ vg/kg, at least about 9×10¹³ vg/kg, at least about 1/10¹⁴ vg/kg, at least about 2×10¹³ vg/kg, at least about 3×10¹⁴ vg/kg, at least about 4×10¹⁴ vg/kg, at least about 5×10¹⁴ vg/kg, at least about 6×10¹⁴ vg/kg, or at least about 7×10¹⁴ vg/kg.

In some embodiments, the AAV vector is administered intravenously at a dose of 1×10¹² vg/kg, 3×10¹² vg/kg, 5×10¹² vg/kg, 7×10¹³ vg/kg, 1×10¹³ vg/kg, 2×10¹³ vg/1g, 3×10¹³ vg/kg, 4×10¹³ vg/kg, 5×10¹³ vg/kg, 6×10¹³ vg/kg, 7×10¹³ vg/kg, 8×10¹³ vg/kg, 9×10¹³ vg/kg, 1×10¹³ vg/kg, 2×10¹² vg/kg, 3×10¹³ vg/kg, 4×10¹³ vg/kg, 5×10¹³ vg/kg, 6×10¹³ vg/kg, 7×10¹⁴ vg/kg, 8×10¹³ vg/kg, 9×10¹³ vg/kg, 1×10¹³ vg/kg, 3×10¹³ vg/kg, 5×10¹³ vg/kg, or 7×10¹³ vg/kg. In certain embodiments, the A AV vector delivered at any of these doses is an AAV9 vector or an AAV rh74 vector, e.g., a vector comprising a sequence disclosed in any of SEQ ID NOs: 8-15 or 89-96. In certain embodiments, an AAV9 vector is administered at a dose of between about 1×10¹³ and about 1×10¹³ vg/kg, e.g., about 1×10¹³, about 2×10¹³, about 3×10¹³, about 4×10¹³, or about 5×10¹³ vg/kg. In certain embodiments, an AAVrh.74 vector is administered at a dose of between about 5/10¹³ and about 5×10¹³ vg/kg, e.g., about 5/10¹³ about 6×10¹³, about 7×10¹³, about 8×10¹³ about 9×10¹³ about 1×10¹³, about 2×10¹³, about 3×10¹³, about 4×10¹³, or about 5×10¹³ vg/kg. In some embodiments, the AAV vector, e.g., an AVrh.74 vector, is administered at a dosage of at least about 5×10¹³ vg/kg, at least about 6×10¹³ vg/kg, at least about 7×10¹³ vg/kg, at least about 8×10¹³ vg/kg, at least about 9×10¹³ vg/kg, at least about 1×10¹⁴ vg/kg, at least about 2×10¹³ vg/kg, at least about 3×10¹⁴ vg/kg, at least about 4×10¹⁴ vg/kg, at least about 5×10¹³ vg/kg, at least about 6×10¹⁴ vg/kg, or at least about 7×10¹⁴ vg/kg.

Evidence of functional improvement, clinical benefit or efficacy in patients may be revealed by change in New York Heart Association functional classification (NYH-A Class), pathological electrocardiogram, cardiac MRI, heart biopsy, decrease in peroxisomal ventricular arrhythmias, decrease in sudden cardiac death, and/or decrease in or lack of further development of fibro-fatty deposits in right ventricular myocardium. Benefit may be observed in electrocardiographic features normally associated with arrhythmogenic right ventricular cardiomyopathy such as T wave inversion, prolonged S-wave upstroke, localized QRS widening, and/or paroxysmal episodes of ventricular tachycardia.

In some embodiments, the method prevents or reduces a decrease in left ventricle ejection fraction percentage (LVEF %), optionally by about 50%, about 60%, about 70%, about 80%, about 90%, or about 100% compared to the decrease observed in an untreated subject suffering from or at risk for disease or disorder related to or caused by loss of function in PKP2.

In some embodiments, the method prevents or reduces a decrease in left ventricle fractional shortening percentage (FS %), optionally by about 50%, about 60%, about 70%, about 80%, about 90%, or about 100% compared to the decrease observed in an untreated subject suffering from or at risk for disease or disorder related to or caused by loss of function in PKP2.

In some embodiments, the method prevents or reduces an increase in right ventricle area in millimeters squared RV Area (mm2), optionally by about 50%, about 60%, about 70%, about 80%, about 90%, or about 100% compared to the increase observed in an untreated subject suffering from or at risk for disease or disorder related to or caused by loss of function in PKP2.

In some embodiments, the method prevents or reduces a decrease in right ventricle velocity time integral in millimeters per second RV VTI (mm/sec), optionally by about 50%, about 60%, about 70%, about 80%, about 90%, or about 100% compared to the decrease observed in an untreated subject suffering from or at risk for disease or disorder related to or caused by loss of function in PKP2

In some embodiments, the method prevents or reduces an increase in left ventricle or right ventricle fibrosis, optionally by about 50%, about 60%, about 70%, about 80%, about 90%, or about 100% compared to the increase observed in an untreated subject suffering from or at risk for disease or disorder related to or caused by loss of function in PKP2.

Administration of an effective dose of the compositions may be by routes standard in the art including, but not limited to, systemic, local, direct injection, intravenous, intracardiac administration. In some cases, administration comprises systemic, local, direct injection, intravenous, intracardiac injection. Administration may be performed by cardiac catheterization,

In some embodiments, the disclosure provides for local administration and systemic administration of an effective dose of rAAV and compositions of the disclosure. For example, systemic administration may be administration into the circulatory system so that the entire body is affected. Systemic administration includes parental administration through injection, infusion or implantation. Routes of administration for the compositions disclosed herein include intravenous (“IV”) administration, intraperitoneal (“IP”) administration, intramuscular (“IM”) administration, intralesional administration, or subcutaneous (“SC”) administration, or the implantation of a slow-release device, e.g. a mini-osmotic pump, a depot formulation, etc. In some embodiments, the methods of the disclosure comprise administering an A AV vector of the disclosure, or pharmaceutical composition thereof by intravenous, intramuscular, intraarterial, intrarenal, intraurethral, intracardiac, intracoronary, intramyocardial, intradermal, epidural, subcutaneous, intraperitoneal, intraventricular, ionophoretic or intracranial administration.

In particular, administration of rAAV of the present disclosure may be accomplished by using any physical method that will transport the rAAV recombinant vector into the target tissue of an animal. Administration includes, but is not limited to, injection into the heart.

In some embodiments, the methods of the disclosure comprise intracardiac delivery. Infusion may be performed using specialized cannula, catheter, syringe/needle using an infusion pump. Administration may comprise delivery of an effective amount of the rAAV virion, or a pharmaceutical composition comprising the rAAV virion, to the heart. These may be achieved, e.g., via intravenous, intramuscular, intraarterial, intrarenal, intraurethral, intracardiac, intracoronary, intramyocardial, intradernal, epidural, subcutaneous, intraperitoneal, intraventricular, ionophoretic or intracranial administration. The compositions of the disclosure may further be administered intravenously.

The method of treatment disclosed herein may reduce and/or prevent one or more symptoms including but not limited to ventricular hypertrophy, ventricular tachycardia, exercise intolerance, angina, and reduced RVEF. Benefit of AAV-mediated PKP2 overexpression may be evidenced by increase in survival, mitigation of the normal progression of cardiomyopathy observed on echocardiograms from left and/or right ventricle (e.g., greater left ventricular ejection fraction, greater left ventricle fractional shortening, and greater right ventricle velocity time interval, compared to PKP eKO formulation buffer control animals).

Electrophysiological evidence of functional benefit of AAV-mediated delivery of PKP2 protein may be demonstrated by mitigation of disease-related disrupted calcium dynamics in affected cardiomyocytes, most notably on measures of L-type calcium current, sarcoplasmic reticulum calcium leak, diastolic calcium leak, as well as standard measures of calcium transients in affected (e.g., PKP2-deficient) cardiomyocytes such as time to peak amplitude and relaxation time constants. Histological analyses may reveal benefit of AAV-mediated PKP2 overexpression by diminished appearance of disease-related collagen deposition (e.g., via trichrome stain) in various regions of the heat including ventricles. Additional benefit may also be revealed by evaluating cardiomyocyte ventricular proteins involved in calcium signaling; pathways, measured by increased (i.e., normalized) relative levels of Casq2, and/or Trdn, and/or Cav 1.2, and/or AnkB and/or RyR2.

Effects of rAAV Administration

In some embodiments, administration of rAAV of the present disclosure may have beneficial effects for the subject.

### Lifespan

In some embodiments, administration of rAAV of the present disclosure may increase lifespan of the subject compared to a subject that is not administered the rAAV of the present disclosure or to baseline.

In some embodiments, administration of rAAV of the present disclosure increases lifespan by at least about 1%, at least about 2%, at least about 3%, at least about 4%, at least about 5%, at least about 6%, at least about 7%, at least about,at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 300%, at least about 400%, or at least about 500% compared to a subject that is not administered the rAAV of the present disclosure or to baseline.

In some embodiments, administration of rAAV of the present disclosure increases lifespan by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, about 95% to about 100%, about 100% to about 200%, about 200% to about 300%, about 300% to about 400%, or by about 400% to about 50% compared to a subject that is not administered the rAAV of the present disclosure or to baseline.

### Ejection Fraction

In some embodiments, administration of rAAV of the present disclosure limits a decrease in, restores, and/or increases the ejection fraction in a subject compared to a subject that is not administered the rAAV of the present disclosure or to baseline. In some embodiments, administration of rAAV of the present disclosure limits a decrease in, restores, and/or increases in the ejection fraction in a subject over time.

In some embodiments, administration of rAAV of the present disclosure restores and/or increases the ejection fraction by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to a subject that is not administered the rAAV of the present disclosure or to baseline.

In some embodiments, administration of rAAV of the present disclosure limits a decrease in the ejection fraction to less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, at least about 75%, less than about 80%, less than about 85%, less than about 90%, less than about 95%, or less than about 100% compared to a subject that is not administered the rAAV of the present disclosure or to baseline.

In some embodiments, administration of rAAV of the present disclosure restores and/or increases the ejection fraction by at least about 11%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% in a subject over time.

In some embodiments, administration of rAAV of the present disclosure limits a decrease in the ejection fraction to less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, at least about 75%, less than about 80%, less than about 85%, less than about 90%, less than about 95%, or less than about 100% in a subject over time.

In some embodiments, administration of rAAV of the present disclosure restores and/or increases the ejection fraction by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% compared to a subject that is not administered the rAAV of the present disclosure or to baseline.

In sone embodiments. administration of rAAV of the present disclosure restores and/or increases the ejection fraction by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% in a subject over time.

In some embodiments, administration of rAAV of the present disclosure limits a decrease in the ejection fraction to less than about 1% to less than about 90%, less than about 20% to less than about 80%, less than about 30% to less than about 80%, less than about 40% to less than about 80%, less than about 50% to less than about 80%, less than about 1% to less than about 2%, less than about 2% to less than about 3%, less than about 3% to less than about 4%, less than about 4% to less than about 5%, less than about 5% to less than about 6%, less than about 6% to less than about 7%, less than about 7% to less than about 8%, less than about 8% to less than about 9%, less than about 9% to 1 0%, less than about 10% to less than about 15%, less than about 15% to less than about 20%, less than about 20% to less than about 35%, less than about 25% to less than about 30%, less than about 30% to less than about 35%, less than about 35% to less than about 40%, less than about 40% to less than about 45%, less than about 45% to less than about 50%, less than about 50% to less than about 55%, less than about 55% to less than about 60%, less than about 60% to less than about 65%, less than about 65% to less than about 70%, less than about 70% to less than about 75%, less than about 75% to less than about 80%, less than about 80% to less than about 85%, less than about 85% to less than about 90%, less than about 90% to less than about 95%, or to less than about 95% to about 100% compared to a subject that is not administered the rAAV of the present disclosure or to baseline.

In some embodiments, administration of rAAV of the present disclosure limits a decrease in the ejection fraction to less than about 1% to less than about 90%, less than about 20% to less than about 80%, less than about 30% to less than about 80%, less than about 40% to less than about 80%, less than about 50% to less than about 80%, less than about 1% to less than about 2%, less than about 2% to less than about 3%, less than about 3% to less than about 4%, less than about 4% to less than about 5%, less than about 5% to less than about 6%, less than about 6% to less than about 7%, less than about 7% to less than about 8%, less than about 8% to less than about 9%, less than about 9% to 10%, less than about 10% to less than about 15%, less than about 15% to less than about 20%, less than about 20% to less than about 35%, less than about 25% to less than about 30%, less than about 30% to less than about 35%, less than about 35% to less than about 40%, less than about 40% to less than about 45%, less than about 45% to less than about 50%, less than about 50% to less than about 55%, less than about 55% to less than about 60%, less than about 60% to less than about 65%, less than about 65% to less than about 70%, less than about 70% to less than about 75%, less than about 75% to less than about 80%, less than about 80% to less than about 85%, less than about 85% to less than about 90%, less than about 90% to less than about 95%, or to less than about 95% to about 100% in a subject overtime. Left Ventricular Ejection Fraction (LVEF)

In some embodiments, administration of rAAV of the present disclosure prevents a decrease in, restores, and/or increases in the LVEF in a subject compared to a subject that is not administered the rAAV of the present disclosure or to baseline. In some embodiments, administration of rAAV of the present disclosure limits a decrease in, restores, and/or increases in the LVEF in a subject overtime.

In some embodiments, administration of rAAV of the present disclosure restores and/or increases the LVEF by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to a subject that is not administered the rAAV of the present disclosure or to baseline.

In some embodiments, administration of rAAV of the present disclosure limits a decrease in the LVEF to less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, at least about 75%, less than about 80%, less than about 85%, less than about 90%, less than about 95%, or less than about 100% compared to a subject that is not administered the rAAV of the present disclosure or to baseline.

In some embodiments, administration of rAAV of the present disclosure restores and/or increases the LVEF by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% in a subject over time.

In some embodiments, administration of rAAV of the present disclosure limits a decrease in the LVEF to less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, at least about 75%, less than about 80%, less than about 85%, less than about 90%, less than about 95%, or less than about 100% in a subject over time.

In some embodiments, administration of rAAV of the present disclosure restores and/or increases the LVEF by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10^{%}, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% compared to a subject that is not administered the rAAV of the present disclosure or to baseline.

In some embodiments, administration of rAAV of the present disclosure restores and/or increases the LVEF by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% in a subject over time.

In some embodiments, administration of rAAV of the present disclosure limits a decrease in the LVEF to less than about 1% to less than about 90%, less than about 20% to less than about 80%, less than about 30% to less than about 80%, less than about 40% to less than about 80%, less than about 50% to less than about 80%, less than about 1% to less than about 2%, less than about 2% to less than about 3%, less than about 3% to less than about 4%, less than about 4% to less than about 5%, less than about 5% to less than about 6%, less than about 6% to less than about 7%, less than about 7% to less than about 8%, less than about 8% to less than about 9%, less than about 9% to 10%, less than about 10% to less than about 15%, less than about 15% to less than about 20%, less than about 20% to less than about 35%, less than about 25% to less than about 30%, less than about 30% to less than about 35%, less than about 35% to less than about 40%, less than about 40% to less than about 45%, less than about 45% to less than about 50%, less than about 50% to less than about 55%, less than about 55% to less than about 60%, less than about 60% to less than about 65%, less than about 65% to less than about 70%, less than about 70% to less than about 75%, less than about 75% to less than about 80%, less than about 80% to less than about 85%, less than about 85% to less than about 90%, less than about 90% to less than about 95%, or to less than about 95% to about 100% compared to a subject that is not administered the rAAV of the present disclosure or to baseline.

In sone embodiments, administration of rAAV of the present disclosure limits a decrease in the LVEF to less than about 1% to less than about 90%, less than about 20% to less than about 80%, less than about 30% to less than about 80%, less than about 40% to less than about 80%, less than about 50% to less than about 80%, less than about 1% to less than about 2%, less than about 2% to less than about 3%, less than about 3% to less than about 4%, less than about 4% to less than about 5%, less than about 5% to less than about 6%, less than about 6% to less than about 7%, less than about 7% to less than about 8%, less than about 8% to less than about 9%, less than about 9% to 10%, less than about 10% to less than about 15%, less than about 15% to less than about 20%, less than about 20% to less than about 35%, less than about 25% to less than about 30%, less than about 30% to less than about 35%, less than about 35% to less than about 40%, less than about 40% to less than about 45%, less than about 45% to less than about 50%, less than about 50% to less than about 55%, less than about 55% to less than about 60%, less than about 60% to less than about 65%, less than about 65% to less than about 70%, less than about 70% to less than about 75%, less than about 75% to less than about 80%, less than about 80% to less than about 85%, less than about 85% to less than about 90%, less than about 90% to less than about 95%, or to less than about 95% to about 100% in a subject over time.

### Right Ventricular Ejection Fraction (RVEF)

In some embodiments, administration of rAAV of the present disclosure prevents a decrease in, restores, and/or increases in the RVEF in a subject compared to a subject that is not administered the rAAV of the present disclosure or to baseline. In some embodiments, administration of rAAV of the present disclosure limits a decrease in, restores, and/or increases in the LVEF in a subject overtime,

In some embodiments, administration of rAAV of the present disclosure restores and/or increases the RVEF by at least about 1%, at least about 2%, at least about 3%, at least about 4%, at least about 5%, at least about 6%, at least about 7%, at least about 8%, at least about 9%, at least about 10N, at least about 15, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to a subject that is not administered the rAAV of the present disclosure or to baseline.

In sone embodiments, administration of rAAV of the present disclosure limits a decrease in the RVEF to less than about 1%. less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, at least about 75%, less than about 80%, less than about 85%, less than about 90%, less than about 95%, or less than about 100% compared to a subject that is not administered the rAAV of the present disclosure or to baseline.

In some embodiments, administration of rAAV of the present disclosure restores and/or increases the RVEF by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% in a subject over time.

In some embodiments, administration of rAAV of the present disclosure limits a decrease in the RVEF to less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, at least about 75%, less than about 80%, less than about 85%, less than about 90%, less than about 95%, or less than about 100% in a subject over time.

In some embodiments, administration of rAAV of the present disclosure restores and/or increases the RVEF by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% compared to a subject that is not administered the rAAV of the present disclosure or to baseline.

In some embodiments, administration of rAAV of the present disclosure restores and/or increases the RVEF by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 70, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% in a subject over time.

In some embodiments, administration of rAAV of the present disclosure limits a decrease in the RVEF to less than about 1% to less than about 90%, less than about 20% to less than about 80%, less than about 30% to less than about 80%, less than about 40% to less than about 80%, less than about 50% to less than about 80%, less than about 1% to less than about 2%, less than about 2% to less than about 3%, less than about 3% to less than about 4%, less than about 4% to less than about 5%, less than about 5% to less than about 6%, less than about 6% to less than about 7%, less than about 7% to less than about 8%, less than about 8% to less than about 9%, less than about 9% to 10%, less than about 10% to less than about 15%, less than about 15% to less than about 20%, less than about 20% to less than about 35%, less than about 25% to less than about 30%, less than about 30% to less than about 35%, less than about 35% to less than about 40%, less than about 40% to less than about 45%, less than about 45% to less than about 50%, less than about 50% to less than about 55%, less than about 55% to less than about 60%, less than about 60% to less than about 65%, less than about 65% to less than about 70%, less than about 70% to less than about 75%, less than about 75% to less than about 80%, less than about 80% to less than about 85%, less than about 85% to less than about 90%, less than about 90% to less than about 95%, or to less than about 95% to about 100% compared to a subject that is not administered the rAAV of the present disclosure or to baseline.

In some embodiments, administration of rAAV of the present disclosure limits a decrease in the RVEF to less than about 1% to less than about 90%, less than about 20% to less than about 80%, less than about 30% to less than about 80%, less than about 40% to less tian about 80%, less than about 50% to less than about 80%, less than about 1% to less than about 2%, less than about 2% to less than about 3%, less than about 3% to less than about 4%, less than about 4% to less than about 5%, less than about 5% to less than about 6%, less than about 6% to less than about 7%, less than about 7% to less than about 8%, less than about 8% to less than about 9%, less than about 9% to 10%, less than about 10% to less than about 15%, less than about 15% to less than about 20%, less than about 20% to less than about 35%, less than about 25% to less than about 30%, less than about 30% to less than about 35%, less than about 35% to less than about 40%, less than about 40% to less than about 45%, less than about 45% to less than about 50%, less than about 50% to less than about 55%, less than about 55% to less than about 60%, less than about 60% to less than about 65%, less than about 65% to less than about 70%, less than about 70% to less than about 75%, less than about 75% to less than about 80%, less than about 80% to less than about 85%, less than about 85% to less than about 90%, less than about 90% to less than about 95%, or to less than about 95% to about 100% in a subject over time.

### Right Ventricle (RV) Area

In some embodiments, administration of rAAV of the present disclosure prevents an increase in, restores, and/or decreases the RV area in a subject compared to a subject that is not administered the rAAV of the present disclosure or to baseline. In some embodiments, administration of rAAV of the present disclosure prevents an increase in, restores, and/or decreases the RV area in a subject over time.

In some embodiments, administration of rAAV of the present disclosure prevents an increase in, restores, and/or decreases the RV area by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to a subject that is not administered the rAAV of the present disclosure or to baseline.

In some embodiments, administration of rAAV of the present disclosure prevents an increase in, restores, and/or decreases the RV area by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% compared to a subject that is not administered the rAAV of the present disclosure or to baseline.

In sone embodiments, administration of rAAV of the present disclosure prevents an increase in, restores, and/or decreases the RV area by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% in a subject over time.

In some embodiments, administration of rAAV of the present disclosure prevents an increase in, restores, and/or decreases the RV area by about 1% to about 90%, about 20% to about 80%, about 30% to about 80%, about. 40% to about 80%, about 50% to about 80%, about 1% to about 2%, about 2% to about 3%, about 3% to about 4%, about 4% to about 5% about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% in a subject over time.

### Left Ventricle (LV) Area

In some embodiments, administration of rAAV of the present disclosure prevents an increase in, restores, and/or decreases the LV area in a subject compared to a subject that is not administered the rAAV of the present disclosure or to baseline. In some embodiments, administration of rAAV of the present disclosure prevents an increase in, restores, and/or decreases the RV area in a subject over time.

In some embodiments, administration of rAAV of the present disclosure prevents an increase in, restores, and/or decreases the LV area by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to a subject that is not administered the rAAV of the present disclosure or to baseline.

In some embodiments, administration of rAAV of the present disclosure prevents an increase in, restores, and/or decreases the LV area by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% compared to a subject that is not administered the rAAV of the present disclosure or to baseline.

In sone embodiments, administration of rAAV of the present disclosure prevents an increase in. restores, and/or decreases the LV area by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% in a subject over time.

In some embodiments, administration of rAAV of the present disclosure prevents an increase in, restores, and/or decreases the LV area by about 1 % to about 90%, about 20% to about 80%, about 30% to about 80% about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20% about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% in a subject over time.

### Right Ventricle (RI) Velocity Time Interaural (VTI)

In some embodiments, administration of rAAV of the present disclosure limits a decrease in, restores, and/or increases in the RV VTI in a subject compared to a subject that is not administered the rAAV of the present disclosure or to baseline. In some embodiments, administration of rAAV of the present disclosure limits a decrease in, restores, and/or increases in the RV VTI in a subject overtime.

In some embodiments, administration of rAAV of the present disclosure restores and/or increases the RV VTI by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to a subject that is not administered the rAAV of the present disclosure or to baseline.

In some embodiments, administration of rAAV of the present disclosure limits a decrease in the RV VTI to less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, at least about 75%, less than about 80%, less than about 85%, less than about 90%, less than about 95%, or less than about 100% compared to a subject that is not administered the rAAV of the present disclosure or to baseline.

In some embodiments, administration of rAAV of the present disclosure restores and/or increases the RV VTI by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% in a subject over time.

In some embodiments, administration of rAAV of the present disclosure limits a decrease in the RV VTI to less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, at least about 75%, less than about 80%, less than about 85%, less than about 90%, less than about 95%, or less than about 100% in a subject overtime.

In some embodiments, administration of rAAV of the present disclosure restores and/or increases the RV VTI by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% compared to a subject that is not administered the rAAV of the present disclosure or to baseline.

In some embodiments, administration of rAAV of the present disclosure restores and/or increases the RV VTI by about 1% to about 90%, about 20% to about 80%, about 30% to about 80%, about 40% to about 80% about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% in a subject over time.

In some embodiments, administration of rAAV of the present disclosure limits a decrease in the RV VTT to less than about % to less than about 90%, less than about 20% to less than about 80%, less than about 30% to less than about 80%, less than about 40% to less than about 80%, less than about 50% to less than about 80%, less than about 1% to less than about 2%, less than about 2% to less than about 3%, less than about 3% to less than about 4%, less than about 4% to less than about 5%, less than about 5% to less than about 6%, less than about 6% to less than about 7%, less than about 7% to less than about 8%, less than about 8% to less than about 9%, less than about 9% to 10%, less than about 10% to less than about 15%, less than about 15% to less than about 20%, less than about 20% to less than about 35%, less than about 25% to less than about 30%, less than about 30% to less than about 35%, less than about 35% to less than about 40%, less than about 40% to less than about 45%, less than about 45% to less than about 50%, less than about 50% to less than about 55%, less than about 55% to less than about 60%, less than about 60% to less than about 65%, less than about 65% to less than about 70%, less than about 70% to less than about 75%, less than about 75% to less than about 80%, less than about 80% to less than about 85%, less than about 85% to less than about 90%, less than about 90% to less than about 95%, or to less than about 95% to about 100% compared to a subject that is not administered the rAAV of the present disclosure or to baseline.

In some embodiments, administration of rAAV of the present disclosure limits a decrease in the RV VTI to less than about 1% to less than about 90%, less than about 20% to less than about 80%, less than about 30% to less than about 80%, less than about 40% to less than about 80%, less than about 50% to less than about 80%, less than about 1% to less than about 2%, less than about 2% to less than about 3%, less than about 3% to less than about 4%, less than about 4% to less than about 5%, less than about 5% to less than about 6%, less than about 6% to less than about 7%, less than about 7% to less than about 8%, less than about 8% to less than about 9%, less than about 9% to 10%, less than about 10% to less than about 15%, less than about 15% to less than about 20%, less than about 20% to less than about 35%, less than about 25% to less than about 30%, less than about 30% to less than about 35%, less than about 35% to less than about 40%, less than about 40% to less than about 45%, less than about 45% to less than about 50%, less than about 50% to less than about 55%, less than about 55% to less than about 60%, less than about 60% to less than about 65%, less than about 65% to less than about 70%, less than about 70% to less than about 75%, less than about 75% to less than about 80%, less than about 80% to less than about 85%, less than about 85% to less than about 90%, less than about 90% to less than about 95%, or to less than about 95% to about 100% in a subject over time.

### Left Ventricle (LV) Velocity Time Integral (VTI)

In some embodiments, administration of rAAV of the present disclosure limits a decrease in, restores, and/or increases in the LV VTI in a subject compared to a subject that is not administered the rAAV of the present disclosure or to baseline. In some embodiments, administration of rAAV of the present disclosure limits a decrease in, restores, and/or increases in the LV VTI in a subject over time. Accordingly, methods disclosed herein may be used to limit a decrease in, restore, and/or increase LV VTI in a subject in need thereof, e.g., a subject in need thereof, e.g., a subject with ACM, e.g., ARVC or ARVD.

In sone embodiments, administration of rAAV of the present disclosure restores and/or increases the LV VTI by at least about 1%, at least about 2X, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to a subject that is not administered the rAAV of the present disclosure or to baseline.

In some embodiments, administration of rAAV of the present disclosure limits a decrease in the LV VTI to less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, at least about 75%, less than about 80%, less than about 85%, less than about 90%, less than about 95%, or less than about 100% compared to a subject that is not administered the rAAV of the present disclosure or to baseline.

In some embodiments, administration of rAAV of the present disclosure restores and/or increases the LV VTI by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% in a subject over time.

In some embodiments, administration of rAAV of the present disclosure limits a decrease in the LV VTI to less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, at least about 75%, less than about 80%, less than about 85%, less than about 90%, less than about 95%, or less than about 100% in a subject over time.

In some embodiments, administration of rAAV of the present disclosure restores and/or increases the LV VTI by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% compared to a subject that is not administered the rAAV of the present disclosure or to baseline.

In some embodiments, administration of rAAV of the present disclosure restores and/or increases the LV VTI by about 1% to about 90%, about 20% to about 80%, about 30% to about 80%, about 40% to about 80% about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% in a subject over time.

In some embodiments. administration of rAAV of the present disclosure limits a decrease in the LV VTI to less than about 1% to less than about 90%, less than about 20% to less than about 80%, less than about 30% to less than about 80%, less than about 40% to less than about 80%, less than about 50% to less than about 80%, less than about 1% to less than about 2%, less than about 2% to less than about 3%, less than about 3% to less than about 4%, less than about 4% to less than about 5%, less than about 5% to less than about 6%, less than about 6% to less than about 7%, less than about 7% to less than about 8%, less than about 8% to less than about 9%, less than about 9% to 10%, less than about 10% to less than about 15%, less than about 15% to less than about 20%, less than about 20% to less than about 35%, less than about 25% to less than about 30%, less than about 30% to less than about 35%, less than about 35% to less than about 40%, less than about 40% to less than about 45%, less than about 45% to less than about 50%, less than about 50% to less than about 55%, less than about 55% to less than about 60%, less than about 60% to less than about 65%, less than about 65% to less than about 70%, less than about 70% to less than about 75%, less than about 75% to less than about 80%, less than about 80% to less than about 85%, less than about 85% to less than about 90%, less than about 90% to less than about 95%, or to less than about 95% to about 100% compared to a subject that is not administered the rAAV of the present disclosure or to baseline.

In sone embodiments, administration of rAAV of the present disclosure limits a decrease in the LV VTT to less than about 1% to less than about 90%, less than about 20% to less than about 80%, less than about 30% to less than about 80%, less than about 40% to less than about 80%, less than about 50% to less than about 80%, less than about 1% to less than about 2%, less than about 2% to less than about 3%, less than about 3% to less than about 4%, less than about 4% to less than about 5%, less than about 5% to less than about 6%, less than about 6% to less than about 7%, less than about 7% to less than about 8%, less than about 8% to less than about 9%, less than about 9% to 10%, less than about 10% to less than about 15%, less than about 15% to less than about 20%, less than about 20% to less than about 35%, less than about 25% to less than about 30%, less than about 30% to less than about 35%, less than about 35% to less than about 40%, less than about 40% to less than about 45%, less than about 45% to less than about 50%, less than about 50% to less than about 55%, less than about 55% to less than about 60%, less than about 60% to less than about 65%, less than about 65% to less than about 70%, less than about 70% to less than about 75%, less than about 75% to less than about 80%, less than about 80% to less than about 85%, less than about 85% to less than about 90%, less than about 90% to less than about 95%, or to less than about 95% to about 100% in a subject overtime,

### Left Ventricle (LV) Fibrosis

In some embodiments, administration of rAAV of the present disclosure prevents an increase in and/or decreases the LV fibrosis in a subject compared to a subject that is not administered the rAAV of the present disclosure or to baseline. In some embodiments, administration of rAAV of the present disclosure prevents an increase in and/or decreases the LV fibrosis in a subject over time. Accordingly, methods disclosed herein may be used to prevent an increase in and/or decrease LV fibrosis in a subject in need thereof, e.g., a subject in need thereof, e.g., a subject with ACM, e.g_, ARVC or ARVD,

In some embodiments, administration of rAAV of the present disclosure prevents an increase in and/or decreases the LV fibrosis by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to a subject that is not administered the rAAV of the present disclosure or to baseline.

In some embodiments, administration of rAAV of the present disclosure prevents an increase in and/or decreases the LV fibrosis by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% compared to a subject that is not administered the rAAV of the present disclosure or to baseline,

In some embodiments, administration of rAAV of the present disclosure prevents an increase in and/or decreases the IV fibrosis by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% in a subject over time.

In some embodiments, administration of rAAV of the present disclosure prevents an increase in and/or decreases the LV fibrosis by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% in a subject over time.

### Right Ventricle (RV) Fibrosis

In some embodiments, administration of rAAV of the present disclosure prevents an increase in and/or decreases the RV fibrosis in a subject compared to a subject that is not administered the rAAV of the present disclosure or to baseline. In some embodiments, administration of rAAV of the present disclosure prevents an increase in and/or decreases the RV fibrosis in a subject over time. Accordingly, methods disclosed herein may be used to prevent an increase in and/or decrease RV fibrosis in a subject in need thereof, e.g., a subject in need thereof, e.g., a subject with ACM, e.g., ARVC or ARVD.

In some embodiments, administration of rAAV of the present disclosure prevents an increase in and/or decreases the RV % fibrosis by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to a subject that is not administered the rAAV of the present disclosure or to baseline.

In some embodiments, administration of rAAV of the present disclosure prevents an increase in and/or decreases the RV fibrosis by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20% about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% compared to a subject that is not administered the rAAV of the present disclosure or to baseline.

In some embodiments, administration of rAAV of the present disclosure prevents an increase in and/or decreases the RV fibrosis by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% in a subject over time.

In some embodiments, administration of rAAV of the present disclosure prevents an increase in and/or decreases the RV fibrosis by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% in a subject over time.

### Premature Ventricular Contractions (PVC)

In some embodiments, administration of rAAV of the present disclosure prevents an increase in and/or decreases PVC in a subject compared to a subject that is not administered the rAAV of the present disclosure or to baseline. In some embodiments, administration of rAAV of the present disclosure prevents an increase in and/or decreases PVC in a subject over time. Accordingly. methods disclosed herein may be used to prevent an increase in and/or decrease PVC in a subject in need thereof, e.g., a subject in need thereof, e.g., a subject with ACM, e.g., ARVC or ARVD.

In some embodiments, administration of rAAV of the present disclosure prevents an increase in and/or decreases PVC by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to a subject that is not administered the rAAV of the present disclosure or to baseline.

In some embodiments, administration of rAAV of the present disclosure prevents an increase in and/or decreases PVC by about 1/to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% compared to a subject that is not administered the rAAV of the present disclosure or to baseline.

In some embodiments. administration of rAAV of the present disclosure prevents an increase in and/or decreases PVC by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% in a subject over time.

In some embodiments, administration of rAAV of the present disclosure prevents an increase in and/or decreases PVC by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% in a subject over time.

### Non-sustained Ventricular Tachycardia (NSVT)

In some embodiments, administration of rAAV of the present disclosure prevents an increase in and/or decreases NSVT in a subject compared to a subject that is not administered the rAAV of the present disclosure or to baseline. In some embodiments, administration of rAAV of the present disclosure prevents an increase in and/or decreases NSVT in a subject over time. Accordingly, methods disclosed herein may be used to prevent an increase in and/or decrease NSVT in a subject in need thereof, e.g., a subject in need thereof, e.g., a subject with ACM, e.g., ARVC or ARVD.

In some embodiments, administration of rAAV of the present disclosure prevents an increase in and/or decreases NSVT by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to a subject that is not administered the rAAV of the present disclosure or to baseline.

In some embodiments, administration of rAAV of the present disclosure prevents an increase in and/or decreases NSVT by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15%) to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% compared to a subject that is not administered the rAAV of the present disclosure or to baseline.

In some embodiments, administration of rAAV of the present disclosure prevents an increase in and/or decreases N SVT by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% in a subject over time.

In some embodiments, administration of rAAV of the present disclosure prevents an increase in and/or decreases NSVT by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15% about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% in a subject over time.

### Ventricular Tachycardia (VT)

In some embodiments, administration of rAAV of the present disclosure prevents an increase in and/or decreases VT in a subject compared to a subject that is not administered the rAAV of the present disclosure or to baseline. In some embodiments, administration of rAAV of the present disclosure prevents an increase in and/or decreases NSVT in a subject over time.

In some embodiments, administration of rAAV of the present disclosure prevents an increase in and/or decreases VT by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to a subject that is not administered the rAAV of the present disclosure or to baseline. Accordingly. methods disclosed herein may be used to pre vent an increase in and/or decrease VT in a subject in need thereof, e.g., a subject in need thereof, e.g., a subject with ACM, e.g., ARVC or ARVD.

In some embodiments, administration of rAAV of the present disclosure prevents an increase in and/or decreases VT by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% compared to a subject that is not administered the rAAV of the present disclosure or to baseline.

In some embodiments, administration of rAAV of the present disclosure prevents an increase in and/or decreases VT by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% in a subject over time.

In some embodiments, administration of rAAV of the present disclosure prevents an increase in and/or decreases VT by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% in a subject over time.

### Ectopic Beats

In some embodiments, administration of rAAV of the present disclosure prevents an increase in and/or decreases ectopic beats in a subject compared to a subject that is not administered the rAAV of the present disclosure or to baseline. In some embodiments, administration of rAAV of the present disclosure prevents an increase in and/or decreases ectopic beats in a subject over time. Accordingly, methods disclosed herein may be used to prevent an increase in and/or decrease ectopic beats in a subject in need thereof, e.g. a subject in need thereof, e.g., a subject with ACM, e.g., ARVC or ARVD.

In some embodiments, administration of rAAV of the present disclosure prevents an increase in and/or decreases ectopic beats by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to a subject that is not administered the rAAV of the present disclosure or to baseline.

In some embodiments, administration of rAAV of the present disclosure prevents an increase in and/or decreases ectopic beats by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% compared to a subject that is not administered the rAAV of the present disclosure or to baseline.

In some embodiments, administration of rAAV of the present disclosure prevents an increase in and/or decreases ectopic beats by at least about 1%, at least about 2%, at least about 3%, at least about 4%), at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% in a subject over time.

In some embodiments, administration of rAAV of the present disclosure prevents an increase in and/or decreases ectopic beats by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, orby about 95% to about 100% in a subject over time.

### Intercalated Discs

In some embodiments, administration of rAAV of the present disclosure limits a decrease in, restores, and/or increases the intercalated discs in a subject compared to a subject that is not administered the rAAV of the present disclosure or to baseline. In cardiac muscles of the heart, connections between neighboring cells are formed by the intercalated discs. To enable the heartbeat, the intercalated disc is highly specialized and allows for coordinated function of the heart cells. In some embodiments, administration of rAAV of the present disclosure limits a decrease in, restores, and/or increases in the intercalated discs in a subject over time. Accordingly, methods disclosed herein may be used to limit a decrease in, restore, and/or increase intercalated discs in a subject in need thereof, e.g., a subject in need thereof, e.g., a subject with ACM, e.g., ARVC or ARVD.

In some embodiments, administration of rAAV of the present disclosure restores and/or increases the intercalated discs by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to a subject that is not administered the rAAV of the present disclosure or to baseline.

In some embodiments, administration of rAAV of the present disclosure limits a decrease in the intercalated discs to less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, at least about 75%, less than about 80%, less than about 85%, less than about 90%, less than about 95%, or less than about 100% compared to a subject that is not administered the rAAV of the present disclosure or to baseline.

In some embodiments, administration of rAAV of the present disclosure restores and/or increases the intercalated discs by at least about 11%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% in a subject over time.

In some embodiments, administration of rAAV of the present disclosure limits a decrease in the intercalated discs to less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, at least about 75%, less than about 80%, less than about 85%, less than about 90%, less than about 95%, or less than about 100% in a subject over time.

In some embodiments, administration of rAAV of the present disclosure restores and/or increases the intercalated discs by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% compared to a subject that is not administered the rAAV of the present disclosure or to baseline.

In some embodiments, administration of rAAV of the present disclosure restores and/or increases the intercalated discs by about 1′% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% in a subject over time.

In some embodiments, administration of rAAV of the present disclosure limits a decrease in the intercalated discs to less than about 1% to less than about 90%, less than about 20% to less than about 80%, less than about 30% to less than about 80%, less than about 40% to less than about 80%, less than about 50% to less than about 80%, less than about 1% to less than about 2%, less than about 2% to less than about 3%, less than about 3% to less than about 4%, less than about 4% to less than about 5%, less than about 5% to less than about 6%, less than about 6% to less than about 7%, less than about 7% to less than about 8%, less than about 8% to less than about 9%, less than about 9% to 10%, less than about 10% to less than about 15%, less than about 15% to less than about 20%, less than about 20% to less than about 35%, less than about 25% to less than about 30%, less than about 30% to less than about 35%, less than about 35% to less than about 40%, less than about 40% to less than about 45%, less than about 45% to less than about 50%, less than about 50% to less than about 55%, less than about 55% to less than about 60%, less than about 60% to less than about 65%, less than about 65% to less than about 70%, less than about 70% to less than about 75%, less than about 75% to less than about 80%, less than about 80% to less than about 85% less than about 85% to less than about 90%, less than about 90% to less than about 95%, or to less than about 95% to about 100% compared to a subject that is not administered the rAAV of the present disclosure or to baseline.

In some embodiments, administration of rAAV of the present disclosure limits a decrease in the intercalated discs to less than about 1% to less than about 90%, less than about 20% to less than about 80%, less than about 30% to less than about 80%, less than about 40% to less than about 80%, less than about 50% to less than about 80%, less than about 1% to less than about 2%, less than about 2% to less than about 3%, less than about 3% to less than about 4%, less than about 4% to less than about 5%, less than about 5% to less than about 6%, less than about 6% to less than about 7%, less than about 7% to less than about 8%, less than about 8% to less than about 9%, less than about 9% to 10%, less than about 10% to less than about 15%, less than about 15% to less than about 20%, less than about 20% to less than about 35%, less than about 25% to less than about 30%, less than about 30% to less than about 35%, less than about 35% to less than about 40%, less than about 40% to less than about 45%, less than about 45% to less than about 50%, less than about 50% to less than about 55%, less than about 55% to less than about 60%, less than about 60% to less than about 65%, less than about 65% to less than about 70%, less than about 70% to less than about 75%, less than about 75% to less than about 80%, less than about 80% to less than about 85%, less than about 85% to less than about 90%, less than about 90% to less than about 95%, or to less than about 95% to about 100% in a subject over time.

### EXAMPLES

### Example 1: In Vitro Testing of Adeno-Associated Virus Vectors

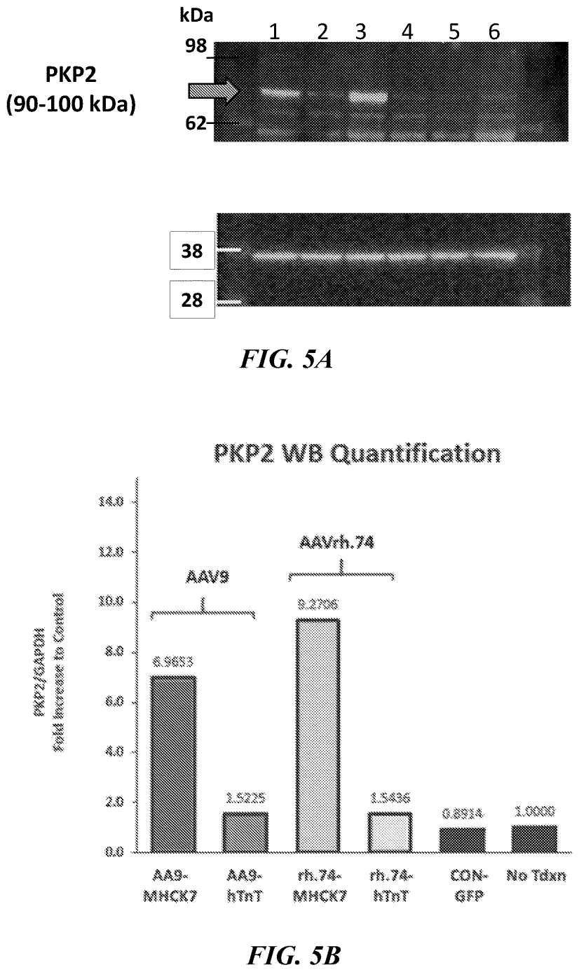

AAV vectors described herein (those having structures as shown in FIG. 1A (e.g., SEQ ID NO: 12), FIG. 2 (e.g., SEQ ID NO: 13 or 97), FIG. 3 (e.g., SEQ ID NO: 14 or 98) and FIG. 4 (e.g., SEQ ID NO: 15 or 99) were prepared and used to transduce CHO-Lee2 cells and expression levels of PKP2 were assessed by Western Blot (FIGS. 5A-5B; individual lanes in the SDS-PAGE gel provided in FIG. 5A correspond to the respective levels of PKP2 depicted in FIG. 5B). Surprisingly, the MHCK7 promoter caused robust expression of PKP2 in cardiomyocytes (Lanes 1 & 3 in FIG. 5A), whereas the hTnnT2 promoter (“hTnT”) generated marginal PKP2 levels above background under these testing conditions (Lanes 2 & 4 in FIG. 5A). The AAVrh.74 serotype induced higher expression of PKP2 than the AAV9 serotype vector (Lanes 3 & 4 in FIG. SA).

These results demonstrate that AAV9 vectors and AAVrh.74 vectors can effectively be used to express PKP2 in cardiomyocytes, and that the MHCK7 promoter is superior to the hTnnT2 promoter when solely evaluating the relative levels of PKP2 expression using this in vitro assay.

### Example 2: In Vivo Efficacy of Adeno-Associated Virus Vectors

Evidence of benefit of AAV mediated overexpression of PKP2 was determined using a cardiomyocyte-specific, tamoxifen-activated, PKP2 knockout murine line (aMHC-Crc-ER(T2)/Pkp2 fl/fl; referred to as “PKP2-cKO”), as described in Cerrone et al., Nat Comm., 2017. This mouse model allows control of the onset of PKP2 loss of expression, limits loss of PKP2 to adult myocytes, and initiates a progression of molecular and functional events leading to an arrhythmogenic cardiomyopathy (ACM) with initial right ventricular predominance, but involves both left and right ventricles in this mouse. The temporal progression of the molecular, structural and functional events as a consequence of PKP2-cKO have been well characterized (Cerrone et al., Nat Comm, 2017). PKP2 deficiency in adult ventricular myocytes is sufficient to cause an arrhythmogenic cardiomyopathy of RV predominance, which includes the ‘hallmark’ functional, molecular, and structural indices consistent with the disease phenotype of ACM.

PKP2-cKO mice were injected with tamoxifen, causing myocyte-specific knockout of PKP2. Mice were injected with AAV vectors (as described below) at 3×10¹³ vg/kg by intravenous (tail vein) injection. Four weeks later, myocyte-specific knockout of PKP2 was induced by treatment of the mice with tamoxifen. The vector genomes tested were: 5′ ITR; MHCK7 promoter (with its enhancer element); SV40 intron; Kozak sequence; PKP2a transgene; WPRE(x); hGH polyadenylation sequence; 3′ ITR (structure diagrammed in FIG. 1B; e.g., SEQ ID NO: 89); and 5′ ITR; hTnnT2 promoter (with exon 1); Kozak sequence; PKP2a transgene; WPRE(x); hGH polyadenylation sequence; 3′ ITR (structure diagrammed in FIG. 2; e.g., SEQ ID NO: 13 or 97),

Each vector genome was tested in an AAV9 serotype or an A AVrh.74 serotype vector.

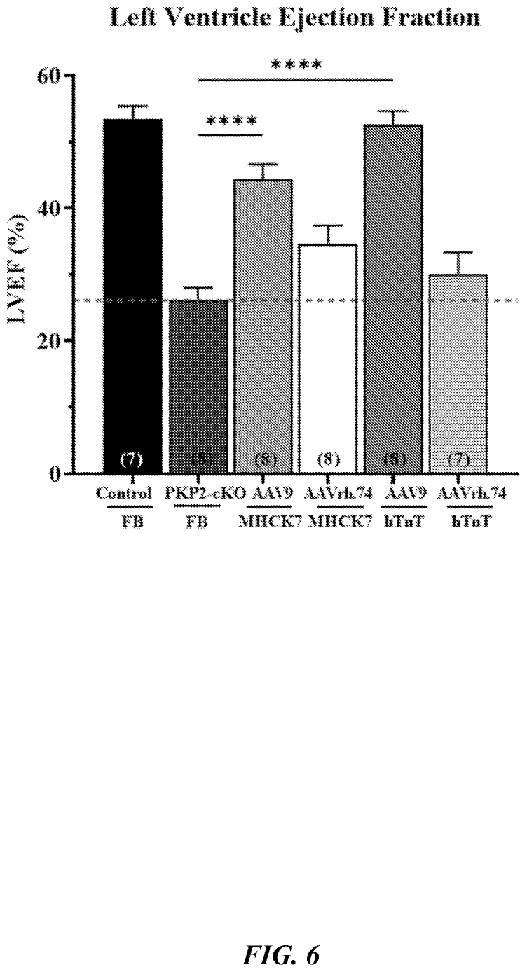

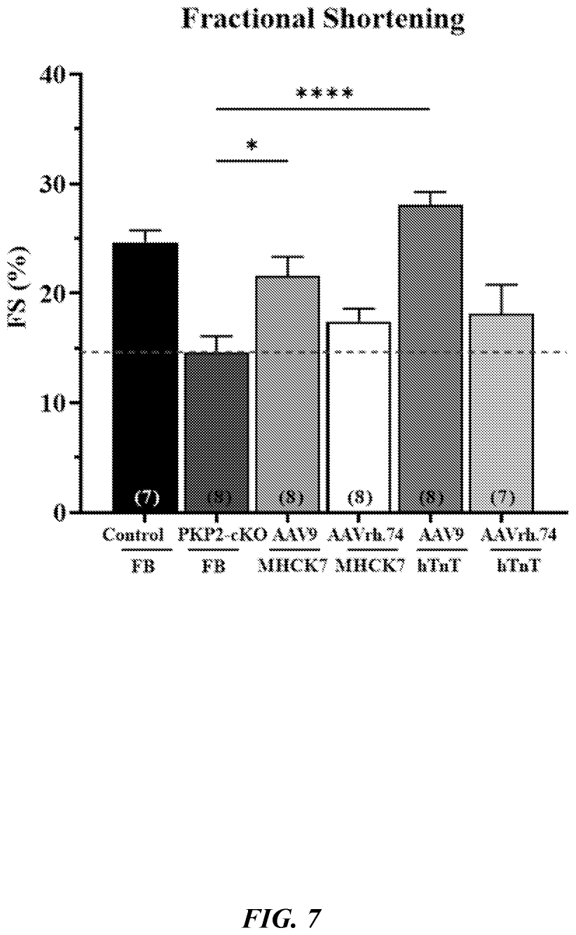

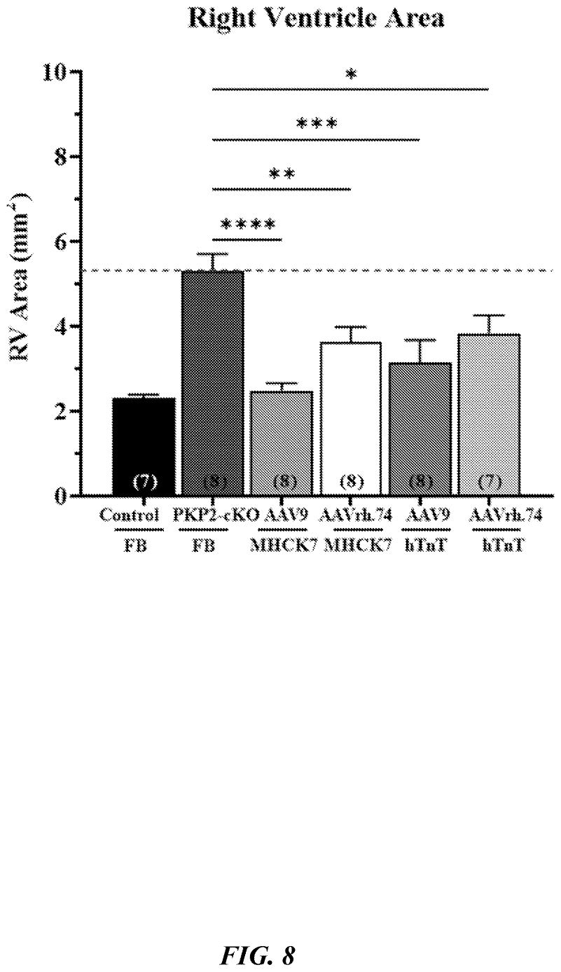

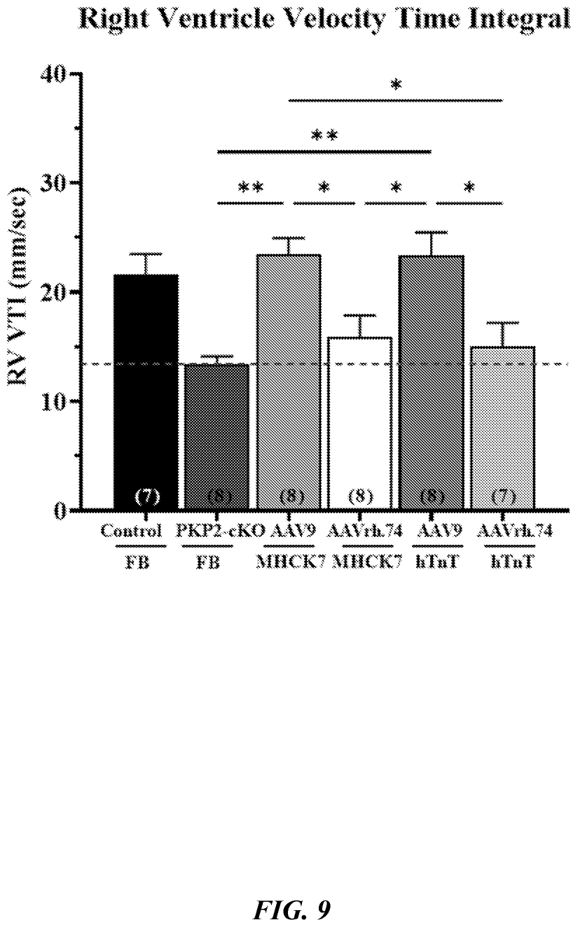

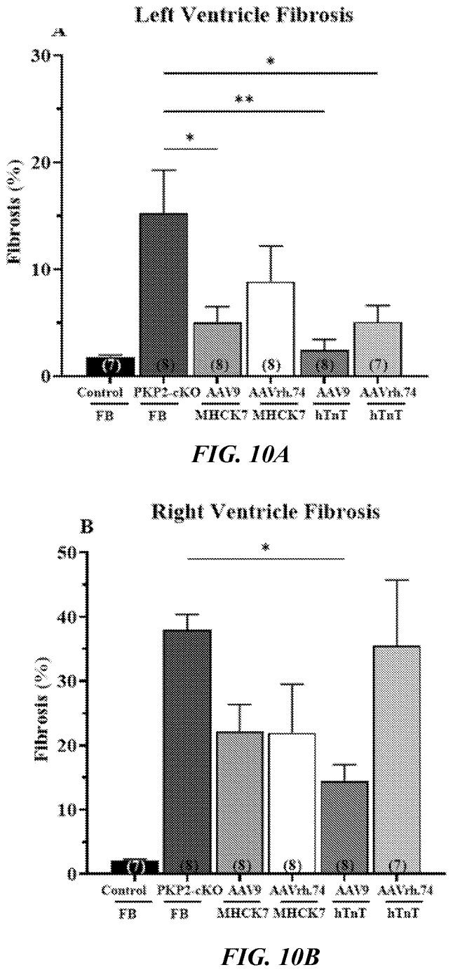

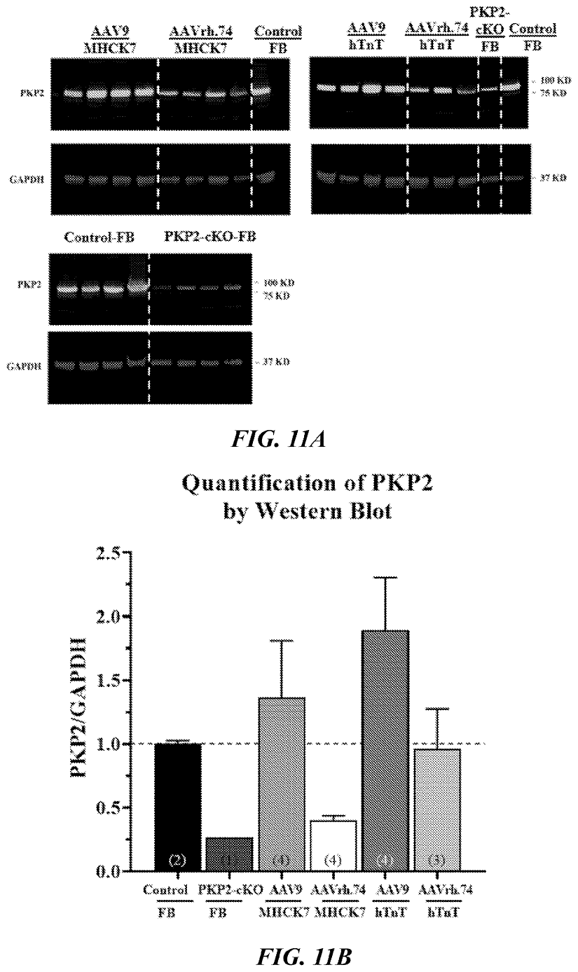

At 28 days after tamoxifen treatment, which is 56 days after AAV treatment, mice were evaluated for various physiology parameters, essentially as described in Cerrone et al., Nat Comm, 2017, or using standard methodologies known in the art. Efficacy in treating disease was assessed by left ventricle ejection fraction percentage (LVEF %) (FIG. 6), left ventricle fractional shortening percentage (FS %) (FIG. 7), right ventricle area in millimeters squared RV Area (mm²) (FIG. 8), right ventricle velocity time integral in millimeters per second RV VTI (mm/sec) (FIG. 9), and degree of fibrosis (FIGS. 10A-10B). These measures are appropriate functional and structural indices to evaluate potential efficacy of AAV-mediated PKP2 overexpression in cardiomyocytes as they are among key parameters indicative of ACM in human disease. Generally, a right ventricle normally has slightly greater amount of fibrosis (irrespective of disease), and this is further exacerbated with lack of PKP2 in the cKO model. Progressive deterioration of these parameters was observed within 28 days of tamoxifen injection, because tamoxifen injection causes myocyte-specific knockout of the PKP gene. The in vivo expression of the transgene was demonstrated using a Western blot to detect the PKP2 levels (FIGS. 11A-11B). Gel images (FIG. 11A) and quantitation of PKP2 normalized to GAPDH (FIG. 11B), quantified from panel A top gels acquired at the same time) detected in the heart at 28 day s post-tamoxifen injection, which was 56 days post-AAV (AAV9-MHCK7-PKP2a, A AV9-hTnT-PKP2a, AAVrh.74-MHCK7-PKP2a or AAVrh.74-hTnT-PKP2a at a dose of 3×10¹³ vg/kg) injection.

Evidence for mitigation of the disease phenotype was observed following both AAV9- and AAVrh.74-mediated PKP expression, to varying degrees. With the dose studied in this example (3×10¹³ vg/kg) using a pre-treatment paradigm (AAV injections 4 weeks prior to tamoxifen-induced PKP cKO). Given the cardiotropism of AAVrh. 74 and given that biological effects were observed with A AVrh.74-mediated overexpression of PKP2 in this model (e.g., LVEF %, FS %, and right ventricular area), optimization of the dose of AAVrh.74 was also tested in combination with the appropriate promoter (i.e., either MHCK7 or hTnnT2).

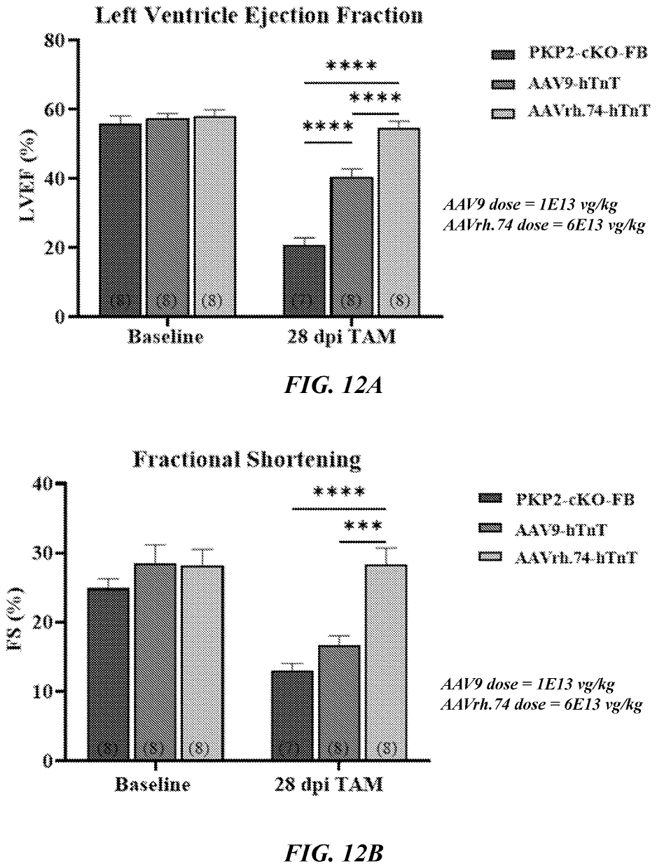

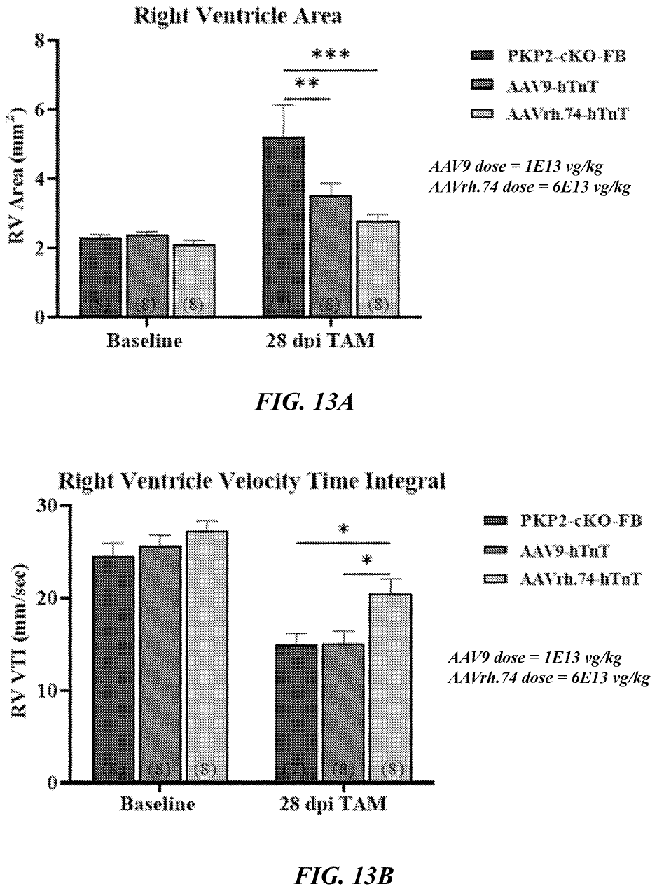

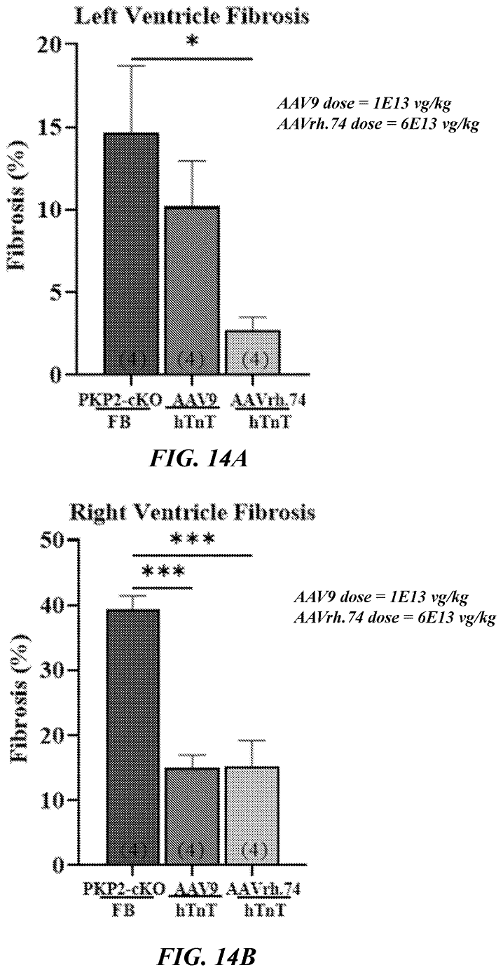

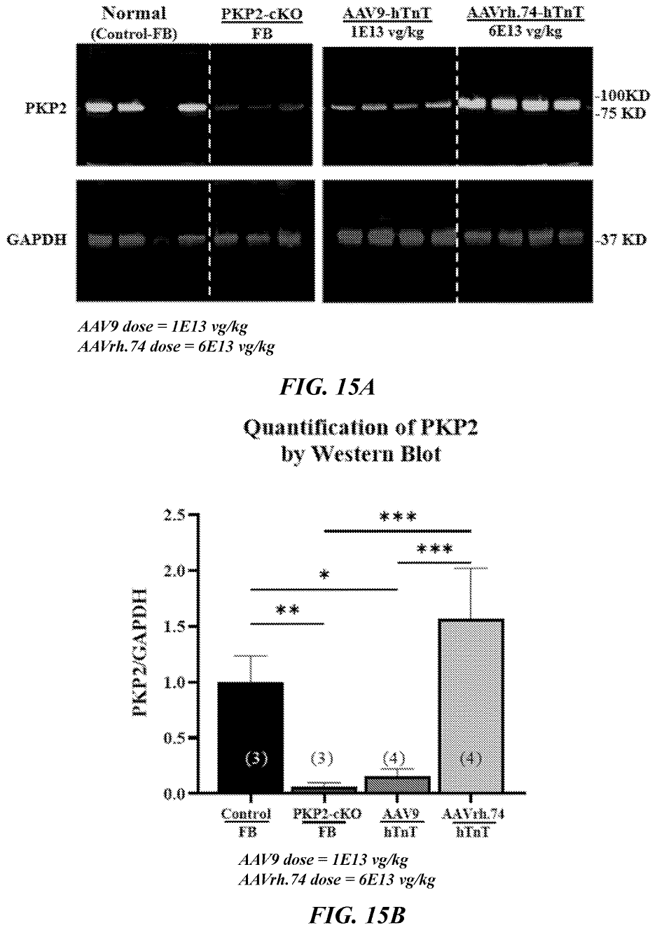

In the next set of experiments, the dose of 6×10¹³ vg/kg was tested using the AAVrh.74-hTnT-PKP2a and the dose of 1×10¹³ vg/kg for AAV9-hTnT-PKP2a. At 28 days after tamoxifen treatment, 56 days after AAV treatment, cardiac parameters were evaluated as in the previous example. Efficacy in treating disease was assessed by left ventricle ejection fraction percentage (LVEF %) (FIG. 12A), left ventricle fractional shortening percentage (FS %) (FIG. 12B), right ventricle area in millimeters squared RV Area (mm²) (FIG. 13A), right ventricle velocity time integral in millimeters per second (RV VTI (mm/sec)) (FIG. 13B), and degree of fibrosis (FIGS. 14A-14B). The protein levels expressed in vivo were demonstrated using Western Blot to detect PKP2 (FIGS. 15A-15B). Gel images (FIG. 15A) and quantitation of PKP2 normalized to GAPDH (FIG. 15B, quantified from panel A top gels acquired at the same time) detected in the heart at 28 days post-tamoxifen injection, which was 56 days post-AAV (AAV9-hTnT-PKP2a at a dose of 1×10¹³ (vg/kg or AAVrh.74-hTnT-PKP2a at a dose of 6×10¹³ vg/kg) injection.

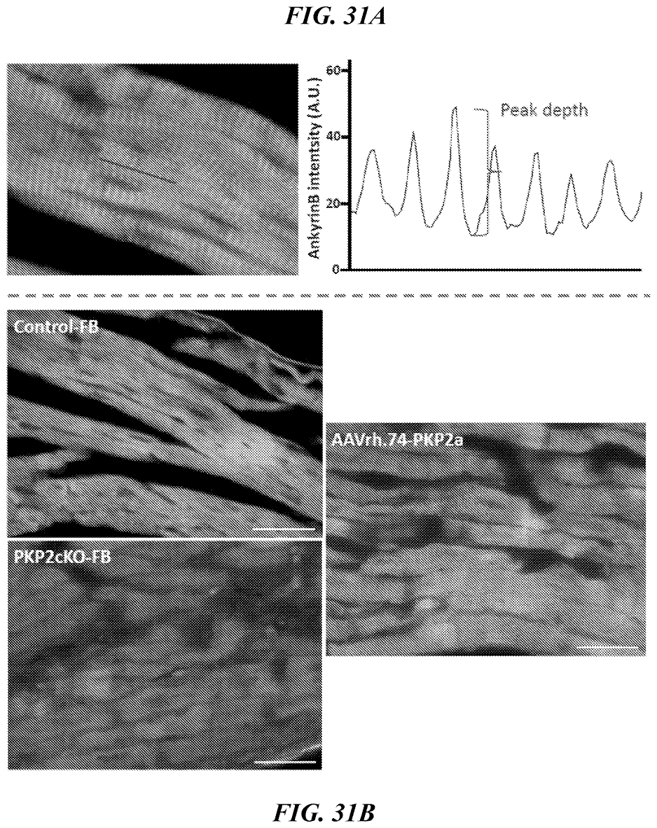

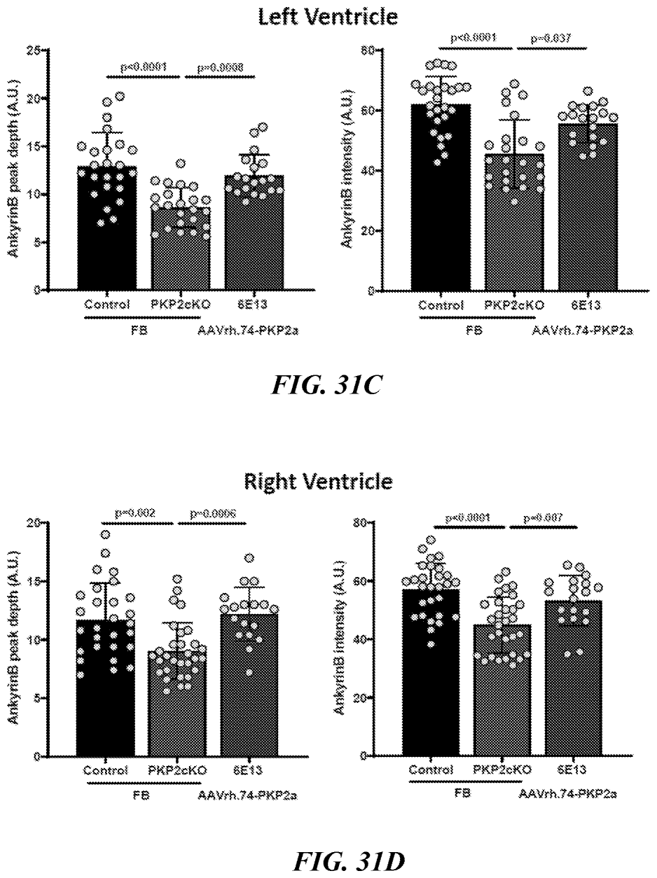

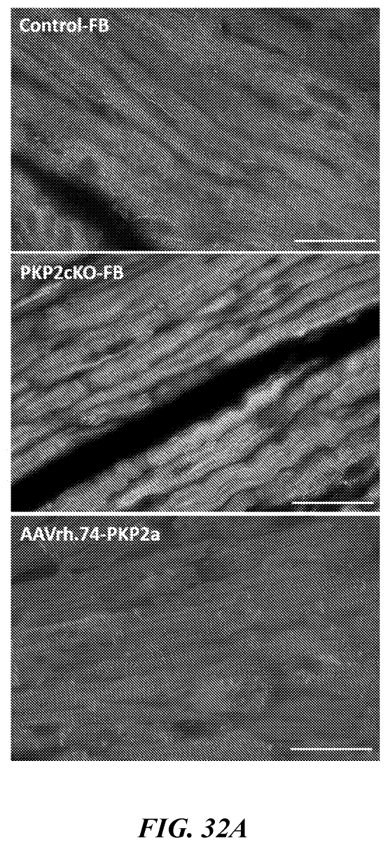

In additional analyses, samples were collected from mice either control (Crc-negative) injected with formulation buffer (Control-FB; n=:4 mice), PKP2cKO injected with formulation buffer (PKP2cKO-FB; n=4 mice) or PKP2-cK0 injected with AAVrh.74-PKP2a (PKP2cKO-AAVrh.74-PKP2a; n=4 mice). Mice were treated with FB or A AVrh.74-PKP2a 28 days before Tamoxifen injection. Immunofluorescence images were obtained from paraffin-embedded tissue slices. The map of fluorescence intensities was collected from a stretch of 5 to 8 sarcomeres deemed to be properly aligned for analysis to obtain a fluorescence intensity map. FIG. 31A shows an example of adult ventricular tissue stained with an antibody for AnkB (Cerrone et al 2017). The red line indicates the range of fluorescence intensities as plotted in the right panel. Two parameters were collected: mean intensity, and peak depth (defined as the range between peak and valley; see FIG. 31A, right panel). Representative images obtained under the three conditions tested are presented in FIG. 31B. Values for each intensity map were averaged and considered one data point. Multiple areas were mapped per tissue section, and data collected separately from samples of the right ventricle (RV) or the left ventricle (LV). Results collected from LV and for RV are presented in FIG. 31C and FIG. 31D, respectively. Statistical analyses were performed by One-way ANOVA followed by Tukey's post-hoc analyses and statistical significance (p values) are noted at the top of the bars. The results show that re-expression of PKP2 in the myocytes of PKP2cK(O hearts reversed a feature of the previously reported molecular phenotype, namely, reduced AnkB expression.

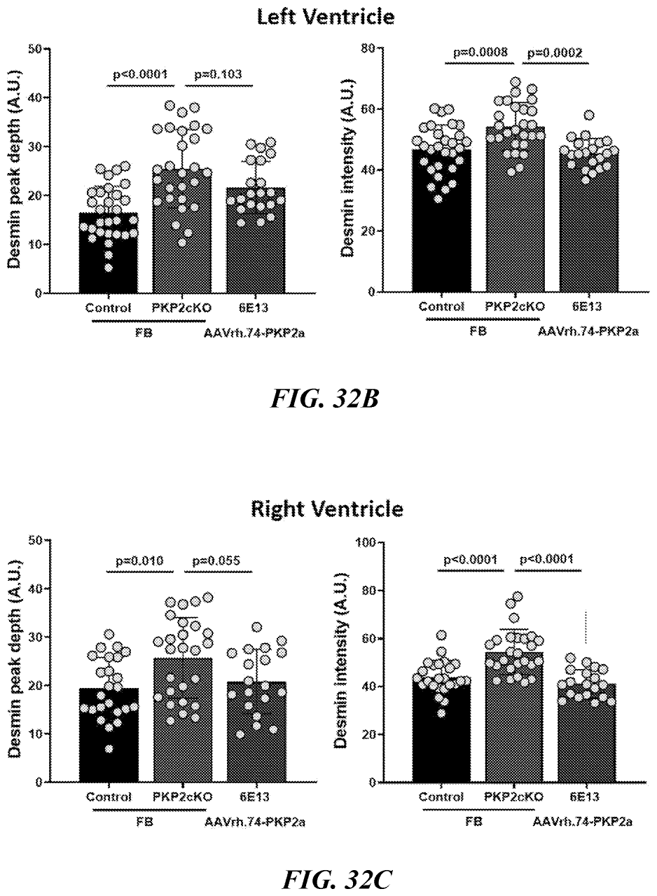

Similarly, tissue sections were immunostained for desmin (FIGS. 32A-32C; methods for immunostaining were the same as those described for PKP2; desmin antibody was the same as that used in Perez-Hernindez et al. 2022; PMID:35959657). Representative examples and results are shown in FIGS. 32A-32C. In this case, the broad data distribution affected statistical power, though the increased intensity obtained from PKP2cKO cardiomyocytes (see Supplemental FIG. 8 of Pdrez-Hemsndez et al. 2022: PMID: 35959657) and a statistically significant recovery of the signal intensity in the AAV-treated mice was confirmed, concurrent with a tendency to recovery of the peak depth in both RV and LV samples. The data show that two molecular features of the PKP2cKO hearts, reduced AnkB and increased desmin, revert in hearts treated with the AAV-delivered PKP2 transgene.

These results demonstrate both AAV9 and AAVrh,74 can provide benefit in a relevant animal model of PKP2-related ACM [also known as Arrhythmogenic Right Ventricular Dysplasia (ARVD) or Arrhythmogenic Cardiomyopathy (ACM)]. Additionally, AAV vectors with either MHCK7 promoter or hTmnT2 promoter have been demonstrated to be effective in treating PKP2-related disease.

### Example 3: IN Vivo Efficacy of Adeno-Associated Virus Vectors

Following evaluation of the in vivo effects of various AAV-PKP2 vector constructs at differing doses in the PKP-cKO) model of ACM, the AAVrh.74-hTnT-PKP2a vector construct (FIG. 2; e.g., SEQ ID NO: 13 or 97) was selected for further R&D and clinical development. This was based on the robust functional and anatomical benefits, safety profile observed to date, and potentially avoiding immune responses to the AAV9 capsid that have been observed in clinical trials employing intravenous delivery of high doses in adults.

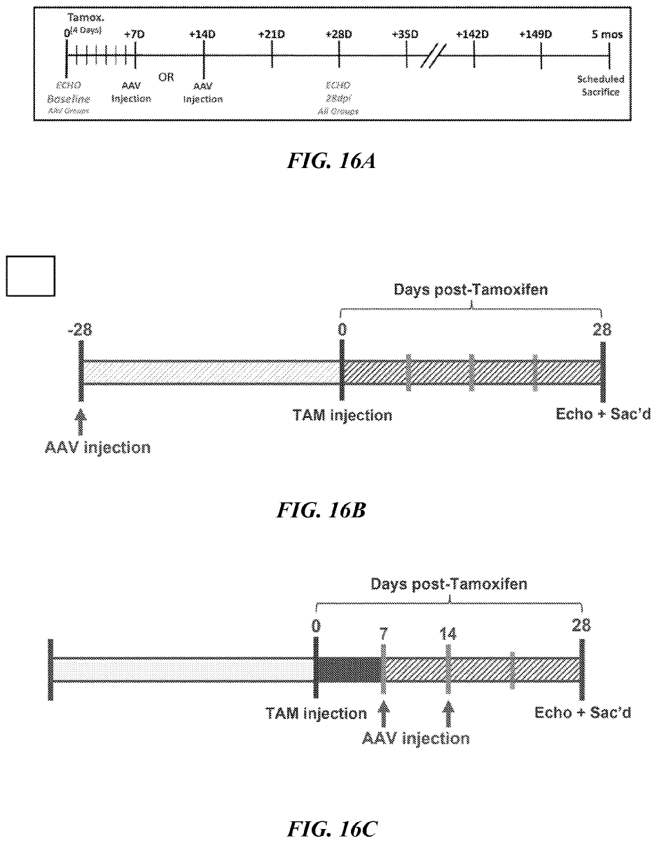

To further evaluate and characterize the potential benefit of the lead vector, AAVrh.74-hTnT-PKP2, further experiments were conducted. Specifically, experiments to evaluate the extent to which benefit in this ACM model could be observed after disease onset using the PKP2-cKO mouse model. Examples of the experimental models and timeline are illustrated shown in FIGS. 16A-C. Animals initially received tamoxifen injections followed by intravenous injections of different doses of AAVrh.74-hTnT-PKP2, or control Formulation Buffer (FB), either −28, +7 or +14 days later. Treatment groups across various studies included control animals, AAV9-hTnT-PKP2, and varying doses of AAVrh.74-hTnT-PKP2 as follows (see Figures and Figure Descriptions for specific doses used in separate experiments):
WT (Cre-): Formulation Buffer (Control);
PKP2-cKO: Formulation Buffer (Negative Control);
PKP2-cKO: AAV9-hTnT-PKP2: 3E13 vg/kg;
PKP2-cKO: AAVrh.74-hTnT-PKP2: 6E13 vg/kg; and
PKP2-cKO: AAVrh 74-hTnT-PKP2: 2E.14 vg/kg.

The expression cassette of the vectors was that depicted in FIG. 2. Echocardiography was performed at either 21, 28 Days, and 5 months following tamoxifen, depending on each experiment.

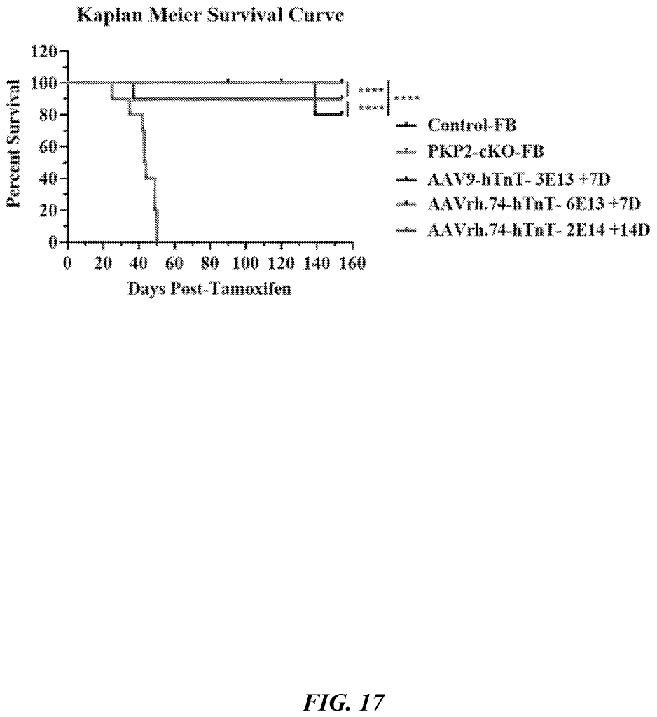

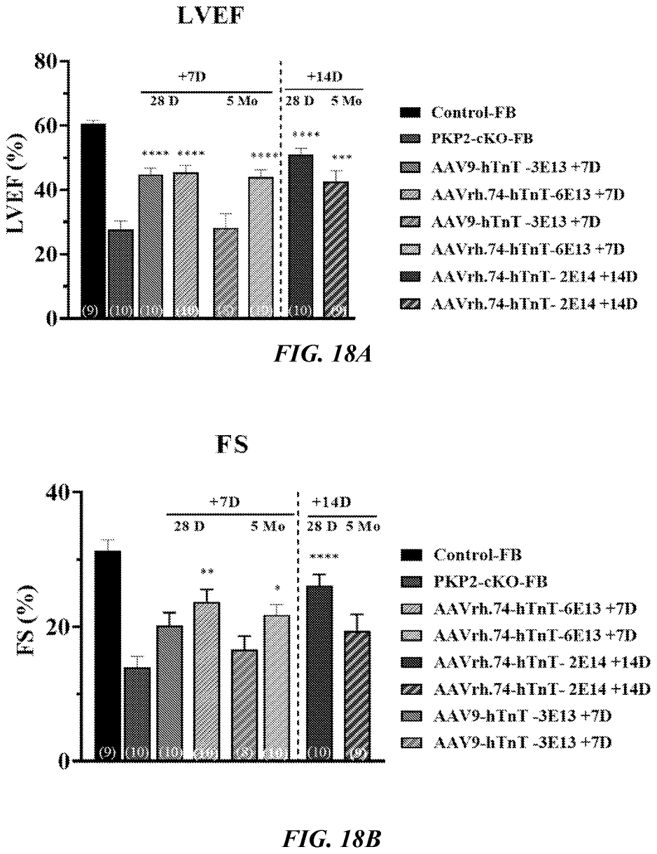

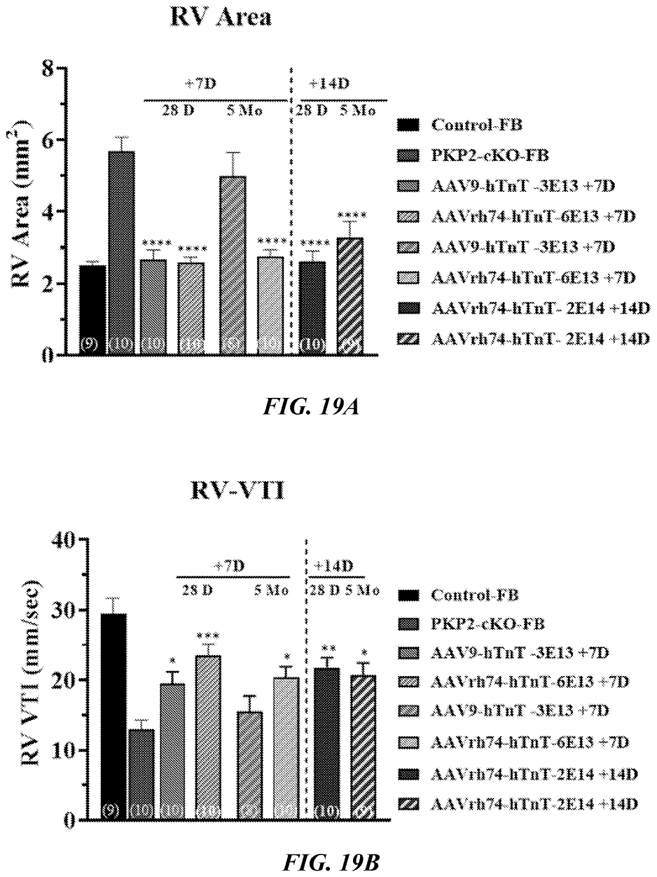

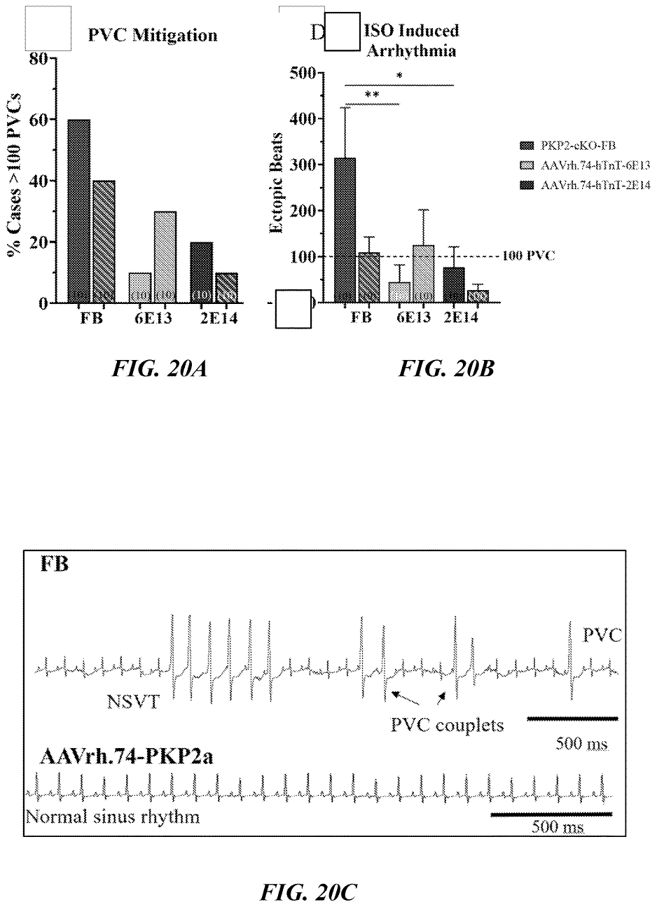

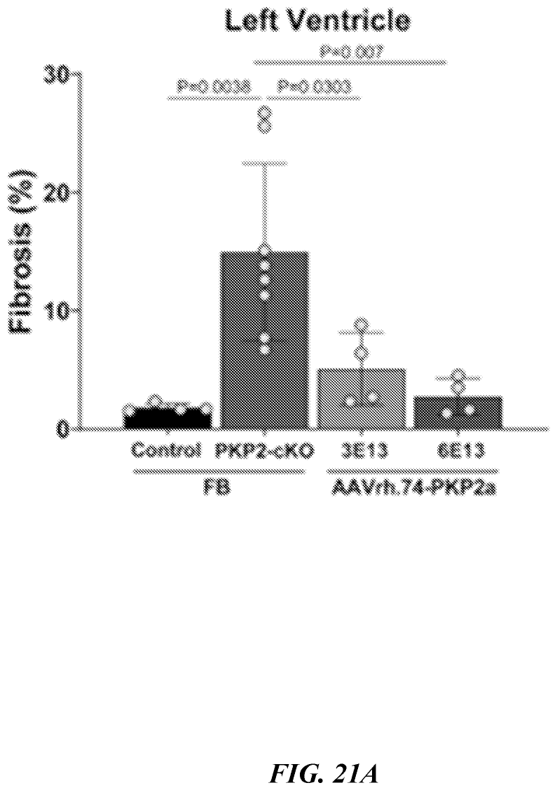

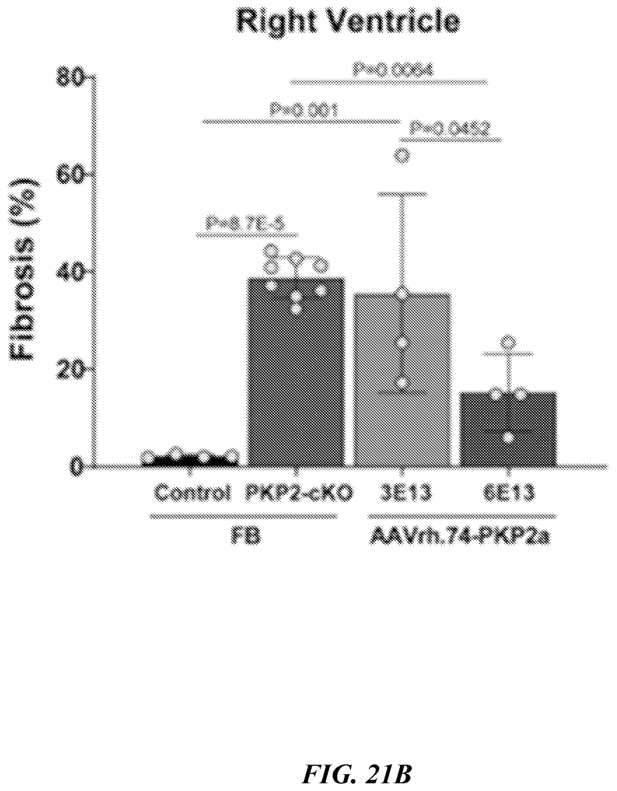

Findings from experiments using the delayed AAVrh.74-hTnT-PKP2a injection paradigm in the PKP2-cKO model are presented in FIGS. 17-20 and FIGS. 27-29. It was found that the previously defined “efficacious dose” of AAVrh.74-hTnT-PKP2, 6E13 vg/kg, resulted in 100% survival at the apriori defined time point of 5 months post-tamoxifen, compared to 100% mortality by day 50 in PKP2-cKO control animals (FIG. 17). The “high dose” of AAVrh.74-hTnT-PKP2a (2E14 vg/kg) resulted in 90% survival, with one animal death found to be unrelated to AAV-hTnT-PKP2a. All AAVrh.74-hTnT-PKP2 injected animals were found to have preserved Ejection Fraction (FIG. 18A), Fractional Shortening (FIG. 18B) and Right Ventricular Area at 28 Days, which sustained to 5 months (FIG. 19A). Importantly, AAVrh.74-hTnT-PKP2a administered after disease onset (14 days post-tamoxifen) resulted in a significant mitigation of isoproterenol-induced premature ventricular contractions normally observed as a consequence of loss of PKP2 in this mouse model as well as in patients with ACM (FIGS. 20A-20C). FIGS. 21A-B show the degree of fibrosis 5 months post-tamoxifen in left (FIG. 21A) and right ventricles (FIG. 21B) based on quantitation of the Percent Collagen following trichrome histological staining of the heart.

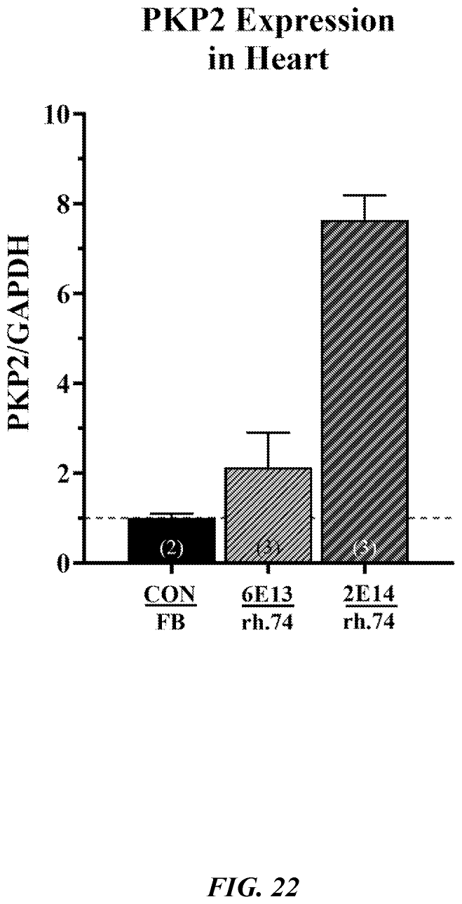

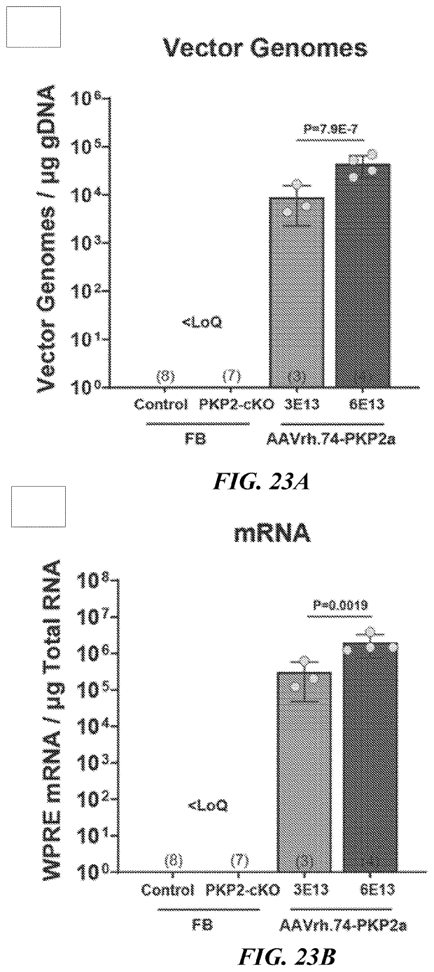

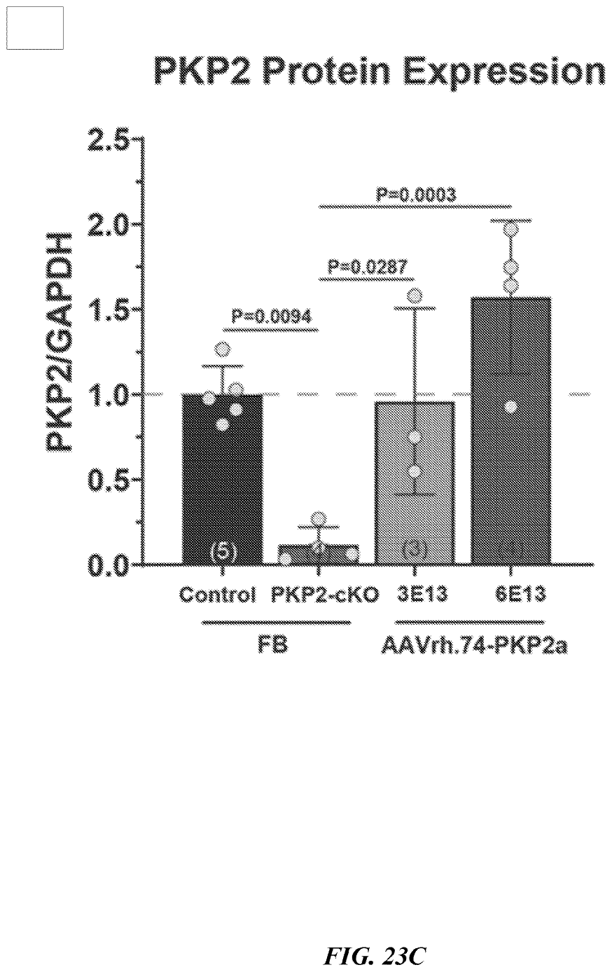

The presence and abundance of AAV vector DNA, transgene mRNA transcripts, and PKP2 protein in the heart were determined by ddPCR, RT-ddPCR and Western blot, respectively of heart lysates. Cumulative results are shown in FIGS. 22 and 23A-C. The results demonstrate efficient transduction and subsequent expression of human PKP2a (hPKP2a) transgene mRNA and PKP2 protein in the heart in all AAVrh.74-PKP2a injected mice.

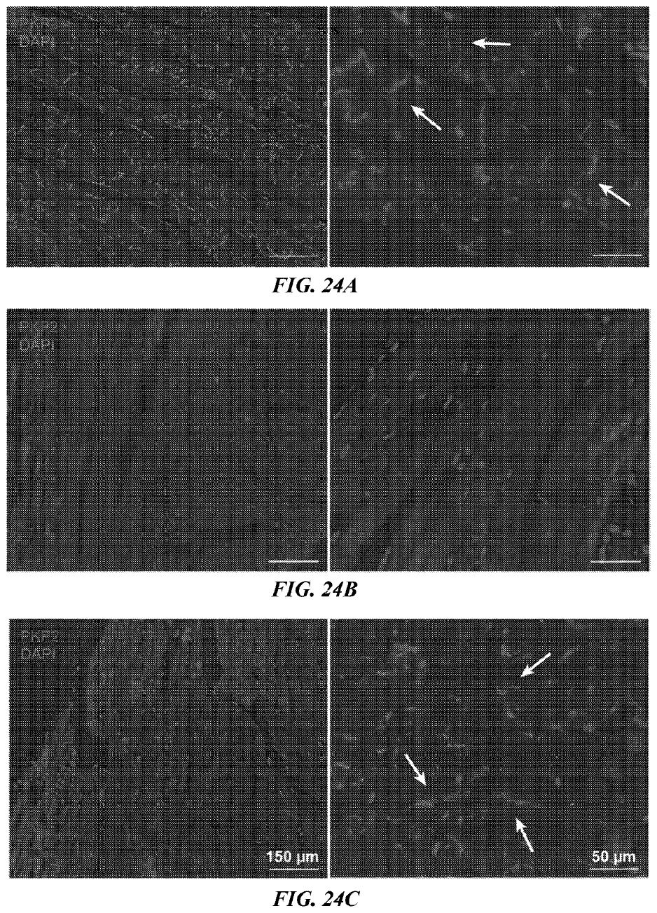

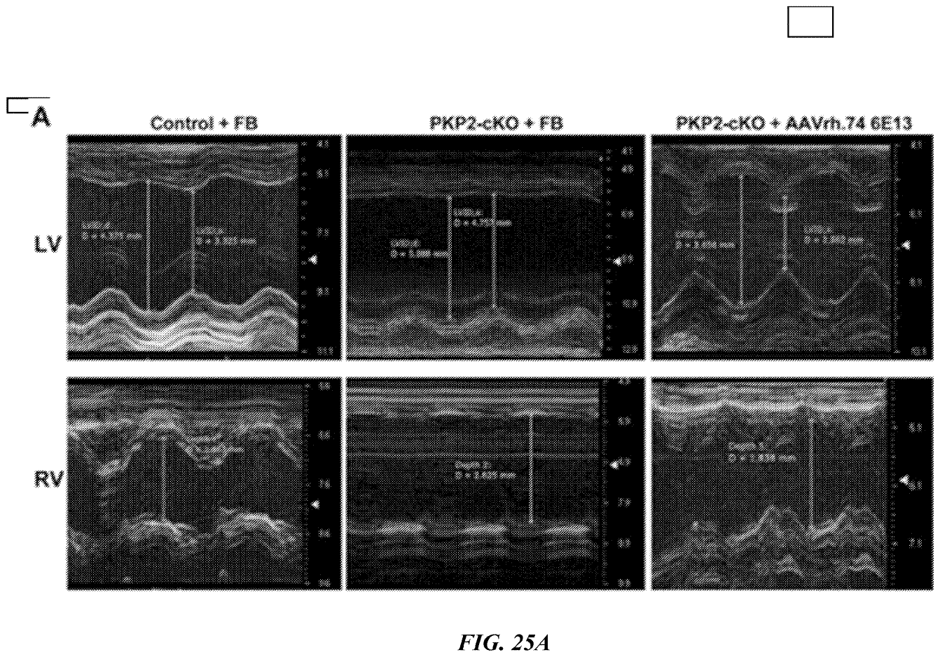

Immunofluorescence images acquired from fixed sections of heart tissue are shown in FIGS. 24A-C. FIG. 24A shows the immunolocalization of native PKP2 in the heart of a control (Cre-negative, TAM-injected) mouse. The image shows a clear immunoreactive PKP2-positive signal, visualized as well-defined plaques that align perpendicular to the direction of the fibers, as expected for an intercalated disc protein (see arrows in FIG. 24A). FIG. 24B and FIG. 24C show cardiac sections of PKP2-cKO mice euthanized 28 days post-TAM and previously injected (28 days pre-TAM) either with FB (FIG. 24B) or with FB+AAVrh.74-PKP2a (FIG. 24C). Immunolabeled PKP2 signal is notably absent in the heart from PKP2-cKO mice injected with FB alone (FIG. 24B). However, dense PKP2 signals oriented perpendicular to the fiber orientation are present in the heart that received the AAVrh.74-PKP2a treatment therapy (FIG. 24C; arrows). These results were confirmed in 4 mice per group. The data show that the exogenous PKP2 gene was transcribed and translated, and the expressed protein was properly localized to the subcellular domain expected for the native PKP2 protein,

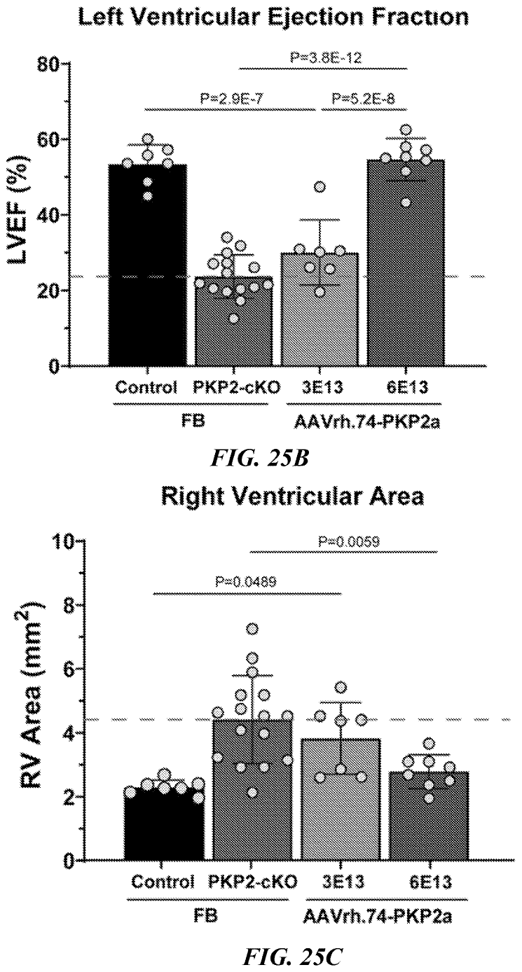

The ability of the exogenous protein to prevent the cardiomyopathic phenotype was also examined. Assessment of cardiac function in treated mice by echocardiography was performed 28-days post-TAM injection. FIGS. 25A-C show echocardiographic images (FIG. 25A) and cumulative data (FIG. 25B and FIG. 25C) obtained from hearts of mice injected with FB alone (control, first bar from a left; PKP2-cKO, second bar from left), or with AAVrh.74-PKP2a (PKP2-cKO+3×10¹³ vg/kg AAVrh 74-PKP2a., third bar from left;-PKP2-cKO+6×10¹³ vg/kg AAVri.74-PKP2a, fourth bar from left). As in FIGS. 25A-C, AAVrh.74-PKP2a or FB were injected 56 days before recording, and 28 days prior to TAM injection. As expected, loss of PKP2 expression caused a significant drop in the left ventricular ejection fraction (LVEF dotted line and second bar in FIG. 25B) and an increase in right ventricular (RV) area (dotted line and second bar from left in FIG. 25C). In contrast, AAVrh.74-mediated expression of hPKP2a mitigated or prevented the loss of contractile function in the LV and the increase in RV area in a dose-dependent manner.

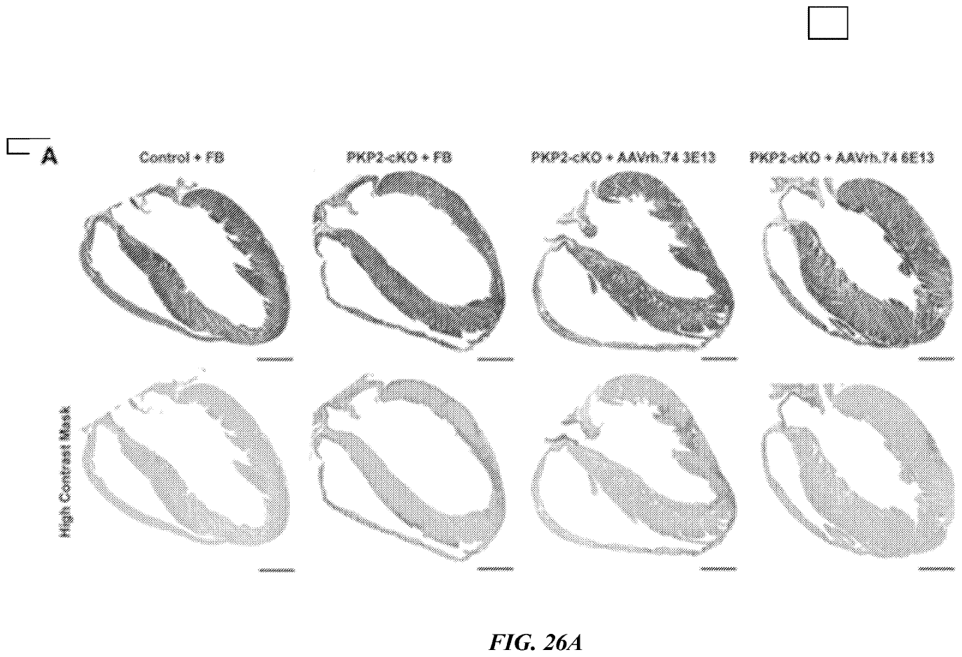

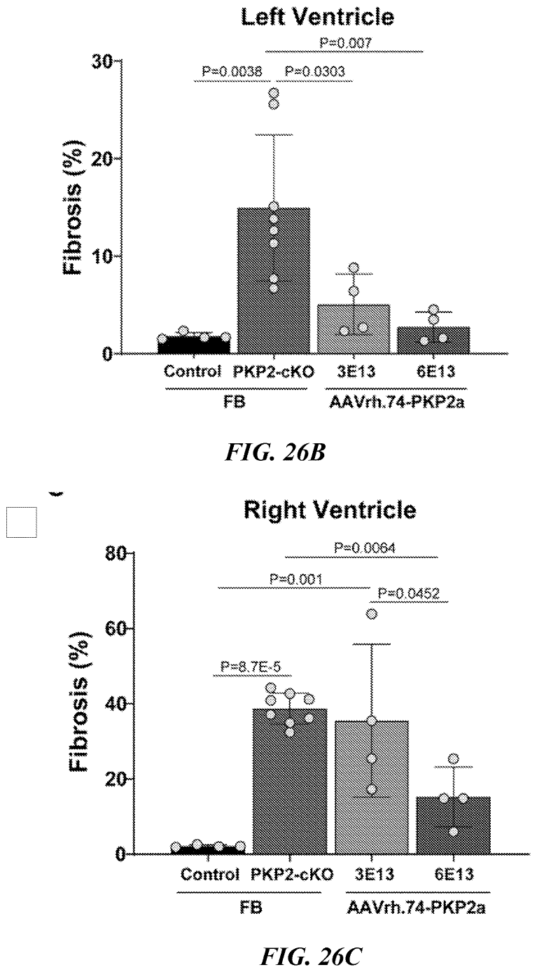

Cardiac fibrosis is a common feature in PKP2 deficient hearts. As shown in FIGS. 26A-C., visualization and quantification of the extent of collagen abundance in the ventricular free walls revealed the presence of extensive fibrosis in PKP2-cKO animals (second bars from left), which was significantly mitigated by AAVrh.74-PKP2a administration (third and fourth bars from left), particularly at the higher (6×10¹⁴ vg/kg) dose (fourth bar from left). Notably, dose-related reduction of cardiac fibrosis was observed in both the left and right ventricles of AAVrh.74-PKP2a injected animals.

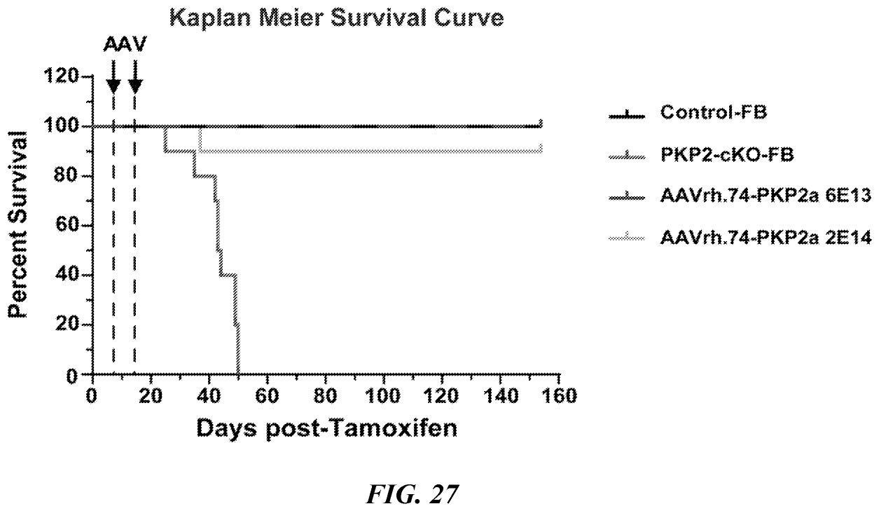

The data presented in the previous figures demonstrated that A AVrh.74-PKP2a treatment prior to TA M-mediated knockout of PKP2 could mitigate the development of the cardiomyopathic phenotype in the PKP2-eKO mice. To evaluate whether AAVrh.74-PKP2a could arrest the progression of the ARVC phenotype when delivered after tamoxifen mediated disease induction, PKP2-cKO mice were injected with AAVrh 74-PKP2a 7 or 14 days after TAM injection (See FIGS. 16B-C). In addition to echocardiography analyses, the mice were followed for long-term survival As illustrated by the Kaplan-Meier curve in FIG. 27, PKP2-cKO animals injected with FB only died between 30 and 50 days after TAM injection, consistent with previous reports. In contrast, all but one of the mice injected with AAVrh.74-PKP2a survived for 5 months (155 days post-TAM), at which time the animals were euthanized to examine protein expression and cardiac structure.

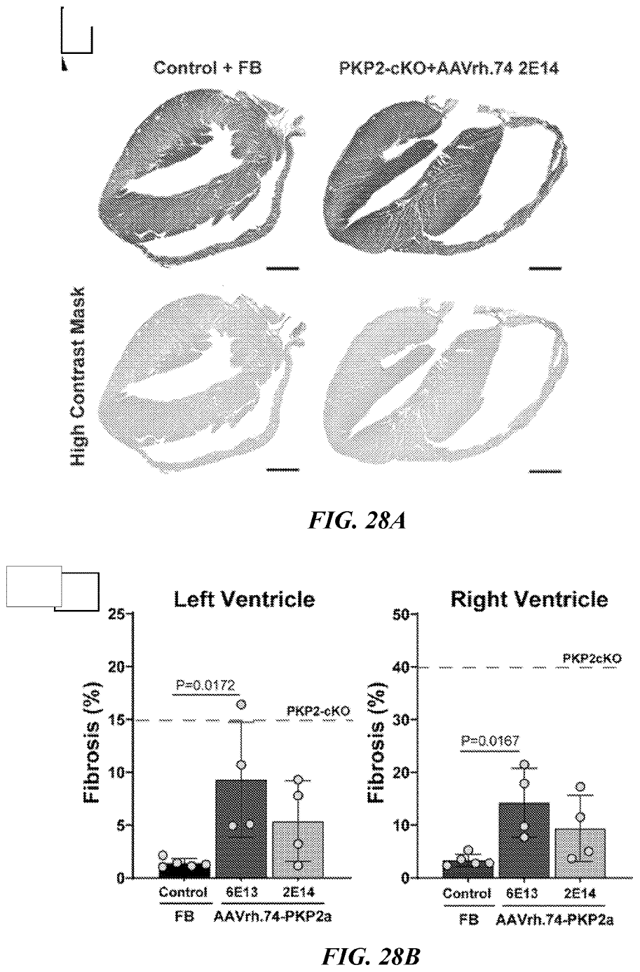

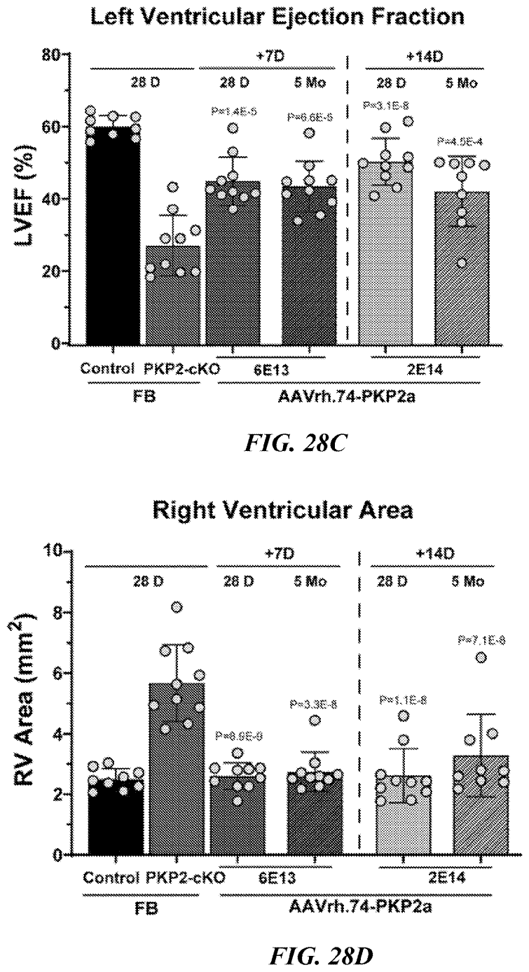

Furthermore, as shown by the representative images and cumulative data in FIGS. 28A-B, trichrome staining analyses revealed that the percent of the free wall of the LV and of the RV occupied by collagen in animals injected with the higher dose of A AVrh.74-PKP2a (2×10¹⁴ vg/kg, third bar from left) was similar (though trending toward higher values) when compared to that observed in control animals injected with FB (first bar from left). The abundance of collagen in hearts from mice that received the lower dose of AAVrh.74-PKP2a (6×10¹³ vg/kg, second bar from left) was higher than in control animals. Importantly, a comparison to collagen abundance in PKP2-cKO animals at the same time point was not possible because of the early lethality in that group. However, the abundance of collagen in the treated animals was less than what has been recorded through historical data from PKP2-eKO mice at 42 days after TAM injection (near their time of death, as per the Kaplan-Meier curve in FIG. 27). Finally, cumulative data obtained from echocardiographic analysis of hearts from mice injected with AAVrh.74-PKP2a 7 days or 14 days after TAM are presented in FIGS. 28C-D. Hearts from FB-injected PKP2-cKO mice presented a drastic reduction in LVEF (FIG. 28C) and an increase in RV area (FIG. 28D) 28 days after TAM injection (compare second bar from left to first bar from left in FIGS. 28C-D), consistent with previous results. These changes were mitigated by AAVrh.74-PKP2a, even when injected 14 days after TAM, and the beneficial effects persisted up to 5 months after TAM injection, the longest time point evaluated.

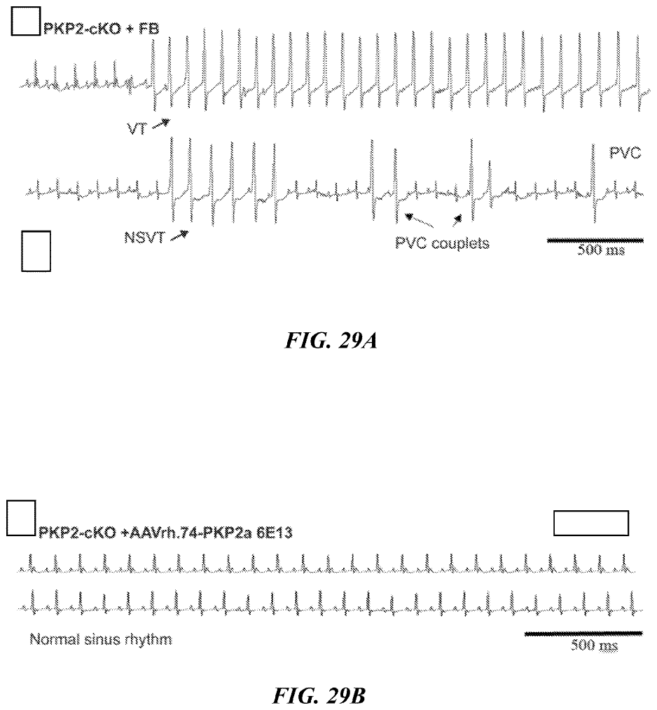

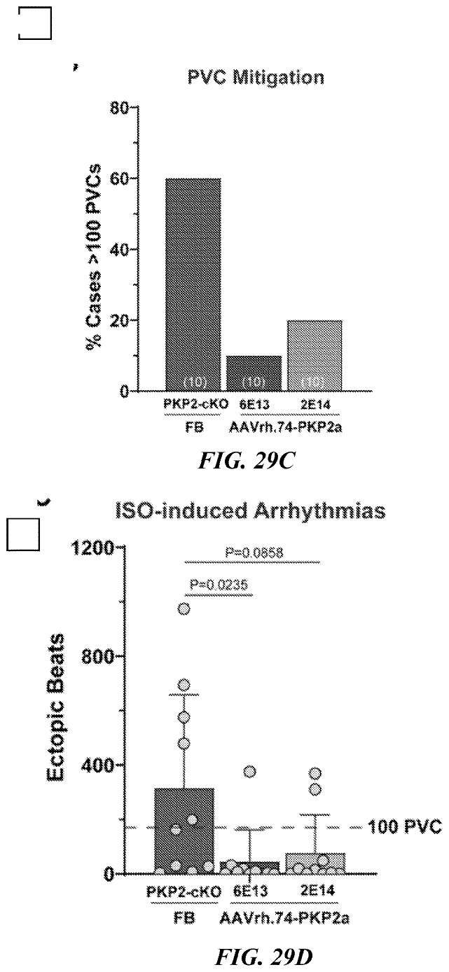

Previous studies have documented that a bolus injection of isoproterenol (ISO) 3 mg/kg leads to premature ventricular contractions in anesthetized PKP2-cKO mice 21 days after TAM injection. Therefore, the ISO challenge protocol was used to determine whether AAVrh 74-PKP2a delivered 14 days after TAM can mitigate arrhythmia burden in PKP2-cKO animals. Representative ECG traces are shown in FIGS. 29A-B. FE-injected PKP2-cKO animals presented multiple PVCs, and 6 out of 10 animals showed more than 100 PVCs within the 30 minutes of recording after ISO injection (FIG. 29C) with a total count of 0.300 PVCs on average (FIG. 29D) In contrast, both parameters of arrhythmia burden were drastically reduced by administration of AAVrh 74-PKP2a. at both doses tested (6×10¹³ vg/kg and 2×10¹⁴ vg/kg purple and orange bars, respectively, in FIGS. 29C-D).

Safety and toxicology studies in mice revealed appropriate vector DNA biodistribution consistent. Transgene mRNA was 10-100-fold enriched in the heart relative to all other organs, including liver. Clinical pathology and histopathology analyses revealed that AAVrh.74-PKP2a was well tolerated and safe across multiple studies up to (and including) doses of 3×10¹⁴ vg/kg in mice.

### Example 4: Phase 1 Clinical Trial

A multi-center dose escalation study in PKP2-associated ACM is conducted. Subjects may include those with mutations leading to truncated PKP2 expression, e.g., a “rightstop-gain” variant mutation. Subjects may have an ICD.

Key Inclusion/Exclusion Criteria include:≥18 y/o; ACM diagnosis, PKP2 genetic mutation, clinically-significant arrhythmia burden, and preserved IN function,

It is expected that treatment with a PKP2 gene therapy vector disclosed herein, e.g., AAV9 or AAVrh,74 will result in improvement of one or more endpoints. Anticipated endpoints include but are not strictly limited to:
Primary: Safety (frequency, severity of AEs/SAEs)
Secondary: Key Exploratory/Preliminary efficacy
   Myocardial PKP2 expression and vector copy number
   PVC reduction on serial Holter, other parameters affecting or any of the parameters affecting LTVA risk based on the ARVC Risk Calculator

### Exploratory Endpoints

VT/VF monitoring (ECG/Holter/AICD)
Reduced incidence of ICD shocks
RV function improvement (MRI/Echo)
LV Ejection fraction (MI/Echo)
Blood biomarkers: BNP, NT-proBNP, Troponin I
Kansas City Cardiomyopathy Questionnaire
Stable LGE on MRI
Left or right atrial volumes
Additional Arrhythmia-related endpoints
Physiologic, Cardiac-related Assessments and Quality of Life Evaluations
Additional Biomarkers

### Example 5: In Vivo Expression of Pkp2 in Non-Human Primates (NHP)

Studies were conducted in non-human primates (NHPs) to demonstrate that the vectors disclosed herein led to expression in the heart of large mammals.

NHPs were intravenously injected with either a “Low” or “1-igh” dose of AAVrh.74-PIP2a (see FIG. 2, e.g., SEQ ID NO: 13 or 97) or formulation buffer (FB; vehicle control). The Low dose corresponded to 8×10¹³ vg/kg, and the High dose corresponded to 3×10¹³ vg/kg The AAVrh.74-PKP2a vector delivers to cardiomyocytes an expression cassette and flanking AAV inverted terminal repeats (LTRs), the expression cassette comprising a polynucleotide comprising:
a polynucleotide sequence encoding a PIKP2, isoform a, of SEQ ID NO: 1;
a hTnnT2 promoter sequence (with exon 1) of SEQ ID NO:32, wherein the promoter sequence is operatively linked to the polynucleotide sequence encoding the PKP2a;
and a polyA sequence.

Three months following a single intravenous administration of the AAVrh 74-PKP2a vector, the animals were humanely sacrificed, and heart tissue was collected. Expression of human PKP2a transgene mRNA and PKP2a protein in the heart tissue was determined by RT-PCR, immunostaining, and Western blot.

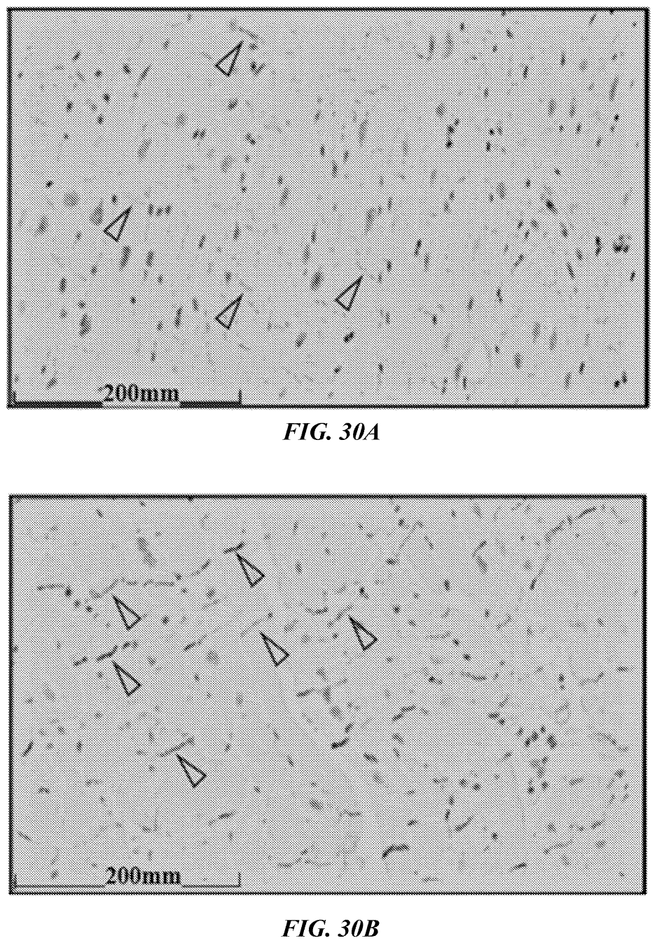

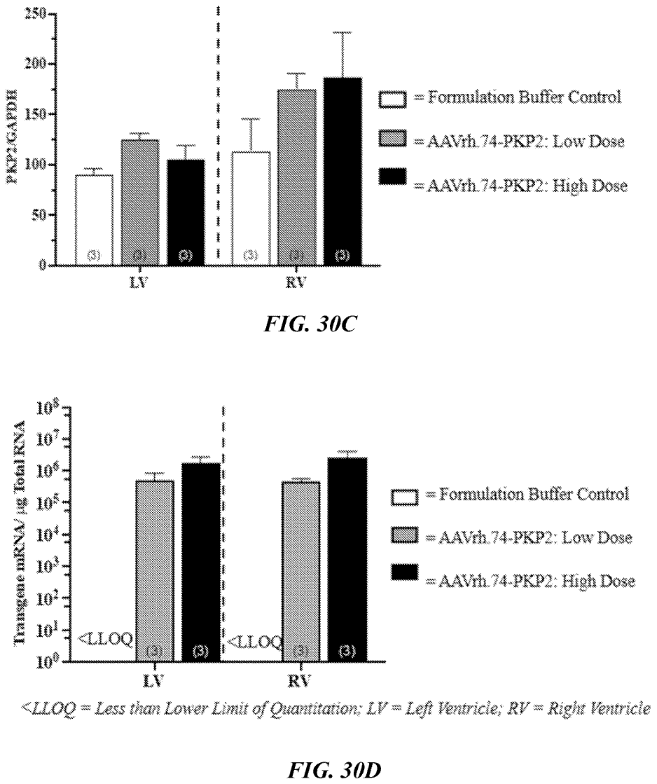

As shown in FIGS. 30A-D. while NHPs administered FB show endogenous PKP2a expression in the heart, NHPs administered AAVrh.74-PKP2a showed increased levels of PKP2a mRNA and protein as compared to the control. The data illustrate detection, across multiple levels of analyses, of transgene PKP2a in the heart of the nonhuman primates. FIGS. 30A-B are photomicrographs following immunohistochemical labeling for PKP2 protein in sections through the nonhuman primate heart of either a control FB (FIG. 30A) or “Low Dose” treated animal (FIG. 30B). A modest level of endogenous PKP2 immunolabeling was observed at the intercalated discs, the inter-junctional spaces between cardiomyocytes, in FB control treated animals (see e.g. open triangles in FIG. 30A). This is in stark contrast to the intense and higher frequency of detected PKP2-positive immunolabeling observed at the intercalated discs of “Low Dose” treated animals (see e.g., open triangles in FIG. 30B). FIG. 30C and FIG. 30D illustrate quantitation of PKP2 protein by Western blot and transgene mRNA (hPKP2a) in the ventricles of the nonhuman primate heart following intravenous infusion of either a “Low” or “High” dose AAVrh.74-PKP2a or FB (control).

These data demonstrate that the AAVrh.74-PKP2a vector transduced and expressed the potentially therapeutic transgene in the heart tissue of larger mammals following intravenous administration.

Safety and toxicology studies on nonhuman primates (NIP) revealed appropriate vector DNA biodistribution. Clinical pathology and histopathology analyses revealed that AAVrh.74-PKP2a was well tolerated and safe across multiple studies up to (and including) doses of 3×10¹³ vg/kg in NHP.

Sequences

| SID | |
|---|---|
| NO: | Sequence |
| 8 | ACCCTTCAGATTAAAAATAACTGAGGTAAGGGCCTGGGTAGGGGAGGTGGTGTGAGACG |
| | CTCCTGTCTCTCCTCTATCTGCCCATCGGCCCTTTGGGGAGGAGGAATGTGCCCAAGGA |
| | CTAAAAAAAGGCCATGGAGCCAGAGGGGCGAGGGCAACAGACCTTTCATGGGCAAACC |
| | TTGGGGCCCTGCTGTCTAGCATGCCCCACTACGGGTCTAGGCTGCCCATGTAAGGAGG |
| | CAAGGCCTGGGGACACCCGAGATGCCTGGTTATAATTAACCCAGACATGTGGCTGCCCC |
| | CCCCCCCCCAACACCTGCTGCCTCTAAAAATAACCCTGTCCCTGGTGGATCCCCTGCAT |
| | GCGAAGATCTTCGAACAAGGCTGTGGGGGACTGAGGGCAGGCTGTAACAGGCTTGGGG |
| | GCCAGGGCTTATACGTGCCTGGGACTCCCAAAGTATTACTGTTCCATGTTCCCGGCGAA |
| | GGGCCAGCTGTCCCCCGCCAGCTAGACTCAGCACTTAGTTTAGGAACCAGTGAGCAAGT |
| | CAGCCCTTGGGGCAGCCCATACAAGGCCATGGGGCTGGGCAAGCTGCACGCCTGGGT |
| | CCGGGGTGGGCACGGTGCCCGGGCAACGAGCTGAAAGCTCATCTGCTCTCAGGGGCC |
| | CCTCCCTGGGGACAGCCCCTCCTGGCTAGTCACACCCTGTAGGCTCCTCTATATAACCC |
| | AGGGGCACAGGGGCTGCCCTCATTCTACCACCACCTCCACAGCACAGACAGACACTCA |
| | GGAGCCAGCCAGGCCACCATGGCAGCCCCCGGCGCCCCAGCTGAGTACGGCTACATC |
| | CGGACCGTCCTGGGCCAGCAGATCCTGGGACAACTGGACAGCTCCAGCCTGGCGCTGC |
| | CCTCCGAGGCCAAGCTGAAGCTGGGGGGGAGCAGCGGCCGCGGCGGCCAGACAGTCA |
| | AGAGCCTGCGGATCCAGGAGCAGGTGCAGCAGACCCTCGCCCGGAAGGGCCGCAGCT |
| | CCGTGGGCAACGGAAATCTTCACCGAACCAGCAGTGTTCCTGAGTATGTCTACAACCTA |
| | CACTTGGTTGAAAATGATTTTGTTGGAGGCCGTTCCCCTGTTCCTAAAACCTATGACATG |
| | CTAAAGGCTGGCACAACTGCCACTTATGAAGGTCGCTGGGGAAGAGGAACAGCACAGTA |
| | CAGCTCCCAGAAGTCCGTGGAAGAAAGGTCCTTGAGGCATCCTCTGAGGAGACTGGAG |
| | ATTTCTCCTGACAGCAGCCCGGAGAGGGCTCACTACACGCACAGCGATTACCAGTACAG |
| | CCAGAGAAGCCAGGCTGGGCACACCCTGCACCACCAAGAAAGCAGGGGGGCCGCCCT |
| | CCTAGTGCCACCGAGATATGCTCGTTCCGAGATCGTGGGGGTCAGCCGTGCTGGCACC |
| | ACAAGCAGGCAGCGCCACTTTGACACATACCACAGACAGTACCAGCATGGCTCTGTTAG |
| | CGACACCGTTTTTGACAGCATCCCTGCCAACCCGGCCCTGCTCACGTACCCCAGGCCAG |
| | GGACCAGCCGCAGCATGGGCAACCTCTTGGAGAAGGAGAACTACCTGACGGCAGGGCT |
| | CACTGTCGGGCAGGTCAGGCCGCTGGTGCCCCTGCAGCCCGTCACTCAGAACAGGGCT |
| | TCCAGGTCCTCCTGGCATCAGAGCTCCTTCCACAGCACCCGCACGCTGAGGGAAGCTG |
| | GGCCCAGTGTCGCCGTGGATTCCAGCGGGAGGAGAGCGCACTTGACTGTCGGCCAGG |
| | CGGCCGCAGGGGGAAGTGGGAATCTGCTCACTGAGAGAAGCACTTTCACTGACTCCCA |
| | GCTGGGGAATGCAGACATGGAGATGACTCTGGAGCGAGCAGTGAGTATGCTCGAGGCA |
| | GACCACATGCTGCCATCCAGGATTTCTGCTGCAGCTACTTTCATACAGCACGAGTGCTTC |
| | CAGAAATCTGAAGCTCGGAAGAGGGTTAACCAGCTTCGTGGCATCCTCAAGCTTCTGCA |
| | GCTCCTAAAAGTTCAGAATGAAGACGTTCAGCGAGCTGTGTGTGGGGCCTTGAGAAACT |
| | TAGTATTTGAAGACAATGACAACAAATTGGAGGTGGCTGAACTAAATGGGGTACCTCGG |
| | CTGCTCCAGGTGCTGAAGCAAACCAGAGACTTGGAGACTAAAAAACAAATAACAGGTTT |
| | GCTGTGGAATTTGTCATCTAATGACAAACTCAAGAATCTCATGATAACAGAAGCATTGCTT |
| | ACGCTGACGGAGAATATCATCATCCCCTTTTCTGGGTGGCCTGAAGGAGACTACCCAAA |
| | AGCAAATGGTTTGCTCGATTTTGACATATTCTACAACGTCACTGGATGCCTAAGAAACAT |
| | GAGTTCTGCTGGCGCTGATGGGAGAAAAGCGATGAGAAGATGTGACGGACTCATTGACT |
| | CACTGGTCCATTATGTCAGAGGAACCATTGCAGATTACCAGCCAGATGACAAGGCCACG |
| | GAGAATTGTGTGTGCATTCTTCATAACCTCTCCTACCAGCTGGAGGCAGAGCTCCCAGA |
| | GAAATATTCCCAGAATATCTATATTCAAAACCGGAATATCCAGACTGACAACAACAAAAGT |
| | ATTGGATGTTTTGGCAGTCGAAGCAGGAAAGTAAAAGAGCAATACCAGGACGTGCCGAT |
| | GCCGGAGGAAAAGAGCAACCCCAAGGGCGTGGAGTGGCTGTGGCATTCCATTGTTATA |
| | AGGATGTATCTGTCCTTGATCGCCAAAAGTGTCCGCAACTACACACAAGAAGCATCCTTA |
| | GGAGCTCTGCAGAACCTCACGGCCGGAAGTGGACCAATGCCGACATCAGTGGCTCAGA |
| | CAGTTGTCCAGAAGGAAAGTGGCCTGCAGCACACCCGAAAGATGCTGCATGTTGGTGAC |
| | CCAAGTGTGAAAAAGACAGCCATCTCGCTGCTGAGGAATCTGTCCCGGAATCTTTCTCT |
| | GCAGAATGAAATTGCCAAAGAAACTCTCCCTGATTTGGTTTCCATCATTCCTGACACAGT |
| | CCCGAGTACTGACCTTCTCATTGAAACTACAGCCTCTGCCTGTTACACATTGAACAACAT |
| | AATCCAAAACAGTTACCAGAATGCACGCGACCTTCTAAACACCGGGGGCATCCAGAAAA |
| | TTATGGCCATTAGTGCAGGCGATGCCTATGCCTCCAACAAAGCAAGTAAAGCTGCTTCC |
| | GTCCTTCTGTATTCTCTGTGGGCACACACGGAACTGCATCATGCCTACAAGAAGGCTCA |
| | GTTTAAGAAGACAGATTTTGTCAACAGCCGGACTGCCAAAGCCTACCACTCCCTTAAAGA |
| | CTGATCAACCTCTGGATTACAAAATTTGTGAAAGATTGACTGGTATTCTTAACTATGTTGC |
| | TCCTTTTACGCTATGTGGATACGCTGCTTTAATGCCTTTGTATCATGCTATTGCTTCCCGT |
| | ATGGCTTTCATTTTCTCCTCCTTGTATAAATCCTGGTTGCTGTCTCTTTATGAGGAGTTGT |
| | GGCCCGTTGTCAGGCAACGTGGCGTGGTGTGCACTGTGTTTGCTGACGCAACCCCCAC |
| | TGGTTGGGGCATTGCCACCACCTGTCAGCTCCTTTCCGGGACTTTCGCTTTCCCCCTCC |
| | CTATTGCCACGGCGGAACTCATCGCCGCCTGCCTTGCCCGCTGCTGGACAGGGGCTCG |
| | GCTGTTGGGCACTGACAATTCCGTGGTGTTGTCGGGGAAATCATCGTCCTTTCCTTGGC |
| | TGCTCGCCTGTGTTGCCACCTGGATTCTGCGCGGGACGTCCTTCTGCTACGTCCCTTCG |
| | GCCCTCAATCCAGCGGACCTTCCTTCCCGCGGCCTGCTGCCGGCTCTGCGGCCTCTTC |
| | CGCGTCTTCGCCTTCGCCCTCAGACGAGTCGGATCTCCCTTTGGGCCGCCTCCCCGCA |
| | CTGCCCGGGTGGCATCCCTGTGACCCCTCCCCAGTGCCTCTCCTGGCCCTGGAAGTTG |
| | CCACTCCAGTGCCCACCAGCCTTGTCCTAATAAAATTAAGTTGCATCATTTTGTCTGACTA |
| | GGTGTCCTTCTATAATATTATGGGGTGGAGGGGGGTGGTATGGAGCAAGGGGCCCAAG |
| | TTGGGAAGAAACCTGTAGGGCCTGC |
| 9 | CTCAGTCCATTAGGAGCCAGTAGCCTGGAAGATGTCTTTACCCCCAGCATCAGTTCAAGT |
| | GGAGCAGCACATAACTCTTGCCCTCTGCCTTCCAAGATTCTGGTGCTGAGACTTATGGA |
| | GTGTCTTGGAGGTTGCCTTCTGCCCCCCAACCCTGCTCCCAGCTGGCCCTCCCAGGCCT |
| | GGGTTGCTGGCCTCTGCTTTATCAGGATTCTCAAGAGGGACAGCTGGTTTATGTTGCAT |
| | GACTGTTCCCTGCATATCTGCTCTGGTTTTAAATAGCTTATCTGAGCAGCTGGAGGACCA |
| | CATGGGCTTATATGGCGTGGGGTACATGTTCCTGTAGCCTTGTCCCTGGCACCTGCCAA |
| | AATAGCAGCCAACACCCCCCACCCCCACCGCCATCCCCCTGCCCCACCCGTCCCCTGT |
| | CGCACATTCCTCCCTCCGCAGGGCTGGCTCACCAGGCCCCAGCCCACATGCCTGCTTA |
| | AAGCCCTCTCCATCCTCTGCCTCACCCAGTCCCCGCTGAGACTGAGCAGACGCCTCCAG |
| | GATCTGTCGGCAGGCCACCATGGCAGCCCCCGGCGCCCCAGCTGAGTACGGCTACATC |
| | CGGACCGTCCTGGGCCAGCAGATCCTGGGACAACTGGACAGCTCCAGCCTGGCGCTGC |
| | CCTCCGAGGCCAAGCTGAAGCTGGGGGGGAGCAGCGGCCGCGGCGGCCAGACAGTCA |
| | AGAGCCTGCGGATCCAGGAGCAGGTGCAGCAGACCCTCGCCCGGAAGGGCCGCAGCT |
| | CCGTGGGCAACGGAAATCTTCACCGAACCAGCAGTGTTCCTGAGTATGTCTACAACCTA |
| | CACTTGGTTGAAAATGATTTTGTTGGAGGCCGTTCCCCTGTTCCTAAAACCTATGACATG |
| | CTAAAGGCTGGCACAACTGCCACTTATGAAGGTCGCTGGGGAAGAGGAACAGCACAGTA |
| | CAGCTCCCAGAAGTCCGTGGAAGAAAGGTCCTTGAGGCATCCTCTGAGGAGACTGGAG |
| | ATTTCTCCTGACAGCAGCCCGGAGAGGGCTCACTACACGCACAGCGATTACCAGTACAG |
| | CCAGAGAAGCCAGGCTGGGCACACCCTGCACCACCAAGAAAGCAGGCGGGCCGCCCT |
| | CCTAGTGCCACCGAGATATGCTCGTTCCGAGATCGTGGGGGTCAGCCGTGCTGGCACC |
| | ACAAGCAGGCAGCGCCACTTTGACACATACCACAGACAGTACCAGCATGGCTCTGTTAG |
| | CGACACCGTTTTTGACAGCATCCCTGCCAACCCGGCCCTGCTCACGTACCCCAGGCCAG |
| | GGACCAGCCGCAGCATGGGCAACCTCTTGGAGAAGGAGAACTACCTGACGGCAGGGCT |
| | CACTGTCGGGCAGGTCAGGCCGCTGGTGCCCCTGCAGCCCGTCACTCAGAACAGGGCT |
| | TCCAGGTCCTCCTGGCATCAGAGCTCCTTCCACAGCACCCGCACGCTGAGGGAAGCTG |
| | GGCCCAGTGTCGCCGTGGATTCCAGCGGGAGGAGAGCGCACTTGACTGTCGGCCAGG |
| | CGGCCGCAGGGGGAAGTGGGAATCTGCTCACTGAGAGAAGCACTTTCACTGACTCCCA |
| | GCTGGGGAATGCAGACATGGAGATGACTCTGGAGCGAGCAGTGAGTATGCTCGAGGCA |
| | GACCACATGCTGCCATCCAGGATTTCTGCTGCAGCTACTTTCATACAGCACGAGTGCTTC |
| | CAGAAATCTGAAGCTCGGAAGAGGGTTAACCAGCTTCGTGGCATCCTCAAGCTTCTGCA |
| | GCTCCTAAAAGTTCAGAATGAAGACGTTCAGCGAGCTGTGTGTGGGGCCTTGAGAAACT |
| | TAGTATTTGAAGACAATGACAACAAATTGGAGGTGGCTGAACTAAATGGGGTACCTCGG |
| | CTGCTCCAGGTGCTGAAGCAAACCAGAGACTTGGAGACTAAAAAACAAATAACAGGTTT |
| | GCTGTGGAATTTGTCATCTAATGACAAACTCAAGAATCTCATGATAACAGAAGCATTGCTT |
| | ACGCTGACGGAGAATATCATCATCCCCTTTTCTGGGTGGCCTGAAGGAGACTACCCAAA |
| | AGCAAATGGTTTGCTCGATTTTGACATATTCTACAACGTCACTGGATGCCTAAGAAACAT |
| | GAGTTCTGCTGGCGCTGATGGGAGAAAAGCGATGAGAAGATGTGACGGACTCATTGACT |
| | CACTGGTCCATTATGTCAGAGGAACCATTGCAGATTACCAGCCAGATGACAAGGCCACG |
| | GAGAATTGTGTGTGCATTCTTCATAACCTCTCCTACCAGCTGGAGGCAGAGCTCCCAGA |
| | GAAATATTCCCAGAATATCTATATTCAAAACCGGAATATCCAGACTGACAACAACAAAAGT |
| | ATTGGATGTTTTGGCAGTCGAAGCAGGAAAGTAAAAGAGCAATACCAGGACGTGCCGAT |
| | GCCGGAGGAAAAGAGCAACCCCAAGGGCGTGGAGTGGCTGTGGCATTCCATTGTTATA |
| | AGGATGTATCTGTCCTTGATCGCCAAAAGTGTCCGCAACTACACACAAGAAGCATCCTTA |
| | GGAGCTCTGCAGAACCTCACGGCCGGAAGTGGACCAATGCCGACATCAGTGGCTCAGA |
| | CAGTTGTCCAGAAGGAAAGTGGCCTGCAGCACACCCGAAAGATGCTGCATGTTGGTGAC |
| | CCAAGTGTGAAAAAGACAGCCATCTCGCTGCTGAGGAATCTGTCCCGGAATCTTTCTCT |
| | GCAGAATGAAATTGCCAAAGAAACTCTCCCTGATTTGGTTTCCATCATTCCTGACACAGT |
| | CCCGAGTACTGACCTTCTCATTGAAACTACAGCCTCTGCCTGTTACACATTGAACAACAT |
| | AATCCAAAACAGTTACCAGAATGCACGCGACCTTCTAAACACCGGGGGCATCCAGAAAA |
| | TTATGGCCATTAGTGCAGGCGATGCCTATGCCTCCAACAAAGCAAGTAAAGCTGCTTCC |
| | GTCCTTCTGTATTCTCTGTGGGCACACACGGAACTGCATCATGCCTACAAGAAGGCTCA |
| | GTTTAAGAAGACAGATTTTGTCAACAGCCGGACTGCCAAAGCCTACCACTCCCTTAAAGA |
| | CTGATCAACCTCTGGATTACAAAATTTGTGAAAGATTGACTGGTATTCTTAACTATGTTGC |
| | TCCTTTTACGCTATGTGGATACGCTGCTTTAATGCCTTTGTATCATGCTATTGCTTCCCGT |
| | ATGGCTTTCATTTTCTCCTCCTTGTATAAATCCTGGTTGCTGTCTCTTTATGAGGAGTTGT |
| | GGCCCGTTGTCAGGCAACGTGGCGTGGTGTGCACTGTGTTTGCTGACGCAACCCCCAC |
| | TGGTTGGGGCATTGCCACCACCTGTCAGCTCCTTTCCGGGACTTTCGCTTTCCCCCTCC |
| | CTATTGCCACGGCGGAACTCATCGCCGCCTGCCTTGCCCGCTGCTGGACAGGGGCTCG |
| | GCTGTTGGGCACTGACAATTCCGTGGTGTTGTCGGGGAAATCATCGTCCTTTCCTTGGC |
| | TGCTCGCCTGTGTTGCCACCTGGATTCTGCGCGGGACGTCCTTCTGCTACGTCCCTTCG |
| | GCCCTCAATCCAGCGGACCTTCCTTCCCGCGGCCTGCTGCCGGCTCTGCGGCCTCTTC |
| | CGCGTCTTCGCCTTCGCCCTCAGACGAGTCGGATCTCCCTTTGGGCCGCCTCCCCGCA |
| | CTGCCCGGGTGGCATCCCTGTGACCCCTCCCCAGTGCCTCTCCTGGCCCTGGAAGTTG |
| | CCACTCCAGTGCCCACCAGCCTTGTCCTAATAAAATTAAGTTGCATCATTTTGTCTGACTA |
| | GGTGTCCTTCTATAATATTATGGGGTGGAGGGGGGTGGTATGGAGCAAGGGGCCCAAG |
| | TTGGGAAGAAACCTGTAGGGCCTGC |
| 10 | ACCCTTCAGATTAAAAATAACTGAGGTAAGGGCCTGGGTAGGGGAGGTGGTGTGAGACG |
| | CTCCTGTCTCTCCTCTATCTGCCCATCGGCCCTTTGGGGAGGAGGAATGTGCCCAAGGA |
| | CTAAAAAAAGGCCATGGAGCCAGAGGGGCGAGGGCAACAGACCTTTCATGGGCAAACC |
| | TTGGGGCCCTGCTGTCTAGCATGCCCCACTACGGGTCTAGGCTGCCCATGTAAGGAGG |
| | CAAGGCCTGGGGACACCCGAGATGCCTGGTTATAATTAACCCAGACATGTGGCTGCCCC |
| | CCCCCCCCCAACACCTGCTGCCTCTAAAAATAACCCTGTCCCTGGTGGATCCCCTGCAT |
| | GCGAAGATCTTCGAACAAGGCTGTGGGGGACTGAGGGCAGGCTGTAACAGGCTTGGGG |
| | GCCAGGGCTTATACGTGCCTGGGACTCCCAAAGTATTACTGTTCCATGTTCCCGGCGAA |
| | GGGCCAGCTGTCCCCCGCCAGCTAGACTCAGCACTTAGTTTAGGAACCAGTGAGCAAGT |
| | CAGCCCTTGGGGCAGCCCATACAAGGCCATGGGGCTGGGCAAGCTGCACGCCTGGGT |
| | CCGGGGTGGGCACGGTGCCCGGGCAACGAGCTGAAAGCTCATCTGCTCTCAGGGGCC |
| | CCTCCCTGGGGACAGCCCCTCCTGGCTAGTCACACCCTGTAGGCTCCTCTATATAACCC |
| | AGGGGCACAGGGGCTGCCCTCATTCTACCACCACCTCCACAGCACAGACAGACACTCA |
| | GGAGCCAGCCAGGCCACCATGGCAGCCCCCGGCGCCCCAGCTGAGTACGGCTACATC |
| | CGGACCGTCCTGGGCCAGCAGATCCTGGGACAACTGGACAGCTCCAGCCTGGCGCTGC |
| | CCTCCGAGGCCAAGCTGAAGCTGGCGGGGAGCAGCGGCCGCGGCGGCCAGACAGTCA |
| | AGAGCCTGCGGATCCAGGAGCAGGTGCAGCAGACCCTCGCCCGGAAGGGCCGCAGCT |
| | CCGTGGGCAACGGAAATCTTCACCGAACCAGCAGTGTTCCTGAGTATGTCTACAACCTA |
| | CACTTGGTTGAAAATGATTTTGTTGGAGGCCGTTCCCCTGTTCCTAAAACCTATGACATG |
| | CTAAAGGCTGGCACAACTGCCACTTATGAAGGTCGCTGGGGAAGAGGAACAGCACAGTA |
| | CAGCTCCCAGAAGTCCGTGGAAGAAAGGTCCTTGAGGCATCCTCTGAGGAGACTGGAG |
| | ATTTCTCCTGACAGCAGCCCGGAGAGGGCTCACTACACGCACAGCGATTACCAGTACAG |
| | CCAGAGAAGCCAGGCTGGGCACACCCTGCACCACCAAGAAAGCAGGCGGGCCGCCCT |
| | CCTAGTGCCACCGAGATATGCTCGTTCCGAGATCGTGGGGGTCAGCCGTGCTGGCACC |
| | ACAAGCAGGCAGCGCCACTTTGACACATACCACAGACAGTACCAGCATGGCTCTGTTAG |
| | CGACACCGTTTTTGACAGCATCCCTGCCAACCCGGCCCTGCTCACGTACCCCAGGCCAG |
| | GGACCAGCCGCAGCATGGGCAACCTCTTGGAGAAGGAGAACTACCTGACGGCAGGGCT |
| | CACTGTCGGGCAGGTCAGGCCGCTGGTGCCCCTGCAGCCCGTCACTCAGAACAGGGCT |
| | TCCAGGTCCTCCTGGCATCAGAGCTCCTTCCACAGCACCCGCACGCTGAGGGAAGCTG |
| | GGCCCAGTGTCGCCGTGGATTCCAGCGGGAGGAGAGCGCACTTGACTGTCGGCCAGG |
| | CGGCCGCAGGGGGAAGTGGGAATCTGCTCACTGAGAGAAGCACTTTCACTGACTCCCA |
| | GCTGGGGAATGCAGACATGGAGATGACTCTGGAGCGAGCAGTGAGTATGCTCGAGGCA |
| | GACCACATGCTGCCATCCAGGATTTCTGCTGCAGCTACTTTCATACAGCACGAGTGCTTC |
| | CAGAAATCTGAAGCTCGGAAGAGGGTTAACCAGCTTCGTGGCATCCTCAAGCTTCTGCA |
| | GCTCCTAAAAGTTCAGAATGAAGACGTTCAGCGAGCTGTGTGTGGGGCCTTGAGAAACT |
| | TAGTATTTGAAGACAATGACAACAAATTGGAGGTGGCTGAACTAAATGGGGTACCTCGG |
| | CTGCTCCAGGTGCTGAAGCAAACCAGAGACTTGGAGACTAAAAAACAAATAACAGACCA |
| | TACAGTCAATTTAAGAAGTAGGAATGGCTGGCCGGGCGCGGTGGCTCACGCCTGTAATC |
| | CCAGCACTTTGGGAGGCCAAGGCGGGCGGATCACGAGGTCAGGAGTTCGAGACCAGC |
| | CTGACCAACATGGTTTGCTGTGGAATTTGTCATCTAATGACAAACTCAAGAATCTCATGAT |
| | AACAGAAGCATTGCTTACGCTGACGGAGAATATCATCATCCCCTTTTCTGGGTGGCCTGA |
| | AGGAGACTACCCAAAAGCAAATGGTTTGCTCGATTTTGACATATTCTACAACGTCACTGG |
| | ATGCCTAAGAAACATGAGTTCTGCTGGCGCTGATGGGAGAAAAGCGATGAGAAGATGTG |
| | ACGGACTCATTGACTCACTGGTCCATTATGTCAGAGGAACCATTGCAGATTACCAGCCAG |
| | ATGACAAGGCCACGGAGAATTGTGTGTGCATTCTTCATAACCTCTCCTACCAGCTGGAG |
| | GCAGAGCTCCCAGAGAAATATTCCCAGAATATCTATATTCAAAACCGGAATATCCAGACT |
| | GACAACAACAAAAGTATTGGATGTTTTGGCAGTCGAAGCAGGAAAGTAAAAGAGCAATAC |
| | CAGGACGTGCCGATGCCGGAGGAAAAGAGCAACCCCAAGGGCGTGGAGTGGCTGTGG |
| | CATTCCATTGTTATAAGGATGTATCTGTCCTTGATCGCCAAAAGTGTCCGCAACTACACA |
| | CAAGAAGCATCCTTAGGAGCTCTGCAGAACCTCACGGCCGGAAGTGGACCAATGCCGA |
| | CATCAGTGGCTCAGACAGTTGTCCAGAAGGAAAGTGGCCTGCAGCACACCCGAAAGATG |
| | CTGCATGTTGGTGACCCAAGTGTGAAAAAGACAGCCATCTCGCTGCTGAGGAATCTGTC |
| | CCGGAATCTTTCTCTGCAGAATGAAATTGCCAAAGAAACTCTCCCTGATTTGGTTTCCAT |
| | CATTCCTGACACAGTCCCGAGTACTGACCTTCTCATTGAAACTACAGCCTCTGCCTGTTA |
| | CACATTGAACAACATAATCCAAAACAGTTACCAGAATGCACGCGACCTTCTAAACACCGG |
| | GGGCATCCAGAAAATTATGGCCATTAGTGCAGGCGATGCCTATGCCTCCAACAAAGCAA |
| | GTAAAGCTGCTTCCGTCCTTCTGTATTCTCTGTGGGCACACACGGAACTGCATCATGCCT |
| | ACAAGAAGGCTCAGTTTAAGAAGACAGATTTTGTCAACAGCCGGACTGCCAAAGCCTAC |
| | CACTCCCTTAAAGACTGATCAACCTCTGGATTACAAAATTTGTGAAAGATTGACTGGTATT |
| | CTTAACTATGTTGCTCCTTTTACGCTATGTGGATACGCTGCTTTAATGCCTTTGTATCATG |
| | CTATTGCTTCCCGTATGGCTTTCATTTTCTCCTCCTTGTATAAATCCTGGTTGCTGTCTCT |
| | TTATGAGGAGTTGTGGCCCGTTGTCAGGCAACGTGGCGTGGTGTGCACTGTGTTTGCTG |
| | ACGCAACCCCCACTGGTTGGGGCATTGCCACCACCTGTCAGCTCCTTTCCGGGACTTTC |
| | GCTTTCCCCCTCCCTATTGCCACGGCGGAACTCATCGCCGCCTGCCTTGCCCGCTGCTG |
| | GACAGGGGCTCGGCTGTTGGGCACTGACAATTCCGTGGTGTTGTCGGGGAAATCATCG |
| | TCCTTTCCTTGGCTGCTCGCCTGTGTTGCCACCTGGATTCTGCGCGGGACGTCCTTCTG |
| | CTACGTCCCTTCGGCCCTCAATCCAGCGGACCTTCCTTCCCGCGGCCTGCTGCCGGCT |
| | CTGCGGCCTCTTCCGCGTCTTCGCCTTCGCCCTCAGACGAGTCGGATCTCCCTTTGGGC |
| | CGCCTCCCCGCACTGCCCGGGTGGCATCCCTGTGACCCCTCCCCAGTGCCTCTCCTGG |
| | CCCTGGAAGTTGCCACTCCAGTGCCCACCAGCCTTGTCCTAATAAAATTAAGTTGCATCA |
| | TTTTGTCTGACTAGGTGTCCTTCTATAATATTATGGGGTGGAGGGGGGTGGTATGGAGC |
| | AAGGGGCCCAAGTTGGGAAGAAACCTGTAGGGCCTGC |
| 11 | gcgcgctcgctcgctcactgaggccgcccgggcaaagcccgggcgtcgggcgacctttggtcgcccggcctcagtgagcgagc |
| | gagcgcgcagagagggagtggccaactccatcactaggggttccttgtagttaatgattaacccgccatgctacttatctacgt |
| | aCTCAGTCCATTAGGAGCCAGTAGCCTGGAAGATGTCTTTACCCCCAGCATCAGTTCAAGT |
| | GGAGCAGCACATAACTCTTGCCCTCTGCCTTCCAAGATTCTGGTGCTGAGACTTATGGA |
| | GTGTCTTGGAGGTTGCCTTCTGCCCCCCAACCCTGCTCCCAGCTGGCCCTCCCAGGCCT |
| | GGGTTGCTGGCCTCTGCTTTATCAGGATTCTCAAGAGGGACAGCTGGTTTATGTTGCAT |
| | GACTGTTCCCTGCATATCTGCTCTGGTTTTAAATAGCTTATCTGAGCAGCTGGAGGACCA |
| | CATGGGCTTATATGGCGTGGGGTACATGTTCCTGTAGCCTTGTCCCTGGCACCTGCCAA |
| | AATAGCAGCCAACACCCCCCACCCCCACCGCCATCCCCCTGCCCCACCCGTCCCCTGT |
| | CGCACATTCCTCCCTCCGCAGGGCTGGCTCACCAGGCCCCAGCCCACATGCCTGCTTA |
| | AAGCCCTCTCCATCCTCTGCCTCACCCAGTCCCCGCTGAGACTGAGCAGACGCCTCCAG |
| | GATCTGTCGGCAGGCCACCATGGCAGCCCCCGGCGCCCCAGCTGAGTACGGCTACATC |
| | CGGACCGTCCTGGGCCAGCAGATCCTGGGACAACTGGACAGCTCCAGCCTGGCGCTGC |
| | CCTCCGAGGCCAAGCTGAAGCTGGCGGGGAGCAGCGGCCGCGGCGGCCAGACAGTCA |
| | AGAGCCTGCGGATCCAGGAGCAGGTGCAGCAGACCCTCGCCCGGAAGGGCCGCAGCT |
| | CCGTGGGCAACGGAAATCTTCACCGAACCAGCAGTGTTCCTGAGTATGTCTACAACCTA |
| | CACTTGGTTGAAAATGATTTTGTTGGAGGCCGTTCCCCTGTTCCTAAAACCTATGACATG |
| | CTAAAGGCTGGCACAACTGCCACTTATGAAGGTCGCTGGGGAAGAGGAACAGCACAGTA |
| | CAGCTCCCAGAAGTCCGTGGAAGAAAGGTCCTTGAGGCATCCTCTGAGGAGACTGGAG |
| | ATTTCTCCTGACAGCAGCCCGGAGAGGGCTCACTACACGCACAGCGATTACCAGTACAG |
| | CCAGAGAAGCCAGGCTGGGCACACCCTGCACCACCAAGAAAGCAGGCGGGCCGCCCT |
| | CCTAGTGCCACCGAGATATGCTCGTTCCGAGATCGTGGGGGTCAGCCGTGCTGGCACC |
| | ACAAGCAGGCAGCGCCACTTTGACACATACCACAGACAGTACCAGCATGGCTCTGTTAG |
| | CGACACCGTTTTTGACAGCATCCCTGCCAACCCGGCCCTGCTCACGTACCCCAGGCCAG |
| | GGACCAGCCGCAGCATGGGCAACCTCTTGGAGAAGGAGAACTACCTGACGGCAGGGCT |
| | CACTGTCGGGCAGGTCAGGCCGCTGGTGCCCCTGCAGCCCGTCACTCAGAACAGGGCT |
| | TCCAGGTCCTCCTGGCATCAGAGCTCCTTCCACAGCACCCGCACGCTGAGGGAAGCTG |
| | GGCCCAGTGTCGCCGTGGATTCCAGCGGGAGGAGAGCGCACTTGACTGTCGGCCAGG |
| | CGGCCGCAGGGGGAAGTGGGAATCTGCTCACTGAGAGAAGCACTTTCACTGACTCCCA |
| | GCTGGGGAATGCAGACATGGAGATGACTCTGGAGCGAGCAGTGAGTATGCTCGAGGCA |
| | GACCACATGCTGCCATCCAGGATTTCTGCTGCAGCTACTTTCATACAGCACGAGTGCTTC |
| | CAGAAATCTGAAGCTCGGAAGAGGGTTAACCAGCTTCGTGGCATCCTCAAGCTTCTGCA |
| | GCTCCTAAAAGTTCAGAATGAAGACGTTCAGCGAGCTGTGTGTGGGGCCTTGAGAAACT |
| | TAGTATTTGAAGACAATGACAACAAATTGGAGGTGGCTGAACTAAATGGGGTACCTCGG |
| | CTGCTCCAGGTGCTGAAGCAAACCAGAGACTTGGAGACTAAAAAACAAATAACAGACCA |
| | TACAGTCAATTTAAGAAGTAGGAATGGCTGGCCGGGCGCGGTGGCTCACGCCTGTAATC |
| | CCAGCACTTTGGGAGGCCAAGGCGGGGGGATCACGAGGTCAGGAGTTCGAGACCAGC |
| | CTGACCAACATGGTTTGCTGTGGAATTTGTCATCTAATGACAAACTCAAGAATCTCATGAT |
| | AACAGAAGCATTGCTTACGCTGACGGAGAATATCATCATCCCCTTTTCTGGGTGGCCTGA |
| | AGGAGACTACCCAAAAGCAAATGGTTTGCTCGATTTTGACATATTCTACAACGTCACTGG |
| | ATGCCTAAGAAACATGAGTTCTGCTGGCGCTGATGGGAGAAAAGCGATGAGAAGATGTG |
| | ACGGACTCATTGACTCACTGGTCCATTATGTCAGAGGAACCATTGCAGATTACCAGCCAG |
| | ATGACAAGGCCACGGAGAATTGTGTGTGCATTCTTCATAACCTCTCCTACCAGCTGGAG |
| | GCAGAGCTCCCAGAGAAATATTCCCAGAATATCTATATTCAAAACCGGAATATCCAGACT |
| | GACAACAACAAAAGTATTGGATGTTTTGGCAGTCGAAGCAGGAAAGTAAAAGAGCAATAC |
| | CAGGACGTGCCGATGCCGGAGGAAAAGAGCAACCCCAAGGGCGTGGAGTGGCTGTGG |
| | CATTCCATTGTTATAAGGATGTATCTGTCCTTGATCGCCAAAAGTGTCCGCAACTACACA |
| | CAAGAAGCATCCTTAGGAGCTCTGCAGAACCTCACGGCCGGAAGTGGACCAATGCCGA |
| | CATCAGTGGCTCAGACAGTTGTCCAGAAGGAAAGTGGCCTGCAGCACACCCGAAAGATG |
| | CTGCATGTTGGTGACCCAAGTGTGAAAAAGACAGCCATCTCGCTGCTGAGGAATCTGTC |
| | CCGGAATCTTTCTCTGCAGAATGAAATTGCCAAAGAAACTCTCCCTGATTTGGTTTCCAT |
| | CATTCCTGACACAGTCCCGAGTACTGACCTTCTCATTGAAACTACAGCCTCTGCCTGTTA |
| | CACATTGAACAACATAATCCAAAACAGTTACCAGAATGCACGCGACCTTCTAAACACCGG |
| | GGGCATCCAGAAAATTATGGCCATTAGTGCAGGCGATGCCTATGCCTCCAACAAAGCAA |
| | GTAAAGCTGCTTCCGTCCTTCTGTATTCTCTGTGGGCACACACGGAACTGCATCATGCCT |
| | ACAAGAAGGCTCAGTTTAAGAAGACAGATTTTGTCAACAGCCGGACTGCCAAAGCCTAC |
| | CACTCCCTTAAAGACTGATCAACCTCTGGATTACAAAATTTGTGAAAGATTGACTGGTATT |
| | CTTAACTATGTTGCTCCTTTTACGCTATGTGGATACGCTGCTTTAATGCCTTTGTATCATG |
| | CTATTGCTTCCCGTATGGCTTTCATTTTCTCCTCCTTGTATAAATCCTGGTTGCTGTCTCT |
| | TTATGAGGAGTTGTGGCCCGTTGTCAGGCAACGTGGCGTGGTGTGCACTGTGTTTGCTG |
| | ACGCAACCCCCACTGGTTGGGGCATTGCCACCACCTGTCAGCTCCTTTCCGGGACTTTC |
| | GCTTTCCCCCTCCCTATTGCCACGGCGGAACTCATCGCCGCCTGCCTTGCCCGCTGCTG |
| | GACAGGGGCTCGGCTGTTGGGCACTGACAATTCCGTGGTGTTGTCGGGGAAATCATCG |
| | TCCTTTCCTTGGCTGCTCGCCTGTGTTGCCACCTGGATTCTGCGCGGGACGTCCTTCTG |
| | CTACGTCCCTTCGGCCCTCAATCCAGCGGACCTTCCTTCCCGCGGCCTGCTGCCGGCT |
| | CTGCGGCCTCTTCCGCGTCTTCGCCTTCGCCCTCAGACGAGTCGGATCTCCCTTTGGGC |
| | CGCCTCCCCGCACTGCCCGGGTGGCATCCCTGTGACCCCTCCCCAGTGCCTCTCCTGG |
| | CCCTGGAAGTTGCCACTCCAGTGCCCACCAGCCTTGTCCTAATAAAATTAAGTTGCATCA |
| | TTTTGTCTGACTAGGTGTCCTTCTATAATATTATGGGGTGGAGGGGGGTGGTATGGAGC |
| | AAGGGGCCCAAGTTGGGAAGAAACCTGTAGGGCCTGCtacgtagataagtagcatgggggttaatcat |
| | taactacaaggaacccctagtgatggagttggccactccctctctgcgcgctcgctcgctcactgaggccgggcgaccaaaggt |
| | cgcccgacgcccgggctttgcccgggcggcctcagtgagcgagcgagcgcgc |
| 12 | gcgcgctcgctcgctcactgaggccgcccgggcaaagcccgggcgtcgggcgacctttggtcgcccggcctcagtgagcgagc |
| | gagcgcgcagagagggagtggccaactccatcactaggggttccttgtagttaatgattaacccgccatgctacttatctacgt |
| | aACCCTTCAGATTAAAAATAACTGAGGTAAGGGCCTGGGTAGGGGAGGTGGTGTGAGACG |
| | CTCCTGTCTCTCCTCTATCTGCCCATCGGCCCTTTGGGGAGGAGGAATGTGCCCAAGGA |
| | CTAAAAAAAGGCCATGGAGCCAGAGGGGCGAGGGCAACAGACCTTTCATGGGCAAACC |
| | TTGGGGCCCTGCTGTCTAGCATGCCCCACTACGGGTCTAGGCTGCCCATGTAAGGAGG |
| | CAAGGCCTGGGGACACCCGAGATGCCTGGTTATAATTAACCCAGACATGTGGCTGCCCC |
| | CCCCCCCCCAACACCTGCTGCCTCTAAAAATAACCCTGTCCCTGGTGGATCCCCTGCAT |
| | GCGAAGATCTTCGAACAAGGCTGTGGGGGACTGAGGGCAGGCTGTAACAGGCTTGGGG |
| | GCCAGGGCTTATACGTGCCTGGGACTCCCAAAGTATTACTGTTCCATGTTCCCGGCGAA |
| | GGGCCAGCTGTCCCCCGCCAGCTAGACTCAGCACTTAGTTTAGGAACCAGTGAGCAAGT |
| | CAGCCCTTGGGGCAGCCCATACAAGGCCATGGGGCTGGGCAAGCTGCACGCCTGGGT |
| | CCGGGGTGGGCACGGTGCCCGGGCAACGAGCTGAAAGCTCATCTGCTCTCAGGGGCC |
| | CCTCCCTGGGGACAGCCCCTCCTGGCTAGTCACACCCTGTAGGCTCCTCTATATAACCC |
| | AGGGGCACAGGGGCTGCCCTCATTCTACCACCACCTCCACAGCACAGACAGACACTCA |
| | GGAGCCAGCCAGGCCACCATGGCAGCCCCCGGCGCCCCAGCTGAGTACGGCTACATC |
| | CGGACCGTCCTGGGCCAGCAGATCCTGGGACAACTGGACAGCTCCAGCCTGGCGCTGC |
| | CCTCCGAGGCCAAGCTGAAGCTGGCGGGGAGCAGCGGCCGCGGCGGCCAGACAGTCA |
| | AGAGCCTGCGGATCCAGGAGCAGGTGCAGCAGACCCTCGCCCGGAAGGGCCGCAGCT |
| | CCGTGGGCAACGGAAATCTTCACCGAACCAGCAGTGTTCCTGAGTATGTCTACAACCTA |
| | CACTTGGTTGAAAATGATTTTGTTGGAGGCCGTTCCCCTGTTCCTAAAACCTATGACATG |
| | CTAAAGGCTGGCACAACTGCCACTTATGAAGGTCGCTGGGGAAGAGGAACAGCACAGTA |
| | CAGCTCCCAGAAGTCCGTGGAAGAAAGGTCCTTGAGGCATCCTCTGAGGAGACTGGAG |
| | ATTTCTCCTGACAGCAGCCCGGAGAGGGCTCACTACACGCACAGCGATTACCAGTACAG |
| | CCAGAGAAGCCAGGCTGGGCACACCCTGCACCACCAAGAAAGCAGGCGGGCCGCCCT |
| | CCTAGTGCCACCGAGATATGCTCGTTCCGAGATCGTGGGGGTCAGCCGTGCTGGCACC |
| | ACAAGCAGGCAGCGCCACTTTGACACATACCACAGACAGTACCAGCATGGCTCTGTTAG |
| | CGACACCGTTTTTGACAGCATCCCTGCCAACCCGGCCCTGCTCACGTACCCCAGGCCAG |
| | GGACCAGCCGCAGCATGGGCAACCTCTTGGAGAAGGAGAACTACCTGACGGCAGGGCT |
| | CACTGTCGGGCAGGTCAGGCCGCTGGTGCCCCTGCAGCCCGTCACTCAGAACAGGGCT |
| | TCCAGGTCCTCCTGGCATCAGAGCTCCTTCCACAGCACCCGCACGCTGAGGGAAGCTG |
| | GGCCCAGTGTCGCCGTGGATTCCAGCGGGAGGAGAGCGCACTTGACTGTCGGCCAGG |
| | CGGCCGCAGGGGGAAGTGGGAATCTGCTCACTGAGAGAAGCACTTTCACTGACTCCCA |
| | GCTGGGGAATGCAGACATGGAGATGACTCTGGAGCGAGCAGTGAGTATGCTCGAGGCA |
| | GACCACATGCTGCCATCCAGGATTTCTGCTGCAGCTACTTTCATACAGCACGAGTGCTTC |
| | CAGAAATCTGAAGCTCGGAAGAGGGTTAACCAGCTTCGTGGCATCCTCAAGCTTCTGCA |
| | GCTCCTAAAAGTTCAGAATGAAGACGTTCAGCGAGCTGTGTGTGGGGCCTTGAGAAACT |
| | TAGTATTTGAAGACAATGACAACAAATTGGAGGTGGCTGAACTAAATGGGGTACCTCGG |
| | CTGCTCCAGGTGCTGAAGCAAACCAGAGACTTGGAGACTAAAAAACAAATAACAGGTTT |
| | GCTGTGGAATTTGTCATCTAATGACAAACTCAAGAATCTCATGATAACAGAAGCATTGCTT |
| | ACGCTGACGGAGAATATCATCATCCCCTTTTCTGGGTGGCCTGAAGGAGACTACCCAAA |
| | AGCAAATGGTTTGCTCGATTTTGACATATTCTACAACGTCACTGGATGCCTAAGAAACAT |
| | GAGTTCTGCTGGCGCTGATGGGAGAAAAGCGATGAGAAGATGTGACGGACTCATTGACT |
| | CACTGGTCCATTATGTCAGAGGAACCATTGCAGATTACCAGCCAGATGACAAGGCCACG |
| | GAGAATTGTGTGTGCATTCTTCATAACCTCTCCTACCAGCTGGAGGCAGAGCTCCCAGA |
| | GAAATATTCCCAGAATATCTATATTCAAAACCGGAATATCCAGACTGACAACAACAAAAGT |
| | ATTGGATGTTTTGGCAGTCGAAGCAGGAAAGTAAAAGAGCAATACCAGGACGTGCCGAT |
| | GCCGGAGGAAAAGAGCAACCCCAAGGGCGTGGAGTGGCTGTGGCATTCCATTGTTATA |
| | AGGATGTATCTGTCCTTGATCGCCAAAAGTGTCCGCAACTACACACAAGAAGCATCCTTA |
| | GGAGCTCTGCAGAACCTCACGGCCGGAAGTGGACCAATGCCGACATCAGTGGCTCAGA |
| | CAGTTGTCCAGAAGGAAAGTGGCCTGCAGCACACCCGAAAGATGCTGCATGTTGGTGAC |
| | CCAAGTGTGAAAAAGACAGCCATCTCGCTGCTGAGGAATCTGTCCCGGAATCTTTCTCT |
| | GCAGAATGAAATTGCCAAAGAAACTCTCCCTGATTTGGTTTCCATCATTCCTGACACAGT |
| | CCCGAGTACTGACCTTCTCATTGAAACTACAGCCTCTGCCTGTTACACATTGAACAACAT |
| | AATCCAAAACAGTTACCAGAATGCACGCGACCTTCTAAACACCGGGGGCATCCAGAAAA |
| | TTATGGCCATTAGTGCAGGCGATGCCTATGCCTCCAACAAAGCAAGTAAAGCTGCTTCC |
| | GTCCTTCTGTATTCTCTGTGGGCACACACGGAACTGCATCATGCCTACAAGAAGGCTCA |
| | GTTTAAGAAGACAGATTTTGTCAACAGCCGGACTGCCAAAGCCTACCACTCCCTTAAAGA |
| | CTGATCAACCTCTGGATTACAAAATTTGTGAAAGATTGACTGGTATTCTTAACTATGTTGC |
| | TCCTTTTACGCTATGTGGATACGCTGCTTTAATGCCTTTGTATCATGCTATTGCTTCCCGT |
| | ATGGCTTTCATTTTCTCCTCCTTGTATAAATCCTGGTTGCTGTCTCTTTATGAGGAGTTGT |
| | GGCCCGTTGTCAGGCAACGTGGCGTGGTGTGCACTGTGTTTGCTGACGCAACCCCCAC |
| | TGGTTGGGGCATTGCCACCACCTGTCAGCTCCTTTCCGGGACTTTCGCTTTCCCCCTCC |
| | CTATTGCCACGGCGGAACTCATCGCCGCCTGCCTTGCCCGCTGCTGGACAGGGGCTCG |
| | GCTGTTGGGCACTGACAATTCCGTGGTGTTGTCGGGGAAATCATCGTCCTTTCCTTGGC |
| | TGCTCGCCTGTGTTGCCACCTGGATTCTGCGCGGGACGTCCTTCTGCTACGTCCCTTCG |
| | GCCCTCAATCCAGCGGACCTTCCTTCCCGCGGCCTGCTGCCGGCTCTGCGGCCTCTTC |
| | CGCGTCTTCGCCTTCGCCCTCAGACGAGTCGGATCTCCCTTTGGGCCGCCTCCCCGCA |
| | CTGCCCGGGTGGCATCCCTGTGACCCCTCCCCAGTGCCTCTCCTGGCCCTGGAAGTTG |
| | CCACTCCAGTGCCCACCAGCCTTGTCCTAATAAAATTAAGTTGCATCATTTTGTCTGACTA |
| | GGTGTCCTTCTATAATATTATGGGGTGGAGGGGGGTGGTATGGAGCAAGGGGCCCAAG |
| | TTGGGAAGAAACCTGTAGGGCCTGCtacgtagataagtagcatgggggttaatcattaactacaaggaacccct |
| | agtgatggagttggccactccctctctgcgcgctcgctcgctcactgaggccgggcgaccaaaggtcgcccgacgcccgggctt |
| | tgcccgggggcctcagtgagcgagcgagcgcgc |
| 13 | gcgcgctcgctcgctcactgaggccgcccgggcaaagcccgggcgtcgggcgacctttggtcgcccggcctcagtgagcgagc |
| | gagcgcgcagagagggagtggccaactccatcactaggggttccttgtagttaatgattaacccgccatgctacttatctacgt |
| | aCTCAGTCCATTAGGAGCCAGTAGCCTGGAAGATGTCTTTACCCCCAGCATCAGTTCAAGT |
| | GGAGCAGCACATAACTCTTGCCCTCTGCCTTCCAAGATTCTGGTGCTGAGACTTATGGA |
| | GTGTCTTGGAGGTTGCCTTCTGCCCCCCAACCCTGCTCCCAGCTGGCCCTCCCAGGCCT |
| | GGGTTGCTGGCCTCTGCTTTATCAGGATTCTCAAGAGGGACAGCTGGTTTATGTTGCAT |
| | GACTGTTCCCTGCATATCTGCTCTGGTTTTAAATAGCTTATCTGAGCAGCTGGAGGACCA |
| | CATGGGCTTATATGGCGTGGGGTACATGTTCCTGTAGCCTTGTCCCTGGCACCTGCCAA |
| | AATAGCAGCCAACACCCCCCACCCCCACCGCCATCCCCCTGCCCCACCCGTCCCCTGT |
| | CGCACATTCCTCCCTCCGCAGGGCTGGCTCACCAGGCCCCAGCCCACATGCCTGCTTA |
| | AAGCCCTCTCCATCCTCTGCCTCACCCAGTCCCCGCTGAGACTGAGCAGACGCCTCCAG |
| | GATCTGTCGGCAGGCCACCATGGCAGCCCCCGGCGCCCCAGCTGAGTACGGCTACATC |
| | CGGACCGTCCTGGGCCAGCAGATCCTGGGACAACTGGACAGCTCCAGCCTGGCGCTGC |
| | CCTCCGAGGCCAAGCTGAAGCTGGGGGGGAGCAGCGGCCGCGGCGGCCAGACAGTCA |
| | AGAGCCTGCGGATCCAGGAGCAGGTGCAGCAGACCCTCGCCCGGAAGGGCCGCAGCT |
| | CCGTGGGCAACGGAAATCTTCACCGAACCAGCAGTGTTCCTGAGTATGTCTACAACCTA |
| | CACTTGGTTGAAAATGATTTTGTTGGAGGCCGTTCCCCTGTTCCTAAAACCTATGACATG |
| | CTAAAGGCTGGCACAACTGCCACTTATGAAGGTCGCTGGGGAAGAGGAACAGCACAGTA |
| | CAGCTCCCAGAAGTCCGTGGAAGAAAGGTCCTTGAGGCATCCTCTGAGGAGACTGGAG |
| | ATTTCTCCTGACAGCAGCCCGGAGAGGGCTCACTACACGCACAGCGATTACCAGTACAG |
| | CCAGAGAAGCCAGGCTGGGCACACCCTGCACCACCAAGAAAGCAGGCGGGCCGCCCT |
| | CCTAGTGCCACCGAGATATGCTCGTTCCGAGATCGTGGGGGTCAGCCGTGCTGGCACC |
| | ACAAGCAGGCAGCGCCACTTTGACACATACCACAGACAGTACCAGCATGGCTCTGTTAG |
| | CGACACCGTTTTTGACAGCATCCCTGCCAACCCGGCCCTGCTCACGTACCCCAGGCCAG |
| | GGACCAGCCGCAGCATGGGCAACCTCTTGGAGAAGGAGAACTACCTGACGGCAGGGCT |
| | CACTGTCGGGCAGGTCAGGCCGCTGGTGCCCCTGCAGCCCGTCACTCAGAACAGGGCT |
| | TCCAGGTCCTCCTGGCATCAGAGCTCCTTCCACAGCACCCGCACGCTGAGGGAAGCTG |
| | GGCCCAGTGTCGCCGTGGATTCCAGCGGGAGGAGAGCGCACTTGACTGTCGGCCAGG |
| | CGGCCGCAGGGGGAAGTGGGAATCTGCTCACTGAGAGAAGCACTTTCACTGACTCCCA |
| | GCTGGGGAATGCAGACATGGAGATGACTCTGGAGCGAGCAGTGAGTATGCTCGAGGCA |
| | GACCACATGCTGCCATCCAGGATTTCTGCTGCAGCTACTTTCATACAGCACGAGTGCTTC |
| | CAGAAATCTGAAGCTCGGAAGAGGGTTAACCAGCTTCGTGGCATCCTCAAGCTTCTGCA |
| | GCTCCTAAAAGTTCAGAATGAAGACGTTCAGCGAGCTGTGTGTGGGGCCTTGAGAAACT |
| | TAGTATTTGAAGACAATGACAACAAATTGGAGGTGGCTGAACTAAATGGGGTACCTCGG |
| | CTGCTCCAGGTGCTGAAGCAAACCAGAGACTTGGAGACTAAAAAACAAATAACAGGTTT |
| | GCTGTGGAATTTGTCATCTAATGACAAACTCAAGAATCTCATGATAACAGAAGCATTGCTT |
| | ACGCTGACGGAGAATATCATCATCCCCTTTTCTGGGTGGCCTGAAGGAGACTACCCAAA |
| | AGCAAATGGTTTGCTCGATTTTGACATATTCTACAACGTCACTGGATGCCTAAGAAACAT |
| | GAGTTCTGCTGGCGCTGATGGGAGAAAAGCGATGAGAAGATGTGACGGACTCATTGACT |
| | CACTGGTCCATTATGTCAGAGGAACCATTGCAGATTACCAGCCAGATGACAAGGCCACG |
| | GAGAATTGTGTGTGCATTCTTCATAACCTCTCCTACCAGCTGGAGGCAGAGCTCCCAGA |
| | GAAATATTCCCAGAATATCTATATTCAAAACCGGAATATCCAGACTGACAACAACAAAAGT |
| | ATTGGATGTTTTGGCAGTCGAAGCAGGAAAGTAAAAGAGCAATACCAGGACGTGCCGAT |
| | GCCGGAGGAAAAGAGCAACCCCAAGGGCGTGGAGTGGCTGTGGCATTCCATTGTTATA |
| | AGGATGTATCTGTCCTTGATCGCCAAAAGTGTCCGCAACTACACACAAGAAGCATCCTTA |
| | GGAGCTCTGCAGAACCTCACGGCCGGAAGTGGACCAATGCCGACATCAGTGGCTCAGA |
| | CAGTTGTCCAGAAGGAAAGTGGCCTGCAGCACACCCGAAAGATGCTGCATGTTGGTGAC |
| | CCAAGTGTGAAAAAGACAGCCATCTCGCTGCTGAGGAATCTGTCCCGGAATCTTTCTCT |
| | GCAGAATGAAATTGCCAAAGAAACTCTCCCTGATTTGGTTTCCATCATTCCTGACACAGT |
| | CCCGAGTACTGACCTTCTCATTGAAACTACAGCCTCTGCCTGTTACACATTGAACAACAT |
| | AATCCAAAACAGTTACCAGAATGCACGCGACCTTCTAAACACCGGGGGCATCCAGAAAA |
| | TTATGGCCATTAGTGCAGGCGATGCCTATGCCTCCAACAAAGCAAGTAAAGCTGCTTCC |
| | GTCCTTCTGTATTCTCTGTGGGCACACACGGAACTGCATCATGCCTACAAGAAGGCTCA |
| | GTTTAAGAAGACAGATTTTGTCAACAGCCGGACTGCCAAAGCCTACCACTCCCTTAAAGA |
| | CTGATCAACCTCTGGATTACAAAATTTGTGAAAGATTGACTGGTATTCTTAACTATGTTGC |
| | TCCTTTTACGCTATGTGGATACGCTGCTTTAATGCCTTTGTATCATGCTATTGCTTCCCGT |
| | ATGGCTTTCATTTTCTCCTCCTTGTATAAATCCTGGTTGCTGTCTCTTTATGAGGAGTTGT |
| | GGCCCGTTGTCAGGCAACGTGGCGTGGTGTGCACTGTGTTTGCTGACGCAACCCCCAC |
| | TGGTTGGGGCATTGCCACCACCTGTCAGCTCCTTTCCGGGACTTTCGCTTTCCCCCTCC |
| | CTATTGCCACGGCGGAACTCATCGCCGCCTGCCTTGCCCGCTGCTGGACAGGGGCTCG |
| | GCTGTTGGGCACTGACAATTCCGTGGTGTTGTCGGGGAAATCATCGTCCTTTCCTTGGC |
| | TGCTCGCCTGTGTTGCCACCTGGATTCTGCGCGGGACGTCCTTCTGCTACGTCCCTTCG |
| | GCCCTCAATCCAGCGGACCTTCCTTCCCGCGGCCTGCTGCCGGCTCTGCGGCCTCTTC |
| | CGCGTCTTCGCCTTCGCCCTCAGACGAGTCGGATCTCCCTTTGGGCCGCCTCCCCGCA |
| | CTGCCCGGGTGGCATCCCTGTGACCCCTCCCCAGTGCCTCTCCTGGCCCTGGAAGTTG |
| | CCACTCCAGTGCCCACCAGCCTTGTCCTAATAAAATTAAGTTGCATCATTTTGTCTGACTA |
| | GGTGTCCTTCTATAATATTATGGGGTGGAGGGGGGTGGTATGGAGCAAGGGGCCCAAG |
| | TTGGGAAGAAACCTGTAGGGCCTGCtacgtagataagtagcatgggggttaatcattaactacaaggaacccct |
| | agtgatggagttggccactccctctctgcgcgctcgctcgctcactgaggccgggcgaccaaaggtcgcccgacgcccgggctt |
| | tgcccgggcggcctcagtgagcgagcgagcgcgc |
| 14 | gcgcgctcgctcgctcactgaggccgccgggcaaagcccgggcgtcgggcgacctttggtcgcccggcctcagtgagcgagc |
| | gagcgcgcagagagggagtggccaactccatcactaggggttccttgtagttaatgattaacccgccatgctacttatctacgt |
| | aACCCTTCAGATTAAAAATAACTGAGGTAAGGGCCTGGGTAGGGGAGGTGGTGTGAGACG |
| | CTCCTGTCTCTCCTCTATCTGCCCATCGGCCCTTTGGGGAGGAGGAATGTGCCCAAGGA |
| | CTAAAAAAAGGCCATGGAGCCAGAGGGGGGAGGGCAACAGACCTTTCATGGGCAAACC |
| | TTGGGGCCCTGCTGTCTAGCATGCCCCACTACGGGTCTAGGCTGCCCATGTAAGGAGG |
| | CAAGGCCTGGGGACACCCGAGATGCCTGGTTATAATTAACCCAGACATGTGGCTGCCCC |
| | CCCCCCCCCAACACCTGCTGCCTCTAAAAATAACCCTGTCCCTGGTGGATCCCCTGCAT |
| | GCGAAGATCTTCGAACAAGGCTGTGGGGGACTGAGGGCAGGCTGTAACAGGCTTGGGG |
| | GCCAGGGCTTATACGTGCCTGGGACTCCCAAAGTATTACTGTTCCATGTTCCCGGCGAA |
| | GGGCCAGCTGTCCCCCGCCAGCTAGACTCAGCACTTAGTTTAGGAACCAGTGAGCAAGT |
| | CAGCCCTTGGGGCAGCCCATACAAGGCCATGGGGCTGGGCAAGCTGCACGCCTGGGT |
| | CCGGGGTGGGCACGGTGCCCGGGCAACGAGCTGAAAGCTCATCTGCTCTCAGGGGCC |
| | CCTCCCTGGGGACAGCCCCTCCTGGCTAGTCACACCCTGTAGGCTCCTCTATATAACCC |
| | AGGGGCACAGGGGCTGCCCTCATTCTACCACCACCTCCACAGCACAGACAGACACTCA |
| | GGAGCCAGCCAGGCCACCATGGCAGCCCCCGGCGCCCCAGCTGAGTACGGCTACATC |
| | CGGACCGTCCTGGGCCAGCAGATCCTGGGACAACTGGACAGCTCCAGCCTGGCGCTGC |
| | CCTCCGAGGCCAAGCTGAAGCTGGGGGGGAGCAGCGGCCGCGGCGGCCAGACAGTCA |
| | AGAGCCTGCGGATCCAGGAGCAGGTGCAGCAGACCCTCGCCCGGAAGGGCCGCAGCT |
| | CCGTGGGCAACGGAAATCTTCACCGAACCAGCAGTGTTCCTGAGTATGTCTACAACCTA |
| | CACTTGGTTGAAAATGATTTTGTTGGAGGCCGTTCCCCTGTTCCTAAAACCTATGACATG |
| | CTAAAGGCTGGCACAACTGCCACTTATGAAGGTCGCTGGGGAAGAGGAACAGCACAGTA |
| | CAGCTCCCAGAAGTCCGTGGAAGAAAGGTCCTTGAGGCATCCTCTGAGGAGACTGGAG |
| | ATTTCTCCTGACAGCAGCCCGGAGAGGGCTCACTACACGCACAGCGATTACCAGTACAG |
| | CCAGAGAAGCCAGGCTGGGCACACCCTGCACCACCAAGAAAGCAGGCGGGCCGCCCT |
| | CCTAGTGCCACCGAGATATGCTCGTTCCGAGATCGTGGGGGTCAGCCGTGCTGGCACC |
| | ACAAGCAGGCAGCGCCACTTTGACACATACCACAGACAGTACCAGCATGGCTCTGTTAG |
| | CGACACCGTTTTTGACAGCATCCCTGCCAACCCGGCCCTGCTCACGTACCCCAGGCCAG |
| | GGACCAGCCGCAGCATGGGCAACCTCTTGGAGAAGGAGAACTACCTGACGGCAGGGCT |
| | CACTGTCGGGCAGGTCAGGCCGCTGGTGCCCCTGCAGCCCGTCACTCAGAACAGGGCT |
| | TCCAGGTCCTCCTGGCATCAGAGCTCCTTCCACAGCACCCGCACGCTGAGGGAAGCTG |
| | GGCCCAGTGTCGCCGTGGATTCCAGCGGGAGGAGAGCGCACTTGACTGTCGGCCAGG |
| | CGGCCGCAGGGGGAAGTGGGAATCTGCTCACTGAGAGAAGCACTTTCACTGACTCCCA |
| | GCTGGGGAATGCAGACATGGAGATGACTCTGGAGCGAGCAGTGAGTATGCTCGAGGCA |
| | GACCACATGCTGCCATCCAGGATTTCTGCTGCAGCTACTTTCATACAGCACGAGTGCTTC |
| | CAGAAATCTGAAGCTCGGAAGAGGGTTAACCAGCTTCGTGGCATCCTCAAGCTTCTGCA |
| | GCTCCTAAAAGTTCAGAATGAAGACGTTCAGCGAGCTGTGTGTGGGGCCTTGAGAAACT |
| | TAGTATTTGAAGACAATGACAACAAATTGGAGGTGGCTGAACTAAATGGGGTACCTCGG |
| | CTGCTCCAGGTGCTGAAGCAAACCAGAGACTTGGAGACTAAAAAACAAATAACAGACCA |
| | TACAGTCAATTTAAGAAGTAGGAATGGCTGGCCGGGCGCGGTGGCTCACGCCTGTAATC |
| | CCAGCACTTTGGGAGGCCAAGGGGGGGGATCACGAGGTCAGGAGTTCGAGACCAGC |
| | CTGACCAACATGGTTTGCTGTGGAATTTGTCATCTAATGACAAACTCAAGAATCTCATGAT |
| | AACAGAAGCATTGCTTACGCTGACGGAGAATATCATCATCCCCTTTTCTGGGTGGCCTGA |
| | AGGAGACTACCCAAAAGCAAATGGTTTGCTCGATTTTGACATATTCTACAACGTCACTGG |
| | ATGCCTAAGAAACATGAGTTCTGCTGGCGCTGATGGGAGAAAAGCGATGAGAAGATGTG |
| | ACGGACTCATTGACTCACTGGTCCATTATGTCAGAGGAACCATTGCAGATTACCAGCCAG |
| | ATGACAAGGCCACGGAGAATTGTGTGTGCATTCTTCATAACCTCTCCTACCAGCTGGAG |
| | GCAGAGCTCCCAGAGAAATATTCCCAGAATATCTATATTCAAAACCGGAATATCCAGACT |
| | GACAACAACAAAAGTATTGGATGTTTTGGCAGTCGAAGCAGGAAAGTAAAAGAGCAATAC |
| | CAGGACGTGCCGATGCCGGAGGAAAAGAGCAACCCCAAGGGCGTGGAGTGGCTGTGG |
| | CATTCCATTGTTATAAGGATGTATCTGTCCTTGATCGCCAAAAGTGTCCGCAACTACACA |
| | CAAGAAGCATCCTTAGGAGCTCTGCAGAACCTCACGGCCGGAAGTGGACCAATGCCGA |
| | CATCAGTGGCTCAGACAGTTGTCCAGAAGGAAAGTGGCCTGCAGCACACCCGAAAGATG |
| | CTGCATGTTGGTGACCCAAGTGTGAAAAAGACAGCCATCTCGCTGCTGAGGAATCTGTC |
| | CCGGAATCTTTCTCTGCAGAATGAAATTGCCAAAGAAACTCTCCCTGATTTGGTTTCCAT |
| | CATTCCTGACACAGTCCCGAGTACTGACCTTCTCATTGAAACTACAGCCTCTGCCTGTTA |
| | CACATTGAACAACATAATCCAAAACAGTTACCAGAATGCACGCGACCTTCTAAACACCGG |
| | GGGCATCCAGAAAATTATGGCCATTAGTGCAGGCGATGCCTATGCCTCCAACAAAGCAA |
| | GTAAAGCTGCTTCCGTCCTTCTGTATTCTCTGTGGGCACACACGGAACTGCATCATGCCT |
| | ACAAGAAGGCTCAGTTTAAGAAGACAGATTTTGTCAACAGCCGGACTGCCAAAGCCTAC |
| | CACTCCCTTAAAGACTGATCAACCTCTGGATTACAAAATTTGTGAAAGATTGACTGGTATT |
| | CTTAACTATGTTGCTCCTTTTACGCTATGTGGATACGCTGCTTTAATGCCTTTGTATCATG |
| | CTATTGCTTCCCGTATGGCTTTCATTTTCTCCTCCTTGTATAAATCCTGGTTGCTGTCTCT |
| | TTATGAGGAGTTGTGGCCCGTTGTCAGGCAACGTGGCGTGGTGTGCACTGTGTTTGCTG |
| | ACGCAACCCCCACTGGTTGGGGCATTGCCACCACCTGTCAGCTCCTTTCCGGGACTTTC |
| | GCTTTCCCCCTCCCTATTGCCACGGCGGAACTCATCGCCGCCTGCCTTGCCCGCTGCTG |
| | GACAGGGGCTCGGCTGTTGGGCACTGACAATTCCGTGGTGTTGTCGGGGAAATCATCG |
| | TCCTTTCCTTGGCTGCTCGCCTGTGTTGCCACCTGGATTCTGCGCGGGACGTCCTTCTG |
| | CTACGTCCCTTCGGCCCTCAATCCAGCGGACCTTCCTTCCCGCGGCCTGCTGCCGGCT |
| | CTGCGGCCTCTTCCGCGTCTTCGCCTTCGCCCTCAGACGAGTCGGATCTCCCTTTGGGC |
| | CGCCTCCCCGCACTGCCCGGGTGGCATCCCTGTGACCCCTCCCCAGTGCCTCTCCTGG |
| | CCCTGGAAGTTGCCACTCCAGTGCCCACCAGCCTTGTCCTAATAAAATTAAGTTGCATCA |
| | TTTTGTCTGACTAGGTGTCCTTCTATAATATTATGGGGTGGAGGGGGGTGGTATGGAGC |
| | AAGGGGCCCAAGTTGGGAAGAAACCTGTAGGGCCTGCtacgtagataagtagcatggcgggttaatcat |
| | taactacaaggaacccctagtgatggagttggccactccctctctgcgcgctcgctcgctcactgaggccgggcgaccaaagg |
| | tcgcccgacgcccgggctttgcccgggcggcctcagtgagcgagcgagcgcgc |
| 15 | CTCAGTCCATTAGGAGCCAGTAGCCTGGAAGATGTCTTTACCCCCAGCATCAGTTCAAGT |
| | GGAGCAGCACATAACTCTTGCCCTCTGCCTTCCAAGATTCTGGTGCTGAGACTTATGGA |
| | GTGTCTTGGAGGTTGCCTTCTGCCCCCCAACCCTGCTCCCAGCTGGCCCTCCCAGGCCT |
| | GGGTTGCTGGCCTCTGCTTTATCAGGATTCTCAAGAGGGACAGCTGGTTTATGTTGCAT |
| | GACTGTTCCCTGCATATCTGCTCTGGTTTTAAATAGCTTATCTGAGCAGCTGGAGGACCA |
| | CATGGGCTTATATGGCGTGGGGTACATGTTCCTGTAGCCTTGTCCCTGGCACCTGCCAA |
| | AATAGCAGCCAACACCCCCCACCCCCACCGCCATCCCCCTGCCCCACCCGTCCCCTGT |
| | CGCACATTCCTCCCTCCGCAGGGCTGGCTCACCAGGCCCCAGCCCACATGCCTGCTTA |
| | AAGCCCTCTCCATCCTCTGCCTCACCCAGTCCCCGCTGAGACTGAGCAGACGCCTCCAG |
| | GATCTGTCGGCAGGCCACCATGGCAGCCCCCGGCGCCCCAGCTGAGTACGGCTACATC |
| | CGGACCGTCCTGGGCCAGCAGATCCTGGGACAACTGGACAGCTCCAGCCTGGCGCTGC |
| | CCTCCGAGGCCAAGCTGAAGCTGGCGGGGAGCAGCGGCCGCGGCGGCCAGACAGTCA |
| | AGAGCCTGCGGATCCAGGAGCAGGTGCAGCAGACCCTCGCCCGGAAGGGCCGCAGCT |
| | CCGTGGGCAACGGAAATCTTCACCGAACCAGCAGTGTTCCTGAGTATGTCTACAACCTA |
| | CACTTGGTTGAAAATGATTTTGTTGGAGGCCGTTCCCCTGTTCCTAAAACCTATGACATG |
| | CTAAAGGCTGGCACAACTGCCACTTATGAAGGTCGCTGGGGAAGAGGAACAGCACAGTA |
| | CAGCTCCCAGAAGTCCGTGGAAGAAAGGTCCTTGAGGCATCCTCTGAGGAGACTGGAG |
| | ATTTCTCCTGACAGCAGCCCGGAGAGGGCTCACTACACGCACAGCGATTACCAGTACAG |
| | CCAGAGAAGCCAGGCTGGGCACACCCTGCACCACCAAGAAAGCAGGCGGGCCGCCCT |
| | CCTAGTGCCACCGAGATATGCTCGTTCCGAGATCGTGGGGGTCAGCCGTGCTGGCACC |
| | ACAAGCAGGCAGCGCCACTTTGACACATACCACAGACAGTACCAGCATGGCTCTGTTAG |
| | CGACACCGTTTTTGACAGCATCCCTGCCAACCCGGCCCTGCTCACGTACCCCAGGCCAG |
| | GGACCAGCCGCAGCATGGGCAACCTCTTGGAGAAGGAGAACTACCTGACGGCAGGGCT |
| | CACTGTCGGGCAGGTCAGGCCGCTGGTGCCCCTGCAGCCCGTCACTCAGAACAGGGCT |
| | TCCAGGTCCTCCTGGCATCAGAGCTCCTTCCACAGCACCCGCACGCTGAGGGAAGCTG |
| | GGCCCAGTGTCGCCGTGGATTCCAGCGGGAGGAGAGCGCACTTGACTGTCGGCCAGG |
| | CGGCCGCAGGGGGAAGTGGGAATCTGCTCACTGAGAGAAGCACTTTCACTGACTCCCA |
| | GCTGGGGAATGCAGACATGGAGATGACTCTGGAGCGAGCAGTGAGTATGCTCGAGGCA |
| | GACCACATGCTGCCATCCAGGATTTCTGCTGCAGCTACTTTCATACAGCACGAGTGCTTC |
| | CAGAAATCTGAAGCTCGGAAGAGGGTTAACCAGCTTCGTGGCATCCTCAAGCTTCTGCA |
| | GCTCCTAAAAGTTCAGAATGAAGACGTTCAGCGAGCTGTGTGTGGGGCCTTGAGAAACT |
| | TAGTATTTGAAGACAATGACAACAAATTGGAGGTGGCTGAACTAAATGGGGTACCTCGG |
| | CTGCTCCAGGTGCTGAAGCAAACCAGAGACTTGGAGACTAAAAAACAAATAACAGACCA |
| | TACAGTCAATTTAAGAAGTAGGAATGGCTGGCCGGGCGCGGTGGCTCACGCCTGTAATC |
| | CCAGCACTTTGGGAGGCCAAGGCGGGCGGATCACGAGGTCAGGAGTTCGAGACCAGC |
| | CTGACCAACATGGTTTGCTGTGGAATTTGTCATCTAATGACAAACTCAAGAATCTCATGAT |
| | AACAGAAGCATTGCTTACGCTGACGGAGAATATCATCATCCCCTTTTCTGGGTGGCCTGA |
| | AGGAGACTACCCAAAAGCAAATGGTTTGCTCGATTTTGACATATTCTACAACGTCACTGG |
| | ATGCCTAAGAAACATGAGTTCTGCTGGCGCTGATGGGAGAAAAGCGATGAGAAGATGTG |
| | ACGGACTCATTGACTCACTGGTCCATTATGTCAGAGGAACCATTGCAGATTACCAGCCAG |
| | ATGACAAGGCCACGGAGAATTGTGTGTGCATTCTTCATAACCTCTCCTACCAGCTGGAG |
| | GCAGAGCTCCCAGAGAAATATTCCCAGAATATCTATATTCAAAACCGGAATATCCAGACT |
| | GACAACAACAAAAGTATTGGATGTTTTGGCAGTCGAAGCAGGAAAGTAAAAGAGCAATAC |
| | CAGGACGTGCCGATGCCGGAGGAAAAGAGCAACCCCAAGGGCGTGGAGTGGCTGTGG |
| | CATTCCATTGTTATAAGGATGTATCTGTCCTTGATCGCCAAAAGTGTCCGCAACTACACA |
| | CAAGAAGCATCCTTAGGAGCTCTGCAGAACCTCACGGCCGGAAGTGGACCAATGCCGA |
| | CATCAGTGGCTCAGACAGTTGTCCAGAAGGAAAGTGGCCTGCAGCACACCCGAAAGATG |
| | CTGCATGTTGGTGACCCAAGTGTGAAAAAGACAGCCATCTCGCTGCTGAGGAATCTGTC |
| | CCGGAATCTTTCTCTGCAGAATGAAATTGCCAAAGAAACTCTCCCTGATTTGGTTTCCAT |
| | CATTCCTGACACAGTCCCGAGTACTGACCTTCTCATTGAAACTACAGCCTCTGCCTGTTA |
| | CACATTGAACAACATAATCCAAAACAGTTACCAGAATGCACGCGACCTTCTAAACACCGG |
| | GGGCATCCAGAAAATTATGGCCATTAGTGCAGGCGATGCCTATGCCTCCAACAAAGCAA |
| | GTAAAGCTGCTTCCGTCCTTCTGTATTCTCTGTGGGCACACACGGAACTGCATCATGCCT |
| | ACAAGAAGGCTCAGTTTAAGAAGACAGATTTTGTCAACAGCCGGACTGCCAAAGCCTAC |
| | CACTCCCTTAAAGACTGATCAACCTCTGGATTACAAAATTTGTGAAAGATTGACTGGTATT |
| | CTTAACTATGTTGCTCCTTTTACGCTATGTGGATACGCTGCTTTAATGCCTTTGTATCATG |
| | CTATTGCTTCCCGTATGGCTTTCATTTTCTCCTCCTTGTATAAATCCTGGTTGCTGTCTCT |
| | TTATGAGGAGTTGTGGCCCGTTGTCAGGCAACGTGGCGTGGTGTGCACTGTGTTTGCTG |
| | ACGCAACCCCCACTGGTTGGGGCATTGCCACCACCTGTCAGCTCCTTTCCGGGACTTTC |
| | GCTTTCCCCCTCCCTATTGCCACGGCGGAACTCATCGCCGCCTGCCTTGCCCGCTGCTG |
| | GACAGGGGCTCGGCTGTTGGGCACTGACAATTCCGTGGTGTTGTCGGGGAAATCATCG |
| | TCCTTTCCTTGGCTGCTCGCCTGTGTTGCCACCTGGATTCTGCGCGGGACGTCCTTCTG |
| | CTACGTCCCTTCGGCCCTCAATCCAGCGGACCTTCCTTCCCGCGGCCTGCTGCCGGCT |
| | CTGCGGCCTCTTCCGCGTCTTCGCCTTCGCCCTCAGACGAGTCGGATCTCCCTTTGGGC |
| | CGCCTCCCCGCACTGCCCGGGTGGCATCCCTGTGACCCCTCCCCAGTGCCTCTCCTGG |
| | CCCTGGAAGTTGCCACTCCAGTGCCCACCAGCCTTGTCCTAATAAAATTAAGTTGCATCA |
| | TTTTGTCTGACTAGGTGTCCTTCTATAATATTATGGGGTGGAGGGGGGTGGTATGGAGC |
| | AAGGGGCCCAAGTTGGGAAGAAACCTGTAGGGCCTGC |
| 89 | gcgcgctcgctcgctcactgaggccgcccgggcaaagcccgggcgtcgggcgacctttggtcgcccggcctcagtgagcgagc |
| | gagcgcgcagagagggagtggccaactccatcactaggggttccttgtagttaatgattaacccgccatgctacttatctacg |
| | tactctggagacgcgtACCCTTCAGATTAAAAATAACTGAGGTAAGGGCCTGGGTAGGGGAGGTGGT |
| | GTGAGACGCTCCTGTCTCTCCTCTATCTGCCCATCGGCCCTTTGGGGAGGAGGAATGTG |
| | CCCAAGGACTAAAAAAAGGCCATGGAGCCAGAGGGGCGAGGGCAACAGACCTTTCATG |
| | GGCAAACCTTGGGGCCCTGCTGTCTAGCATGCCCCACTACGGGTCTAGGCTGCCCATG |
| | TAAGGAGGCAAGGCCTGGGGACACCCGAGATGCCTGGTTATAATTAACCCAGACATGTG |
| | GCTGCCCCCCCCCCCCCAACACCTGCTGCCTCTAAAAATAACCCTGTCCCTGGTGGATC |
| | CCCTGCATGCGAAGATCTTCGAACAAGGCTGTGGGGGACTGAGGGCAGGCTGTAACAG |
| | GCTTGGGGGCCAGGGCTTATACGTGCCTGGGACTCCCAAAGTATTACTGTTCCATGTTC |
| | CCGGCGAAGGGCCAGCTGTCCCCCGCCAGCTAGACTCAGCACTTAGTTTAGGAACCAG |
| | TGAGCAAGTCAGCCCTTGGGGCAGCCCATACAAGGCCATGGGGCTGGGCAAGCTGCAC |
| | GCCTGGGTCCGGGGGGGCACGGTGCCCGGGCAACGAGCTGAAAGCTCATCTGCTCTC |
| | AGGGGCCCCTCCCTGGGGACAGCCCCTCCTGGCTAGTCACACCCTGTAGGCTCCTCTA |
| | TATAACCCAGGGGCACAGGGGCTGCCCTCATTCTACCACCACCTCCACAGCACAGACAG |
| | ACACTCAGGAGCCAGCCAGggtaagtttagtctttttgtcttttatttcaggtcccggatccggtggtggtgcaaatcaaag |
| | aactgctcctcagtggatgttgcctttacttctaggcctgtacggaagtgttacttctgctctaaaagctgcggaattgtacc |
| | cgcGCCACCATGGCAGCCCCCGGCGCCCCAGCTGAGTACGGCTACATCCGGACCGTCCTGGGCC |
| | AGCAGATCCTGGGACAACTGGACAGCTCCAGCCTGGCGCTGCCCTCCGAGGCCAAGCT |
| | GAAGCTGGCGGGGAGCAGCGGCCGCGGCGGCCAGACAGTCAAGAGCCTGCGGATCCA |
| | GGAGCAGGTGCAGCAGACCCTCGCCCGGAAGGGCCGCAGCTCCGTGGGCAACGGAAA |
| | TCTTCACCGAACCAGCAGTGTTCCTGAGTATGTCTACAACCTACACTTGGTTGAAAATGA |
| | TTTTGTTGGAGGCCGTTCCCCTGTTCCTAAAACCTATGACATGCTAAAGGCTGGCACAAC |
| | TGCCACTTATGAAGGTCGCTGGGGAAGAGGAACAGCACAGTACAGCTCCCAGAAGTCC |
| | GTGGAAGAAAGGTCCTTGAGGCATCCTCTGAGGAGACTGGAGATTTCTCCTGACAGCAG |
| | CCCGGAGAGGGCTCACTACACGCACAGCGATTACCAGTACAGCCAGAGAAGCCAGGCT |
| | GGGCACACCCTGCACCACCAAGAAAGCAGGGGGGCCGCCCTCCTAGTGCCACCGAGAT |
| | ATGCTCGTTCCGAGATCGTGGGGGTCAGCCGTGCTGGCACCACAAGCAGGCAGCGCCA |
| | CTTTGACACATACCACAGACAGTACCAGCATGGCTCTGTTAGCGACACCGTTTTTGACAG |
| | CATCCCTGCCAACCCGGCCCTGCTCACGTACCCCAGGCCAGGGACCAGCCGCAGCATG |
| | GGCAACCTCTTGGAGAAGGAGAACTACCTGACGGCAGGGCTCACTGTCGGGCAGGTCA |
| | GGCCGCTGGTGCCCCTGCAGCCCGTCACTCAGAACAGGGCTTCCAGGTCCTCCTGGCA |
| | TCAGAGCTCCTTCCACAGCACCCGCACGCTGAGGGAAGCTGGGCCCAGTGTCGCCGTG |
| | GATTCCAGCGGGAGGAGAGCGCACTTGACTGTCGGCCAGGCGGCCGCAGGGGGAAGT |
| | GGGAATCTGCTCACTGAGAGAAGCACTTTCACTGACTCCCAGCTGGGGAATGCAGACAT |
| | GGAGATGACTCTGGAGCGAGCAGTGAGTATGCTCGAGGCAGACCACATGCTGCCATCC |
| | AGGATTTCTGCTGCAGCTACTTTCATACAGCACGAGTGCTTCCAGAAATCTGAAGCTCGG |
| | AAGAGGGTTAACCAGCTTCGTGGCATCCTCAAGCTTCTGCAGCTCCTAAAAGTTCAGAAT |
| | GAAGACGTTCAGCGAGCTGTGTGTGGGGCCTTGAGAAACTTAGTATTTGAAGACAATGA |
| | CAACAAATTGGAGGTGGCTGAACTAAATGGGGTACCTCGGCTGCTCCAGGTGCTGAAGC |
| | AAACCAGAGACTTGGAGACTAAAAAACAAATAACAGGTTTGCTGTGGAATTTGTCATCTA |
| | ATGACAAACTCAAGAATCTCATGATAACAGAAGCATTGCTTACGCTGACGGAGAATATCA |
| | TCATCCCCTTTTCTGGGTGGCCTGAAGGAGACTACCCAAAAGCAAATGGTTTGCTCGATT |
| | TTGACATATTCTACAACGTCACTGGATGCCTAAGAAACATGAGTTCTGCTGGCGCTGATG |
| | GGAGAAAAGCGATGAGAAGATGTGACGGACTCATTGACTCACTGGTCCATTATGTCAGA |
| | GGAACCATTGCAGATTACCAGCCAGATGACAAGGCCACGGAGAATTGTGTGTGCATTCT |
| | TCATAACCTCTCCTACCAGCTGGAGGCAGAGCTCCCAGAGAAATATTCCCAGAATATCTA |
| | TATTCAAAACCGGAATATCCAGACTGACAACAACAAAAGTATTGGATGTTTTGGCAGTCG |
| | AAGCAGGAAAGTAAAAGAGCAATACCAGGACGTGCCGATGCCGGAGGAAAAGAGCAAC |
| | CCCAAGGGCGTGGAGTGGCTGTGGCATTCCATTGTTATAAGGATGTATCTGTCCTTGAT |
| | CGCCAAAAGTGTCCGCAACTACACACAAGAAGCATCCTTAGGAGCTCTGCAGAACCTCA |
| | CGGCCGGAAGTGGACCAATGCCGACATCAGTGGCTCAGACAGTTGTCCAGAAGGAAAG |
| | TGGCCTGCAGCACACCCGAAAGATGCTGCATGTTGGTGACCCAAGTGTGAAAAAGACAG |
| | CCATCTCGCTGCTGAGGAATCTGTCCCGGAATCTTTCTCTGCAGAATGAAATTGCCAAAG |
| | AAACTCTCCCTGATTTGGTTTCCATCATTCCTGACACAGTCCCGAGTACTGACCTTCTCAT |
| | TGAAACTACAGCCTCTGCCTGTTACACATTGAACAACATAATCCAAAACAGTTACCAGAAT |
| | GCACGCGACCTTCTAAACACCGGGGGCATCCAGAAAATTATGGCCATTAGTGCAGGCGA |
| | TGCCTATGCCTCCAACAAAGCAAGTAAAGCTGCTTCCGTCCTTCTGTATTCTCTGTGGGC |
| | ACACACGGAACTGCATCATGCCTACAAGAAGGCTCAGTTTAAGAAGACAGATTTTGTCAA |
| | CAGCCGGACTGCCAAAGCCTACCACTCCCTTAAAGACTGATCAACCTCTGGATTACAAAA |
| | TTTGTGAAAGATTGACTGGTATTCTTAACTATGTTGCTCCTTTTACGCTATGTGGATACGC |
| | TGCTTTAATGCCTTTGTATCATGCTATTGCTTCCCGTATGGCTTTCATTTTCTCCTCCTTGT |
| | ATAAATCCTGGTTGCTGTCTCTTTATGAGGAGTTGTGGCCCGTTGTCAGGCAACGTGGC |
| | GTGGTGTGCACTGTGTTTGCTGACGCAACCCCCACTGGTTGGGGCATTGCCACCACCTG |
| | TCAGCTCCTTTCCGGGACTTTCGCTTTCCCCCTCCCTATTGCCACGGCGGAACTCATCG |
| | CCGCCTGCCTTGCCCGCTGCTGGACAGGGGCTCGGCTGTTGGGCACTGACAATTCCGT |
| | GGTGTTGTCGGGGAAATCATCGTCCTTTCCTTGGCTGCTCGCCTGTGTTGCCACCTGGA |
| | TTCTGCGCGGGACGTCCTTCTGCTACGTCCCTTCGGCCCTCAATCCAGCGGACCTTCCT |
| | TCCCGCGGCCTGCTGCCGGCTCTGCGGCCTCTTCCGCGTCTTCGCCTTCGCCCTCAGA |
| | CGAGTCGGATCTCCCTTTGGGCCGCCTCCCCGCAtcattgcCTGCCCGGGTGGCATCCCTG |
| | TGACCCCTCCCCAGTGCCTCTCCTGGCCCTGGAAGTTGCCACTCCAGTGCCCACCAGC |
| | CTTGTCCTAATAAAATTAAGTTGCATCATTTTGTCTGACTAGGTGTCCTTCTATAATATTAT |
| | GGGGTGGAGGGGGGTGGTATGGAGCAAGGGGCCCAAGTTGGGAAGAAACCTGTAGGG |
| | CCTGCgttacccaggctggagtgcagtggcacatttctgctcactgcaacctcctcctccctgggttctacgtagataagtag |
| | catgggggttaatcattaactacaaggaacccctagtgatggagttggccactccctctctgcgcgctcgctcgctcactgag |
| | gccgggcgaccaaaggtcgcccgacgcccgggctttgcccgggcggcctcagtgagcgagcgagcgcgc |
| 90 | gcgcgctcgctcgctcactgaggccgcccgggcaaagcccgggcgtcgggcgacctttggtcgcccggcctcagtgagcgagc |
| | gagcgcgcagagagggagtggccaactccatcactaggggttccttgtagttaatgattaacccgccatgctacttatctacg |
| | tactctggagacgcgtCTCAGTCCATTAGGAGCCAGTAGCCTGGAAGATGTCTTTACCCCCAGCATCA |
| | GTTCAAGTGGAGCAGCACATAACTCTTGCCCTCTGCCTTCCAAGATTCTGGTGCTGAGA |
| | CTTATGGAGTGTCTTGGAGGTTGCCTTCTGCCCCCCAACCCTGCTCCCAGCTGGCCCTC |
| | CCAGGCCTGGGTTGCTGGCCTCTGCTTTATCAGGATTCTCAAGAGGGACAGCTGGTTTA |
| | TGTTGCATGACTGTTCCCTGCATATCTGCTCTGGTTTTAAATAGCTTATCTGAGCAGCTG |
| | GAGGACCACATGGGCTTATATGGCGTGGGGTACATGTTCCTGTAGCCTTGTCCCTGGCA |
| | CCTGCCAAAATAGCAGCCAACACCCCCCACCCCCACCGCCATCCCCCTGCCCCACCCG |
| | TCCCCTGTCGCACATTCCTCCCTCCGCAGGGCTGGCTCACCAGGCCCCAGCCCACATG |
| | CCTGCTTAAAGCCCTCTCCATCCTCTGCCTCACCCAGTCCCCGCTGAGACTGAGCAGAC |
| | GCCTCCAGGATCTGTCGGCAGGCCACCATGGCAGCCCCCGGCGCCCCAGCTGAGTAC |
| | GGCTACATCCGGACCGTCCTGGGCCAGCAGATCCTGGGACAACTGGACAGCTCCAGCC |
| | TGGCGCTGCCCTCCGAGGCCAAGCTGAAGCTGGCGGGGAGCAGCGGCCGCGGCGGC |
| | CAGACAGTCAAGAGCCTGCGGATCCAGGAGCAGGTGCAGCAGACCCTCGCCCGGAAG |
| | GGCCGCAGCTCCGTGGGCAACGGAAATCTTCACCGAACCAGCAGTGTTCCTGAGTATGT |
| | CTACAACCTACACTTGGTTGAAAATGATTTTGTTGGAGGCCGTTCCCCTGTTCCTAAAAC |
| | CTATGACATGCTAAAGGCTGGCACAACTGCCACTTATGAAGGTCGCTGGGGAAGAGGAA |
| | CAGCACAGTACAGCTCCCAGAAGTCCGTGGAAGAAAGGTCCTTGAGGCATCCTCTGAGG |
| | AGACTGGAGATTTCTCCTGACAGCAGCCCGGAGAGGGCTCACTACACGCACAGCGATTA |
| | CCAGTACAGCCAGAGAAGCCAGGCTGGGCACACCCTGCACCACCAAGAAAGCAGGCGG |
| | GCCGCCCTCCTAGTGCCACCGAGATATGCTCGTTCCGAGATCGTGGGGGTCAGCCGTG |
| | CTGGCACCACAAGCAGGCAGCGCCACTTTGACACATACCACAGACAGTACCAGCATGGC |
| | TCTGTTAGCGACACCGTTTTTGACAGCATCCCTGCCAACCCGGCCCTGCTCACGTACCC |
| | CAGGCCAGGGACCAGCCGCAGCATGGGCAACCTCTTGGAGAAGGAGAACTACCTGACG |
| | GCAGGGCTCACTGTCGGGCAGGTCAGGCCGCTGGTGCCCCTGCAGCCCGTCACTCAGA |
| | ACAGGGCTTCCAGGTCCTCCTGGCATCAGAGCTCCTTCCACAGCACCCGCACGCTGAG |
| | GGAAGCTGGGCCCAGTGTCGCCGTGGATTCCAGGGGGAGGAGAGCGCACTTGACTGTC |
| | GGCCAGGCGGCCGCAGGGGGAAGTGGGAATCTGCTCACTGAGAGAAGCACTTTCACTG |
| | ACTCCCAGCTGGGGAATGCAGACATGGAGATGACTCTGGAGCGAGCAGTGAGTATGCT |
| | CGAGGCAGACCACATGCTGCCATCCAGGATTTCTGCTGCAGCTACTTTCATACAGCACG |
| | AGTGCTTCCAGAAATCTGAAGCTCGGAAGAGGGTTAACCAGCTTCGTGGCATCCTCAAG |
| | CTTCTGCAGCTCCTAAAAGTTCAGAATGAAGACGTTCAGCGAGCTGTGTGTGGGGCCTT |
| | GAGAAACTTAGTATTTGAAGACAATGACAACAAATTGGAGGTGGCTGAACTAAATGGGGT |
| | ACCTCGGCTGCTCCAGGTGCTGAAGCAAACCAGAGACTTGGAGACTAAAAAACAAATAA |
| | CAGGTTTGCTGTGGAATTTGTCATCTAATGACAAACTCAAGAATCTCATGATAACAGAAG |
| | CATTGCTTACGCTGACGGAGAATATCATCATCCCCTTTTCTGGGTGGCCTGAAGGAGACT |
| | ACCCAAAAGCAAATGGTTTGCTCGATTTTGACATATTCTACAACGTCACTGGATGCCTAA |
| | GAAACATGAGTTCTGCTGGCGCTGATGGGAGAAAAGCGATGAGAAGATGTGACGGACTC |
| | ATTGACTCACTGGTCCATTATGTCAGAGGAACCATTGCAGATTACCAGCCAGATGACAAG |
| | GCCACGGAGAATTGTGTGTGCATTCTTCATAACCTCTCCTACCAGCTGGAGGCAGAGCT |
| | CCCAGAGAAATATTCCCAGAATATCTATATTCAAAACCGGAATATCCAGACTGACAACAA |
| | CAAAAGTATTGGATGTTTTGGCAGTCGAAGCAGGAAAGTAAAAGAGCAATACCAGGACG |
| | TGCCGATGCCGGAGGAAAAGAGCAACCCCAAGGGCGTGGAGTGGCTGTGGCATTCCAT |
| | TGTTATAAGGATGTATCTGTCCTTGATCGCCAAAAGTGTCCGCAACTACACACAAGAAGC |
| | ATCCTTAGGAGCTCTGCAGAACCTCACGGCCGGAAGTGGACCAATGCCGACATCAGTG |
| | GCTCAGACAGTTGTCCAGAAGGAAAGTGGCCTGCAGCACACCCGAAAGATGCTGCATGT |
| | TGGTGACCCAAGTGTGAAAAAGACAGCCATCTCGCTGCTGAGGAATCTGTCCCGGAATC |
| | TTTCTCTGCAGAATGAAATTGCCAAAGAAACTCTCCCTGATTTGGTTTCCATCATTCCTGA |
| | CACAGTCCCGAGTACTGACCTTCTCATTGAAACTACAGCCTCTGCCTGTTACACATTGAA |
| | CAACATAATCCAAAACAGTTACCAGAATGCACGCGACCTTCTAAACACCGGGGGCATCC |
| | AGAAAATTATGGCCATTAGTGCAGGCGATGCCTATGCCTCCAACAAAGCAAGTAAAGCT |
| | GCTTCCGTCCTTCTGTATTCTCTGTGGGCACACACGGAACTGCATCATGCCTACAAGAAG |
| | GCTCAGTTTAAGAAGACAGATTTTGTCAACAGCCGGACTGCCAAAGCCTACCACTCCCTT |
| | AAAGACTGATCAACCTCTGGATTACAAAATTTGTGAAAGATTGACTGGTATTCTTAACTAT |
| | GTTGCTCCTTTTACGCTATGTGGATACGCTGCTTTAATGCCTTTGTATCATGCTATTGCTT |
| | CCCGTATGGCTTTCATTTTCTCCTCCTTGTATAAATCCTGGTTGCTGTCTCTTTATGAGGA |
| | GTTGTGGCCCGTTGTCAGGCAACGTGGCGTGGTGTGCACTGTGTTTGCTGACGCAACC |
| | CCCACTGGTTGGGGCATTGCCACCACCTGTCAGCTCCTTTCCGGGACTTTCGCTTTCCC |
| | CCTCCCTATTGCCACGGCGGAACTCATCGCCGCCTGCCTTGCCCGCTGCTGGACAGGG |
| | GCTCGGCTGTTGGGCACTGACAATTCCGTGGTGTTGTCGGGGAAATCATCGTCCTTTCC |
| | TTGGCTGCTCGCCTGTGTTGCCACCTGGATTCTGCGCGGGACGTCCTTCTGCTACGTCC |
| | CTTCGGCCCTCAATCCAGCGGACCTTCCTTCCCGCGGCCTGCTGCCGGCTCTGCGGCC |
| | TCTTCCGCGTCTTCGCCTTCGCCCTCAGACGAGTCGGATCTCCCTTTGGGCCGCCTCCC |
| | CGCAtcattgcCTGCCCGGGTGGCATCCCTGTGACCCCTCCCCAGTGCCTCTCCTGGCCCT |
| | GGAAGTTGCCACTCCAGTGCCCACCAGCCTTGTCCTAATAAAATTAAGTTGCATCATTTT |
| | GTCTGACTAGGTGTCCTTCTATAATATTATGGGGTGGAGGGGGGTGGTATGGAGCAAGG |
| | GGCCCAAGTTGGGAAGAAACCTGTAGGGCCTGCgttacccaggctggagtgcagtggcacatttctgctca |
| | ctgcaacctcctcctccctgggttctacgtagataagtagcatgggggttaatcattaactacaaggaacccctagtgatgga |
| | gttggccactccctctctgcgcgctcgctcgctcactgaggccgggcgaccaaaggtcgcccgacgcccgggctttgcccgggg |
| | gcctcagtgagcgagcgagcgcgc |
| 91 | gcgcgctcgctcgctcactgaggccgcccgggcaaagcccgggcgtcgggcgacctttggtcgcccggcctcagtgagcgagc |
| | gagcgcgcagagagggagtggccaactccatcactaggggttccttgtagttaatgattaacccgccatgctacttatctac |
| | gtactctggagacgcgtACCCTTCAGATTAAAAATAACTGAGGTAAGGGCCTGGGTAGGGGAGGTGGT |
| | GTGAGACGCTCCTGTCTCTCCTCTATCTGCCCATCGGCCCTTTGGGGAGGAGGAATGTG |
| | CCCAAGGACTAAAAAAAGGCCATGGAGCCAGAGGGGCGAGGGCAACAGACCTTTCATG |
| | GGCAAACCTTGGGGCCCTGCTGTCTAGCATGCCCCACTACGGGTCTAGGCTGCCCATG |
| | TAAGGAGGCAAGGCCTGGGGACACCCGAGATGCCTGGTTATAATTAACCCAGACATGTG |
| | GCTGCCCCCCCCCCCCCAACACCTGCTGCCTCTAAAAATAACCCTGTCCCTGGTGGATC |
| | CCCTGCATGCGAAGATCTTCGAACAAGGCTGTGGGGGACTGAGGGCAGGCTGTAACAG |
| | GCTTGGGGGCCAGGGCTTATACGTGCCTGGGACTCCCAAAGTATTACTGTTCCATGTTC |
| | CCGGCGAAGGGCCAGCTGTCCCCCGCCAGCTAGACTCAGCACTTAGTTTAGGAACCAG |
| | TGAGCAAGTCAGCCCTTGGGGCAGCCCATACAAGGCCATGGGGCTGGGCAAGCTGCAC |
| | GCCTGGGTCCGGGGTGGGCACGGTGCCCGGGCAACGAGCTGAAAGCTCATCTGCTCTC |
| | AGGGGCCCCTCCCTGGGGACAGCCCCTCCTGGCTAGTCACACCCTGTAGGCTCCTCTA |
| | TATAACCCAGGGGCACAGGGGCTGCCCTCATTCTACCACCACCTCCACAGCACAGACAG |
| | ACACTCAGGAGCCAGCCAGggtaagtttagtctttttgtcttttatttcaggtcccggatccggtggtggtgcaaatcaaag |
| | aactgctcctcagtggatgttgcctttacttctaggcctgtacggaagtgttacttctgctctaaaagctgcggaattgtacc |
| | cgcGCCACCATGGCAGCCCCCGGCGCCCCAGCTGAGTACGGCTACATCCGGACCGTCCTGGGCC |
| | AGCAGATCCTGGGACAACTGGACAGCTCCAGCCTGGCGCTGCCCTCCGAGGCCAAGCT |
| | GAAGCTGGGGGGAGCAGCGGCCGCGGCGGCCAGACAGTCAAGAGCCTGCGGATCCA |
| | GGAGCAGGTGCAGCAGACCCTCGCCCGGAAGGGCCGCAGCTCCGTGGGCAACGGAAA |
| | TCTTCACCGAACCAGCAGTGTTCCTGAGTATGTCTACAACCTACACTTGGTTGAAAATGA |
| | TTTTGTTGGAGGCCGTTCCCCTGTTCCTAAAACCTATGACATGCTAAAGGCTGGCACAAC |
| | TGCCACTTATGAAGGTCGCTGGGGAAGAGGAACAGCACAGTACAGCTCCCAGAAGTCC |
| | GTGGAAGAAAGGTCCTTGAGGCATCCTCTGAGGAGACTGGAGATTTCTCCTGACAGCAG |
| | CCCGGAGAGGGCTCACTACACGCACAGCGATTACCAGTACAGCCAGAGAAGCCAGGCT |
| | GGGCACACCCTGCACCACCAAGAAAGCAGGCGGGCCGCCCTCCTAGTGCCACCGAGAT |
| | ATGCTCGTTCCGAGATCGTGGGGGTCAGCCGTGCTGGCACCACAAGCAGGCAGCGCCA |
| | CTTTGACACATACCACAGACAGTACCAGCATGGCTCTGTTAGCGACACCGTTTTTGACAG |
| | CATCCCTGCCAACCCGGCCCTGCTCACGTACCCCAGGCCAGGGACCAGCCGCAGCATG |
| | GGCAACCTCTTGGAGAAGGAGAACTACCTGACGGCAGGGCTCACTGTCGGGCAGGTCA |
| | GGCCGCTGGTGCCCCTGCAGCCCGTCACTCAGAACAGGGCTTCCAGGTCCTCCTGGCA |
| | TCAGAGCTCCTTCCACAGCACCCGCACGCTGAGGGAAGCTGGGCCCAGTGTCGCCGTG |
| | GATTCCAGCGGGAGGAGAGCGCACTTGACTGTCGGCCAGGCGGCCGCAGGGGGAAGT |
| | GGGAATCTGCTCACTGAGAGAAGCACTTTCACTGACTCCCAGCTGGGGAATGCAGACAT |
| | GGAGATGACTCTGGAGCGAGCAGTGAGTATGCTCGAGGCAGACCACATGCTGCCATCC |
| | AGGATTTCTGCTGCAGCTACTTTCATACAGCACGAGTGCTTCCAGAAATCTGAAGCTCGG |
| | AAGAGGGTTAACCAGCTTCGTGGCATCCTCAAGCTTCTGCAGCTCCTAAAAGTTCAGAAT |
| | GAAGACGTTCAGCGAGCTGTGTGTGGGGCCTTGAGAAACTTAGTATTTGAAGACAATGA |
| | CAACAAATTGGAGGTGGCTGAACTAAATGGGGTACCTCGGCTGCTCCAGGTGCTGAAGC |
| | AAACCAGAGACTTGGAGACTAAAAAACAAATAACAGACCATACAGTCAATTTAAGAAGTA |
| | GGAATGGCTGGCCGGGCGCGGTGGCTCACGCCTGTAATCCCAGCACTTTGGGAGGCCA |
| | AGGCGGGCGGATCACGAGGTCAGGAGTTCGAGACCAGCCTGACCAACATGGTTTGCTG |
| | TGGAATTTGTCATCTAATGACAAACTCAAGAATCTCATGATAACAGAAGCATTGCTTACGC |
| | TGACGGAGAATATCATCATCCCCTTTTCTGGGTGGCCTGAAGGAGACTACCCAAAAGCA |
| | AATGGTTTGCTCGATTTTGACATATTCTACAACGTCACTGGATGCCTAAGAAACATGAGTT |
| | CTGCTGGCGCTGATGGGAGAAAAGCGATGAGAAGATGTGACGGACTCATTGACTCACTG |
| | GTCCATTATGTCAGAGGAACCATTGCAGATTACCAGCCAGATGACAAGGCCACGGAGAA |
| | TTGTGTGTGCATTCTTCATAACCTCTCCTACCAGCTGGAGGCAGAGCTCCCAGAGAAATA |
| | TTCCCAGAATATCTATATTCAAAACCGGAATATCCAGACTGACAACAACAAAAGTATTGGA |
| | TGTTTTGGCAGTCGAAGCAGGAAAGTAAAAGAGCAATACCAGGACGTGCCGATGCCGGA |
| | GGAAAAGAGCAACCCCAAGGGCGTGGAGTGGCTGTGGCATTCCATTGTTATAAGGATGT |
| | ATCTGTCCTTGATCGCCAAAAGTGTCCGCAACTACACACAAGAAGCATCCTTAGGAGCTC |
| | TGCAGAACCTCACGGCCGGAAGTGGACCAATGCCGACATCAGTGGCTCAGACAGTTGT |
| | CCAGAAGGAAAGTGGCCTGCAGCACACCCGAAAGATGCTGCATGTTGGTGACCCAAGT |
| | GTGAAAAAGACAGCCATCTCGCTGCTGAGGAATCTGTCCCGGAATCTTTCTCTGCAGAAT |
| | GAAATTGCCAAAGAAACTCTCCCTGATTTGGTTTCCATCATTCCTGACACAGTCCCGAGT |
| | ACTGACCTTCTCATTGAAACTACAGCCTCTGCCTGTTACACATTGAACAACATAATCCAAA |
| | ACAGTTACCAGAATGCACGCGACCTTCTAAACACCGGGGGCATCCAGAAAATTATGGCC |
| | ATTAGTGCAGGCGATGCCTATGCCTCCAACAAAGCAAGTAAAGCTGCTTCCGTCCTTCTG |
| | TATTCTCTGTGGGCACACACGGAACTGCATCATGCCTACAAGAAGGCTCAGTTTAAGAAG |
| | ACAGATTTTGTCAACAGCCGGACTGCCAAAGCCTACCACTCCCTTAAAGACTGATCAACC |
| | TCTGGATTACAAAATTTGTGAAAGATTGACTGGTATTCTTAACTATGTTGCTCCTTTTACG |
| | CTATGTGGATACGCTGCTTTAATGCCTTTGTATCATGCTATTGCTTCCCGTATGGCTTTCA |
| | TTTTCTCCTCCTTGTATAAATCCTGGTTGCTGTCTCTTTATGAGGAGTTGTGGCCCGTTGT |
| | CAGGCAACGTGGCGTGGTGTGCACTGTGTTTGCTGACGCAACCCCCACTGGTTGGGGC |
| | ATTGCCACCACCTGTCAGCTCCTTTCCGGGACTTTCGCTTTCCCCCTCCCTATTGCCACG |
| | GCGGAACTCATCGCCGCCTGCCTTGCCCGCTGCTGGACAGGGGCTCGGCTGTTGGGCA |
| | CTGACAATTCCGTGGTGTTGTCGGGGAAATCATCGTCCTTTCCTTGGCTGCTCGCCTGT |
| | GTTGCCACCTGGATTCTGCGCGGGACGTCCTTCTGCTACGTCCCTTCGGCCCTCAATCC |
| | AGCGGACCTTCCTTCCCGCGGCCTGCTGCCGGCTCTGCGGCCTCTTCCGCGTCTTCGC |
| | CTTCGCCCTCAGACGAGTCGGATCTCCCTTTGGGCCGCCTCCCCGCAtcattgcCTGCCCG |
| | GGTGGCATCCCTGTGACCCCTCCCCAGTGCCTCTCCTGGCCCTGGAAGTTGCCACTCCA |
| | GTGCCCACCAGCCTTGTCCTAATAAAATTAAGTTGCATCATTTTGTCTGACTAGGTGTCCT |
| | TCTATAATATTATGGGGTGGAGGGGGGTGGTATGGAGCAAGGGGCCCAAGTTGGGAAG |
| | AAACCTGTAGGGCCTGCgttacccaggctggagtgcagtggcacatttctgctcactgcaacctcctcctccctgggttct |
| | acgtagataagtagcatgggggttaatcattaactacaaggaacccctagtgatggagttggccactccctctctgcgcgctc |
| | gctcgctcactgaggccgggcgaccaaaggtcgcccgacgcccgggctttgcccgggcggcctcagtgagcgagcgagcgcgc |
| 92 | gcgcgctcgctcgctcactgaggccgcccgggcaaagcccgggcgtcgggcgacctttggtcgcccggcctcagtgagcgagc |
| | gagcgcgcagagagggagtggccaactccatcactaggggttccttgtagttaatgattaacccgccatgctacttatctac |
| | gtactctggagacgcgtCTCAGTCCATTAGGAGCCAGTAGCCTGGAAGATGTCTTTACCCCCAGCATCA |
| | GTTCAAGTGGAGCAGCACATAACTCTTGCCCTCTGCCTTCCAAGATTCTGGTGCTGAGA |
| | CTTATGGAGTGTCTTGGAGGTTGCCTTCTGCCCCCCAACCCTGCTCCCAGCTGGCCCTC |
| | CCAGGCCTGGGTTGCTGGCCTCTGCTTTATCAGGATTCTCAAGAGGGACAGCTGGTTTA |
| | TGTTGCATGACTGTTCCCTGCATATCTGCTCTGGTTTTAAATAGCTTATCTGAGCAGCTG |
| | GAGGACCACATGGGCTTATATGGCGTGGGGTACATGTTCCTGTAGCCTTGTCCCTGGCA |
| | CCTGCCAAAATAGCAGCCAACACCCCCCACCCCCACCGCCATCCCCCTGCCCCACCCG |
| | TCCCCTGTCGCACATTCCTCCCTCCGCAGGGCTGGCTCACCAGGCCCCAGCCCACATG |
| | CCTGCTTAAAGCCCTCTCCATCCTCTGCCTCACCCAGTCCCCGCTGAGACTGAGCAGAC |
| | GCCTCCAGGATCTGTCGGCAGGCCACCATGGCAGCCCCCGGCGCCCCAGCTGAGTAC |
| | GGCTACATCCGGACCGTCCTGGGCCAGCAGATCCTGGGACAACTGGACAGCTCCAGCC |
| | TGGCGCTGCCCTCCGAGGCCAAGCTGAAGCTGGCGGGGAGCAGCGGCCGCGGCGGC |
| | CAGACAGTCAAGAGCCTGCGGATCCAGGAGCAGGTGCAGCAGACCCTCGCCCGGAAG |
| | GGCCGCAGCTCCGTGGGCAACGGAAATCTTCACCGAACCAGCAGTGTTCCTGAGTATGT |
| | CTACAACCTACACTTGGTTGAAAATGATTTTGTTGGAGGCCGTTCCCCTGTTCCTAAAAC |
| | CTATGACATGCTAAAGGCTGGCACAACTGCCACTTATGAAGGTCGCTGGGGAAGAGGAA |
| | CAGCACAGTACAGCTCCCAGAAGTCCGTGGAAGAAAGGTCCTTGAGGCATCCTCTGAGG |
| | AGACTGGAGATTTCTCCTGACAGCAGCCCGGAGAGGGCTCACTACACGCACAGCGATTA |
| | CCAGTACAGCCAGAGAAGCCAGGCTGGGCACACCCTGCACCACCAAGAAAGCAGGCGG |
| | GCCGCCCTCCTAGTGCCACCGAGATATGCTCGTTCCGAGATCGTGGGGGTCAGCCGTG |
| | CTGGCACCACAAGCAGGCAGCGCCACTTTGACACATACCACAGACAGTACCAGCATGGC |
| | TCTGTTAGCGACACCGTTTTTGACAGCATCCCTGCCAACCCGGCCCTGCTCACGTACCC |
| | CAGGCCAGGGACCAGCCGCAGCATGGGCAACCTCTTGGAGAAGGAGAACTACCTGACG |
| | GCAGGGCTCACTGTCGGGCAGGTCAGGCCGCTGGTGCCCCTGCAGCCCGTCACTCAGA |
| | ACAGGGCTTCCAGGTCCTCCTGGCATCAGAGCTCCTTCCACAGCACCCGCACGCTGAG |
| | GGAAGCTGGGCCCAGTGTCGCCGTGGATTCCAGCGGGAGGAGAGCGCACTTGACTGTC |
| | GGCCAGGCGGCCGCAGGGGGAAGTGGGAATCTGCTCACTGAGAGAAGCACTTTCACTG |
| | ACTCCCAGCTGGGGAATGCAGACATGGAGATGACTCTGGAGCGAGCAGTGAGTATGCT |
| | CGAGGCAGACCACATGCTGCCATCCAGGATTTCTGCTGCAGCTACTTTCATACAGCACG |
| | AGTGCTTCCAGAAATCTGAAGCTCGGAAGAGGGTTAACCAGCTTCGTGGCATCCTCAAG |
| | CTTCTGCAGCTCCTAAAAGTTCAGAATGAAGACGTTCAGCGAGCTGTGTGTGGGGCCTT |
| | GAGAAACTTAGTATTTGAAGACAATGACAACAAATTGGAGGTGGCTGAACTAAATGGGGT |
| | ACCTCGGCTGCTCCAGGTGCTGAAGCAAACCAGAGACTTGGAGACTAAAAAACAAATAA |
| | CAGACCATACAGTCAATTTAAGAAGTAGGAATGGCTGGCCGGGCGCGGTGGCTCACGC |
| | CTGTAATCCCAGCACTTTGGGAGGCCAAGGCGGGCGGATCACGAGGTCAGGAGTTCGA |
| | GACCAGCCTGACCAACATGGTTTGCTGTGGAATTTGTCATCTAATGACAAACTCAAGAAT |
| | CTCATGATAACAGAAGCATTGCTTACGCTGACGGAGAATATCATCATCCCCTTTTCTGGG |
| | TGGCCTGAAGGAGACTACCCAAAAGCAAATGGTTTGCTCGATTTTGACATATTCTACAAC |
| | GTCACTGGATGCCTAAGAAACATGAGTTCTGCTGGCGCTGATGGGAGAAAAGCGATGAG |
| | AAGATGTGACGGACTCATTGACTCACTGGTCCATTATGTCAGAGGAACCATTGCAGATTA |
| | CCAGCCAGATGACAAGGCCACGGAGAATTGTGTGTGCATTCTTCATAACCTCTCCTACCA |
| | GCTGGAGGCAGAGCTCCCAGAGAAATATTCCCAGAATATCTATATTCAAAACCGGAATAT |
| | CCAGACTGACAACAACAAAAGTATTGGATGTTTTGGCAGTCGAAGCAGGAAAGTAAAAGA |
| | GCAATACCAGGACGTGCCGATGCCGGAGGAAAAGAGCAACCCCAAGGGCGTGGAGTG |
| | GCTGTGGCATTCCATTGTTATAAGGATGTATCTGTCCTTGATCGCCAAAAGTGTCCGCAA |
| | CTACACACAAGAAGCATCCTTAGGAGCTCTGCAGAACCTCACGGCCGGAAGTGGACCAA |
| | TGCCGACATCAGTGGCTCAGACAGTTGTCCAGAAGGAAAGTGGCCTGCAGCACACCCG |
| | AAAGATGCTGCATGTTGGTGACCCAAGTGTGAAAAAGACAGCCATCTCGCTGCTGAGGA |
| | ATCTGTCCCGGAATCTTTCTCTGCAGAATGAAATTGCCAAAGAAACTCTCCCTGATTTGG |
| | TTTCCATCATTCCTGACACAGTCCCGAGTACTGACCTTCTCATTGAAACTACAGCCTCTG |
| | CCTGTTACACATTGAACAACATAATCCAAAACAGTTACCAGAATGCACGCGACCTTCTAA |
| | ACACCGGGGGCATCCAGAAAATTATGGCCATTAGTGCAGGCGATGCCTATGCCTCCAAC |
| | AAAGCAAGTAAAGCTGCTTCCGTCCTTCTGTATTCTCTGTGGGCACACACGGAACTGCAT |
| | CATGCCTACAAGAAGGCTCAGTTTAAGAAGACAGATTTTGTCAACAGCCGGACTGCCAAA |
| | GCCTACCACTCCCTTAAAGACTGATCAACCTCTGGATTACAAAATTTGTGAAAGATTGACT |
| | GGTATTCTTAACTATGTTGCTCCTTTTACGCTATGTGGATACGCTGCTTTAATGCCTTTGT |
| | ATCATGCTATTGCTTCCCGTATGGCTTTCATTTTCTCCTCCTTGTATAAATCCTGGTTGCT |
| | GTCTCTTTATGAGGAGTTGTGGCCCGTTGTCAGGCAACGTGGCGTGGTGTGCACTGTGT |
| | TTGCTGACGCAACCCCCACTGGTTGGGGCATTGCCACCACCTGTCAGCTCCTTTCCGGG |
| | ACTTTCGCTTTCCCCCTCCCTATTGCCACGGCGGAACTCATCGCCGCCTGCCTTGCCCG |
| | CTGCTGGACAGGGGCTCGGCTGTTGGGCACTGACAATTCCGTGGTGTTGTCGGGGAAA |
| | TCATCGTCCTTTCCTTGGCTGCTCGCCTGTGTTGCCACCTGGATTCTGCGCGGGACGTC |
| | CTTCTGCTACGTCCCTTCGGCCCTCAATCCAGCGGACCTTCCTTCCCGCGGCCTGCTGC |
| | CGGCTCTGCGGCCTCTTCCGCGTCTTCGCCTTCGCCCTCAGACGAGTCGGATCTCCCTT |
| | TGGGCCGCCTCCCCGCAtcattgcCTGCCCGGGTGGCATCCCTGTGACCCCTCCCCAGTGC |
| | CTCTCCTGGCCCTGGAAGTTGCCACTCCAGTGCCCACCAGCCTTGTCCTAATAAAATTAA |
| | GTTGCATCATTTTGTCTGACTAGGTGTCCTTCTATAATATTATGGGGTGGAGGGGGGTGG |
| | TATGGAGCAAGGGGCCCAAGTTGGGAAGAAACCTGTAGGGCCTGCgttacccaggctggagtgc |
| | agtggcacatttctgctcactgcaacctcctcctccctgggttctacgtagataagtagcatgggggttaatcattaactaca |
| | aggaacccctagtgatggagttggccactccctctctgcgcgctcgctcgctcactgaggccgggcgaccaaaggtcgcccga |
| | cgcccgggctttgcccgggcggcctcagtgagcgagcgagcgcgc |
| 93 | ACCCTTCAGATTAAAAATAACTGAGGTAAGGGCCTGGGTAGGGGAGGTGGTGTGAGACG |
| | CTCCTGTCTCTCCTCTATCTGCCCATCGGCCCTTTGGGGAGGAGGAATGTGCCCAAGGA |
| | CTAAAAAAAGGCCATGGAGCCAGAGGGGCGAGGGCAACAGACCTTTCATGGGCAAACC |
| | TTGGGGCCCTGCTGTCTAGCATGCCCCACTACGGGTCTAGGCTGCCCATGTAAGGAGG |
| | CAAGGCCTGGGGACACCCGAGATGCCTGGTTATAATTAACCCAGACATGTGGCTGCCCC |
| | CCCCCCCCCAACACCTGCTGCCTCTAAAAATAACCCTGTCCCTGGTGGATCCCCTGCAT |
| | GCGAAGATCTTCGAACAAGGCTGTGGGGGACTGAGGGCAGGCTGTAACAGGCTTGGGG |
| | GCCAGGGCTTATACGTGCCTGGGACTCCCAAAGTATTACTGTTCCATGTTCCCGGCGAA |
| | GGGCCAGCTGTCCCCCGCCAGCTAGACTCAGCACTTAGTTTAGGAACCAGTGAGCAAGT |
| | CAGCCCTTGGGGCAGCCCATACAAGGCCATGGGGCTGGGCAAGCTGCACGCCTGGGT |
| | CCGGGGTGGGCACGGTGCCCGGGCAACGAGCTGAAAGCTCATCTGCTCTCAGGGGCC |
| | CCTCCCTGGGGACAGCCCCTCCTGGCTAGTCACACCCTGTAGGCTCCTCTATATAACCC |
| | AGGGGCACAGGGGCTGCCCTCATTCTACCACCACCTCCACAGCACAGACAGACACTCA |
| | GGAGCCAGCCAGggtaagtttagtctttttgtcttttatttcaggtcccggatccggtggtggtgcaaatcaaagaactgctc |
| | ctcagtggatgttgcctttacttctaggcctgtacggaagtgttacttctgctctaaaagctgcggaattgtacccgcGCCAC |
| | CATGGCAGCCCCCGGCGCCCCAGCTGAGTACGGCTACATCCGGACCGTCCTGGGCCAGCAG |
| | ATCCTGGGACAACTGGACAGCTCCAGCCTGGCGCTGCCCTCCGAGGCCAAGCTGAAGC |
| | TGGCGGGGAGCAGCGGCCGCGGCGGCCAGACAGTCAAGAGCCTGCGGATCCAGGAGC |
| | AGGTGCAGCAGACCCTCGCCCGGAAGGGCCGCAGCTCCGTGGGCAACGGAAATCTTCA |
| | CCGAACCAGCAGTGTTCCTGAGTATGTCTACAACCTACACTTGGTTGAAAATGATTTTGT |
| | TGGAGGCCGTTCCCCTGTTCCTAAAACCTATGACATGCTAAAGGCTGGCACAACTGCCA |
| | CTTATGAAGGTCGCTGGGGAAGAGGAACAGCACAGTACAGCTCCCAGAAGTCCGTGGA |
| | AGAAAGGTCCTTGAGGCATCCTCTGAGGAGACTGGAGATTTCTCCTGACAGCAGCCCGG |
| | AGAGGGCTCACTACACGCACAGCGATTACCAGTACAGCCAGAGAAGCCAGGCTGGGCA |
| | CACCCTGCACCACCAAGAAAGCAGGCGGGCCGCCCTCCTAGTGCCACCGAGATATGCT |
| | CGTTCCGAGATCGTGGGGGTCAGCCGTGCTGGCACCACAAGCAGGCAGCGCCACTTTG |
| | ACACATACCACAGACAGTACCAGCATGGCTCTGTTAGCGACACCGTTTTTGACAGCATCC |
| | CTGCCAACCCGGCCCTGCTCACGTACCCCAGGCCAGGGACCAGCCGCAGCATGGGCAA |
| | CCTCTTGGAGAAGGAGAACTACCTGACGGCAGGGCTCACTGTCGGGCAGGTCAGGCCG |
| | CTGGTGCCCCTGCAGCCCGTCACTCAGAACAGGGCTTCCAGGTCCTCCTGGCATCAGA |
| | GCTCCTTCCACAGCACCCGCACGCTGAGGGAAGCTGGGCCCAGTGTCGCCGTGGATTC |
| | CAGCGGGAGGAGAGCGCACTTGACTGTCGGCCAGGCGGCCGCAGGGGGAAGTGGGAA |
| | TCTGCTCACTGAGAGAAGCACTTTCACTGACTCCCAGCTGGGGAATGCAGACATGGAGA |
| | TGACTCTGGAGCGAGCAGTGAGTATGCTCGAGGCAGACCACATGCTGCCATCCAGGATT |
| | TCTGCTGCAGCTACTTTCATACAGCACGAGTGCTTCCAGAAATCTGAAGCTCGGAAGAG |
| | GGTTAACCAGCTTCGTGGCATCCTCAAGCTTCTGCAGCTCCTAAAAGTTCAGAATGAAGA |
| | CGTTCAGCGAGCTGTGTGTGGGGCCTTGAGAAACTTAGTATTTGAAGACAATGACAACA |
| | AATTGGAGGTGGCTGAACTAAATGGGGTACCTCGGCTGCTCCAGGTGCTGAAGCAAACC |
| | AGAGACTTGGAGACTAAAAAACAAATAACAGGTTTGCTGTGGAATTTGTCATCTAATGAC |
| | AAACTCAAGAATCTCATGATAACAGAAGCATTGCTTACGCTGACGGAGAATATCATCATC |
| | CCCTTTTCTGGGTGGCCTGAAGGAGACTACCCAAAAGCAAATGGTTTGCTCGATTTTGAC |
| | ATATTCTACAACGTCACTGGATGCCTAAGAAACATGAGTTCTGCTGGCGCTGATGGGAG |
| | AAAAGCGATGAGAAGATGTGACGGACTCATTGACTCACTGGTCCATTATGTCAGAGGAA |
| | CCATTGCAGATTACCAGCCAGATGACAAGGCCACGGAGAATTGTGTGTGCATTCTTCATA |
| | ACCTCTCCTACCAGCTGGAGGCAGAGCTCCCAGAGAAATATTCCCAGAATATCTATATTC |
| | AAAACCGGAATATCCAGACTGACAACAACAAAAGTATTGGATGTTTTGGCAGTCGAAGCA |
| | GGAAAGTAAAAGAGCAATACCAGGACGTGCCGATGCCGGAGGAAAAGAGCAACCCCAA |
| | GGGCGTGGAGTGGCTGTGGCATTCCATTGTTATAAGGATGTATCTGTCCTTGATCGCCA |
| | AAAGTGTCCGCAACTACACACAAGAAGCATCCTTAGGAGCTCTGCAGAACCTCACGGCC |
| | GGAAGTGGACCAATGCCGACATCAGTGGCTCAGACAGTTGTCCAGAAGGAAAGTGGCC |
| | TGCAGCACACCCGAAAGATGCTGCATGTTGGTGACCCAAGTGTGAAAAAGACAGCCATC |
| | TCGCTGCTGAGGAATCTGTCCCGGAATCTTTCTCTGCAGAATGAAATTGCCAAAGAAACT |
| | CTCCCTGATTTGGTTTCCATCATTCCTGACACAGTCCCGAGTACTGACCTTCTCATTGAA |
| | ACTACAGCCTCTGCCTGTTACACATTGAACAACATAATCCAAAACAGTTACCAGAATGCA |
| | CGCGACCTTCTAAACACCGGGGGCATCCAGAAAATTATGGCCATTAGTGCAGGCGATGC |
| | CTATGCCTCCAACAAAGCAAGTAAAGCTGCTTCCGTCCTTCTGTATTCTCTGTGGGCACA |
| | CACGGAACTGCATCATGCCTACAAGAAGGCTCAGTTTAAGAAGACAGATTTTGTCAACAG |
| | CCGGACTGCCAAAGCCTACCACTCCCTTAAAGACTGATCAACCTCTGGATTACAAAATTT |
| | GTGAAAGATTGACTGGTATTCTTAACTATGTTGCTCCTTTTACGCTATGTGGATACGCTGC |
| | TTTAATGCCTTTGTATCATGCTATTGCTTCCCGTATGGCTTTCATTTTCTCCTCCTTGTATA |
| | AATCCTGGTTGCTGTCTCTTTATGAGGAGTTGTGGCCCGTTGTCAGGCAACGTGGCGTG |
| | GTGTGCACTGTGTTTGCTGACGCAACCCCCACTGGTTGGGGCATTGCCACCACCTGTCA |
| | GCTCCTTTCCGGGACTTTCGCTTTCCCCCTCCCTATTGCCACGGCGGAACTCATCGCCG |
| | CCTGCCTTGCCCGCTGCTGGACAGGGGCTCGGCTGTTGGGCACTGACAATTCCGTGGT |
| | GTTGTCGGGGAAATCATCGTCCTTTCCTTGGCTGCTCGCCTGTGTTGCCACCTGGATTCT |
| | GCGCGGGACGTCCTTCTGCTACGTCCCTTCGGCCCTCAATCCAGCGGACCTTCCTTCCC |
| | GCGGCCTGCTGCCGGCTCTGCGGCCTCTTCCGCGTCTTCGCCTTCGCCCTCAGACGAG |
| | TCGGATCTCCCTTTGGGCCGCCTCCCCGCAtcattgcCTGCCCGGGTGGCATCCCTGTGAC |
| | CCCTCCCCAGTGCCTCTCCTGGCCCTGGAAGTTGCCACTCCAGTGCCCACCAGCCTTGT |
| | CCTAATAAAATTAAGTTGCATCATTTTGTCTGACTAGGTGTCCTTCTATAATATTATGGGG |
| | TGGAGGGGGGTGGTATGGAGCAAGGGGCCCAAGTTGGGAAGAAACCTGTAGGGCCTG |
| | C |
| 94 | CTCAGTCCATTAGGAGCCAGTAGCCTGGAAGATGTCTTTACCCCCAGCATCAGTTCAAGT |
| | GGAGCAGCACATAACTCTTGCCCTCTGCCTTCCAAGATTCTGGTGCTGAGACTTATGGA |
| | GTGTCTTGGAGGTTGCCTTCTGCCCCCCAACCCTGCTCCCAGCTGGCCCTCCCAGGCCT |
| | GGGTTGCTGGCCTCTGCTTTATCAGGATTCTCAAGAGGGACAGCTGGTTTATGTTGCAT |
| | GACTGTTCCCTGCATATCTGCTCTGGTTTTAAATAGCTTATCTGAGCAGCTGGAGGACCA |
| | CATGGGCTTATATGGCGTGGGGTACATGTTCCTGTAGCCTTGTCCCTGGCACCTGCCAA |
| | AATAGCAGCCAACACCCCCCACCCCCACCGCCATCCCCCTGCCCCACCCGTCCCCTGT |
| | CGCACATTCCTCCCTCCGCAGGGCTGGCTCACCAGGCCCCAGCCCACATGCCTGCTTA |
| | AAGCCCTCTCCATCCTCTGCCTCACCCAGTCCCCGCTGAGACTGAGCAGACGCCTCCAG |
| | GATCTGTCGGCAGGCCACCATGGCAGCCCCCGGCGCCCCAGCTGAGTACGGCTACATC |
| | CGGACCGTCCTGGGCCAGCAGATCCTGGGACAACTGGACAGCTCCAGCCTGGCGCTGC |
| | CCTCCGAGGCCAAGCTGAAGCTGGCGGGGAGCAGCGGCCGCGGCGGCCAGACAGTCA |
| | AGAGCCTGCGGATCCAGGAGCAGGTGCAGCAGACCCTCGCCCGGAAGGGCCGCAGCT |
| | CCGTGGGCAACGGAAATCTTCACCGAACCAGCAGTGTTCCTGAGTATGTCTACAACCTA |
| | CACTTGGTTGAAAATGATTTTGTTGGAGGCCGTTCCCCTGTTCCTAAAACCTATGACATG |
| | CTAAAGGCTGGCACAACTGCCACTTATGAAGGTCGCTGGGGAAGAGGAACAGCACAGTA |
| | CAGCTCCCAGAAGTCCGTGGAAGAAAGGTCCTTGAGGCATCCTCTGAGGAGACTGGAG |
| | ATTTCTCCTGACAGCAGCCCGGAGAGGGCTCACTACACGCACAGCGATTACCAGTACAG |
| | CCAGAGAAGCCAGGCTGGGCACACCCTGCACCACCAAGAAAGCAGGCGGGCCGCCCT |
| | CCTAGTGCCACCGAGATATGCTCGTTCCGAGATCGTGGGGGTCAGCCGTGCTGGCACC |
| | ACAAGCAGGCAGCGCCACTTTGACACATACCACAGACAGTACCAGCATGGCTCTGTTAG |
| | CGACACCGTTTTTGACAGCATCCCTGCCAACCCGGCCCTGCTCACGTACCCCAGGCCAG |
| | GGACCAGCCGCAGCATGGGCAACCTCTTGGAGAAGGAGAACTACCTGACGGCAGGGCT |
| | CACTGTCGGGCAGGTCAGGCCGCTGGTGCCCCTGCAGCCCGTCACTCAGAACAGGGCT |
| | TCCAGGTCCTCCTGGCATCAGAGCTCCTTCCACAGCACCCGCACGCTGAGGGAAGCTG |
| | GGCCCAGTGTCGCCGTGGATTCCAGCGGGAGGAGAGCGCACTTGACTGTCGGCCAGG |
| | CGGCCGCAGGGGGAAGTGGGAATCTGCTCACTGAGAGAAGCACTTTCACTGACTCCCA |
| | GCTGGGGAATGCAGACATGGAGATGACTCTGGAGCGAGCAGTGAGTATGCTCGAGGCA |
| | GACCACATGCTGCCATCCAGGATTTCTGCTGCAGCTACTTTCATACAGCACGAGTGCTTC |
| | CAGAAATCTGAAGCTCGGAAGAGGGTTAACCAGCTTCGTGGCATCCTCAAGCTTCTGCA |
| | GCTCCTAAAAGTTCAGAATGAAGACGTTCAGCGAGCTGTGTGTGGGGCCTTGAGAAACT |
| | TAGTATTTGAAGACAATGACAACAAATTGGAGGTGGCTGAACTAAATGGGGTACCTCGG |
| | CTGCTCCAGGTGCTGAAGCAAACCAGAGACTTGGAGACTAAAAAACAAATAACAGGTTT |
| | GCTGTGGAATTTGTCATCTAATGACAAACTCAAGAATCTCATGATAACAGAAGCATTGCTT |
| | ACGCTGACGGAGAATATCATCATCCCCTTTTCTGGGTGGCCTGAAGGAGACTACCCAAA |
| | AGCAAATGGTTTGCTCGATTTTGACATATTCTACAACGTCACTGGATGCCTAAGAAACAT |
| | GAGTTCTGCTGGCGCTGATGGGAGAAAAGCGATGAGAAGATGTGACGGACTCATTGACT |
| | CACTGGTCCATTATGTCAGAGGAACCATTGCAGATTACCAGCCAGATGACAAGGCCACG |
| | GAGAATTGTGTGTGCATTCTTCATAACCTCTCCTACCAGCTGGAGGCAGAGCTCCCAGA |
| | GAAATATTCCCAGAATATCTATATTCAAAACCGGAATATCCAGACTGACAACAACAAAAGT |
| | ATTGGATGTTTTGGCAGTCGAAGCAGGAAAGTAAAAGAGCAATACCAGGACGTGCCGAT |
| | GCCGGAGGAAAAGAGCAACCCCAAGGGCGTGGAGTGGCTGTGGCATTCCATTGTTATA |
| | AGGATGTATCTGTCCTTGATCGCCAAAAGTGTCCGCAACTACACACAAGAAGCATCCTTA |
| | GGAGCTCTGCAGAACCTCACGGCCGGAAGTGGACCAATGCCGACATCAGTGGCTCAGA |
| | CAGTTGTCCAGAAGGAAAGTGGCCTGCAGCACACCCGAAAGATGCTGCATGTTGGTGAC |
| | CCAAGTGTGAAAAAGACAGCCATCTCGCTGCTGAGGAATCTGTCCCGGAATCTTTCTCT |
| | GCAGAATGAAATTGCCAAAGAAACTCTCCCTGATTTGGTTTCCATCATTCCTGACACAGT |
| | CCCGAGTACTGACCTTCTCATTGAAACTACAGCCTCTGCCTGTTACACATTGAACAACAT |
| | AATCCAAAACAGTTACCAGAATGCACGCGACCTTCTAAACACCGGGGGCATCCAGAAAA |
| | TTATGGCCATTAGTGCAGGCGATGCCTATGCCTCCAACAAAGCAAGTAAAGCTGCTTCC |
| | GTCCTTCTGTATTCTCTGTGGGCACACACGGAACTGCATCATGCCTACAAGAAGGCTCA |
| | GTTTAAGAAGACAGATTTTGTCAACAGCCGGACTGCCAAAGCCTACCACTCCCTTAAAGA |
| | CTGATCAACCTCTGGATTACAAAATTTGTGAAAGATTGACTGGTATTCTTAACTATGTTGC |
| | TCCTTTTACGCTATGTGGATACGCTGCTTTAATGCCTTTGTATCATGCTATTGCTTCCCGT |
| | ATGGCTTTCATTTTCTCCTCCTTGTATAAATCCTGGTTGCTGTCTCTTTATGAGGAGTTGT |
| | GGCCCGTTGTCAGGCAACGTGGCGTGGTGTGCACTGTGTTTGCTGACGCAACCCCCAC |
| | TGGTTGGGGCATTGCCACCACCTGTCAGCTCCTTTCCGGGACTTTCGCTTTCCCCCTCC |
| | CTATTGCCACGGCGGAACTCATCGCCGCCTGCCTTGCCCGCTGCTGGACAGGGGCTCG |
| | GCTGTTGGGCACTGACAATTCCGTGGTGTTGTCGGGGAAATCATCGTCCTTTCCTTGGC |
| | TGCTCGCCTGTGTTGCCACCTGGATTCTGCGCGGGACGTCCTTCTGCTACGTCCCTTCG |
| | GCCCTCAATCCAGCGGACCTTCCTTCCCGCGGCCTGCTGCCGGCTCTGCGGCCTCTTC |
| | CGCGTCTTCGCCTTCGCCCTCAGACGAGTCGGATCTCCCTTTGGGCCGCCTCCCCGCAt |
| | cattgcCTGCCCGGGTGGCATCCCTGTGACCCCTCCCCAGTGCCTCTCCTGGCCCTGGAA |
| | GTTGCCACTCCAGTGCCCACCAGCCTTGTCCTAATAAAATTAAGTTGCATCATTTTGTCT |
| | GACTAGGTGTCCTTCTATAATATTATGGGGTGGAGGGGGGTGGTATGGAGCAAGGGGC |
| | CCAAGTTGGGAAGAAACCTGTAGGGCCTGC |
| 95 | ACCCTTCAGATTAAAAATAACTGAGGTAAGGGCCTGGGTAGGGGAGGTGGTGTGAGACG |
| | CTCCTGTCTCTCCTCTATCTGCCCATCGGCCCTTTGGGGAGGAGGAATGTGCCCAAGGA |
| | CTAAAAAAAGGCCATGGAGCCAGAGGGGCGAGGGCAACAGACCTTTCATGGGCAAACC |
| | TTGGGGCCCTGCTGTCTAGCATGCCCCACTACGGGTCTAGGCTGCCCATGTAAGGAGG |
| | CAAGGCCTGGGGACACCCGAGATGCCTGGTTATAATTAACCCAGACATGTGGCTGCCCC |
| | CCCCCCCCCAACACCTGCTGCCTCTAAAAATAACCCTGTCCCTGGTGGATCCCCTGCAT |
| | GCGAAGATCTTCGAACAAGGCTGTGGGGGACTGAGGGCAGGCTGTAACAGGCTTGGGG |
| | GCCAGGGCTTATACGTGCCTGGGACTCCCAAAGTATTACTGTTCCATGTTCCCGGCGAA |
| | GGGCCAGCTGTCCCCCGCCAGCTAGACTCAGCACTTAGTTTAGGAACCAGTGAGCAAGT |
| | CAGCCCTTGGGGCAGCCCATACAAGGCCATGGGGCTGGGCAAGCTGCACGCCTGGGT |
| | CCGGGGTGGGCACGGTGCCCGGGCAACGAGCTGAAAGCTCATCTGCTCTCAGGGGCC |
| | CCTCCCTGGGGACAGCCCCTCCTGGCTAGTCACACCCTGTAGGCTCCTCTATATAACCC |
| | AGGGGCACAGGGGCTGCCCTCATTCTACCACCACCTCCACAGCACAGACAGACACTCA |
| | GGAGCCAGCCAGggtaagtttagtctttttgtcttttatttcaggtcccggatccggtggtggtgcaaatcaaagaactgctc |
| | ctcagtggatgttgcctttacttctaggcctgtacggaagtgttacttctgctctaaaagctgcggaattgtacccgcGCCAC |
| | CATGGCAGCCCCCGGCGCCCCAGCTGAGTACGGCTACATCCGGACCGTCCTGGGCCAGCAG |
| | ATCCTGGGACAACTGGACAGCTCCAGCCTGGCGCTGCCCTCCGAGGCCAAGCTGAAGC |
| | TGGCGGGGAGCAGCGGCCGCGGCGGCCAGACAGTCAAGAGCCTGCGGATCCAGGAGC |
| | AGGTGCAGCAGACCCTCGCCCGGAAGGGCCGCAGCTCCGTGGGCAACGGAAATCTTCA |
| | CCGAACCAGCAGTGTTCCTGAGTATGTCTACAACCTACACTTGGTTGAAAATGATTTTGT |
| | TGGAGGCCGTTCCCCTGTTCCTAAAACCTATGACATGCTAAAGGCTGGCACAACTGCCA |
| | CTTATGAAGGTCGCTGGGGAAGAGGAACAGCACAGTACAGCTCCCAGAAGTCCGTGGA |
| | AGAAAGGTCCTTGAGGCATCCTCTGAGGAGACTGGAGATTTCTCCTGACAGCAGCCCGG |
| | AGAGGGCTCACTACACGCACAGCGATTACCAGTACAGCCAGAGAAGCCAGGCTGGGCA |
| | CACCCTGCACCACCAAGAAAGCAGGCGGGCCGCCCTCCTAGTGCCACCGAGATATGCT |
| | CGTTCCGAGATCGTGGGGGTCAGCCGTGCTGGCACCACAAGCAGGCAGCGCCACTTTG |
| | ACACATACCACAGACAGTACCAGCATGGCTCTGTTAGCGACACCGTTTTTGACAGCATCC |
| | CTGCCAACCCGGCCCTGCTCACGTACCCCAGGCCAGGGACCAGCCGCAGCATGGGCAA |
| | CCTCTTGGAGAAGGAGAACTACCTGACGGCAGGGCTCACTGTCGGGCAGGTCAGGCCG |
| | CTGGTGCCCCTGCAGCCCGTCACTCAGAACAGGGCTTCCAGGTCCTCCTGGCATCAGA |
| | GCTCCTTCCACAGCACCCGCACGCTGAGGGAAGCTGGGCCCAGTGTCGCCGTGGATTC |
| | CAGCGGGAGGAGAGCGCACTTGACTGTCGGCCAGGCGGCCGCAGGGGGAAGTGGGAA |
| | TCTGCTCACTGAGAGAAGCACTTTCACTGACTCCCAGCTGGGGAATGCAGACATGGAGA |
| | TGACTCTGGAGCGAGCAGTGAGTATGCTCGAGGCAGACCACATGCTGCCATCCAGGATT |
| | TCTGCTGCAGCTACTTTCATACAGCACGAGTGCTTCCAGAAATCTGAAGCTCGGAAGAG |
| | GGTTAACCAGCTTCGTGGCATCCTCAAGCTTCTGCAGCTCCTAAAAGTTCAGAATGAAGA |
| | CGTTCAGCGAGCTGTGTGTGGGGCCTTGAGAAACTTAGTATTTGAAGACAATGACAACA |
| | AATTGGAGGTGGCTGAACTAAATGGGGTACCTCGGCTGCTCCAGGTGCTGAAGCAAACC |
| | AGAGACTTGGAGACTAAAAAACAAATAACAGACCATACAGTCAATTTAAGAAGTAGGAAT |
| | GGCTGGCCGGGCGCGGTGGCTCACGCCTGTAATCCCAGCACTTTGGGAGGCCAAGGC |
| | GGGCGGATCACGAGGTCAGGAGTTCGAGACCAGCCTGACCAACATGGTTTGCTGTGGA |
| | ATTTGTCATCTAATGACAAACTCAAGAATCTCATGATAACAGAAGCATTGCTTACGCTGAC |
| | GGAGAATATCATCATCCCCTTTTCTGGGTGGCCTGAAGGAGACTACCCAAAAGCAAATG |
| | GTTTGCTCGATTTTGACATATTCTACAACGTCACTGGATGCCTAAGAAACATGAGTTCTG |
| | CTGGCGCTGATGGGAGAAAAGCGATGAGAAGATGTGACGGACTCATTGACTCACTGGTC |
| | CATTATGTCAGAGGAACCATTGCAGATTACCAGCCAGATGACAAGGCCACGGAGAATTG |
| | TGTGTGCATTCTTCATAACCTCTCCTACCAGCTGGAGGCAGAGCTCCCAGAGAAATATTC |
| | CCAGAATATCTATATTCAAAACCGGAATATCCAGACTGACAACAACAAAAGTATTGGATGT |
| | TTTGGCAGTCGAAGCAGGAAAGTAAAAGAGCAATACCAGGACGTGCCGATGCCGGAGG |
| | AAAAGAGCAACCCCAAGGGCGTGGAGTGGCTGTGGCATTCCATTGTTATAAGGATGTAT |
| | CTGTCCTTGATCGCCAAAAGTGTCCGCAACTACACACAAGAAGCATCCTTAGGAGCTCT |
| | GCAGAACCTCACGGCCGGAAGTGGACCAATGCCGACATCAGTGGCTCAGACAGTTGTC |
| | CAGAAGGAAAGTGGCCTGCAGCACACCCGAAAGATGCTGCATGTTGGTGACCCAAGTGT |
| | GAAAAAGACAGCCATCTCGCTGCTGAGGAATCTGTCCCGGAATCTTTCTCTGCAGAATG |
| | AAATTGCCAAAGAAACTCTCCCTGATTTGGTTTCCATCATTCCTGACACAGTCCCGAGTA |
| | CTGACCTTCTCATTGAAACTACAGCCTCTGCCTGTTACACATTGAACAACATAATCCAAAA |
| | CAGTTACCAGAATGCACGCGACCTTCTAAACACCGGGGGCATCCAGAAAATTATGGCCA |
| | TTAGTGCAGGCGATGCCTATGCCTCCAACAAAGCAAGTAAAGCTGCTTCCGTCCTTCTGT |
| | ATTCTCTGTGGGCACACACGGAACTGCATCATGCCTACAAGAAGGCTCAGTTTAAGAAG |
| | ACAGATTTTGTCAACAGCCGGACTGCCAAAGCCTACCACTCCCTTAAAGACTGATCAACC |
| | TCTGGATTACAAAATTTGTGAAAGATTGACTGGTATTCTTAACTATGTTGCTCCTTTTACG |
| | CTATGTGGATACGCTGCTTTAATGCCTTTGTATCATGCTATTGCTTCCCGTATGGCTTTCA |
| | TTTTCTCCTCCTTGTATAAATCCTGGTTGCTGTCTCTTTATGAGGAGTTGTGGCCCGTTGT |
| | CAGGCAACGTGGCGTGGTGTGCACTGTGTTTGCTGACGCAACCCCCACTGGTTGGGGC |
| | ATTGCCACCACCTGTCAGCTCCTTTCCGGGACTTTCGCTTTCCCCCTCCCTATTGCCACG |
| | GCGGAACTCATCGCCGCCTGCCTTGCCCGCTGCTGGACAGGGGCTCGGCTGTTGGGCA |
| | CTGACAATTCCGTGGTGTTGTCGGGGAAATCATCGTCCTTTCCTTGGCTGCTCGCCTGT |
| | GTTGCCACCTGGATTCTGCGCGGGACGTCCTTCTGCTACGTCCCTTCGGCCCTCAATCC |
| | AGCGGACCTTCCTTCCCGCGGCCTGCTGCCGGCTCTGCGGCCTCTTCCGCGTCTTCGC |
| | CTTCGCCCTCAGACGAGTCGGATCTCCCTTTGGGCCGCCTCCCCGCAtcattgcCTGCCCG |
| | GGTGGCATCCCTGTGACCCCTCCCCAGTGCCTCTCCTGGCCCTGGAAGTTGCCACTCCA |
| | GTGCCCACCAGCCTTGTCCTAATAAAATTAAGTTGCATCATTTTGTCTGACTAGGTGTCCT |
| | TCTATAATATTATGGGGTGGAGGGGGGTGGTATGGAGCAAGGGGCCCAAGTTGGGAAG |
| | AAACCTGTAGGGCCTGC |
| 96 | CTCAGTCCATTAGGAGCCAGTAGCCTGGAAGATGTCTTTACCCCCAGCATCAGTTCAAGT |
| | GGAGCAGCACATAACTCTTGCCCTCTGCCTTCCAAGATTCTGGTGCTGAGACTTATGGA |
| | GTGTCTTGGAGGTTGCCTTCTGCCCCCCAACCCTGCTCCCAGCTGGCCCTCCCAGGCCT |
| | GGGTTGCTGGCCTCTGCTTTATCAGGATTCTCAAGAGGGACAGCTGGTTTATGTTGCAT |
| | GACTGTTCCCTGCATATCTGCTCTGGTTTTAAATAGCTTATCTGAGCAGCTGGAGGACCA |
| | CATGGGCTTATATGGCGTGGGGTACATGTTCCTGTAGCCTTGTCCCTGGCACCTGCCAA |
| | AATAGCAGCCAACACCCCCCACCCCCACCGCCATCCCCCTGCCCCACCCGTCCCCTGT |
| | CGCACATTCCTCCCTCCGCAGGGCTGGCTCACCAGGCCCCAGCCCACATGCCTGCTTA |
| | AAGCCCTCTCCATCCTCTGCCTCACCCAGTCCCCGCTGAGACTGAGCAGACGCCTCCAG |
| | GATCTGTCGGCAGGCCACCATGGCAGCCCCCGGCGCCCCAGCTGAGTACGGCTACATC |
| | CGGACCGTCCTGGGCCAGCAGATCCTGGGACAACTGGACAGCTCCAGCCTGGCGCTGC |
| | CCTCCGAGGCCAAGCTGAAGCTGGCGGGGAGCAGCGGCCGCGGCGGCCAGACAGTCA |
| | AGAGCCTGCGGATCCAGGAGCAGGTGCAGCAGACCCTCGCCCGGAAGGGCCGCAGCT |
| | CCGTGGGCAACGGAAATCTTCACCGAACCAGCAGTGTTCCTGAGTATGTCTACAACCTA |
| | CACTTGGTTGAAAATGATTTTGTTGGAGGCCGTTCCCCTGTTCCTAAAACCTATGACATG |
| | CTAAAGGCTGGCACAACTGCCACTTATGAAGGTCGCTGGGGAAGAGGAACAGCACAGTA |
| | CAGCTCCCAGAAGTCCGTGGAAGAAAGGTCCTTGAGGCATCCTCTGAGGAGACTGGAG |
| | ATTTCTCCTGACAGCAGCCCGGAGAGGGCTCACTACACGCACAGCGATTACCAGTACAG |
| | CCAGAGAAGCCAGGCTGGGCACACCCTGCACCACCAAGAAAGCAGGCGGGCCGCCCT |
| | CCTAGTGCCACCGAGATATGCTCGTTCCGAGATCGTGGGGGTCAGCCGTGCTGGCACC |
| | ACAAGCAGGCAGCGCCACTTTGACACATACCACAGACAGTACCAGCATGGCTCTGTTAG |
| | CGACACCGTTTTTGACAGCATCCCTGCCAACCCGGCCCTGCTCACGTACCCCAGGCCAG |
| | GGACCAGCCGCAGCATGGGCAACCTCTTGGAGAAGGAGAACTACCTGACGGCAGGGCT |
| | CACTGTCGGGCAGGTCAGGCCGCTGGTGCCCCTGCAGCCCGTCACTCAGAACAGGGCT |
| | TCCAGGTCCTCCTGGCATCAGAGCTCCTTCCACAGCACCCGCACGCTGAGGGAAGCTG |
| | GGCCCAGTGTCGCCGTGGATTCCAGCGGGAGGAGAGCGCACTTGACTGTCGGCCAGG |
| | CGGCCGCAGGGGGAAGTGGGAATCTGCTCACTGAGAGAAGCACTTTCACTGACTCCCA |
| | GCTGGGGAATGCAGACATGGAGATGACTCTGGAGCGAGCAGTGAGTATGCTCGAGGCA |
| | GACCACATGCTGCCATCCAGGATTTCTGCTGCAGCTACTTTCATACAGCACGAGTGCTTC |
| | CAGAAATCTGAAGCTCGGAAGAGGGTTAACCAGCTTCGTGGCATCCTCAAGCTTCTGCA |
| | GCTCCTAAAAGTTCAGAATGAAGACGTTCAGCGAGCTGTGTGTGGGGCCTTGAGAAACT |
| | TAGTATTTGAAGACAATGACAACAAATTGGAGGTGGCTGAACTAAATGGGGTACCTCGG |
| | CTGCTCCAGGTGCTGAAGCAAACCAGAGACTTGGAGACTAAAAAACAAATAACAGACCA |
| | TACAGTCAATTTAAGAAGTAGGAATGGCTGGCCGGGCGCGGTGGCTCACGCCTGTAATC |
| | CCAGCACTTTGGGAGGCCAAGGCGGGCGGATCACGAGGTCAGGAGTTCGAGACCAGC |
| | CTGACCAACATGGTTTGCTGTGGAATTTGTCATCTAATGACAAACTCAAGAATCTCATGAT |
| | AACAGAAGCATTGCTTACGCTGACGGAGAATATCATCATCCCCTTTTCTGGGTGGCCTGA |
| | AGGAGACTACCCAAAAGCAAATGGTTTGCTCGATTTTGACATATTCTACAACGTCACTGG |
| | ATGCCTAAGAAACATGAGTTCTGCTGGCGCTGATGGGAGAAAAGCGATGAGAAGATGTG |
| | ACGGACTCATTGACTCACTGGTCCATTATGTCAGAGGAACCATTGCAGATTACCAGCCAG |
| | ATGACAAGGCCACGGAGAATTGTGTGTGCATTCTTCATAACCTCTCCTACCAGCTGGAG |
| | GCAGAGCTCCCAGAGAAATATTCCCAGAATATCTATATTCAAAACCGGAATATCCAGACT |
| | GACAACAACAAAAGTATTGGATGTTTTGGCAGTCGAAGCAGGAAAGTAAAAGAGCAATAC |
| | CAGGACGTGCCGATGCCGGAGGAAAAGAGCAACCCCAAGGGCGTGGAGTGGCTGTGG |
| | CATTCCATTGTTATAAGGATGTATCTGTCCTTGATCGCCAAAAGTGTCCGCAACTACACA |
| | CAAGAAGCATCCTTAGGAGCTCTGCAGAACCTCACGGCCGGAAGTGGACCAATGCCGA |
| | CATCAGTGGCTCAGACAGTTGTCCAGAAGGAAAGTGGCCTGCAGCACACCCGAAAGATG |
| | CTGCATGTTGGTGACCCAAGTGTGAAAAAGACAGCCATCTCGCTGCTGAGGAATCTGTC |
| | CCGGAATCTTTCTCTGCAGAATGAAATTGCCAAAGAAACTCTCCCTGATTTGGTTTCCAT |
| | CATTCCTGACACAGTCCCGAGTACTGACCTTCTCATTGAAACTACAGCCTCTGCCTGTTA |
| | CACATTGAACAACATAATCCAAAACAGTTACCAGAATGCACGCGACCTTCTAAACACCGG |
| | GGGCATCCAGAAAATTATGGCCATTAGTGCAGGCGATGCCTATGCCTCCAACAAAGCAA |
| | GTAAAGCTGCTTCCGTCCTTCTGTATTCTCTGTGGGCACACACGGAACTGCATCATGCCT |
| | ACAAGAAGGCTCAGTTTAAGAAGACAGATTTTGTCAACAGCCGGACTGCCAAAGCCTAC |
| | CACTCCCTTAAAGACTGATCAACCTCTGGATTACAAAATTTGTGAAAGATTGACTGGTATT |
| | CTTAACTATGTTGCTCCTTTTACGCTATGTGGATACGCTGCTTTAATGCCTTTGTATCATG |
| | CTATTGCTTCCCGTATGGCTTTCATTTTCTCCTCCTTGTATAAATCCTGGTTGCTGTCTCT |
| | TTATGAGGAGTTGTGGCCCGTTGTCAGGCAACGTGGCGTGGTGTGCACTGTGTTTGCTG |
| | ACGCAACCCCCACTGGTTGGGGCATTGCCACCACCTGTCAGCTCCTTTCCGGGACTTTC |
| | GCTTTCCCCCTCCCTATTGCCACGGCGGAACTCATCGCCGCCTGCCTTGCCCGCTGCTG |
| | GACAGGGGCTCGGCTGTTGGGCACTGACAATTCCGTGGTGTTGTCGGGGAAATCATCG |
| | TCCTTTCCTTGGCTGCTCGCCTGTGTTGCCACCTGGATTCTGCGCGGGACGTCCTTCTG |
| | CTACGTCCCTTCGGCCCTCAATCCAGGGGACCTTCCTTCCCGCGGCCTGCTGCCGGCT |
| | CTGCGGCCTCTTCCGCGTCTTCGCCTTCGCCCTCAGACGAGTCGGATCTCCCTTTGGGC |
| | CGCCTCCCCGCAtcattgcCTGCCCGGGTGGCATCCCTGTGACCCCTCCCCAGTGCCTCTC |
| | CTGGCCCTGGAAGTTGCCACTCCAGTGCCCACCAGCCTTGTCCTAATAAAATTAAGTTGC |
| | ATCATTTTGTCTGACTAGGTGTCCTTCTATAATATTATGGGGTGGAGGGGGGTGGTATGG |
| | AGCAAGGGGCCCAAGTTGGGAAGAAACCTGTAGGGCCTGC |
| 97 | gcgcgctcgctcgctcactgaggccgcccgggcaaagcccgggcgtcgggcgacctttggtcgcccggcctcagtgagcgagc |
| | gagcgcgcagagagggagtggccaactccatcactaggggttccttgtagttaatgattaacccgccatgctacttatctacgta |
| | ctctggagacgcgtctcagtccattaggagccagtagcctggaagatgtctttacccccagcatcagttcaagtggagcagcaca |
| | taactcttgccctctgccttccaagattctggtgctgagacttatggagtgtcttggaggttgccttctgccccccaaccctgct |
| | cccagctggccctcccaggcctgggttgctggcctctgctttatcaggattctcaagagggacagctggtttatgttgcatgact |
| | gttccctgcatatctgctctggttttaaatagcttatctgagcagctggaggaccacatgggcttatatggcgtggggtacatgt |
| | tcctgtagccttgtccctggcacctgccaaaatagcagccaacaccccccacccccaccgccatccccctgccccacccgtcccc |
| | tgtcgcacattcctccctccgcagggctggctcaccaggccccagcccacatgcctgcttaaagccctctccatcctctgcctca |
| | cccagtccccgctgagactgagcagacgcctccaggatctgtcggcaggccaccatggcagcccccggcgccccagctgagtacg |
| | gctacatccggaccgtcctgggccagcagatcctgggacaactggacagctccagcctggcgctgccctccgaggccaagctgaa |
| | gctggcggggagcagcggccgcggcggccagacagtcaagagcctgcggatccaggagcaggtgcagcagaccctcgcccggaag |
| | ggccgcagctccgtgggcaacggaaatcttcaccgaaccagcagtgttcctgagtatgtctacaacctacacttggttgaaaatg |
| | attttgttggaggccgttcccctgttcctaaaacctatgacatgctaaaggctggcacaactgccacttatgaaggtcgctgggg |
| | aagaggaacagcacagtacagctcccagaagtccgtggaagaaaggtccttgaggcatcctctgaggagactggagatttctcct |
| | gacagcagcccggagagggctcactacacgcacagcgattaccagtacagccagagaagccaggctgggcacaccctgcaccacc |
| | aagaaagcaggcgggccgccctcctagtgccaccgagatatgctcgttccgagatcgtgggggtcagccgtgctggcaccacaag |
| | caggcagcgccactttgacacataccacagacagtaccagcatggctctgttagcgacaccgtttttgacagcatccctgccaac |
| | ccggccctgctcacgtaccccaggccagggaccagccgcagcatgggcaacctcttggagaaggagaactacctgacggcagggc |
| | tcactgtcgggcaggtcaggccgctggtgcccctgcagcccgtcactcagaacagggcttccaggtcctcctggcatcagagctc |
| | cttccacagcacccgcacgctgagggaagctgggcccagtgtcgccgtggattccagcgggaggagagcgcacttgactgtcggc |
| | caggcggccgcagggggaagtgggaatctgctcactgagagaagcactttcactgactcccagctggggaatgcagacatggaga |
| | tgactctggagcgagcagtgagtatgctcgaggcagaccacatgctgccatccaggatttctgctgcagctactttcatacagca |
| | cgagtgcttccagaaatctgaagctcggaagagggttaaccagcttcgtggcatcctcaagcttctgcagctcctaaaagttcag |
| | aatgaagacgttcagcgagctgtgtgtggggccttgagaaacttagtatttgaagacaatgacaacaaattggaggtggctgaac |
| | taaatggggtacctcggctgctccaggtgctgaagcaaaccagagacttggagactaaaaaacaaataacaggtttgctgtggaa |
| | tttgtcatctaatgacaaactcaagaatctcatgataacagaagcattgcttacgctgacggagaatatcatcatccccttttct |
| | gggggcctgaaggagactacccaaaagcaaatggtttgctcgattttgacatattctacaacgtcactggatgcctaagaaacat |
| | gagttctgctggcgctgatgggagaaaagcgatgagaagatgtgacggactcattgactcactggtccattatgtcagaggaacc |
| | attgcagattaccagccagatgacaaggccacggagaattgtgtgtgcattcttcataacctctcctaccagctggaggcagagc |
| | tcccagagaaatattcccagaatatctatattcaaaaccggaatatccagactgacaacaacaaaagtattggatgttttggcag |
| | tcgaagcaggaaagtaaaagagcaataccaggacgtgccgatgccggaggaaaagagcaaccccaagggcgtggagtggctgtgg |
| | cattccattgttataaggatgtatctgtccttgatcgccaaaagtgtccgcaactacacacaagaagcatccttaggagctctgc |
| | agaacctcacggccggaagtggaccaatgccgacatcagtggctcagacagttgtccagaaggaaagtggcctgcagcacacccg |
| | aaagatgctgcatgttggtgacccaagtgtgaaaaagacagccatctcgctgctgaggaatctgtcccggaatctttctctgcag |
| | aatgaaattgccaaagaaactctccctgatttggtttccatcattcctgacacagtcccgagtactgaccttctcattgaaacta |
| | cagcctctgcctgttacacattgaacaacataatccaaaacagttaccagaatgcacgcgaccttctaaacaccgggggcatcca |
| | gaaaattatggccattagtgcaggcgatgcctatgcctccaacaaagcaagtaaagctgcttccgtccttctgtattctctgtgg |
| | gcacacacggaactgcatcatgcctacaagaaggctcagtttaagaagacagattttgtcaacagccggactgccaaagcctacc |
| | actcccttaaagactgatcaacctctggattacaaaatttgtgaaagattgactggtattcttaactatgttgctccttttacgc |
| | tatgtggatacgctgctttaatgcctttgtatcatgctattgcttcccgtatggctttcattttctcctccttgtataaatcctg |
| | gttgctgtctctttatgaggagttgtggcccgttgtcaggcaacgtggcgtggtgtgcactgtgtttgctgacgcaacccccact |
| | ggttggggcattgccaccacctgtcagctcctttccgggactttcgctttccccctccctattgccacggcggaactcatcgccg |
| | cctgccttgcccgctgctggacaggggctcggctgttgggcactgacaattccgtggtgttgtcggggaaatcatcgtcctttcc |
| | ttggctgctcgcctgtgttgccacctggattctgcgcgggacgtccttctgctacgtcccttcggccctcaatccagcggacctt |
| | ccttcccgcggcctgctgccggctctgcggcctcttccgcgtcttcgccttcgccctcagacgagtcggatctccctttgggccg |
| | cctccccgcatcattgcctgcccgggggcatccctgtgacccctccccagtgcctctcctggccctggaagttgccactccagtg |
| | cccaccagccttgtcctaataaaattaagttgcatcattttgtctgactaggtgtccttctataatattatggggtggagggggg |
| | tggtatggagcaaggggcccaagttgggaagaaacctgtagggcctgcgttacccaggctggagtgcagtggcacatttctgctc |
| | actgcaacctcctcctccctgggttctacgtagataagtagcatgggggttaatcattaactacaaggaacccctagtgatggag |
| | ttggccactccctctctgcgcgctcgctcgctcactgaggccgggcgaccaaaggtcgcccgacgcccgggctttgcccgggcgg |
| | cctcagtgagcgagcgagcgcgc |
| 98 | gcgcgctcgctcgctcactgaggccgcccgggcaaagcccgggcgtcgggcgacctttggtcgcccggcctcagtgagcgagc |
| | gagcgcgcagagagggagtggccaactccatcactaggggttccttgtagttaatgattaacccgccatgctacttatctacgtac |
| | tctggagacgcgtacccttcagattaaaaataactgaggtaagggcctgggtaggggaggtggtgtgagacgctcctgtctctcct |
| | tctactgcccatcggccctttggggaggaggaatgtgcccaaggactaaaaaaaggccatggagccagaggggcgagggcaaca |
| | gacctttcatgggcaaaccttggggccctgctgtctagcatgccccactacgggtctaggctgcccatgtaaggaggcaaggcctg |
| | gggacacccgagatgcctggttataattaacccagacatgtggctgcccccccccccccaacacctgctgcctctaaaaataacc |
| | ctgtccctggtggatcccctgcatgcgaagatcttcgaacaaggctgtgggggactgagggcaggctgtaacaggcttgggggcc |
| | agggcttatacgtgcctgggactcccaaagtattactgttccatgttcccggcgaagggccagctgtcccccgccagctagactca |
| | gcacttagtttaggaaccagtgagcaagtcagcccttggggcagcccatacaaggccatggggctgggcaagctgcacgcctggg |
| | tccgggggggcacggtgcccgggcaacgagctgaaagctcatctgctctcaggggcccctccctggggacagcccctcctggc |
| | tagtcacaccctgtaggctcctctatataacccaggggcacaggggctgccctcattctaccaccacctccacagcacagacaga |
| | cactcaggagccagccaggccaccatggcagcccccggcgccccagctgagtacggctacatccggaccgtcctgggccagc |
| | agatcctgggacaactggacagctccagcctggcgctgccctccgaggccaagctgaagctggcggggagcagcggccgcg |
| | gcggccagacagtcaagagcctgcggatccaggagcaggtgcagcagaccctcgcccggaagggccgcagctcgtgggc |
| | aacggaaatcttcaccgaaccagcagtgttcctgagtatgtctacaacctacacttggttgaaaatgattttgttggaggccgttc |
| | ccctgttcctaaaacctatgacatgctaaaggctggcacaactgccacttatgaaggtcgctggggaagaggaacagcacagtaca |
| | gctcccagaagtccgtggaagaaaggtccttgaggcatcctctgaggagactggagatttctcctgacagcagcccggagagggc |
| | tcactacacgcacagcgattaccagtacagccagagaagccaggctgggcacaccctgcaccaccaagaaagcaggcgggc |
| | cgccctcctagtgccaccgagatatgctcgttccgagatcgtgggggtcagccgtgctggcaccacaagcaggcagcgccacttt |
| | gacacataccacagacagtaccagcatggctctgttagcgacaccgtttttgacagcatccctgccaacccggccctgctcacgta |
| | ccccaggccagggaccagccgcagcatgggcaacctcttggagaaggagaactacctgacggcagggctcactgtcgggca |
| | ggtcaggccgctggtgcccctgcagcccgtcactcagaacagggcttccaggtcctcctggcatcagagctccttccacagcacc |
| | cgcacgctgagggaagctgggcccagtgtcgccgtggattccagcgggaggagagcgcacttgactgtcggccaggcggccg |
| | cagggggaagtgggaatctgctcactgagagaagcactttcactgactcccagctggggaatgcagacatggagatgactctgg |
| | agcgagcagtgagtatgctcgaggcagaccacatgctgccatccaggatttctgctgcagctactttcatacagcacgagtgcttc |
| | cagaaatctgaagctcggaagagggttaaccagcttgtggcatcctcaagcttctgcagctcctaaaagttcagaatgaagacgtt |
| | cagcgagctgtgtgtggggccttgagaaacttagtatttgaagacaatgacaacaaattggaggtggctgaactaaatggggtacc |
| | tcggctgctccaggtgctgaagcaaaccagagacttggagactaaaaaacaaataacagaccatacagtcaatttaagaagta |
| | ggaatggctggccgggcgcggtggctcacgcctgtaatcccagcactttgggaggccaaggcgggcggatcacgaggtcagg |
| | agttcgagaccagcctgaccaacatggtttgctgtggaatttgtcatctaatgacaaactcaagaatctcatgataacagaagcat |
| | tgcttacgctgacggagaatatcatcatccccttttctgggtggcctgaaggagactacccaaaagcaaatggtttgctcgatttt |
| | gacatattctacaacgtcactggatgcctaagaaacatgagttctgctggcgctgatgggagaaaagcgatgagaagatgtgacgg |
| | actcattgactcactggtccattatgtcagaggaaccattgcagattaccagccagatgacaaggccacggagaattgtgtgtgca |
| | ttcttcataacctctcctaccagctggaggcagagctcccagagaaatattcccagaatatctatattcaaaaccggaatatccag |
| | actgacaacaacaaaagtattggatgttttggcagtcgaagcaggaaagtaaaagagcaataccaggacgtgccgatgccggagga |
| | aaagagcaaccccaagggcgtggagtggctgtggcattccattgttataaggatgtatctgtccttgatcgccaaaagtgtccgca |
| | actacacacaagaagcatccttaggagctctgcagaacctcacggccggaagtggaccaatgccgacatcagtggctcagacagtt |
| | gtccagaaggaaagtggcctgcagcacacccgaaagatgctgcatgttggtgacccaagtgtgaaaaagacagccatctcgct |
| | gctgaggaatctgtcccggaatctttctctgcagaatgaaattgccaaagaaactctccctgatttggtttccatcattcctgaca |
| | cagtcccgagtactgaccttctcattgaaactacagcctctgcctgttacacattgaacaacataatccaaaacagttaccagaat |
| | gcacgcgaccttctaaacaccgggggcatccagaaaattatggccattagtgcaggcgatgcctatgcctccaacaaagcaagtaa |
| | agctgcttccgtccttctgtattctctgtgggcacacacggaactgcatcatgcctacaagaaggctcagtttaagaagacagatt |
| | ttgtcaacagccggactgccaaagcctaccactcccttaaagactgatcaacctctggattacaaaatttgtgaaagattgactgg |
| | tattcttaactatgttgctccttttacgctatgtggatacgctgctttaatgcctttgtatcatgctattgcttcccgtatggctt |
| | tcattttctcctccttgtataaatcctggttgctgtctctttatgaggagttgtggcccgttgtcaggcaacgtggcgtggtgtgc |
| | actgtgtttgctgacgcaacccccactggttggggcattgccaccacctgtcagctcctttccgggactttcgctttccccctccc |
| | tattgccacggcggaactcatcgccgcctgccttgcccgctgctggacaggggctcggctgttgggcactgacaattccgtggtgt |
| | tgtcggggaaatcatcgtcctttccttggctgctcgcctgtgttgccacctggattctgcgcgggacgtccttctgctacgtccct |
| | tcggccctcaatccagcggaccttccttcccgcggcctgctgccggctctgcggcctcttccgcgtcttcgccttcgccctcagac |
| | gagtcggatctccctttgggccgcctccccgcatcattgcctgcccgggtggcatccctgtgacccctccccagtgcctctcctgg |
| | ccctggaagttgccactccagtgcccaccagccttgtcctaataaaattaagttgcatcattttgtctgactaggtgtccttctat |
| | aatattatggggtggaggggggtggtatggagcaaggggcccaagttgggaagaaacctgtagggcctgcgttacccaggctgga |
| | acgtagataagtagcatgggggttaatcattaactacaaggaacccctagtgatggagttggccactccctctctgcgcgctcgct |
| | gtgcagtggcacatttctgctcactgcaacctcctcctccctgggttctcgctcactgaggccgggcgaccaaaggtcgcccgacg |
| | cccgggctttgcccgggcggcctcagtgagcgagcgagcgcgc |
| 99 | gcgcgctcgctcgctcactgaggccgcccgggcaaagcccgggcgtcgggcgacctttggtcgcccggcctcagtgagcgagc |
| | gagcgcgcagagagggagtggccaactccatcactaggggttccttgtagttaatgattaacccgccatgctacttatctacgtac |
| | tctggagacgcgtctcagtccattaggagccagtagcctggaagatgtctttacccccagcatcagttcaagtggagcagcacata |
| | actcttgccctctgccttccaagattctggtgctgagacttatggagtgtcttggaggttgccttctgccccccaaccctgctccc |
| | agctggccctcccaggcctgggttgctggcctctgctttatcaggattctcaagagggacagctggtttatgttgcatgactgttc |
| | cctgcatatctgctctggttttaaatagcttatctgagcagctggaggaccacatgggcttatatggcgtggggtacatgttcctg |
| | tagccttgtccctggcacctgccaaaatagcagccaacaccccccacccccaccgccatccccctgccccacccgtcccctgtcgc |
| | acattcctccctccgcagggctggctcaccaggccccagcccacatgcctgcttaaagccctctccatcctctgcctcacccagtc |
| | cccgctgagactgagcagacgcctccaggatctgtcggcaggccaccatggcagcccccggcgccccagctgagtacggctacatc |
| | cggaccgtcctgggccagcagatcctgggacaactggacagctccagcctggcgctgccctccgaggccaagctgaagctgggggg |
| | agcagcggccgcggcggccagacagtcaagagcctgcggatccaggagcaggtgcagcagaccctcgcccggaagggccgcagctc |
| | cgtgggcaacggaaatcttcaccgaaccagcagtgttcctgagtatgtctacaacctacacttggttgaaaatgattttgttggag |
| | gccgttcccctgttcctaaaacctatgacatgctaaaggctggcacaactgccacttatgaaggtcgctggggaagaggaacagca |
| | cagtacagctcccagaagtccgtggaagaaaggtccttgaggcatcctctgaggagactggagatttctcctgacagcagcccgg |
| | agagggctcactacacgcacagcgattaccagtacagccagagaagccaggctgggcacaccctgcaccaccaagaaagc |
| | aggcgggccgccctcctagtgccaccgagatatgctcgttccgagatcgtgggggtcagccgtgctggcaccacaagcaggca |
| | gcgccactttgacacataccacagacagtaccagcatggctctgttagcgacaccgtttttgacagcatccctgccaacccggccc |
| | tgctcacgtaccccaggccagggaccagccgcagcatgggcaacctcttggagaaggagaactacctgacggcagggctcac |
| | tgtcgggcaggtcaggccgctggtgcccctgcagcccgtcactcagaacagggcttccaggtcctcctggcatcagagctccttcc |
| | acagcacccgcacgctgagggaagctgggcccagtgtcgccgtggattccagcgggaggagagcgcacttgactgtcggcca |
| | ggcggccgcagggggaagtgggaatctgctcactgagagaagcactttcactgactcccagctggggaatgcagacatggaga |
| | tgactctggagcgagcagtgagtatgctcgaggcagaccacatgctgccatccaggatttctgctgcagctactttcatacagcac |
| | gagtgcttccagaaatctgaagctcggaagagggttaaccagcttcgtggcatcctcaagcttctgcagctcctaaaagttcagaa |
| | tgaagacgttcagcgagctgtgtgtggggccttgagaaacttagtatttgaagacaatgacaacaaattggaggtggctgaactaa |
| | atggggtacctcggctgctccaggtgctgaagcaaaccagagacttggagactaaaaaacaaataacagaccatacagtcaattta |
| | agaagtaggaatggctggccgggcgcggtggctcacgcctgtaatcccagcactttgggaggccaaggcgggcggatcacga |
| | ggtcaggagttcgagaccagcctgaccaacatggtttgctgtggaatttgtcatctaatgacaaactcaagaatctcatgataaca |
| | gaagcattgcttacgctgacggagaatatcatcatccccttttctgggggcctgaaggagactacccaaaagcaaatggtttgctc |
| | gattttgacatattctacaacgtcactggatgcctaagaaacatgagttctgctggcgctgatgggagaaaagcgatgagaagatg |
| | tgacggactcattgactcactggtccattatgtcagaggaaccattgcagattaccagccagatgacaaggccacggagaattgtg |
| | tgtgcattcttcataacctctcctaccagctggaggcagagctcccagagaaatattcccagaatatctatattcaaaaccggaat |
| | atccagactgacaacaacaaaagtattggatgttttggcagtcgaagcaggaaagtaaaagagcaataccaggacgtgccgatgcc |
| | ggaggaaaagagcaaccccaagggcgtggagtggctgtggcattccattgttataaggatgtatctgtccttgatcgccaaaagtg |
| | tccgcaactacacacaagaagcatccttaggagctctgcagaacctcacggccggaagtggaccaatgccgacatcagtggctc |
| | agacagttgtccagaaggaaagtggcctgcagcacacccgaaagatgctgcatgttggtgacccaagtgtgaaaaagacagcc |
| | atctcgctgctgaggaatctgtcccggaatctttctctgcagaatgaaattgccaaagaaactctccctgatttggtttccatcat |
| | tcctgacacagtcccgagtactgaccttctcattgaaactacagcctctgcctgttacacattgaacaacataatccaaaacagtt |
| | accagaatgcacgcgaccttctaaacaccgggggcatccagaaaattatggccattagtgcaggcgatgcctatgcctccaacaaa |
| | gcaagtaaagctgcttccgtccttctgtattctctgtgggcacacacggaactgcatcatgcctacaagaaggctcagtttaagaa |
| | gacagattttgtcaacagccggactgccaaagcctaccactcccttaaagactgatcaacctctggattacaaaatttgtgaaaga |
| | ttgactggtattcttaactatgttgctccttttacgctatgtggatacgctgctttaatgcctttgtatcatgctattgcttcccg |
| | tatggctttcattttctcctccttgtataaatcctggttgctgtctctttatgaggagttgtggcccgttgtcaggcaacgtggcg |
| | tggtgtgcactgtgtttgctgacgcaacccccactggttggggcattgccaccacctgtcagctcctttccgggactttcgctttc |
| | cccctccctattgccacggcggaactcatcgccgcctgccttgcccgctgctggacaggggctcggctgttgggcactgacaattc |
| | cgtggtgttgtcggggaaatcatcgtcctttccttggctgctcgcctgtgttgccacctggattctgcgcgggacgtccttctgct |
| | acgtcccttcggccctcaatccagcggaccttccttcccgcggcctgctgccggctctgcggcctcttccgcgtcttcgccttcgc |
| | cctcagacgagtcggatctccctttgggccgcctccccgcatcattgcctgcccgggggcatccctgtgacccctccccagtgcct |
| | ctcctggccctggaagttgccactccagtgcccaccagccttgtcctaataaaattaagttgcatcattttgtctgactaggtgtc |
| | cttctataatattatggggtggaggggggggtatggagcaaggggcccaagttgggaagaaacctgtagggcctgcgttacccagg |
| | ctggagtgcagtggcacatttctgctcactgcaacctcctcctccctgggttctacgtagataagtagcatgggggttaatcatta |
| | actacaaggaacccctagtgatggagttggccactccctctctgcgcgctcgctcgctcactgaggccgggcgaccaaaggtcgcc |
| | cgacgcccgggctttgcccgggcggcctcagtgagcgagcgagcgcgc |
| 100 | ctcagtccattaggagccagtagcctggaagatgtctttacccccagcatcagttcaagtggagcagcacataactcttgccctct |
| | gccttccaagattctggtgctgagacttatggagtgtcttggaggttgccttctgccccccaaccctgctcccagctggccctccc |
| | aggcctgggttgctggcctctgctttatcaggattctcaagagggacagctggtttatgttgcatgactgttccctgcatatctg |
| | ctctggttttaaatagcttatctgagcagctggaggaccacatgggcttatatggcgtggggtacatgttcctgtagccttgtccc |
| | tggcacctgccaaaatagcagccaacaccccccacccccaccgccatccccctgccccacccgtcccctgtcgcacattcctccct |
| | ccgcagggctggctcaccaggccccagcccacatgcctgcttaaagccctctccatcctctgcctcacccagtccccgctgagact |
| | gagcagacgcctccaggatctgtcggcaggccaccatggcagcccccggcgccccagctgagtacggctacatccggaccgtcctg |
| | ggccagcagatcctgggacaactggacagctccagcctggcgctgccctccgaggccaagctgaagctggcggggagcagcggccg |
| | cggcggccagacagtcaagagcctgcggatccaggagcaggtgcagcagaccctcgcccggaagggccgcagctccgtgggcaac |
| | ggaaatcttcaccgaaccagcagtgttcctgagtatgtctacaacctacacttggttgaaaatgattttgttggaggccgttcccc |
| | tgttcctaaaacctatgacatgctaaaggctggcacaactgccacttatgaaggtcgctggggaagaggaacagcacagtacagct |
| | cccagaagtccgtggaagaaaggtccttgaggcatcctctgaggagactggagatttctcctgacagcagcccggagagggctca |
| | ctacacgcacagcgattaccagtacagccagagaagccaggctgggcacaccctgcaccaccaagaaagcaggcgggccg |
| | ccctcctagtgccaccgagatatgctcgttccgagatcgtgggggtcagccgtgctggcaccacaagcaggcagcgccactttga |
| | cacataccacagacagtaccagcatggctctgttagcgacaccgtttttgacagcatccctgccaacccggccctgctcacgtacc |
| | ccaggccagggaccagccgcagcatgggcaacctcttggagaaggagaactacctgacggcagggctcactgtcgggcaggt |
| | caggccgctggtgcccctgcagcccgtcactcagaacagggcttccaggtcctcctggcatcagagctccttccacagcacccgc |
| | acgctgagggaagctgggcccagtgtcgccgtggattccagcgggaggagagcgcacttgactgtcggccaggcggccgcag |
| | ggggaagtgggaatctgctcactgagagaagcactttcactgactcccagctggggaatgcagacatggagatgactctggagc |
| | gagcagtgagtatgctcgaggcagaccacatgctgccatccaggatttctgctgcagctactttcatacagcacgagtgcttccag |
| | aaatctgaagctcggaagagggttaaccagcttcgtggcatcctcaagcttctgcagctcctaaaagttcagaatgaagacgttca |
| | gcgagctgtgtgtggggccttgagaaacttagtatttgaagacaatgacaacaaattggaggtggctgaactaaatggggtacctc |
| | ggctgctccaggtgctgaagcaaaccagagacttggagactaaaaaacaaataacaggtttgctgtggaatttgtcatctaatgac |
| | aaactcaagaatctcatgataacagaagcattgcttacgctgacggagaatatcatcatccccttttctgggtggcctgaaggaga |
| | ctacccaaaagcaaatggtttgctcgattttgacatattctacaacgtcactggatgcctaagaaacatgagttctgctggcgctg |
| | atgggagaaaagcgatgagaagatgtgacggactcattgactcactggtccattatgtcagaggaaccattgcagattaccagcca |
| | gatgacaaggccacggagaattgtgtgtgcattcttcataacctctcctaccagctggaggcagagctcccagagaaatattccca |
| | gaatatctatattcaaaaccggaatatccagactgacaacaacaaaagtattggatgttttggcagtcgaagcaggaaagtaaaa |
| | gagcaataccaggacgtgccgatgccggaggaaaagagcaaccccaagggcgtggagtggctgtggcattccattgttataaggat |
| | gtatctgtccttgatcgccaaaagtgtccgcaactacacacaagaagcatccttaggagctctgcagaacctcacggccggaagtg |
| | gaccaatgccgacatcagtggctcagacagttgtccagaaggaaagtggcctgcagcacacccgaaagatgctgcatgttggtg |
| | acccaagtgtgaaaaagacagccatctcgctgctgaggaatctgtcccggaatctttctctgcagaatgaaattgccaaagaaact |
| | ctccctgatttggtttccatcattcctgacacagtcccgagtactgaccttctcattgaaactacagcctctgcctgttacacatt |
| | gaacaacataatccaaaacagttaccagaatgcacgcgaccttctaaacaccgggggcatccagaaaattatggccattagtgcag |
| | gcgatgcctatgcctccaacaaagcaagtaaagctgcttccgtccttctgtattctctgtgggcacacacggaactgcatcatgcc |
| | tacaagaaggctcagtttaagaagacagattttgtcaacagccggactgccaaagcctaccactcccttaaagactgatcaacct |
| | ctggattacaaaatttgtgaaagattgactggtattcttaactatgttgctccttttacgctatgtggatacgctgctttaatgcc |
| | tttgtatcatgctattgcttcccgtatggctttcattttctcctccttgtataaatcctggttgctgtctctttatgaggagttg |
| | tggcccgttgtcaggcaacgtggcgtggtgtgcactgtgtttgctgacgcaacccccactggttggggcattgccaccacctgtca |
| | gctcctttccgggactttcgctttccccctccctattgccacggcggaactcatcgccgcctgccttgcccgctgctggacagggg |
| | ctcggctgttgggcactgacaattccgtggtgttgtcggggaaatcatcgtcctttccttggctgctcgcctgtgttgccacctgg |
| | attctgcgcgggacgtccttctgctacgtcccttcggccctcaatccagcggaccttccttcccgcggcctgctgccggctctgcg |
| | gcctcttccgcgtcttcgccttcgccctcagacgagtcggatctccctttgggccgcctccccgcatcattgcctgcccgggtgg |
| | catccctgtgacccctccccagtgcctctcctggccctggaagttgccactccagtgcccaccagccttgtcctaataaaattaag |
| | ttgcatcattttgtctgactaggtgtccttctataatattatggggtggaggggggtggtatggagcaaggggcccaagttgggaa |
| | gaaacctgtagggcctgc |
| 101 | acccttcagattaaaaataactgaggtaagggcctgggtaggggaggtggtgtgagacgctcctgtctctcctctatctgcccatc |
| | ggccctttggggaggaggaatgtgcccaaggactaaaaaaaggccatggagccagaggggcgagggcaacagacctttcatg |
| | ggcaaaccttggggccctgctgtctagcatgccccactacgggtctaggctgcccatgtaaggaggcaaggcctggggacaccc |
| | gagatgcctggttataattaacccagacatgtggctgcccccccccccccaacacctgctgcctctaaaaataaccctgtccctgg |
| | tggatcccctgcatgcgaagatcttcgaacaaggctgtgggggactgagggcaggctgtaacaggcttgggggccagggcttata |
| | cgtgcctgggactcccaaagtattactgttccatgttcccggcgaagggccagctgtcccccgccagctagactcagcacttagtt |
| | taggaaccagtgagcaagtcagcccttggggcagcccatacaaggccatggggctgggcaagctgcacgcctgggtccggggtg |
| | ggcacggtgcccgggcaacgagctgaaagctcatctgctctcaggggcccctccctggggacagcccctcctggctagtcacac |
| | cctgtaggctcctctatataacccaggggcacaggggctgccctcattctaccaccacctccacagcacagacagacactcagg |
| | agccagccaggccaccatggcagcccccggcgccccagctgagtacggctacatccggaccgtcctgggccagcagatcctg |
| | ggacaactggacagctccagcctggcgctgccctccgaggccaagctgaagctggcggggagcagcggccgcggcggccag |
| | acagtcaagagcctgcggatccaggagcaggtgcagcagaccctcgcccggaagggccgcagctccgtgggcaacggaaat |
| | cttcaccgaaccagcagtgttcctgagtatgtctacaacctacacttggttgaaaatgattttgttggaggccgttcccctgttcc |
| | taaaacctatgacatgctaaaggctggcacaactgccacttatgaaggtcgctggggaagaggaacagcacagtacagctcccag |
| | aagtccgtggaagaaaggtccttgaggcatcctctgaggagactggagatttctcctgacagcagcccggagagggctcactaca |
| | cgcacagcgattaccagtacagccagagaagccaggctgggcacaccctgcaccaccaagaaagcaggcgggccgccctcctag |
| | tgccaccgagatatgctcgttccgagatcgtgggggtcagccgtgctggcaccacaagcaggcagcgccactttgacacatacc |
| | acagacagtaccagcatggctctgttagcgacaccgtttttgacagcatccctgccaacccggccctgctcacgtaccccaggcca |
| | gggaccagccgcagcatgggcaacctcttggagaaggagaactacctgacggcagggctcactgtcgggcaggtcaggccgc |
| | tggtgcccctgcagcccgtcactcagaacagggcttccaggtcctcctggcatcagagctccttccacagcacccgcacgctgag |
| | ggaagctgggcccagtgtcgccgtggattccagcgggaggagagcgcacttgactgtcggccaggcggccgcagggggaagt |
| | gggaatctgctcactgagagaagcactttcactgactcccagctggggaatgcagacatggagatgactctggagcgagcagtg |
| | agtatgctcgaggcagaccacatgctgccatccaggatttctgctgcagctactttcatacagcacgagtgcttccagaaatctga |
| | agctcggaagagggttaaccagcttcgtggcatcctcaagcttctgcagctcctaaaagttcagaatgaagacgttcagcgagctg |
| | tgtgtggggccttgagaaacttagtatttgaagacaatgacaacaaattggaggtggctgaactaaatggggtacctcggctgctc |
| | caggtgctgaagcasaccagagacttggagactaaaaaacaaataacagaccatacagtcaatttaagaagtaggaatggctgg |
| | ccgggcgcggtggctcacgcctgtaatcccagcactttgggaggccaagggggcggatcacgaggtcaggagttcgagacca |
| | gcctgaccaacatggtttgctgtggaatttgtcatctaatgacaaactcaagaatctcatgataacagaagcattgcttacgctga |
| | cggagaatatcatcatccccttttctgggggcctgaaggagactacccaaaagcaaatggtttgctcgattttgacatattctaca |
| | acgtcactggatgcctaagaaacatgagttctgctggcgctgatgggagaaaagcgatgagaagatgtgacggactcattgactc |
| | actggtccattatgtcagaggaaccattgcagattaccagccagatgacaaggccacggagaattgtgtgtgcattcttcataacc |
| | tctcctaccagctggaggcagagctcccagagaaatattcccagaatatctatattcaaaaccggaatatccagactgacaacaac |
| | aaaagtattggatgttttggcagtcgaagcaggaaagtaaaagagcaataccaggacgtgccgatgccggaggaaaagagcaacc |
| | ccaagggcgtggagtggctgtggcattccattgttataaggatgtatctgtccttgatcgccaaaagtgtccgcaactacacacaa |
| | gaagcatccttaggagctctgcagaacctcacggccggaagtggaccaatgccgacatcagtggctcagacagttgtccagaagg |
| | aaagtggcctgcagcacacccgaaagatgctgcatgttggtgacccaagtgtgaaaaagacagccatctcgctgctgaggaatc |
| | tgtcccggaatctttctctgcagaatgaaattgccaaagaaactctccctgatttggtttccatcattcctgacacagtcccgagt |
| | actgaccttctcattgaaactacagcctctgcctgttacacattgaacaacataatccaaaacagttaccagaatgcacgcgacct |
| | tctaaacaccgggggcatccagaaaattatggccattagtgcaggcgatgcctatgcctccaacaaagcaagtaaagctgcttccg |
| | tccttctgtattctctgtgggcacacacggaactgcatcatgcctacaagaaggctcagtttaagaagacagattttgtcaacagc |
| | cggactgccaaagcctaccactcccttaaagactgatcaacctctggattacaaaatttgtgaaagattgactggtattcttaact |
| | atgttgctccttttacgctatgtggatacgctgctttaatgcctttgtatcatgctattgcttcccgtatggctttcattttctcc |
| | tccttgtataaatcctggttgctgtctctttatgaggagttgtggcccgttgtcaggcaacgtggcgtggtgtgcactgtgtttgc |
| | tgacgcaacccccactggttggggcattgccaccacctgtcagctcctttccgggactttcgctttccccctccctattgccacgg |
| | cggaactcatcgccgcctgccttgcccgctgctggacaggggctcggctgttgggcactgacaattccgtggtgttgtcggggaa |
| | atcatcgtcctttccttggctgctcgcctgtgttgccacctggattctgcgcgggacgtccttctgctacgtcccttcggccctc |
| | aatccagcggaccttccttcccgcggcctgctgccggctctgcggcctcttccgcgtcttcgccttcgccctcagacgagtcggat |
| | ctccctttgggccgcctccccgcatcattgcctgcccgggtggcatccctgtgacccctccccagtgcctctcctggccctggaag |
| | ttgccactccagtgcccaccagccttgtcctaataaaattaagttgcatcattttgtctgactaggtgtccttctataatattatg |
| | gggtggaggggggggtatggagcaaggggcccaagttgggaagaaacctgtagggcctgc |
| 102 | ctcagtccattaggagccagtagcctggaagatgtctttacccccagcatcagttcaagtggagcagcacataactcttgccctct |
| | gccttccaagattctggtgctgagacttatggagtgtcttggaggttgccttctgccccccaaccctgctcccagctggccctccc |
| | aggcctgggttgctggcctctgctttatcaggattctcaagagggacagctggtttatgttgcatgactgttccctgcatatctgc |
| | tctggttttaaatagcttatctgagcagctggaggaccacatgggcttatatggcgtggggtacatgttcctgtagccttgtccct |
| | ggcacctgccaaaatagcagccaacaccccccacccccaccgccatccccctgccccacccgtcccctgtcgcacattcctccctc |
| | cgcagggctggctcaccaggccccagcccacatgcctgcttaaagccctctccatcctctgcctcacccagtccccgctgagactg |
| | agcagacgcctccaggatctgtcggcaggccaccatggcagcccccggcgccccagctgagtacggctacatccggaccgtcctgg |
| | gccagcagatcctgggacaactggacagctccagcctggcgctgccctccgaggccaagctgaagctggggggagcagcggccgcg |
| | gcggccagacagtcaagagcctgcggatccaggagcaggtgcagcagaccctcgcccggaagggccgcagctccgtgggcaac |
| | ggaaatcttcaccgaaccagcagtgttcctgagtatgtctacaacctacacttggttgaaaatgattttgttggaggccgttcccc |
| | tgttcctaaaacctatgacatgctaaaggctggcacaactgccacttatgaaggtcgctggggaagaggaacagcacagtacagct |
| | cccagaagtccgtggaagaaaggtccttgaggcatcctctgaggagactggagatttctcctgacagcagcccggagagggctca |
| | ctacacgcacagcgattaccagtacagccagagaagccaggctgggcacaccctgcaccaccaagaaagcagggggccg |
| | ccctcctagtgccaccgagatatgctcgttccgagatcgtgggggtcagccgtgctggcaccacaagcaggcagcgccactttga |
| | cacataccacagacagtaccagcatggctctgttagcgacaccgtttttgacagcatccctgccaacccggccctgctcacgtacc |
| | ccaggccagggaccagccgcagcatgggcaacctcttggagaaggagaactacctgacggcagggctcactgtcgggcaggt |
| | caggccgctggtgcccctgcagcccgtcactcagaacagggcttccaggtcctcctggcatcagagctccttccacagcacccgc |
| | acgctgagggaagctgggcccagtgtcgccgtggattccagcgggaggagagcgcacttgactgtcggccaggcggccgcag |
| | ggggaagtgggaatctgctcactgagagaagcactttcactgactcccagctggggaatgcagacatggagatgactctggagc |
| | gagcagtgagtatgctcgaggcagaccacatgctgccatccaggatttctgctgcagctactttcatacagcacgagtgcttccag |
| | aaatctgaagctcggaagagggttaaccagcttcgtggcatcctcaagcttctgcagctcctaaaagttcagaatgaagacgttca |
| | gcgagctgtgtgtggggccttgagaaacttagtatttgaagacaatgacaacaaattggaggtggctgaactaaatggggtacctc |
| | ggctgctccaggtgctgaagcaaaccagagacttggagactaaaaaacaaataacagaccatacagtcaatttaagaagtagga |
| | atggctggccgggcgcggtggctcacgcctgtaatcccagcactttgggaggccaaggcgggcggatcacgaggtcaggagttc |
| | gagaccagcctgaccaacatggtttgctgtggaatttgtcatctaatgacaaactcaagaatctcatgataacagaagcattgctt |
| | acgctgacggagaatatcatcatccccttttctgggggcctgaaggagactacccaaaagcaaatggtttgctcgattttgacata |
| | ttctacaacgtcactggatgcctaagaaacatgagttctgctggcgctgatgggagaaaagcgatgagaagatgtgacggactcat |
| | tgactcactggtccattatgtcagaggaaccattgcagattaccagccagatgacaaggccacggagaattgtgtgtgcattcttc |
| | ataacctctcctaccagctggaggcagagctcccagagaaatattcccagaatatctatattcaaaaccggaatatccagactgac |
| | aacaacaaaagtattggatgttttggcagtcgaagcaggaaagtaaaagagcaataccaggacgtgccgatgccggaggaaaag |
| | agcaaccccaagggcgtggagtggctgtggcattccattgttataaggatgtatctgtccttgatcgccaaaagtgtccgcaacta |
| | cacacaagaagcatccttaggagctctgcagaacctcacggccggaagtggaccaatgccgacatcagtggctcagacagttgtc |
| | cagaaggaaagtggcctgcagcacacccgaaagatgctgcatgttggtgacccaagtgtgaaaaagacagccatctcgctgct |
| | gaggaatctgtcccggaatctttctctgcagaatgaaattgccaaagaaactctccctgatttggtttccatcattcctgacaca |
| | gtcccgagtactgaccttctcattgaaactacagcctctgcctgttacacattgaacaacataatccaaaacagttaccagaatg |
| | cacgcgaccttctaaacaccgggggcatccagaaaattatggccattagtgcaggcgatgcctatgcctccaacasagcaagtaa |
| | agctgcttccgtccttctgtattctctgtgggcacacacggaactgcatcatgcctacaagaaggctcagtttaagaagacagat |
| | tttgtcaacagccggactgccaaagcctaccactcccttaaagactgatcaacctctggattacaaaatttgtgaaagattgact |
| | ggtattcttaactatgttgctccttttacgctatgtggatacgctgctttaatgcctttgtatcatgctattgcttcccgtatgg |
| | ctttcattttctcctccttgtataaatcctggttgctgtctctttatgaggagttgtggcccgttgtcaggcaacgtggcgtggtg |
| | tgcactgtgtttgctgacgcaacccccactggttggggcattgccaccacctgtcagctcctttccgggactttcgctttccccct |
| | ccctattgccacggcggaactcatcgccgcctgccttgcccgctgctggacaggggctcggctgttgggcactgacaattccgtgg |
| | tgttgtcggggaaatcatcgtcctttccttggctgctcgcctgtgttgccacctggattctgcgcgggacgtccttctgctacgtc |
| | ccttcggccctcaatccagcggaccttccttcccgcggcctgctgccggctctgcggcctcttccgcgtcttcgccttcgccctca |
| | gacgagtcggatctccctttgggccgcctccccgcatcattgcctgcccgggtggcatccctgtgacccctccccagtgcctctc |
| | ctggccctggaagttgccactccagtgcccaccagccttgtcctaataaaattaagttgcatcattttgtctgactaggtgtcctt |
| | ctataatattatggggtggaggggggtggtatggagcaaggggcccaagttgggaagaaacctgtagggcctgc |

1-90. (canceled)

91. A polynucleotide, comprising an expression cassette and flanking adeno-associated virus (AAV) inverted terminal repeats (ITRs), wherein the polynucleotide comprises a polynucleotide sequence encoding a Plakophilin-2 (PKP2), or a functional variant thereof, operatively linked to a promoter, wherein the polynucleotide shares at least 75% identity with any one of SEQ ID NOs: 8-15, 89-96, or 97-102.

92. The polynucleotide of claim 91, wherein the promoter is a cardiac-specific promoter, a muscle specific promoter, or a cardiomyocyte-specific promoter.

93. The polynucleotide of claim 91, wherein the promoter is a Myosin Heavy-chain Creatine Kinase 7 (MHCK7) promoter, wherein the MHCK7 promoter shares at least 75% with SEQ ID NO: 31.

94. The polynucleotide of claim 91, wherein the promoter is a cardiac troponin T (hTNNT2) promoter, wherein the hTNNT2 promoter shares at least 75% identity with SEQ ID NO: 32.

95. The polynucleotide of claim 91, wherein the expression cassette comprises exon 1 of the cardiac troponin T (hTNNT2) gene, wherein the hTNNT2 promoter and exon 1 together share at least 75% identity with SEQ ID NO: 32.

96. The polynucleotide of claim 91, wherein the promoter is a ubiquitous promoter.

97. The polynucleotide of claim 91, wherein the expression cassette comprises a polyA signal, wherein the polyA signal is a human growth hormone (hGH) polyA.

98. The polynucleotide of claim 91, wherein the expression cassette comprises a Woodchuck Hepatitis Virus Posttranscriptional Regulatory Element (WPRE).

99. The polynucleotide of claim 91, wherein the Plakophilin-2 (PKP2) or functional variant thereof is a human PKP2.

100. The polynucleotide of claim 91, wherein the PKP2 is PKP2 isoform A, wherein the PKP2 isoform A shares at least 75% identity with SEQ ID NO: 1.

101. The polynucleotide of claim 91, wherein the PKP2 is PKP2 isoform B, wherein the PKP2 shares at least 75% identity with SEQ ID NO: 2.

102. The polynucleotide of claim 91, wherein the polynucleotide sequence encoding PKP2 shares at least 75% identity with SEQ ID NO: 3 or SEQ ID NO: 4.

103. The polynucleotide of claim 91, wherein the polynucleotide comprises at least 4.0 kb and at most 4.6 kb.

104. The polynucleotide of claim 91, wherein the expression cassette is flanked by 5′ and 3′ inverted terminal repeats (ITRs), wherein the ITRs share at least 75% identity with any one of SEQ ID NO: 20-26.

105. A gene therapy vector, comprising the polynucleotide of claim 104, wherein the gene therapy vector comprises a sequence of any one of SEQ ID NOs: 8-15 89-96, or 97-102.

106. The vector of claim 105, wherein the gene therapy vector is a recombinant adeno-associated virus (rAAV) vector, wherein the rAAV vector is an AAV9 or a functional variant thereof, wherein the rAAV vector comprises a capsid protein that shares at least 90% identity to SEQ ID NO: 77.

107. The vector of claim 105, wherein the rAAV vector is an AAVrh,10 or a functional variant thereof, wherein the rAAV vector comprises a capsid protein that shares at least 90% identity to SEQ ID NO: 79.

108. The vector of claim 105, wherein the rAAV vector is an AAV6 or a functional variant thereof, wherein the rAAV vector comprises a capsid protein that shares at least 90% identity to SEQ ID NO: 78.

109. The vector of claim 105, wherein the rAAV vector is an AAVrh.74 or a functional variant thereof; wherein the rAAV vector comprises a capsid protein that shares at least 90% identity to SEQ ID NOs: 81-83.

110. A method of treating or preventing a disease or disorder in a subject in need thereof, comprising administering the vector of claim 105 to the subject.

111. The method of claim 110, wherein the disease or disorder is:
a) a cardiac disorder;
b) a cardiomyopathy, wherein the cardiomyopathy is arrhythmogenic right ventricular cardiomyopathy (ACM), hypertrophic cardiomyopathy, or dilated cardiomyopathy;
c) a disease or disorder is characterized by fibrofatty infiltration of myocardium; or
d) heart failure.

112. The method of claim 110, wherein the subject is a human.

113. The method of claim 110, wherein the subject has a mutation in a PKP2 gene, wherein the mutation is a STOP-GAIN variant mutation, or wherein the subject has an implantable cardioverter-defibrillator (ICD).

114. The method of claim 110, wherein an effective amount of the vector is to be administered to the subject by intravenous injection, intracardiac injection, intracardiac infusion, or cardiac catheterization.

115. The method of claim 110, wherein the disease or disorder is:
(i) related to or caused by loss of function in PKP2 in the subject; or
(ii) related to or caused by gain of function in PKP2 in the subject.

116. The method of claim 110, wherein the subject has a mutation that causes an amino acid substitution selected from Arg490Trp, Asp26Asn, Thr50_Val51SerfsX60, Arg79X, Tyr86X, GIn133X, Val406SerfsX3, Tyr616X, Trp676X, Cys796Arg, Cys796E, Tyr807X, Glu62Lys, S688P, Trp848X, Y86X, V406X, Y616X, W848X, and Y807X, relative to a human PKP2 gene encoding a human PKP2 having the sequence of SEQ ID NO: 2.

117. The method of claim 110, wherein between 1×10¹¹ vector genomes and 1×10¹⁵ vector genomes of the vector is administered to the subject,
wherein the vector is an AAV9, comprising a sequence having at least 80%, at least 90%, or at least 95% identity to any one of SEQ ID NOs: 8-15, 89-96, or 97-102, comprising a sequence having at least 80%, at least 90%, or at least 95% identity to any one of SEQ ID NOs: 12, 8, 13, 9, 97, 100, 89, 93, 90, or 94, or
wherein the vector is an AAV.rh.74, comprising a sequence having at least 80%, at least 90%, or at least 95% identity to any one of SEQ ID NOs: 8-15, 89-96, or 97-102, comprising a sequence having at least 80%, at least 90%, or at least 95% identity to any one of SEQ ID NOs: 12, 8, 13, 9, 97, 100, 89, 93, 90, or 94.

118. A pharmaceutical composition comprising the vector of claim 105.


SEQUENCE LISTING (102 sequences)

SEQ ID NO: 1 - protein, 837 aa, Homo sapiens
MAAPGAPAEYGYIRTVLGQQILGQLDSSSLALPSEAKLKLAGSSGRGGQTVKSLRIQEQVQQTLARKGRSSVGNGNLHRTSSVPEYVYNLHLVENDFVGGRSPVPKTYDMLKAGTTATYEGRWGRGTAQYSSQKSVEERSLRHPLRRLEISPDSSPERAHYTHSDYQYSQRSQAGHTLHHQESRRAALLVPPRYARSEIVGVSRAGTTSRQRHFDTYHRQYQHGSVSDTVFDSIPANPALLTYPRPGTSRSMGNLLEKENYLTAGLTVGQVRPLVPLQPVTQNRASRSSWHQSSFHSTRTLREAGPSVAVDSSGRRAHLTVGQAAAGGSGNLLTERSTFTDSQLGNADMEMTLERAVSMLEADHMLPSRISAAATFIQHECFQKSEARKRVNQLRGILKLLQLLKVQNEDVQRAVCGALRNLVFEDNDNKLEVAELNGVPRLLQVLKQTRDLETKKQITGLLWNLSSNDKLKNLMITEALLTLTENIIIPFSGWPEGDYPKANGLLDFDIFYNVTGCLRNMSSAGADGRKAMRRCDGLIDSLVHYVRGTIADYQPDDKATENCVCILHNLSYQLEAELPEKYSQNIYIQNRNIQTDNNKSIGCFGSRSRKVKEQYQDVPMPEEKSNPKGVEWLWHSIVIRMYLSLIAKSVRNYTQEASLGALQNLTAGSGPMPTSVAQTVVQKESGLQHTRKMLHVGDPSVKKTAISLLRNLSRNLSLQNEIAKETLPDLVSIIPDTVPSTDLLIETTASACYTLNNIIQNSYQNARDLLNTGGIQKIMAISAGDAYASNKASKAASVLLYSLWAHTELHHAYKKAQFKKTDFVNSRTAKAYHSLKD

SEQ ID NO: 2 - protein, 881 aa, Homo sapiens
MAAPGAPAEYGYIRTVLGQQILGQLDSSSLALPSEAKLKLAGSSGRGGQTVKSLRIQEQVQQTLARKGRSSVGNGNLHRTSSVPEYVYNLHLVENDFVGGRSPVPKTYDMLKAGTTATYEGRWGRGTAQYSSQKSVEERSLRHPLRRLEISPDSSPERAHYTHSDYQYSQRSQAGHTLHHQESRRAALLVPPRYARSEIVGVSRAGTTSRQRHFDTYHRQYQHGSVSDTVFDSIPANPALLTYPRPGTSRSMGNLLEKENYLTAGLTVGQVRPLVPLQPVTQNRASRSSWHQSSFHSTRTLREAGPSVAVDSSGRRAHLTVGQAAAGGSGNLLTERSTFTDSQLGNADMEMTLERAVSMLEADHMLPSRISAAATFIQHECFQKSEARKRVNQLRGILKLLQLLKVQNEDVQRAVCGALRNLVFEDNDNKLEVAELNGVPRLLQVLKQTRDLETKKQITDHTVNLRSRNGWPGAVAHACNPSTLGGQGGRITRSGVRDQPDQHGLLWNLSSNDKLKNLMITEALLTLTENIIIPFSGWPEGDYPKANGLLDFDIFYNVTGCLRNMSSAGADGRKAMRRCDGLIDSLVHYVRGTIADYQPDDKATENCVCILHNLSYQLEAELPEKYSQNIYIQNRNIQTDNNKSIGCFGSRSRKVKEQYQDVPMPEEKSNPKGVEWLWHSIVIRMYLSLIAKSVRNYTQEASLGALQNLTAGSGPMPTSVAQTVVQKESGLQHTRKMLHVGDPSVKKTAISLLRNLSRNLSLQNEIAKETLPDLVSIIPDTVPSTDLLIETTASACYTLNNIIQNSYQNARDLLNTGGIQKIMAISAGDAYASNKASKAASVLLYSLWAHTELHHAYKKAQFKKTDFVNSRTAKAYHSLKD

SEQ ID NO: 3 - DNA, 2511 bp, Homo sapiens
  source 1..2511: mol_type = genomic DNA
atggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacaggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagac

SEQ ID NO: 4 - DNA, 2643 bp, Homo sapiens
  source 1..2643: mol_type = genomic DNA
atggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacagaccatacagtcaatttaagaagtaggaatggctggccgggcgcggtggctcacgcctgtaatcccagcactttgggaggccaaggcgggcggatcacgaggtcaggagttcgagaccagcctgaccaacatggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagac

SEQ ID NO: 5 - DNA, 10 bp, synthetic construct
  source 1..10: mol_type = other DNA
gccaccatgg

SEQ ID NO: 6 - DNA, 2517 bp, synthetic construct
  source 1..2517: mol_type = other DNA
gccaccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacaggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagac

SEQ ID NO: 7 - DNA, 2649 bp, synthetic construct
  source 1..2649: mol_type = other DNA
gccaccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacagaccatacagtcaatttaagaagtaggaatggctggccgggcgcggtggctcacgcctgtaatcccagcactttgggaggccaaggcgggcggatcacgaggtcaggagttcgagaccagcctgaccaacatggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagac

SEQ ID NO: 8 - DNA, 4081 bp, synthetic construct
  source 1..4081: mol_type = other DNA
acccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccaggccaccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacaggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 9 - DNA, 3854 bp, synthetic construct
  source 1..3854: mol_type = other DNA
ctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcaggccaccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacaggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 10 - DNA, 4213 bp, synthetic construct
  source 1..4213: mol_type = other DNA
acccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccaggccaccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacagaccatacagtcaatttaagaagtaggaatggctggccgggcgcggtggctcacgcctgtaatcccagcactttgggaggccaaggcgggcggatcacgaggtcaggagttcgagaccagcctgaccaacatggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 11 - DNA, 4322 bp, synthetic construct
  source 1..4322: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcaggccaccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacagaccatacagtcaatttaagaagtaggaatggctggccgggcgcggtggctcacgcctgtaatcccagcactttgggaggccaaggcgggcggatcacgaggtcaggagttcgagaccagcctgaccaacatggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 12 - DNA, 4417 bp, synthetic construct
  source 1..4417: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtaacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccaggccaccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacaggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 13 - DNA, 4190 bp, synthetic construct
  source 1..4190: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcaggccaccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacaggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 14 - DNA, 4549 bp, synthetic construct
  source 1..4549: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtaacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccaggccaccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacagaccatacagtcaatttaagaagtaggaatggctggccgggcgcggtggctcacgcctgtaatcccagcactttgggaggccaaggcgggcggatcacgaggtcaggagttcgagaccagcctgaccaacatggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 15 - DNA, 3986 bp, synthetic construct
  source 1..3986: mol_type = other DNA
ctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcaggccaccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacagaccatacagtcaatttaagaagtaggaatggctggccgggcgcggtggctcacgcctgtaatcccagcactttgggaggccaaggcgggcggatcacgaggtcaggagttcgagaccagcctgaccaacatggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 16 - RNA, 13 nt, synthetic construct
  source 1..13: mol_type = other RNA
gccgccrccatgg

SEQ ID NO: 17 - skipped

SEQ ID NO: 18 - RNA, 10 nt, synthetic construct
  source 1..10: mol_type = other RNA
gacaccatgg

SEQ ID NO: 19 - skipped

SEQ ID NO: 20 - DNA, 141 bp, unidentified
  source 1..141: mol_type = genomic DNA; note = Adeno-associated virus
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 21 - DNA, 168 bp, unidentified
  source 1..168: mol_type = genomic DNA; note = Adeno-associated virus 2
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgta

SEQ ID NO: 22 - DNA, 170 bp, unidentified
  source 1..170: mol_type = genomic DNA; note = Adeno-associated virus
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgta

SEQ ID NO: 23 - DNA, 145 bp, unidentified
  source 1..145: mol_type = genomic DNA; note = Adeno-associated virus
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 24 - DNA, 141 bp, unidentified
  source 1..141: mol_type = genomic DNA; note = Adeno-associated virus
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 25 - DNA, 168 bp, unidentified
  source 1..168: mol_type = genomic DNA; note = Adeno-associated virus 2
tacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 26 - DNA, 133 bp, unidentified
  source 1..133: mol_type = genomic DNA; note = Adeno-associated virus 2
aggaacccctagtgatggagactccctctctgcgcgctcgctcgctcactgaggccgggcgaccaaaggtcgcccgacgcccgggctttgcccgggcggcctcagtgagcgagcgagcgcgcagagagggagt

SEQ ID NO: 27 - DNA, 124 bp, synthetic construct
  source 1..124: mol_type = other DNA
gcggcaattcagtcgataactataacggtcctaaggtagcgatttaaatacgcgctctcttaaggtagccccgggacgcgtcaattgactacaaaccgagtatctgcagagggccctgcgtatg

SEQ ID NO: 28 - DNA, 84 bp, synthetic construct
  source 1..84: mol_type = other DNA
cttctgaggcggaaagaaccagatcctctcttaaggtagcatcgagatttaaattagggataacagggtaatggcgcgggccgc

SEQ ID NO: 29 - DNA, 63 bp, synthetic construct
  source 1..63: mol_type = other DNA
gttacccaggctggagtgcagtggcacatttctgctcactgcaacctcctcctccctgggttc

SEQ ID NO: 30 - DNA, 573 bp, synthetic construct
  source 1..573: mol_type = other DNA
acttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 31 - DNA, 771 bp, synthetic construct
  source 1..771: mol_type = other DNA
acccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccag

SEQ ID NO: 32 - DNA, 544 bp, Homo sapiens
  source 1..544: mol_type = genomic DNA
ctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcag

SEQ ID NO: 33 - DNA, 502 bp, Homo sapiens
  source 1..502: mol_type = genomic DNA
ctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagt

SEQ ID NO: 34 - DNA, 5464 bp, Mus musculus
  source 1..5464: mol_type = genomic DNA
ggtaccggatcctgcaaggtcacacaagggtctccacccaccaggtgccctagtctcaatttcagtttccatgccttgttctcacaatgctggcctccccagagctaatttggactttgtttttatttcaaaagggcctgaatgaggagtagatcttgtgctacccagctctaagggtgcccgtgaagccctcagacctggagcctttgcaacagccctttaggtggaagcagaataaagcaattttccttaaagccaaaatcctgcctctagactcttcttctctgacctcggtccctgggctctagggtggggaggtggggcttggaagaagaaggtggggaagtggcaaaagccgatccctagggccctgtgaagttcggagccttccctgtacagcactggctcatagatcctcctccagccaaacatagcaagaagtgatacctcctttgtgacttccccaggcccagtacctgtcaggttgaaacaggatttagagaagcctctgaactcacctgaactctgaagctcatccaccaagcaagcacctaggtgccactgctagttagtatcctacgctgataatatgcagagctgggccacagaagtcctggggtgtaggaactgaccagtgacttttcagtcggcaaaggtatgaccccctcagcagatgtagtaatgtccccttagatcccatcccaggcaggtctctaagaggacatgggatgagagatgtagtcatgtggcattccaaacacagctatccacagtgtcccttgccccttccacttagccaggaggacagtaaccttagcctatctttcttcctccccatcctcccaggacacaccccctggtctgcagtattcatttcttccttcacgtcccctctgtgacttccatttgcaaggcttttgacctctgcagctgctggaagatagagtttggccctaggtgtggcaagccatctcaagagaaagcagacaacagggggaccagattttggaaggatcaggaactaaatcactggcgggcctgggggtagaaaaaagagtgagtgagtccgctccagctaagccaagctagtccccgagatactctgccacagctgggctgctcggggtagctttaggaatgtgggtctgaaagacaatgggattggaagacatctctttgagtctcccctcaaccccacctacagacacactcgtgtgtggccagactcctgttcaacagccctctgtgttctgaccactgagctaggcaaccagagcatgggccctgtgctgaggatgaagagttggttaccaatagcaaaaacagcaggggagggagaacagagaacgaaataaggaaggaagaaggaaaggccagtcaatcagatgcagtcagaagagatgggaagccaacacacagcttgagcagaggaaacagaaaagggagagattctgggcataaggaggccacagaaagaagagcccaggccccccaagtctcctctttataccctcatcccgtctcccaattaagcccactcttcttcctagatcagacctgagctgcagcgaagagacccgtagggaggatcacactggatgaaggagatgtgtggagaagtccagggaacctaagagccagagcctaaaagagcaagagataaaggtgcttcaaaggtggccaggctgtgcacacagagggtcgaggactggtggtagagcctcaagataaggatgatgctcagaatgggcggggggggggattctgggggggggagagagaaggtgagaaggagcctggaacagagaatctggaagcgctggaaacgataccataaagggaagaacccaggctacctttagatgtaaatcatgaaagacagggagaagggaagctggagagagtagaaggaccccggggcaagacattgaagcaaggacaagccaggttgagcgctccgtgaaatcagcctgctgaaggcagagccctggtatgagcaccagaacagcagaggctagggttaatgtcgagacagggaacagaaggtagacacaggaacagacagagacgggggagccaggtaacaaaggaatggtccttctcacctgtggccagagcgtccatctgtgtccacatactctagaatgttcatcagactgcagggctggcttgggaggcagctggaaagagtatgtgagagccaggggagacaagggggcctaggaaaggaagaagagggcaaaccaggccacacaagagggcagagcccagaactgagttaactccttccttgttgcatcttccataggaggcagtgggaactctgtgaccaccatcccccatgagcccccactacccataccaagtttggcctgagtggcattctaggttccctgaggacagagcctggcctttgtctcttggacctgacccaagctgacccaatgttctcagtaccttatcatgccctcaagagcttgagaaccaggcagtgacatattaggccatgggctaaccctggagcttgcacacaggagcctcaagtgacctccagggacacagctgcagacaggtggcctttatccccaaagagcaaccatttggcataggtggctgcaaatgggaatgcaaggttgaatcaggtcccttcaagaatactgcatgcaagacctaagacccctggagagaggggtatgctcctgcccccacccaccataaggggagtgaactatcctagggggctggcgaccttggggagacaccacattactgagagtgctgagcccagaaaaactgaccgccctgtgtcctgcccacctccacactctagagctatattgagaggtgacagtagatagggtgggagctggtagcagggagagtgttcctgggtgtgagggtgtaggggaaagccagagcaggggagtctggctttgtctcctgaacacaatgtctacttagttataacaggcatgacctgctaaagacccaacatctacgacctctgaaaagacagcagccctggaggacaggggttgtctctgagccttgggtgcttgatggtgccacaaaggagggcatgagtgtgagtataaggccccaggagcgttagagaagggcacttgggaaggggtcagtctgcagagcccctatccatggaatctggagcctggggccaactggtgtaaatctctgggcctgccaggcattcaaagcagcacctgcatcctctggcagcctggggaggcggaagggagcaaccccccacttataccctttctccctcagccccaggattaacacctctggccttcccccttcccacctcccatcaggagtggagggttgcagagggagggtaaaaacctacatgtccaaacatcatggtgcacgatatatggatcagtatgtgtagaggcaagaaaggaaatctgcaggcttaactgggttaatgtgtaaagtctgtgtgcatgtgtgtgtgtctgactgaaaacgggcatggctgtgcagctgttcagttctgtgcgtgaggttaccagactgcaggtttgtgtgtaaattgcccaaggcaaagtgggtgaatcccttccatggtttaaagagattggatgatggcctgcatctcaaggaccatggaaaatagaatggacactctatatgtgtctctaagctaaggtagcaaggtctttggaggacacctgtctagagatgtgggcaacagagactacagacagtatctgtacagagtaaggagagagaggagggggtgtagaattctcttactatcaaagggaaactgagtcgtgcacctgcaaagtggatgctctccctagacatcatgactttgtctctggggagccagcactgtggaacttcaggtctgagagagtaggaggctcccctcagcctgaagctatgcagatagccagggttgaaagggggaagggagagcctgggatgggagcttgtgtgttggaggcaggggacagatattaagcctggaagagaaggtgacccttacccagttgttcaactcacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtcctcctgtcacctccagagccaagggatcaaaggaggaggagccaggacaggagggaagtgggagggagggtcccagcagaggactccaaatttaggcagcaggcatatgggatgggatataaaggggctggagcactgagagctgtcagagatttctccaacccaggtaagagggagtttcgggtgggggctcttcacccacaccagacctctccccacctagaaggaaactgcctttcctggaagtggggttcaggccggtcagagatctgacagggtggccttccaccagcctgggaagttctcagtggcaggaggtttccacaagaaacactggatgccccttcccttacgctgtcttctccatcttcctcctggggatgctcctccccgtcttggtttatcttggctcttcgtcttcagcaagatttgccctgtgctgtccactccatctttctctactgtctccgtgccttgccttgccttcttgcgtgtccttcctttccacccatttctcacttcaccttttctccccttctcatttgtattcatccttccttccttccttccttccttccttccttccttccttccttcctttctcccttccttccttccttccttccttccttccttccttccttcctgtgtcagagtgctgagaatcacacctggggttcccacccttatgtaaacaatcttccagtgagccacagcttcagtgctgctgggtgctctcttaccttcctcaccccctggcttgtcctgttccatcctggtcaggatctctagattggtctcccagcctctgctactcctcttcctgcctgttcctctctctgtccagctgcgccactgtggtgcctcgttccagctgtggtccacattcttcaggattctctgaaaagttaaccaggtgagaatgtttcccctgtagacagcagatcacgattctcccggaagtcaggcttccagccctctctttctctgcccagctgcccggcactcttagcaaacctcaggcacccttaccccacatagacctctgacagagaagcaggcactttacatggagtcctggtgggagagccataggctacggtgtaaaagaggcagggaagtggtggtgtaggaaagtcaggacttcacatagaagcctagcccacaccagaaatgacagacagatccctcctatctcccccataagagtttgagtcgacccgcggccccgaattg

SEQ ID NO: 35 - DNA, 413 bp, Gallus gallus
  source 1..413: mol_type = genomic DNA
gggataaaagcagtctgggctttcacatgacagcatctggggctgcggcagagggtcgggtccgaagcgctgccttatcagcgtccccagccctgggaggtgacagctggctggcttgtgtcagcccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtggga

SEQ ID NO: 36 - DNA, 1090 bp, Homo sapiens
  source 1..1090: mol_type = genomic DNA
ctctcagccctggaagtccttgctcacagccgaggcgccgagagcgcttgctctgcccagatctgcgcgagtctggcgcccgcgctctgaacggcgtcgctgcccagcccccttccccgggaggtgggagcggccacccagggccccgtggctgcccttgtaaggaggcgaggcccgaggacacccgagacgcccggttataattaaccaggacacgtggcgaacccccctccaacacctgcccccgaacccccccatacccagcgcctcgggtctcggcctttgcggcagaggagacagcaaagcgccctctaaaaataactcctttcccggcgaccgagaccctccctgtcccccgcacagcggaaatctcccagtggcaccgagggggcgagggttaagtgggggggagggtgaccaccgcctcccacccttgccctgagtttgaatctctccaactcagccagcctcagtttcccctccactcagtccctaggaggaaggggcgcccaagcgcgggtttctggggttagactgccctccattgcaattggtccttctcccggcctctgcttcctccagctcacagggtatctgctcctcctggagccacaccttggttccccgaggtgccgctgggactcgggtaggggtgagggcccagggggcacagggggagccgagggccacaggaagggctggtggctgaaggagactcaggggccaggggacggtggcttctacgtgcttgggacgttcccagccaccgtcccatgttcccggcggggggccagctgtccccaccgccagcccaactcagcacttggtcagggtatcagcttggtgggggggcgtgagcccagcccctggggcggctcagcccatacaaggccatggggctgggcgcaaagcatgcctgggttcagggtgggtatggtgcgggagcagggaggtgagaggctcagctgccctccagaactcctccctggggacaacccctcccagccaatagcacagcctaggtccccctatataaggccacggctgctggcccttcctttgggtcagtgtcacctccaggatacagaca

SEQ ID NO: 37 - DNA, 253 bp, Homo sapiens
  source 1..253: mol_type = genomic DNA
gcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagtc

SEQ ID NO: 38 - DNA, 281 bp, Gallus gallus
  source 1..281: mol_type = genomic DNA
ggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggga

SEQ ID NO: 39 - DNA, 220 bp, Human betaherpesvirus 5
  source 1..220: mol_type = genomic DNA
tggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccg

SEQ ID NO: 40 - DNA, 583 bp, Human betaherpesvirus 5
  source 1..583: mol_type = genomic DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgt

SEQ ID NO: 41 - DNA, 508 bp, Human betaherpesvirus 5
  source 1..508: mol_type = genomic DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 42 - DNA, 573 bp, synthetic construct; identical to SEQ ID NO: 30
  source 1..573: mol_type = other DNA

SEQ ID NO: 43 - DNA, 580 bp, synthetic construct
  source 1..580: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 44 - DNA, 455 bp, Homo sapiens
  source 1..455: mol_type = genomic DNA
caacctttggagctaagccagcaatggtagagggaagattctgcacgtcccttccaggcggcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaatagcatgcgtgaggctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgtt

SEQ ID NO: 45-47 - skipped (3 entries)

SEQ ID NO: 48 - DNA, 281 bp, Homo sapiens
  source 1..281: mol_type = genomic DNA
acttgtggacaaagtttgctctattccacctcctccaggccctccttgggtccatcaccccaggggtgctgggtccatcccacccccaggcccacacaggcttgcagtattgtgtgcggtatggtcagggcgtccgagagcaggtttcgcagtggaaggcaggcaggtgttggggaggcagttaccggggcaacgggaacagggcgttttggaggtggttgccatggggacctggatgctgacgaaggctcgcgaggctgtgagcagccacagtgccctgc

SEQ ID NO: 49 - skipped

SEQ ID NO: 50 - DNA, 293 bp, Human betaherpesvirus 5
  source 1..293: mol_type = genomic DNA
acttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattacca

SEQ ID NO: 51 - DNA, 953 bp, Homo sapiens
  source 1..953: mol_type = genomic DNA
cgcgtccgcccgcgagcacagagcctcgcctttgccgatccgccgcccgtccacacccgccgccaggtaagcccggccagccgaccggggcatgcggccgcggcccttcgcccgtgcagagccgccgtctgggccgcagcggggggcgcatggggcggaaccggaccgccgtggggggcgcgggagaagcccctgggcctccggagatgggggacaccccacgccagttcgcaggcgcgaggccgcgctcgggcgggcgcgctccgggggtgccgctctcggggcgggggcaaccggcggggtctttgtctgagccgggctcttgccaatggggatcgcacggtgggcgcggcgtagcccccgtcaggcccggtgggggctggggcgccatgcgcgtgcgcgctggtcctttgggcgctaactgcgtgcgcgctgggaattggcgctaattgcgcgtgcgcgctgggactcaatggcgctaatcgcgcgtgcgttctggggcccgggcgcttgcgccacttcctgcccgagccgctggcgcccgagggtgtggccgctgcgtgcgcgcgcgcgacccggtcgctgtttgaaccgggcggaggcggggctggcgcccggttgggagggggttggggcctggcttcctgccgcgcgccgcggggacgcctccgaccagtgtttgccttttatggtaataacgcggccggcccggcttcctttgtccccaatctgggcgcgcgccggcgccccctggcggcctaaggactcggcgcgccggaagtggccagggcggcagcggctgctcttggcggccccgaggtgactatagccttcttttgtgtcttgatagttcgccagcctctgctaaccatgttcatgccttcttctttttcctacagctcctgggcaacgtgctggttattgtgctgtctcatcattttggcaaagaattc

SEQ ID NO: 52 - DNA, 1068 bp, synthetic construct
  source 1..1068: mol_type = other DNA
gtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattc

SEQ ID NO: 53 - DNA, 149 bp, synthetic construct
  source 1..149: mol_type = other DNA
ggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgc

SEQ ID NO: 54 - DNA, 126 bp, Homo sapiens
  source 1..126: mol_type = genomic DNA
agtctgcggtgggcagcggaggagtcgtgtcgtgcctgagagcgcagctgtgctcctgggcaccgcgcagtccgcccccgcggctcctggccagaccacccctaggaccccctgccccaagtcgca

SEQ ID NO: 55 - DNA, 121 bp, Human betaherpesvirus 5
  source 1..121: mol_type = genomic DNA
tcagatcgcctggagaggccatccacgctgttttgacctccatagtggacaccgggaccgatccagcctccgcggccgggaacggtgcattggaacgcggattccccgtgccaagagtgac

SEQ ID NO: 56 - DNA, 512 bp, synthetic construct
  source 1..512: mol_type = other DNA
ctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccag

SEQ ID NO: 57 - DNA, 956 bp, Homo sapiens
  source 1..956: mol_type = genomic DNA
ctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacgtgctgcgacgctttttttctggcaagatagtcttgtaaatgcgggccaggatctgcacactggtatttcggtttttgggcccgcggccggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctccttggaatttggcctttttgagtttggatcttggttcattctcaagcctcagacagtggttcaaagtttttttcttccatttcag

SEQ ID NO: 58 - DNA, 939 bp, Homo sapiens
  source 1..939: mol_type = genomic DNA
gtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcag

SEQ ID NO: 59 - DNA, 83 bp, Homo sapiens
  source 1..83: mol_type = genomic DNA
tcagaagccccgggctcgtcagtcaaaccggttctctgtttgcactcggcagcacgggcaggcaagtggtccctaggttcggg

SEQ ID NO: 60 - DNA, 476 bp, Homo sapiens
  source 1..476: mol_type = genomic DNA
gtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 61 - DNA, 196 bp, Betapolyomavirus macacae
  source 1..196: mol_type = genomic DNA
tctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgc

SEQ ID NO: 62 - DNA, 589 bp, synthetic construct
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 63 - DNA, 588 bp, synthetic construct
  source 1..588: mol_type = other DNA
tcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgca

SEQ ID NO: 64 - DNA, 755 bp, synthetic construct
  source 1..755: mol_type = other DNA
ttcctgttaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggac

SEQ ID NO: 65 - DNA, 12 bp, synthetic construct
  source 1..12: mol_type = other DNA
tgtgtgataatg

SEQ ID NO: 66 - DNA, 810 bp, Homo sapiens
  source 1..810: mol_type = genomic DNA
ctgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagtta

SEQ ID NO: 67 - DNA, 726 bp, Hepatitis B virus
  source 1..726: mol_type = genomic DNA
ataacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcaaacctcatcgggaccgacaattctgtcgtactctcccgcaagtatacatcgtttccatggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgtccgaccacggggcgcacctctctttacgcggactccccgtctgtgccttctcatctgccggaccgtgtgcacttcgcttcacctctgcacgtcgcatggaggccaccgtgaacgcccaccggaacctgcccaaggtcttgcataagaggactcttggactttcagcaatgtcatc

SEQ ID NO: 68 - DNA, 755 bp, synthetic construct
  source 1..755: mol_type = other DNA
ttcctgtaaacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagctggactggagcaaacctcatcgggaccgacaattctgtcgtactctcccgcaagcactcaccgtttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggac

SEQ ID NO: 69 - DNA, 94 bp, Homo sapiens
  source 1..94: mol_type = genomic DNA
gctggagcctcggtagccgttcctcctgcccgctgggcctcccaacgggccctcctcccctccttgcaccggcccttcctggtctttgaataaa

SEQ ID NO: 70 - DNA, 596 bp, Woodchuck hepatitis virus
  source 1..596: mol_type = genomic DNA
attcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagggcc

SEQ ID NO: 71 - DNA, 387 bp, Oryctolagus cuniculus
  source 1..387: mol_type = genomic DNA
tggctaataaaggaaatttattttcattgcaatagtgtgttggaattttttgtgtctctcactcggaagaacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 72 - DNA, 251 bp, Bos taurus
  source 1..251: mol_type = genomic DNA
ttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaatacaatagcaggcatgctggggatgcggtgggctctatgggtacccaggtgctgaagaattgacccggttcctcctggg

SEQ ID NO: 73 - DNA, 251 bp, Bos taurus
  source 1..251: mol_type = genomic DNA
ttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctggg

SEQ ID NO: 74 - DNA, 225 bp, Bos taurus
  source 1..225: mol_type = genomic DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 75 - DNA, 202 bp, Homo sapiens
  source 1..202: mol_type = genomic DNA
ctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 76 - protein, 735 aa, unidentified [sequence omitted]

SEQ ID NO: 77 - protein, 736 aa, unidentified
  source 1..736: note = Adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 78 - protein, 736 aa, unidentified
  source 1..736: note = Adeno-associated virus 6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 79 - protein, 738 aa, unidentified
  source 1..738: note = Non-human primate adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 80 - DNA, 2217 bp, unidentified
  source 1..2217: mol_type = genomic DNA; note = Non-human primate adeno-associated virus
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaagacggctcctggaaagaagagaccggtagagccatcaccccagcgctctccagactcctctacgggcatcggcaagaaaggccagcagcccgcaaaaaagagactcaattttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaagcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaagaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactcaaagcacgggcggtactgcaggaactcagcagttgctattttctcaggccgggcctaacaacatgtcggctcaggccaagaactggctacccggtccctgctaccggcagcaacgcgtctccacgacactgtcgcagaacaacaacagcaactttgcctggacgggtgccaccaagtatcatctgaatggcagagactctctggtgaatcctggcgttgccatggctacccacaaggacgacgaagagcgattttttccatccagcggagtcttaatgtttgggaaacagggagctggaaaagacaacgtggactatagcagcgtgatgctaaccagcgaggaagaaataaagaccaccaacccagtggccacagaacagtacggcgtggtggccgataacctgcaacagcaaaacgccgctcctattgtaggggccgtcaatagtcaaggagccttacctggcatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgcggatcctccgaccaccttcaatcaggccaagctggcttctttcatcacgcagtacagtaccggccaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctgtaa

SEQ ID NO: 81 - protein, 738 aa, unidentified
  source 1..738: note = Non-human primate adeno-associated virus
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 82 - protein, 601 aa, unidentified
  source 1..601: note = Non-human primate adeno-associated virus
TAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 83 - protein, 535 aa, unidentified
  source 1..535: note = Non-human primate adeno-associated virus
MAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 84 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 85 - protein, 7 aa, synthetic construct
TLAVPFK

SEQ ID NO: 86 - protein, 7 aa, synthetic construct
KFPVALT

SEQ ID NO: 87 - DNA, 2514 bp, synthetic construct
  source 1..2514: mol_type = other DNA
atggctgcacctggtgcgcctgccgaatatggttatataaggacagttttgggtcaacagatacttggacaacttgattcctcttctttggcgctcccttcagaagcaaagttgaaattggcaggttcatcaggtagaggaggacaaactgtgaaatctctgcggattcaggaacaagtacaacagactttggctagaaagggaagatcttccgttggcaatggaaatttgcatagaacgagcagcgtacctgaatacgtatacaatctccatcttgttgaaaacgattttgtaggaggtcggtctccagttccgaaaacatatgatatgcttaaagcaggtactacagctacatacgaaggtagatggggaagaggaactgcacaatattcatctcaaaaatctgttgaagaaaggtcattgcgacatcctctcagaaggctcgaaatttccccggattcatcacctgaaagggctcattatacacacagtgattatcaatatagccaacgatctcaagcaggtcatacacttcatcatcaggaaagtaggcgggcggcattgcttgtaccccctcgatatgccaggtcagaaattgtaggagtttccagagcaggtactactagtcgacaaagacactttgacacatatcataggcaatatcaacacggtagtgtctcagacactgtttttgattcaataccagcaaatccggcattgctgacgtatcctcgacctggtacgtcaaggagcatgggaaatcttctggagaaagaaaattatttgactgctgggcttacagttggacaggtacggccattggttcctcttcaaccagttactcagaatcgagcttccagaagctcttggcatcaatcaagctttcattccacaaggacactgagagaagcaggaccttctgttgccgttgattccagtgggcggagagcccaccttacagtaggtcaagcagctgccggaggatcaggtaatttgcttacagaaaggagtacttttacagatagccaactcgggaatgcggatatggaaatgacattggagagagctgtatcaatgctggaagcggatcatatgcttccttctagaatttccgctgcggcaacttttatacagcacgaatgttttcaaaaatccgaagcgcgaaagcgggttaatcagctgagaggtattctgaaactgttgcaacttttgaaagtgcaaaatgaagatgttcaaagagctgtttgtggagctcttagaaatcttgtttttgaagataatgataataaattggaagttgctgaacttaatggtgtacctagattgcttcaagttttgaaacaaacaagagatttggaaacaaaaaaacaaataacaggacttttgtggaatctttcttctaatgataaacttaaaaatttgatgataacagaagctttgttgacacttacagaaaatattattatacctttttctggatggcctgaaggggattatccaaaagctaatgggttgctggattttgatattttttataatgtgacaggatgtttgaggaatatgtcctcagctggtgctgacggtcggaaagcaatgaggcgatgtgacggtttgatcgattcccttgttcattacgttaggggaacaatagctgattatcaaccagatgataaagcaacagaaaattgtgtttgtattcttcataatctgtcttatcaattggaagccgaacttcctgaaaaatactctcaaaatatctatattcagaatcggaacatacaaactgataataacaaatctataggatgttttggatcaagaagcagaaaagttaaggaacaatatcaagacgttcctatgcccgaagaaaaatcaaatccaaagggtgtagaatggctgtggcattctattgttattagaatgtatttgtcactcatagcaaaatctgtacgaaattatacacaagaagcttctttgggagcccttcagaatcttacagcaggaagtggtcctatgcctacaagtgtagcgcaaacagttgtacagaaagaatctggattgcaacatactagaaagatgctgcatgtaggagatccttctgttaaaaagacagctataagcctcctgaggaacttgagtcgcaacttgtctctccagaatgagattgctaaggaaacgcttcctgaccttgtatctattatacctgacacggtgccttccacagatcttttgattgaaacaactgcatctgcttgttatacattgaataatatcattcaaaatagttatcagaacgcaagagatcttctgaatacaggaggtattcaaaaaataatggctatttcagcaggtgatgcatatgcttctaataaagcgtctaaagctgcatctgttcttttgtattctctttgggcacatacagaacttcaccatgcctacaagaaagctcaattcaagaaaacagattttgttaatagtcgcactgcaaaagcgtatcattcccttaaggattaa

SEQ ID NO: 88 - DNA, 2646 bp, synthetic construct
  source 1..2646: mol_type = other DNA
atggccgcacctggagctccagctgaatatggatatatcaggactgtcttgggtcagcagattctgggccaactggattcttcaagtctggccctcccgagtgaggcaaaactgaaactcgcaggatctagtggaaggggcggacagacagtgaagtcactcaggattcaggagcaggtccaacagacactggcaagaaaagggaggtccagcgtcggcaatggtaacttgcatagaacctcttcagtgcctgagtacgtgtacaacctgcacctggtggaaaacgattttgtgggcggcagatccccggtccctaaaacttatgacatgctgaaagctggcacgacagctacatatgaaggtagatggggccggggcaccgcacaatatagttctcagaagtccgtggaagaaagatctcttaggcacccacttcggcgactggagataagtccagacagcagccccgaaagggcgcattatacacattctgactaccaatacagccaaagatctcaggctggccacactctgcatcatcaggaaagtagaagggccgcccttctcgtccccccaagatacgcacggtcagaaatcgtgggagtgagcagagcaggtaccacctctcgacagaggcatttcgacacataccaccgacaataccaacatggatcagtgtcagatactgtttttgactccataccagcaaatccagcactcctgacatatcctagaccggggactagtcgctccatggggaatctgctcgaaaaagaaaactatctcaccgcaggtctgactgtggggcaggtaaggccgttggtgccccttcagcctgtaacgcagaatagggcttccaggtcatcctggcaccagagtagcttccactccacaaggactctgagagaggcaggccctagtgttgcagttgatagttccggtcgcagggcccatcttacagttggtcaagccgctgcagggggtagcggtaatctgctgaccgaaagaagcacattcacggattcccagctcggaaatgcagacatggagatgacacttgaacgagccgtgagcatgctggaagctgaccatatgctcccttctagaatctccgctgcggccactttcatccagcacgagtgctttcagaaatccgaagctaggaaacgcgtgaaccagctgaggggcatcctgaagctgctgcagctgcttaaggtgcagaatgaggatgtccaacgcgcagtgtgcggtgctctgaggaacctcgtgtttgaagataacgataataagttggaagtcgcagaacttaacggggtacctcgactgctccaggtgttgaaacagacacgggacctggaaacgaaaaaacaaattaccgaccacactgtgaatctcagaagtcggaatgggtggcctggggcagtagctcatgcctgtaacccttctacattgggtggccaaggcgggaggattaccagatcaggggtacgggatcaacctgatcagcacgggctcctgtggaacttgtcctctaatgacaaactgaaaaacctgatgataaccgaggctctgctgaccctgacagaaaacattatcatccccttctccggatggccagaaggagactatcctaaagctaacggcttgctggacttcgatatcttttataatgtcacagggtgccttagaaatatgagtagcgccggtgccgatggaaggaaggcaatgcgccggtgtgatggtcttatcgattccctggtacattacgtgaggggcaccattgccgactaccaaccagacgataaagcaaccgagaattgcgtgtgcatcctgcacaatttgagctaccaactcgaagctgaactgcctgagaaatattcccaaaatatttatatccagaatcggaatatccagaccgataataacaagagcataggatgctttggttctagaagccgcaaggtcaaggaacagtaccaggatgtacctatgccagaggaaaaatcaaaccccaaaggagtggaatggctgtggcattccatcgtgatcaggatgtacctgagtctgattgctaagagcgtgaggaactatacacaggaggcttcccttggcgcactgcaaaatctgaccgccggatctgggccaatgccaacaagcgttgctcaaacagtagtgcaaaaggaatctggccttcagcacactaggaaaatgcttcatgtgggagatccttctgtgaagaagacggctattagcctgctccgcaacttgagcagaaacctttctttgcagaatgagattgcgaaagagacgctccctgacctggtgtccattataccagacacggtcccttccacagatcttctgattgaaacgaccgcttccgcatgttacaccctgaataatattattcaaaactcataccaaaatgctagagacctgctgaacactggggggatccagaagattatggcaatttcagccggcgatgcctatgctagtaataaagcctccaaggccgccagtgtattgctgtatagtctgtgggcacatacagaactgcaccacgcatataagaaggcccagtttaaaaaaaccgatttcgtaaactctcggactgcaaaagcctaccattcactgaaagattga

SEQ ID NO: 89 - DNA, 4650 bp, synthetic construct
  source 1..4650: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgtacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcgccaccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacaggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 90 - DNA, 4274 bp, synthetic construct
  source 1..4274: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgtctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcaggccaccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacaggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 91 - DNA, 4782 bp, synthetic construct
  source 1..4782: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgtacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcgccaccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacagaccatacagtcaatttaagaagtaggaatggctggccgggcgcggtggctcacgcctgtaatcccagcactttgggaggccaaggcgggcggatcacgaggtcaggagttcgagaccagcctgaccaacatggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 92 - DNA, 4406 bp, synthetic construct
  source 1..4406: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgtctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcaggccaccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacagaccatacagtcaatttaagaagtaggaatggctggccgggcgcggtggctcacgcctgtaatcccagcactttgggaggccaaggcgggcggatcacgaggtcaggagttcgagaccagcctgaccaacatggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 93 - DNA, 4237 bp, synthetic construct
  source 1..4237: mol_type = other DNA
acccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcgccaccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacaggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 94 - DNA, 3861 bp, synthetic construct
  source 1..3861: mol_type = other DNA
ctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcaggccaccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacaggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 95 - DNA, 4369 bp, synthetic construct
  source 1..4369: mol_type = other DNA
acccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcgccaccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacagaccatacagtcaatttaagaagtaggaatggctggccgggcgcggtggctcacgcctgtaatcccagcactttgggaggccaaggcgggcggatcacgaggtcaggagttcgagaccagcctgaccaacatggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 96 - DNA, 3993 bp, synthetic construct
  source 1..3993: mol_type = other DNA
ctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcaggccaccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacagaccatacagtcaatttaagaagtaggaatggctggccgggcgcggtggctcacgcctgtaatcccagcactttgggaggccaaggcgggcggatcacgaggtcaggagttcgagaccagcctgaccaacatggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 97 - DNA, 4274 bp, synthetic construct; identical to SEQ ID NO: 90
  source 1..4274: mol_type = other DNA

SEQ ID NO: 98 - DNA, 4633 bp, synthetic construct
  source 1..4633: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgtacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccaggccaccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacagaccatacagtcaatttaagaagtaggaatggctggccgggcgcggtggctcacgcctgtaatcccagcactttgggaggccaaggcgggcggatcacgaggtcaggagttcgagaccagcctgaccaacatggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 99 - DNA, 4406 bp, synthetic construct; identical to SEQ ID NO: 92
  source 1..4406: mol_type = other DNA

SEQ ID NO: 100 - DNA, 3861 bp, synthetic construct; identical to SEQ ID NO: 94
  source 1..3861: mol_type = other DNA

SEQ ID NO: 101 - DNA, 4220 bp, synthetic construct
  source 1..4220: mol_type = other DNA
acccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccaggccaccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacagaccatacagtcaatttaagaagtaggaatggctggccgggcgcggtggctcacgcctgtaatcccagcactttgggaggccaaggcgggcggatcacgaggtcaggagttcgagaccagcctgaccaacatggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 102 - DNA, 3993 bp, synthetic construct; identical to SEQ ID NO: 96
  source 1..3993: mol_type = other DNA